(19) United States
(12) Patent Application Publication
(10) Pub. No.: US 2025/0257100 A1
(43) Pub. Date: Aug. 14, 2025

(54) COMPOSITIONS AND METHODS FOR MUCOSAL VACCINATION AGAINST SARS-COV-2

(71) Applicant: University of Maryland, College Park

(72) Inventors: Xiaoping Zhu, Clarksville, MD (US); Weizhong Li, College Park, MD (US); Tao Wang, Ellicott City, MD (US)

(21) Appl. No.: 19/053,111

(22) Filed: Feb. 13, 2025

Related U.S. Application Data
(60) Provisional application No. 63/553,097, filed on Feb. 13, 2024.

(51) Int. Cl. C07K 14/005; A61K 39/00; A61P 31/14

(52) U.S. Cl. CPC C07K 14/005; A61P 31/14; A61K 39/00; A61K 2039/543; A61K 2039/55561; C07K 2319/02; C07K 2319/30; C07K 2319/70; C12N 2770/20022; C12N 2770/20034

(57) ABSTRACT

Disclosed are peptides comprising a monomeric Fc fragment of an immunoglobulin recognized by a FcRn; SARS-CoV-2 antigen; and a trimerization domain. Disclosed are peptide complexes comprising three peptides, wherein each of the three peptides comprises a monomeric Fc fragment of an immunoglobulin recognized by a FcRn; SARS-CoV-2 antigen; and a trimerization domain. Disclosed are compositions comprising any of the disclosed peptides or peptide complexes. Disclosed are methods for eliciting a protective immune response against SARS-CoV-2 comprising administering to a subject an effective amount of one or more of the compositions disclosed herein. Disclosed are methods of treating a subject exposed to SARS-CoV-2 or at risk of being exposed to SARS-CoV-2 comprising administering to a subject an effective amount of one or more of the compositions disclosed herein.

### CROSS-REFERENCE TO RELATED APPLICATIONS

This application claims the benefit of U.S. Provisional Patent Application No. 63/553,097, filed Feb. 13, 2024, which is incorporated by reference herein in its entirety.

### STATEMENT REGARDING FEDERALLY SPONSORED RESEARCH

This invention was made with government support under R01AI146063 awarded by the National Institutes of Health. The government has certain rights in the invention.

### REFERENCE TO SEQUENCE LISTING

The Sequence Listing submitted Feb. 13, 2025 as a text file named “36429.0034U2.xml,” created on Feb. 13, 2025, and having a size of 88,993 bytes is hereby incorporated by reference pursuant to 37 C.F.R. § 1.52(e)(5).

### BACKGROUND

COVID-19, the disease caused by the virus SARS-CoV-2, is extremely infectious and sustainable in the community. The virus spreads mainly through respiratory droplets, possibly aerosol, produced when an infected person coughs or sneezes. These droplets or aerosols can land in the mouths or noses of nearby people or possibly be inhaled into the lungs. The highly contagious nature is probably due to the virus spreading via asymptomatic patients. Although most patients are not severe, the virus can cause acute, highly lethal pneumonia with a 2-10 day incubation period in the elderly or people with underlying medical conditions. Although children infected with SARS-CoV-2 have fewer symptoms, they can spread the virus easily to others. The SARS-CoV-2 virus infects respiratory epithelial cells through its Spike (S) binding to angiotensin-converting enzyme 2 (ACE2) receptor. Using Spike (S) protein, the SARS-CoV-2 virus binds to ACE2 receptors in nasal, bronchial, alveolar, and other epithelial cells. Host proteases cleave the S protein into S1 and S2 subunits during infection. S1 mainly contains the receptor-binding domain (RBD) which allows viruses to bind to the ACE2 directly, S2 likely mediates membrane fusion with the help of a protease TMPRSS2 in cells.

The neonatal Fc Receptor (FcRn) plays a crucial role in transporting IgG antibodies across the polarized epithelial cells lining the respiratory, intestinal, genital tract, and placenta. FcRn expresses in the cell surface or resides within low-pH endosomes. Normally, IgG enters cells via pinocytotic vesicles that fuse with endosomes. IgG which binds to FcRn is transported to the basolateral surface and released into the submucosa. It has been shown that FcRn in dendritic cells (DCs) and macrophages enhances antigen presentation to CD4 T helper or cross-presentation to CD8 T cells. FcRn in all mammals are structurally and functionally similar.

Presently, most vaccines against respiratory infections are designed for delivery via the muscle or skin but are intended to protect the lungs. Parenteral delivery elicits relatively poor immunity in the respiratory tract even though they often induce robust systemic immunity. A partial reason is that parenteral immunization fails to induce strong mucosal antibody and cell-mediated immunity including T and B cells that reside in the lung. Since SARS-CoV-2 viruses infect the upper or lower respiratory tract and asymptomatic infections frequently occur, the development of a safe and effective mucosal vaccine to prevent the infection and possibly reinfection in the long term is urgently needed. Ideally, a mucosal vaccine mimics the route of natural viral exposure and engenders beneficial nasal and lung immunity. This goal can be best achieved by direct delivery of the SARS-CoV-2 vaccine antigen via the intranasal route.

### BRIEF SUMMARY

Disclosed are peptides comprising a monomeric Fc fragment of an immunoglobulin recognized by a neonatal receptor (FcRn); a SARS-CoV-2 spike (S) antigen, wherein the SARS-CoV-2 S antigen comprises at least three mutations, wherein the mutations stabilize the S antigen in the prefusion form; and a trimerization domain.

Disclosed are peptides comprising a monomeric Fc fragment of an immunoglobulin recognized by a neonatal receptor (FcRn), wherein the monomeric Fc fragment of FcRn comprises at least three mutations in cysteine residues responsible for dimer formation; a SARS-CoV-2 spike (S) antigen, wherein the SARS-CoV-2 S antigen comprises at least two mutations, wherein the mutations stabilize the S antigen in the prefusion form; and a trimerization domain.

Disclosed are peptides comprising a monomeric Fc fragment of an immunoglobulin recognized by a neonatal receptor (FcRn); a SARS-CoV-2 spike (S) antigen, wherein the SARS-CoV-2 S antigen comprises mutations that stabilize the S antigen in the prefusion form, wherein the mutations comprise F817P, A892P, A899P, and A942P; and a trimerization domain.

Disclosed are peptides comprising a monomeric Fc fragment of an immunoglobulin recognized by a neonatal receptor (FcRn); a SARS-CoV-2 spike (S) antigen, wherein the SARS-CoV-2 S antigen comprises mutations that stabilize the S antigen in the prefusion form, wherein the mutations comprise F817P, A892P, A899P, A942P, K986P, and V987P; and a trimerization domain.

Disclosed are peptide complexes comprising three peptides, wherein each of the peptides is one of more of the peptides described herein.

Disclosed are compositions comprising one or more of the peptides disclosed herein.

Disclosed are compositions comprising one or more of the peptide complexes disclosed herein.

Disclosed are methods for eliciting a protective immune response against SARS-CoV-2 comprising administering to a subject an effective amount of one or more of the peptides or one or more of the compositions disclosed herein.

Disclosed are methods for eliciting a protective immune response against SARS-CoV-2 comprising administering to a subject an effective amount of a composition comprising a peptide complex, wherein the peptide complex comprises three peptides forming a trimer, wherein each of the three peptides comprises a monomeric Fc fragment of an immunoglobulin recognized by a neonatal receptor (FcRn); a SARS-CoV-2 spike (S) antigen, wherein the SARS-CoV-2 S antigen comprises at least three mutations, wherein the mutations stabilize the S antigen in the prefusion form; and a trimerization domain, wherein the administering is to a mucosal epithelium.

Disclosed are methods of treating a subject exposed to SARS-CoV-2 or at risk of being exposed to SARS-CoV-2 comprising administering to the subject an effective amount of one or more of the peptides or one or more of the compositions disclosed herein.

Disclosed are methods of treating a subject exposed to SARS-CoV-2 or at risk of being exposed to SARS-CoV-2 comprising administering to the subject an effective amount of a composition comprising a peptide complex, wherein the peptide complex comprises three peptides forming a trimer, wherein each of the three peptides comprises a monomeric Fc fragment of an immunoglobulin recognized by a neonatal receptor (FcRn); a SARS-CoV-2 spike (S) antigen, wherein the SARS-CoV-2 S antigen comprises at least three mutations, wherein the mutations stabilize the S antigen in the prefusion form; and a trimerization domain, wherein the administering is to a mucosal epithelium.

Described herein are compositions and methods for using the FcRn to deliver SARS-CoV-2 spike antigens to induce protective immunity against SARS-CoV-2 virus infection, wherein the SARS-CoV-2 spike antigen is from an Omicron or delta strain or wherein the SARS-CoV-2 virus infection is an SARS-CoV-2 omicron or delta strain infection.

Disclosed are peptides comprising a monomeric Fc fragment of an immunoglobulin recognized by an FcRn; SARS-CoV-2 antigen; and a trimerization domain. In some aspects, the SARS-CoV-2 antigen can be a SARS-CoV-2 spike protein. Thus, disclosed are peptides comprising a monomeric Fc fragment of an immunoglobulin recognized by an FcRn; SARS-CoV-2 spike protein; and a trimerization domain.

Disclosed are peptide complexes comprising three of the disclosed peptides. For example, disclosed are peptide complexes comprising three peptides, wherein each of the three peptides comprises a monomeric Fc fragment of an immunoglobulin recognized by an FcRn; SARS-CoV-2 antigen; and a trimerization domain.

Disclosed are nucleic acid sequences capable of encoding any of the peptides disclosed herein.

Disclosed are compositions comprising any of the disclosed peptides, peptide complexes, nucleic acid sequences, or vectors. In some instances, disclosed are compositions comprising a monomeric Fc fragment of an immunoglobulin recognized by an FcRn; a SARS-CoV-2 antigen; and a trimerization domain, wherein the SARS-CoV-2 spike antigen is from an omicron or delta strain.

Disclosed are methods for eliciting a protective immune response against SARS-CoV-2 comprising administering to a subject an effective amount of a composition comprising a monomeric Fc fragment of an immunoglobulin recognized by an FcRn; a SARS-CoV-2 antigen; and a trimerization domain, wherein the administering is to a mucosal epithelium, wherein the SARS-CoV-2 an Omicron or delta strain.

Disclosed are methods of treating a subject exposed to SARS-CoV-2 or at risk of being exposed to SARS-CoV-2 comprising administering to a subject an effective amount of a composition comprising a monomeric Fc fragment of an immunoglobulin recognized by a FcRn; a SARS-CoV-2 antigen; and a trimerization domain, wherein the administering is to a mucosal epithelium, wherein the SARS-CoV-2 is a Omicron or delta strain.

Disclosed are methods of reducing SARS-CoV-2 viral titers in a subject infected with SARS-CoV-2 comprising administering to a subject an effective amount of a composition comprising a monomeric Fc fragment of an immunoglobulin recognized by an FcRn; a SARS-CoV-2 antigen; and a trimerization domain, wherein the administering is to a mucosal epithelium, wherein the SARS-CoV-2 spike antigen is from an Omicron or delta strain or wherein the SARS-CoV-2 is a SARS-CoV-2 omicron or delta strain.

Additional advantages of the disclosed method and compositions will be set forth in part in the description which follows, and in part will be understood from the description, or may be learned by practice of the disclosed method and compositions. The advantages of the disclosed method and compositions will be realized and attained by means of the elements and combinations particularly pointed out in the appended claims. It is to be understood that both the foregoing general description and the following detailed description are exemplary and explanatory only and are not restrictive of the invention as claimed.

### BRIEF DESCRIPTION OF THE DRAWINGS

The accompanying drawings, which are incorporated in and constitute a part of this specification, illustrate several embodiments of the disclosed method and compositions and together with the description, serve to explain the principles of the disclosed method and compositions.

FIG. 1 shows a schematic representing a proposed model of FcRn-mediated transfer of SARS-CoV-2 vaccine antigens across a respiratory epithelial barrier and target to mucosal antigen-presenting cells (APCs) (e.g. dendritic cells) and B cells.

FIG. 2 shows a schematic illustration of the fusion of S, S1, RBD, the foldon, and Fcγ cDNA to create a trimeric S-Fc fusion gene. S, Spike; SP, signal peptide; RBD, receptor binding domain; FP, fusion peptide; TM, transmembrane domain. Fd, Foldon domain; R685A, cleavage site; R815A, mutation at S2′ cleavage site; K986P/V987P, mutation keeping pre-fusion structure; C226S/C229S, mutation for a monomer hIgG1; K322A, mutation.

FIG. 3 shows a protein gel demonstrating the production of SARS-CoV-2 S, S-Fc, S1-Fc, and RBD-Fc fusion proteins. CHO or 293T cells were transfected with plasmids encoding S, S-Fc/wt, S1-Fc/wt, or RBD-Fc/wt. The stable cell lines were selected and cloned. The proteins in supernatants were purified with anti-His beads for S antigen or Protein A/G-agarose beads. The purified proteins were detected by Coomassie blue.

FIG. 4 shows that intranasal immunization of mice with S-Fc, S1-Fc, or RBD-Fc induced S-specific antibody immune responses. Top panel: Intranasal delivery of both S1-Fc or RBD-Fc antigens induces Spike-specific antibody immune responses. Five g of purified spike S1-Fc, RBD-Fc, or PBS in combination with 10 μg of CpG were intranasally (i.n.) administered to mice (n=5). Spike-specific antibody titers in sera were measured 14 days after boost by ELISA. The data represent mean±S.E.M. Bottom panel: SARS-CoV-2 neutralization by serum antibodies. Neutralization assays were performed by incubating SARS-CoV-2 pseudoviruses (50 ul) with 1:10 dilution of the pooled mouse sera at 37° C. for 1 hr. After incubation, the 100 μL of the sera-pseudovirus mixture was added to ACE2/293T cells. After 72 hr incubation, luciferase activity was measured using luciferin-containing substrate. Controls included cell-only control, and virus without any antibody control. The PBS immunized mice serum was a negative control. The average percentage inhibition (at 1:10 serum dilution) for each group is shown. Data is shown for 5 mice per group.

FIG. 5 shows the immune response is FcRn-dependent. Top. S-specific IgG titers in sera were measured by ELISA 14 days after boosting. Bottom. The neutralization antibody titers in the sera were expressed as the reciprocal of the twofold serial dilution preventing the appearance of the cytopathogenic effect (CPE) in Vero E6 cells. KO: FcRn knockout mice

FIG. 6 shows the mean survival following viral challenge. Two weeks after the boost, groups of 5 mice were i.n. challenged with SARS-CoV-2 virus and weighed daily for 14 days. Mice were humanely euthanized if above 25% of initial body weight was lost. The percentage of mice from protection after the challenge was shown by the Kaplan-Meier survival curve.

FIG. 7 shows the mean of viral titers following viral challenge. The virus titers in the different organs of the mice (n=3) were determined 5 days after challenge. Supernatants of the tissue homogenates were added onto Vero E6 and incubated for three days. The viral titers were measured by 50% reduction of CPE.

FIG. 8 shows an example of the histopathology of the lungs from the infected or normal mice. Lungs were collected from day 5 post-challenge. The sections were stained with H & E to determine the level of inflammation (10×). The representative slides were shown.

FIG. 9 shows a schematic illustration of the fusion of S, S1, RBD, the foldon, and Fcγ cDNA to create a trimeric S-Fc fusion gene and further includes an RBD-Fc fragment fusion without a trimerization domain. S, Spike; SP, signal peptide; RBD, receptor binding domain; FP, fusion peptide; TM, transmembrane domain. Fd; Foldon domain, cleavage site; R816A, mutation at S2′ cleavage site.

FIG. 10 shows a schematic illustration of an FcRn-mediated delivery of SARS-CoV-2 vaccine antigens.

FIGS. 11A-11F shows data using a SARS-CoV-2 Spike peptide fused with Fc. FIG. 11A shows a schematic illustration of the full-length protein sequence of SARS-CoV-2 Spike. SP: signal peptide; RBD: receptor binding domain; TM: transmembrane domain; CT: cytoplasmic tail. FIG. 11A shows a graphic design of the fusion of SARS-CoV-2 Spike with the T4 fibritin foldon domain (Fd) to create a soluble, prefusion-stabilized, and trimeric Spike protein. Mutations were made in the SARS-CoV-2 (strain USA/WA1/2020) spike by replacing Arg 685 and Arg 815, respectively, with an Ala residue to remove the cleavage site and replacing Lys 986 and Val 987, respectively, with a Pro residue to create a prefusion-stabilized form. The diagram of FIG. 11A demonstrates the fusion of a SARS-CoV-2 Spike with the T4 fibritin foldon domain and human Fcγ1 to create a prefusion-stabilized and trimeric S-Fc fusion protein. Mutations were also made in the Fcγ1 fragment by replacing Cys 226 and Cys 229, respectively, with a Ser residue to abolish Fc dimerization, and replacing Glu 318 Lys 320 Lys 322 with Ala residues to remove the complement C1q binding site. FIG. 11B shows the S and S-Fc fusion proteins were purified from the stable CHO cell lines. The soluble S and S-Fc proteins were purified by anti-His and Protein A affinity chromatography, respectively, subjected to SDS-PAGE gel electrophoresis under reducing conditions and visualized with Coomassie blue staining. FIG. 11C-F shows the results of a test of the S-Fc binding to human, mouse, or hamster FcRn/β2m; human, mouse, or hamster FcγRI; human ACE2; and human or mouse C1q. The purified S protein was used as a positive control for ACE2 binding and a negative control for FcRn/β2m or FcγRI binding. Respiratory syncytial virus (RSV) protein F alone or Fc-fused F proteins were used as a negative control. Mouse IgG, human IgG1, hamster IgG2, and a mAb (D25) against RSV F protein were used as the positive control, respectively.

FIGS. 12A-12I shows FcRn-mediated respiratory immunization induces SARS-CoV-2 S-specific antibody immune responses. FIG. 12A shows a design where ten g of S-Fc, S (with the equivalent molar number), or PBS in combination with 10 μg of CpG was i.n. administered into 6-8-week-old wild-type (WT) C57BL/6, or FcRn knockout (KO) mice. FIG. 12A shows where mice were boosted 14 days after the primary immunization. Sampling was performed at the indicated time points. FIG. 12B shows anti-S-specific IgG Ab levels in sera.

FIG. 12C shows the results of a test of neutralizing Ab activity in sera of the immunized animals. FIG. 12D-G shows anti-S-specific Ab titers in nasal washings (FIG. 12D, IgG; f, IgA), and BAL (FIG. 12E, IgG; g, IgA) of mice (n=13 for each group). The data represent a geometric mean with 95% CI. The statistical analyses were done by one-way ANOVA (Kruskal-Wallis test followed by Dunn's multiple comparisons tests) (FIG. 12B-B), and ordinary one-way ANOVA with Dunnett's multiple comparison tests (FIG. 12I). The horizontal dashed lines indicate the limit of detection (LOD).

FIGS. 13A-13H shows the intranasal immunization by the S-Fc induces protective immunity to intranasal (i.n.) challenge with ancestral SARS-CoV-2. FIG. 13A shows ten g of S-Fc or PBS with 10 μg CpG was i.n. administered into 8-week-old hACE-2 mice (n=16 per group). FIG. 13B, C show serum anti-S-specific IgG Ab titers (B) and neutralizing Ab (C) in hACE2 mice (n=16 per group). The S-specific IgG Ab titers were measured by coating ELISA plates with S protein; the micro-neutralization test determined the neutralizing Ab activity in the immunized sera. The data represent a geometric mean with 95% CI. FIG. 13D shows body weight changes following the challenge. The data represent the mean±SD. FIG. 13E shows survival following virus challenge (n=10 per group). The percentage of mice protected was shown by the Kaplan-Meier survival curve. FIG. 13F shows viral titers in the nasal turbinate, lung, and brain at 5 dpi. The data represent a geometric mean with 95% CI. FIG. 13G shows histopathology of the lungs from the challenged mice (n=6 per group). Lungs were collected at 5 dpi. The lungs from uninfected mice were included as standard control (n=6). The lung sections were stained with Hematoxylin-Eosin (H & E). The representative slides are shown. All scale bars represent 200 μm. 13H. The inflammatory responses of each lung section (n=6 per group) were scored blindly and shown as geometric mean with 95% CI. The statistical analyses were performed by Mann-Whitney test (two-tailed) (FIG. 13B, C, F, H) and Log-rank (Mantel-Cox) test (13E). Dash lines indicate LOD or humane endpoint (13D).

FIGS. 14A-14J shows intranasal immunization by the S-Fc induces protective immunity to intranasal challenge with SARS-CoV-2 Delta or Omicron variants. FIG. 14A shows the 8-week-old hACE-2 mice (n=10-11) were i.n. immunized by 10 μg S-Fc or PBS with 10 μg CpG and boosted 14 days later. FIG. 14B shows seventeen days after the boost, S-Fc (n=11) and PBS (n=10) groups were i.n. challenged with Delta strain (2.5×10⁴ TCID50). Six mice in the S-Fc group and 5 mice in the PBS group were subjected to the body weight loss and survival analysis by the Kaplan-Meier survival curve (14C). The remaining five mice in each group were euthanized at 6 dpi for sampling the nasal turbinate, lung, and brain and measuring the Delta virus titers by TCID50 (14D). FIG. 14E shows lungs (n=5 per group) were collected at 6 dpi. The lung sections were stained with Hematoxylin-Eosin (H & E). A representative lung image from 5 lungs was shown. FIG. 14F shows the 8-week-old hACE-2 mice were i.n. immunized by g S-Fc (n=10) or PBS (n=10) with 10 μg CpG and boosted 14 and 28 days later, respectively. FIG. 14H shows groups of 10 mice were weighed daily for 4 or 6 days after the challenge. FIG. 14I, J show the Omicron B.1.1.529 virus titers were determined at 4 or 6 dpi in the nasal turbinate, lung, and brain of the S-Fc immunized and control mice (n=5). Supernatants of the tissue homogenates were added to VAT cells and incubated for 4 days; the viral titers were shown as TCID50. The body weight data in (14B) and (14H) were shown as mean±SD, while the data in 14D, G, I, and j represent a geometric mean with 95% CI. The Mann-Whitney test (two-tailed) was used for the statistical assays (14D, G, I, J), while the Log-rank (Mantel-Cox) test was used for the survival analysis (14C). Dashed lines represent LOD.

FIGS. 15A-15G shows that FcRn-mediated intranasal vaccination significantly induces S-specific local immune responses in the respiratory tract. FIG. 15A shows ten g of S-Fc or PBS in combination with 10 μg of CpG was i.n. administered into 6-8-week-old mice (n=10). FIG. 15B-D shows anti-SARS-CoV-2 S-specific IgA or IgG Ab titers in nasal washings (15B), BAL (15C), and serum (15D) after the boost. S-specific Abs was measured by ELISA 14 days after the boost in 10 representative mouse samples per group. The data represent a geometric mean with 95% CI. FIG. 15E-G shows tissue-resident memory (TRM) T cells in mouse lungs. The unpaired t-test (two-tailed) was used for the statistical analysis of data from experiments (15b) (Nasal IgG) and (15C). The Mann-Whitney test (two-tailed) was used for experiments (15b) (Nasal IgA) and (15D). One-way ANOVA (Kruskal-Wallis test followed by Dunn's multiple comparisons tests) was used for analyzing the results of experiments (15F), while the Welch's ANOVA test (with Dunnett's T3 multiple comparisons test) was used for the experiment (15G). Dashed lines denote LOD. Source data are provided as a Source data file.

FIGS. 16A-16F show intranasal immunizations with the S-Fc protein reduce virus transmission from vaccinated to unvaccinated hamsters or vice versa. FIG. 16A-D shows groups of male hamsters (n=6) were i.n. or i.m. immunized with 30 μg of S-Fc and 30 μg of CpG twice in a 2-week interval. The unimmunized hamsters were used as a negative control. Anti-ancestral SARS-CoV-2 strain S-specific IgA, IgG Ab, and neutralizing Ab titers in sera and nasal washings were measured by ELISA 14 days after the boost. The data represent a geometric mean with 95% CI. The statistical analyses in 16A-D were conducted by one-way ANOVA (Kruskal-Wallis test followed by Dunn's multiple comparisons tests). Dashed lines indicate LOD. FIG. 16E shows a schematic illustration of the experimental design. FIG. 16F shows virus loads in the throat swabs from the donor and recipient hamsters. The infectious live virus in the samples was measured in VAT cells for 4 days of culture. The viral titers were shown as TCID50 from each animal sample. Each open circle represents one animal, and the bar indicates the median value of each group at the indicated time points. Source data are provided as a Source data file.

FIGS. 17A-17E shows expressions of S-Fc, S, hamster FcRn/β2m, and FcγRI proteins. The S-Fc fusion protein (17A, B) or S (17C) protein purified from the stable CHO cell line was identified by Western blot. The S-Fc or S proteins were subjected to SDS-PAGE and Western blot analyses and detected by either anti-S (17A, C) or goat anti-human IgG-Fc Abs (17B). HRP-conjugated secondary Abs and the ECL method visualized the S-Fc or S proteins. The purified hamster FcRn/β2m proteins (17D) or hamster FcγRI proteins (17E) from the recombinant plasmid-transfected 293T cells were visualized by Coomassie blue staining.

FIGS. 18A-18E shows interactions of the purified S or S-Fc with S-specific antibodies. Interactions of the purified S or S-Fc with COVID-19 convalescent human serum (18A), normal human serum (18B), and a set of SARS-CoV-2 S-specific mAbs (18C, D). FIG. 18E shows anti-human IgG1 Fc-specific IgG Ab titers in mouse sera. The data represent a geometric mean with 95% CI. Statistical differences were determined by one-way ANOVA (Kruskal-Wallis test followed by Dunn's multiple comparisons test). Dashed lines indicate LOD.

FIGS. 19A-19H show anti-S-specific Ab titers in nasal washings (19A, B), and BAL (19C, D) after 14 months of the boost. ELISA measured SARS-CoV-2 S-specific Abs from mouse samples (n=6 for S-Fc/WT group, n=5 for other groups). The data represent a geometric mean with 95% CI. FIG. 19E shows SARS-CoV-2 Delta-specific nAbs in human ACE2 mice after the boost. The micro-neutralization test determined the nAb activity in the sera (n=11 for the S-Fc/WT group, n=10 for the PBS group). The data represent a geometric mean with 95% CI. FIG. 19F shows after the Delta strain challenge, the inflammatory responses of each lung section were scored blindly. FIG. 19G shows the S-Fc fusion protein derived from Omicron XBB.1.5 strain was purified from the 293F cell line. The S-Fc proteins were purified by Protein A affinity column, subjected to SDS-PAGE gel electrophoresis under reducing conditions and visualized with Coomassie blue staining. The molecular weight in kDa is marked in the left margin. FIG. 19H shows viral titers in the lungs 4 and 6 days after the challenge of Omicron XBB.1.5 (5×10⁴ TCID50). Supernatants of the lung homogenates were added to VAT cells and incubated for 4 days. The viral titers were shown as TCID50 from each lung. Statistical differences were determined by one-way ANOVA (Kruskal-Wallis test followed by Dunn's multiple comparisons tests) (19A-D) and Mann Whitney test (two-tailed) (19E, F, H). Dashed lines indicate LOD.

FIGS. 20A-20J show intranasal immunizations with the S-Fc induce protection in old mice and a protective memory immune response. FIG. 20A shows the 12-18 months-old hACE2 mice were i.n. immunized by 10 μg S-Fc or PBS with 10 μg CpG and boosted twice at a 2-week interval. FIG. 20B shows anti-S-specific IgG Ab titers in the old mouse sera (n=7 per group) were measured by ELISA 14 days after the first- and second-time boosts. 2×: one-time boost; 3×: two-times boost. FIG. 20C shows the nAb titers against ancestral SARS-CoV-2 were measured 2 weeks after the second boost in the old mouse sera (n=7). FIG. 20D shows body-weight changes following the challenge. Seventeen days after the second boost, the old mice (n=7) were i.n. challenged with ancestral SARS-CoV-2 virus (5×10³ TCID50) and weighed daily for 14 days. FIG. 20E shows the percentage of the old mice protected on the indicated days was shown by the Kaplan-Meier survival curve. FIG. 20F shows throat samples were collected daily for 6 days in each aged mouse after the challenge. The presence of live virus (TCID50) from each animal swab was measured in VAT cells after 4 days. FIG. 20G shows the 8-week-old hACE2 mice were i.n. immunized by 10 μg S-Fc or PBS with 10 μg of CpG and boosted once in a 2-week interval. Six months after the boost, mice were i.n. challenged with the ancestral SARS-CoV-2 (2.5×10⁴ TCID50). FIG. 20H shows anti-S specific IgG Ab titers in sera (n=7) two weeks (2 w) or six months (6 m) following the boost were measured by ELISA. FIG. 20I shows the nAb titers in sera from the immunized mice (n=7) were determined using the TCID50 test 2 weeks or 6 months after the boost. FIG. 20J shows viral titers (n=5) were measured in the nasal turbinate, lung, and brain at 5 dpi. The data represent a geometric mean with 95% CI for 20B, C, F, H, I, J. The statistical analyses were performed by the Unpaired T-test (two-tailed) (20B, H, I), Mann-Whitney test (two-tailed) (20C, F, J), and Log-rank (Mantel-Cox) test (20E). Dashed lines indicate LOD.

FIG. 21 shows a gating strategy for identifying TRM T cells in the lungs.

FIGS. 22A-22E shows FcRn-mediated intranasal vaccination reduces viral replications in the upper respiratory tract. FIG. 22A shows ten g of S-Fc with 10 μg of CpG was i.n. or i.m. administered into 6-8 week-old hACE2 mice (n=11 for i.n group and n=10 for i.m group). FIG. 22B-E shows samples were collected from throat swabs, nasal turbinates, and lung and brain tissues at multiple time points after the challenge, as displayed at the bottom. The live virus titers were determined by calculating TCID50 values. The data in all figs represent a geometric mean with 95% CI. One-way ANOVA (Kruskal-Wallis test followed by Dunn's multiple comparisons tests) was used for the statistical assay (B, C, D, E). Dashed lines indicate LOD

FIGS. 23A-23F shows intranasal immunizations with the S-Fc protein protect Golden Syrian hamsters from SARS-CoV-2 infection. FIG. 23 shows 30 μg of the S-Fc (n=8) or PBS (n=8) in combination with 30 μg of CpG was i.n. administered into female hamsters twice in a 2-week interval. FIG. 23B shows anti-SARS-CoV-2 S-specific IgG Ab titers in the hamster sera.

FIG. 23C shows the nAb in the immunized hamster sera. FIG. 23D shows changes in body weight after the virus challenge. The data represent mean±SD. FIG. 23E shows shedding of the SARS-CoV-2 virus in nasal wash samples of the immunized and controlled hamsters. FIG. 23F shows the viral titers in the nasal turbinate, trachea, lung, brain, and intestine at 5 dpi. The data in B, C, E, and F represent a geometric mean with 95% CI. The viral titers were shown as TCID50 from each animal swab. The statistical analyses were determined by the Mann-Whitney test (two-tailed) (23B, C, F, E at 6 dpi) and the unpaired T-test (two-tailed) (e at 2 dpi and 4 dpi). Dashed lines represent LOD or humane endpoint (D).

FIGS. 24A-24F shows a comparison of virus load and RNA levels in the throat swab. FIG. 24A shows a comparison of virus load in the throat swab of the donor hamsters from different groups after the SARS-CoV-2 challenge (n=6 for groups 2 and 4, n=12 for groups 1+3). FIG. 24B-E shows SARS-CoV-2 RNA levels in throat swabs. Each bar represents one animal. Dashed lines indicate LOD.

FIG. 25 shows transmission of Omicron XBB.1.5 subvariant in hamsters.

FIGS. 26A-26D shows the effect S-Fc i.n. immunization on pre-existing immunity. Ten hamsters were i.n. infected with the Omicron B.1.1.529 isolate (1×10³ TCID50). Two weeks later, five of the infected hamsters were i.n. immunized with 30 μg S-Fc plus 30 μg CpG, while the other five animals received only PBS and CpG (26A). Two weeks after the i.n. booster, the IgG or IgA levels in the blood, and nasal washes were measured by ELISA and shown as geometric mean with 95% CI (26B-D). The statistical analyses were performed using the Mann-Whitney test (two-tailed) (26B, C) or the unpaired T-test (two-tailed) (26D). Dash lines indicate LOD.

FIGS. 27A-27G shows the measurement of neutralizing Abs against different SARS-CoV-2 variants or Omicron subvariants. The 6-8-week-old WT C57BL/6 mice (n=10 for 27A, n=11 for 27C and 27E) were i.n. immunized by 10 μg S-Fc, or PBS in combination with 10 μg of CpG); Female hamsters were i.n. immunized with 30 μg of the S-Fc (n=10), or PBS (n=10) in combination with 30 μg of CpG (27B, D, F). FIG. 27G shows anti-SARS-CoV-2 S2-specific IgG Ab titers in mouse sera. The statistical differences in the anti-S2 IgG levels between 2× and 3× immunizations were analyzed by an Unpaired T-test with Welch's correction (two-tailed). Dashed lines indicate LOD.

FIGS. 28A-28C shows an evaluation of the passive protection by serum transfer from the immunized mice. FIG. 28A shows hACE-2 mice (n=5 or 6/group) i.n. challenged with ancestral SARS-CoV-2 (2.5×10⁴ TCID50); FIG. 28B shows daily weight for 14 days; FIG. 28C shows survival following the virus challenge and is plotted as a Kaplan-Meier curve.

FIG. 29 shows a proposed model of FcRn-targeted mucosal delivery of the S-Fc vaccine antigen.

FIG. 30 shows (top) the infection number of recipient hamsters after exposure to the infected hamsters (donor) was summarized. D: day. (bottom) Statistical differences in the infection rate among different recipient groups were determined by Fisher's exact test (one-tailed). P: positive number; T: total number. Values marked with asterisks: *, p<0.05.

FIG. 31 shows a Western Blot image showing that the SARS-CoV-2 Omicron XBB.1.5 S-Fc protein comprising mutations replacing Arg 683, Arg 685 and Arg 815, respectively, with an Ala residue to remove the cleavage site and replacing Phe 817, Ala 892, Ala 899, Ala 942, Lys 986, and Val 987, respectively, with a Pro residue to create a prefusion-stabilized form and mutations in the IgG1 Fc fragment replacing Cys 220, Cys 226, and Cys 229 can be expressed, secreted, and recognized by a spike antibody.

FIGS. 32A-32C show S-Fcs. FIG. 32A shows schematic diagrams of wild type and mutated S-Fcs. FIGS. 32B and 32C show gels of the S-Fc with two cysteine mutations or three cysteine mutations, respectively.

FIGS. 33A-33C show an in vivo mouse experiment of vaccinating with an S-Fc and challenging with SARS-CoV2. FIG. 33A shows a schematic of the experiment. FIG. 33B shows the weight change % in the experiment using BA.2.86 strain challenge. FIG. 33C shows the weight change % in the experiment using EG.5.1 strain challenge.

FIGS. 34A-34C show virus load in the throat after challenge with BA.2.86 (A), EG.5.1 (B), or virus load in the lung (C).

### DETAILED DESCRIPTION

The disclosed method and compositions may be understood more readily by reference to the following detailed description of particular embodiments and the Example included therein and to the Figures and their previous and following description.

It is to be understood that the disclosed method and compositions are not limited to specific synthetic methods, specific analytical techniques, or particular reagents unless otherwise specified, and, as such, may vary. It is also to be understood that the terminology used herein is to describe particular embodiments only and is not intended to be limiting.

Disclosed are materials, compositions, and components that can be used for, can be used in conjunction with, can be used in preparation for, or are products of the disclosed method and compositions. These and other materials are disclosed herein, and it is understood that when combinations, subsets, interactions, groups, etc. of these materials are disclosed specific reference of each various individual and collective combinations and permutation of these compounds may not be explicitly disclosed, each is specifically contemplated and described herein. For example, if a peptide is disclosed and discussed and a number of modifications that can be made to a number of molecules including the amino acids are discussed, each and every combination and permutation of peptide and the modifications that are possible are specifically contemplated unless specifically indicated to the contrary. Thus, if a class of molecules A, B, and C are disclosed as well as a class of molecules D, E, and F and an example of a combination molecule, A-D is disclosed, then even if each is not individually recited, each is individually and collectively contemplated. Thus, in this example, each of the combinations A-E, A-F, B-D, B-E, B-F, C-D, C-E, and C-F are specifically contemplated and should be considered disclosed from disclosure of A, B, and C; D, E, and F; and the example combination A-D. Likewise, any subset or combination of these is also specifically contemplated and disclosed. Thus, for example, the sub-group of A-E, B-F, and C-E are specifically contemplated and should be considered disclosed from disclosure of A, B, and C; D, E, and F; and the example combination A-D. This concept applies to all aspects of this application including, but not limited to, steps in methods of making and using the disclosed compositions. Thus, if there are a variety of additional steps that can be performed it is understood that each of these additional steps can be performed with any specific embodiment or combination of embodiments of the disclosed methods, and that each such combination is specifically contemplated and should be considered disclosed.

### A. Definitions

It is understood that the disclosed method and compositions are not limited to the particular methodology, protocols, and reagents described as these may vary. It is also to be understood that the terminology used herein is for the purpose of describing particular embodiments only, and is not intended to limit the scope of the present invention which will be limited only by the appended claims.

It must be noted that as used herein and in the appended claims, the singular forms “a”, “an”, and “the” include plural reference unless the context clearly dictates otherwise. Thus, for example, a reference to “a peptide” includes a plurality of such peptides, a reference to “the composition” is a reference to one or more compositions and equivalents thereof known to those skilled in the art, and so forth.

As used herein, the term “therapeutically effective amount” means an amount of a therapeutic, prophylactic, and/or diagnostic agent that is sufficient, when administered to a subject suffering from or susceptible to a disease, disorder, and/or condition, to treat, alleviate, ameliorate, relieve, alleviate symptoms of, prevent, delay onset of, inhibit progression of, reduce severity of, and/or reduce incidence of the disease, disorder, and/or condition.

As used herein, the term “treating” refers to partially or completely alleviating, ameliorating, relieving, delaying the onset of, inhibiting progression of, reducing the severity of, and/or reducing the incidence of one or more symptoms or features of a particular disease, disorder, and/or condition (e.g. SARS-CoV-2 infection). For example, “treating” SARS-CoV-2 may refer to inhibiting survival, growth, and/or spread of the virus. Treatment may be administered to a subject who does not exhibit signs of a disease, disorder, and/or condition and/or to a subject who exhibits only early signs of a disease, disorder, and/or condition for the purpose of decreasing the risk of developing pathology associated with the disease, disorder, and/or condition.

As used herein, “subject” refers to the target of administration, e.g. an animal. Thus the subject of the disclosed methods can be a vertebrate, such as a mammal. For example, the subject can be a human. The term does not denote a particular age or sex. Subject can be used interchangeably with “individual” or “patient”.

The term ‘peptide’ refers to a polymer of amino acids and does not refer to a specific length of the product; thus, polypeptides, oligopeptides, and proteins are included within the definition of peptide. This term also does not refer to or exclude post-expression modifications of the peptide, for example, glycosylations, acetylations, phosphorylations, and the like. Included within the definition are, for example, peptides containing one or more analogs of an amino acid (including, for example, unnatural amino acids, PNA, etc.), peptides with substituted linkages, as well as other modifications known in the art, both naturally occurring and non-naturally occurring.

The term ‘promoter’ is a nucleotide sequence which is comprised of consensus sequences which allow the binding of RNA polymerase to the DNA template in a manner such that mRNA production initiates at the normal transcription initiation site for the adjacent structural gene.

The expression ‘operably linked’ refers to a juxtaposition wherein the components so described are in a relationship permitting them to function in their intended manner. A control sequence ‘operably linked’ to a coding sequence is ligated in such a way that expression of the coding sequence is achieved under conditions compatible with the control sequences.

“Optional” or “optionally” means that the subsequently described event, circumstance, or material may or may not occur or be present, and that the description includes instances where the event, circumstance, or material occurs or is present and instances where it does not occur or is not present.

Ranges may be expressed herein as from “about” one particular value, and/or to “about” another particular value. When such a range is expressed, also specifically contemplated and considered disclosed is the range from the one particular value and/or to the other particular value unless the context specifically indicates otherwise. Similarly, when values are expressed as approximations, by use of the antecedent “about,” it will be understood that the particular value forms another, specifically contemplated embodiment that should be considered disclosed unless the context specifically indicates otherwise. It will be further understood that the endpoints of each of the ranges are significant both in relation to the other endpoint, and independently of the other endpoint unless the context specifically indicates otherwise. Finally, it should be understood that all of the individual values and sub-ranges of values contained within an explicitly disclosed range are also specifically contemplated and should be considered disclosed unless the context specifically indicates otherwise. The foregoing applies regardless of whether in particular cases some or all of these embodiments are explicitly disclosed.

As used herein, “coronavirus” refers to a group of RNA viruses of the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives. In some aspects, the coronavirus is Middle East respiratory syndrome coronavirus (MERS-CoV), Human Coronavirus (HCoV), SARS-CoV, or SARS-CoV-2.

The term “subject” refers to the target of administration, e.g. an animal. Thus, the subject of the disclosed methods can be a vertebrate, such as a mammal. For example, the subject can be a human. The term does not denote a particular age or sex. Subject can be used interchangeably with “individual” or “patient.” For example, the subject of administration can mean the recipient of the alternating electrical field.

By “prevent” is meant to minimize or decrease the chance that a subject will develop a coronavirus infection.

As used herein, the terms “administering” and “administration” refer to any method of providing a therapeutic, such as an antiviral agent or coronavirus therapeutic (e.g., a peptide or peptide complex as disclosed herein), to a subject. Such methods are well known to those skilled in the art and include, but are not limited to: oral administration, transdermal administration, administration by inhalation, nasal administration, topical administration, intravaginal administration, ophthalmic administration, intramural administration, intracerebral administration, rectal administration, sublingual administration, buccal administration, and parenteral administration, including injectable such as intravenous administration, intra-arterial administration, intramuscular administration, and subcutaneous administration. Administration can be continuous or intermittent. In various aspects, a preparation can be administered therapeutically; that is, administered to treat an existing disease or condition. In further various aspects, a preparation can be administered prophylactically; that is, administered for the prevention of a disease or condition. In an aspect, the skilled person can determine an efficacious dose, an efficacious schedule, or an efficacious route of administration so as to treat a subject. In some aspects, administering comprises exposing. Thus, in some aspects, exposing a subject to alternating electrical fields means administering alternating electrical fields to the subject.

“Optional” or “optionally” means that the subsequently described event, circumstance, or material may or may not occur or be present, and that the description includes instances where the event, circumstance, or material occurs or is present and instances where it does not occur or is not present.

Unless defined otherwise, all technical and scientific terms used herein have the same meanings as commonly understood by one of skill in the art to which the disclosed method and compositions belong. Although any methods and materials similar or equivalent to those described herein can be used in the practice or testing of the present method and compositions, the particularly useful methods, devices, and materials are as described. Publications cited herein and the material for which they are cited are hereby specifically incorporated by reference. Nothing herein is to be construed as an admission that the present invention is not entitled to antedate such disclosure by virtue of prior invention. No admission is made that any reference constitutes prior art. The discussion of references states what their authors assert, and applicants reserve the right to challenge the accuracy and pertinency of the cited documents. It will be clearly understood that, although a number of publications are referred to herein, such reference does not constitute an admission that any of these documents form part of the common general knowledge in the art.

Throughout the description and claims of this specification, the word “comprise” and variations of the word, such as “comprising” and “comprises,” means “including but not limited to,” and is not intended to exclude, for example, other additives, components, integers or steps. In particular, in methods stated as comprising one or more steps or operations it is specifically contemplated that each step comprises what is listed (unless that step includes a limiting term such as “consisting of”), meaning that each step is not intended to exclude, for example, other additives, components, integers or steps that are not listed in the step.

### B. Coronaviruses

Coronaviruses are a group of RNA viruses that cause diseases in mammals and birds. In humans and birds, they cause respiratory tract infections that can range from mild to lethal. Mild illnesses in humans include some cases of the common cold (which is also caused by other viruses, predominantly rhinoviruses), while more lethal varieties can cause SARS, MERS, and COVID-19. In cows and pigs they cause diarrhea, while in mice they cause hepatitis and encephalomyelitis.

Coronaviruses are members of the subfamily Orthocoronavirinae, in the family Coronaviridae, order Nidovirales, and realm Riboviria. They are enveloped viruses with a positive-sense single-stranded RNA genome and a nucleocapsid of helical symmetry. The genome size of coronaviruses ranges from approximately 26 to 32 kilobases, one of the largest among RNA viruses. They have characteristic club-shaped spikes that project from their surface, which in electron micrographs create an image reminiscent of the solar corona, from which their name derives.

Over the past two decades, emerging pathogenic coronaviruses capable of causing life-threatening diseases in humans and animals have been identified, namely severe acute respiratory syndrome coronavirus (SARS-CoV) and Middle Eastern respiratory syndrome coronavirus (MERS-CoV). In December 2019, the Wuhan Municipal Health Committee (Wuhan, China) identified an outbreak of viral pneumonia cases of unknown cause. Coronavirus RNA was identified in some of these patients. This novel coronavirus has been named SARS-CoV-2, and the disease caused by this virus has been named COVID-19. Currently there are approximately 50 million confirmed cases of COVID-19 and over 1.2 million deaths globally.

Individuals of all ages are at risk for infection and severe disease. However, the probability of serious COVID-19 disease is higher in people aged>60 years, those living in a nursing home or long-term care facility, and those with chronic medical conditions. The spectrum of illness can range from asymptomatic infection to severe pneumonia with acute respiratory distress syndrome (ARDS) and death. Although COVID-19 patients can present with many different symptoms the main symptoms are fever, cough or shortness of breath. The abnormalities seen in chest X-rays vary, but bilateral multi-focal opacities are the most common. The abnormalities seen in computed tomography (CT) of the chest also vary, but the most common are bilateral peripheral ground-glass opacities, with areas of consolidation developing later in the clinical course. In the early phase of the disease and in an asymptomatic presentation the imaging of both X-ray and CT can be normal. Virologic testing (i.e., using a molecular diagnostic or antigen test to detect SARS-CoV-2) is recommended by the NIH for diagnosing SARS-CoV-2 in patients with suspected COVID-19 symptoms.

COVID-19 patients can be grouped into the following groups by illness severity—asymptomatic or presymptomatic, mild, moderate, severe, and critical illness, where patients with severe illness are individuals who have respiratory frequency>30 breaths per minute, SpO2<94% on room air at sea level, ratio of arterial partial pressure of oxygen to fraction of inspired oxygen (PaO2/FiO2)<300 mmHg, or lung infiltrates>50%. The management of a COVID-19 patient with severe illness includes pulmonary imagining and ECG, if indicated. Laboratory evaluation includes a complete blood count (CBC) with differential and a metabolic profile, including liver and renal function tests. Measurements of inflammatory markers such as C-reactive protein (CRP), D-dimer, and ferritin, while not part of standard care, may have prognostic value.

SARS-CoV-2 continues to evolve. Since the beginning of the COVID-19 pandemic, multiple variants of concern (VOCs) and variants of interest (VOIs) have been designated by the World Health Organization (WHO) based on their assessed potential for expansion and replacement of prior variants, for causing new waves with increased circulation, and for the need for adjustments to public health actions.

Based on comparisons of antigenic cross-reactivity using animal sera, replication studies in experimental models of the human respiratory tract, and evidence from clinical and epidemiological studies in humans, there is consensus among experts in WHO's Technical Advisory Group on SARS-CoV-2 Virus Evolution (TAG-VE) that compared to previous variants, Omicron represents the most divergent VOC seen to date. Since its emergence, Omicron viruses have continued to evolve genetically and antigenically with an expanding range of sublineages, which so far have all been characterized by properties of evasion of existing population immunity and a preference to infect the upper respiratory tract (versus lower respiratory tract), as compared to pre-Omicron VOCs.

The Omicron viruses account for over 98% of the publicly available sequences since February 2022 and constitute the genetic background from which new SARS-CoV-2 variants will likely emerge, although the emergence of variants derived from previously circulating VOCs or of completely new variants remains possible. The previous system classified all Omicron sublineages as part of the Omicron VOC and thus did not have the granularity needed to compare new descendent lineages with altered phenotypes to the Omicron parent lineages (BA.1, BA.2, BA.4/BA.5).

With these changes factored in, Alpha, Beta, Gamma, Delta as well as the Omicron parent lineage (B.1.1.529) are considered previously circulating VOCs. WHO has now classified XBB.1.5 as a VOL.

Although it has been years since the first case of COVID-19 pneumonia, current treatment options are limited and involve the treatment of symptoms, supportive care, isolation, and experimental measures. Therefore, there is an urgent unmet need to develop new therapies for the treatment of COVID-19 and other coronavirus infections.

### C. Peptides

### 1. Peptides Comprising a Trimerization Domain

Disclosed are peptides comprising a monomeric Fc fragment of an immunoglobulin recognized by an FcRn; a coronavirus antigen; and a trimerization domain. In some aspects, the coronavirus antigen can be any coronavirus spike protein, or antigenic fragment thereof. In some aspects, the coronavirus is Middle East respiratory syndrome coronavirus (MERS-CoV), Human Coronavirus (HCoV), SARS-CoV, or SARS-CoV-2. Thus, in some aspects, the coronavirus spike protein can be a MERS-CoV, HCoV-EMC, SARS-CoV, or SARS-CoV-2 spike protein, or antigenic fragment thereof.

Disclosed are peptides comprising a monomeric Fc fragment of an immunoglobulin recognized by an FcRn; SARS-CoV-2 antigen; and a trimerization domain. In some aspects, the SARS-CoV-2 antigen can be a SARS-CoV-2 spike protein. Thus, disclosed are peptides comprising a monomeric Fc fragment of an immunoglobulin recognized by an FcRn; SARS-CoV-2 spike protein; and a trimerization domain.

In some instances, the monomeric Fc fragment of an immunoglobulin recognized by an FcRn is conjugated to the amino or carboxy terminal end of a trimerization domain. In some aspects, the SARS-CoV-2 antigen is conjugated to the amino or carboxy terminal end of a trimerization domain. In some aspects, the monomeric Fc fragment of an immunoglobulin recognized by a FcRn is conjugated to the C-terminal end of a trimerization domain and the N-terminal end of the trimerization domain is conjugated to the C-terminal end of the SARS-CoV-2 antigen. In some instances, the monomeric Fc fragment of an immunoglobulin recognized by a FcRn is conjugated to the amino or carboxy terminal end of a SARS-CoV-2 antigen.

As described herein, the disclosed peptides can comprise a monomeric Fc fragment of an immunoglobulin recognized by a FcRn; a coronavirus antigen; and a trimerization domain. In some aspects, the order, from the N-terminus to the C-terminus of the peptide can be 1) coronavirus antigen, trimerization domain, monomeric Fc fragment of an immunoglobulin recognized by a FcRn; 2) monomeric Fc fragment of an immunoglobulin recognized by a FcRn, trimerization domain, coronavirus antigen; or 3) monomeric Fc fragment of an immunoglobulin recognized by a FcRn, coronavirus antigen, trimerization domain.

The conjugation can be direct or indirect. Indirect conjugation can be due to the presence of a linker, for example, a linker can be present in between the SARS-CoV-2 antigen and a trimerization domain.

Disclosed are peptides encoded by one or more of the nucleic acid sequences provided herein.

Disclosed are peptides comprising a monomeric Fc fragment of an immunoglobulin G recognized by an FcRn; a SARS-CoV-2 spike (S) antigen, wherein the SARS-CoV-2 S antigen comprises at least three mutations, wherein the mutations stabilize the S antigen in the prefusion form; and a trimerization domain. In some aspects, the monomeric Fc fragment of immunoglobulin recognized by an FcRn comprises two or three cysteine mutations at cysteines responsible for dimer formation.

Disclosed are peptides comprising a monomeric Fc fragment of an immunoglobulin recognized by an FcRn, wherein the monomeric Fc fragment of an immunoglobulin G recognized by an FcRn comprises at least two or three mutations in cysteine residues responsible for dimer formation; a SARS-CoV-2 S antigen, wherein the SARS-CoV-2 S antigen comprises at least two mutations, wherein the mutations stabilize the S antigen in the prefusion form; and a trimerization domain.

Disclosed are peptides comprising a monomeric Fc fragment of an immunoglobulin recognized by an FcRn; a SARS-CoV-2 S antigen, wherein the SARS-CoV-2 S antigen comprises mutations that stabilize the S antigen in the prefusion form, wherein the mutations comprise F817P, A892P, A899P, and A942P; and a trimerization domain. In some aspects, the monomeric Fc fragment of a immunoglobulin recognized by an FcRn comprises at least two or three cysteine mutations at cysteines responsible for dimer formation.

Disclosed are peptides comprising a monomeric Fc fragment of an immunoglobulin recognized by a neonatal receptor (FcRn); a SARS-CoV-2 S antigen, wherein the SARS-CoV-2 S antigen comprises mutations that stabilize the S antigen in the prefusion form, wherein the mutations comprise F817P, A892P, A899P, A942P, K986P, and V987P; and a trimerization domain. In some aspects, the monomeric Fc fragment of an immunoglobulin recognized by an FcRn comprises two or three cysteine mutations at cysteines responsible for dimer formation.

i. Monomeric Fc Fragment

The disclosed peptides can comprise any of the disclosed monomeric Fc fragments of an immunoglobulin recognized by an FcRn. In some aspects, the disclosed monomeric Fc fragments of an immunoglobulin recognized by an FcRn can be monomeric Fc fragments of an immunoglobulin G recognized by an FcRn.

A monomeric Fc fragment of an immunoglobulin recognized by a FcRn, as disclosed herein, can be any Fc fragment of an immunoglobulin recognized by an FcRn. In some aspects, a monomeric Fc fragment of an immunoglobulin recognized by an FcRn can comprise only the Fc portion of an immunoglobulin. In some aspects, a monomeric Fc fragment of an immunoglobulin recognized by an FcRn comprises at least one mutation in a cysteine residue responsible for dimer formation.

The disclosed monomeric Fc fragments of an immunoglobulin recognized by an FcRn are altered or mutated in order to make them monomeric. The monomeric Fc fragments of an immunoglobulin recognized by an FcRn cannot form dimers as found in an antibody. In some instances the monomeric Fc fragment of an immunoglobulin comprises a mutation in the Fc region of an immunoglobulin recognized by the FcRn sequence that results in the prevention of dimer formation. In some aspects, the monomeric Fc fragment of an immunoglobulin recognized by an FcRn comprises at least one mutation in a cysteine residue responsible for dimer formation. For example, mutations can be at one or more of positions 226 and 229 of the full-length sequence of the wild-type sequence of human IgG1. In some aspects, the Cys at positions 226 and 229 of full-length human wild-type IgG1 can be mutated to Ser in order to prevent dimer formation. In some aspects, the cysteine mutations to serine can be found at positions 11 and 14 of a sequence comprising only the hinge region, CH2, and CH3 domains of wild-type IgG. For example, the cysteine mutations to serine can be found at positions 11 and 14 of SEQ ID NO:7. In some aspects, positions 11 and 14 of SEQ ID NO:7 are located in the hinge region of monomeric Fc fragments of an immunoglobulin recognized by an FcRn. In another example, there can be three cysteine mutations in the monomeric Fc fragments of an immunoglobulin recognized by an FcRn. In some aspects, the cysteine mutations can be found at positions 5, 11, and 14 of SEQ ID NO:7, which can correspond to positions 220, 226, and 229 of full-length human wild-type IgG1. In some aspects, at least one, two, or all three cysteine mutations are cysteine to serine mutations.

In some instances, corresponding mutations can be made in other IgG Fc fragments and Fc fragments from other isotypes in order to mutate the cysteine residues responsible for dimer formation. In some instances, other mutations can be made throughout the Fc fragment of an immunoglobulin recognized by an FcRn so long as the FcRn binding region is not affected.

In some aspects, the C1q binding site can be ablated in the monomeric Fc fragment. This can be effective in helping avoid clearance of the Fc fragments via the complement pathway and thus allowing the disclosed peptides comprising a monomeric Fc fragment to remain in a subject and provide their therapeutic effect. In some aspects, C1q is known to bind to the CH2 domain of an immunoglobulin, particularly IgG. In some aspects, substituting the lysine at position 322 can ablate or eliminate the complement C1q binding site. For example, replacing Lys322 of full length human IgG with an Ala residue can ablate or eliminate the complement C1q binding site. In some aspects, replacing one or more of Glu318, Lys320, and Lys322 of full-length mouse IgG with an Ala residue can ablate or eliminate the complement C1q binding site. In some aspects, ablating C1q binding to the disclosed monomeric Fc fragments comprises mutation position 107 of a monomeric Fc fragment of an immunoglobulin recognized by an FcRn. For example, a mutation of lysine to alanine shown at position 107 of SEQ ID NO:7 can ablate C1q binding to a human monomeric Fc fragment of an immunoglobulin recognized by a FcRn.

In some aspects, the FcRn binding sites are known to be His310 and His433 or His310/Gln311 (HQ) and His433/Asn434 (HN) of full-length wild-type IgG. The region of the Fc-fragment of IgG that binds to the FcRn receptor in humans has been described based upon X-ray crystallography (Burmaister, W. P. et al., Nature, 1994; 372:379-378; incorporated by reference in its entirety herein). The major contact area of Fc with the FcRn receptor is near the junction of the CH2 and CH3 domains. Potential contacts are residues 248, 250-257, 272, 285, 288, 290-291, 308-311 and 314 in CH2 and 385-387, 428 and 433-436 in CH3. In some aspects, no mutations would be present in the FcRn binding sites. Given the foregoing information, those of ordinary skill in the art will readily recognize that the monomeric Fc fragment of IgG can be modified according to well-recognized procedures such as site-directed mutagenesis and the like to yield modified monomeric Fc fragments or portions thereof that will be bound by the FcRn receptor. Such modifications include modifications remote from the FcRn contact sites as well as modifications within the contact sites that preserve or even enhance binding.

In some aspects, the monomeric Fc fragment of an immunoglobulin recognized by an FcRn can be derived from any isotype that binds FcRn. The Fc-fragment should be chosen from an immunoglobulin known to bind the FcRn in the mucosa of the subject receiving the antigen-Fc vaccine. Immunoglobulin subclasses recognized by FcRn in different epithelial mucosa of animal subjects are known to a person in the art and can be found in Ober, R. J. et al, 2001, Int. Immunol. 13, 1551-9, incorporated by reference in its entirety herein. In some aspects, the monomeric Fc fragment of an immunoglobulin recognized by a FcRn is derived from a mammalian immunoglobulin. For example, the monomeric Fc fragment of an immunoglobulin recognized by a FcRn can be a human immunoglobulin sequence.

In some aspects, the amino acid sequence of a monomeric Fc fragment of a human IgG1 can be EPKSCDKTHTsPPsPAPELLGGPSVFLFPPKPKDTLMISRTPEVTCVVVDVSHEDPEVKFN WYVDGVEVHNAKTKPREEQYNSTYRVVSVLTVLHQDWLNGKEYKCaVSNKALPAPIE KTISKAKGQPREPQVYTLPPSRDELTKNQVSLTCLVKGFYPSDIAVEWESNGQPENNYK TTPPVLDSDGSFFLYSKLTVDKSRWQQGNVFSCSVMHEALHNHYTQKSLSLSPGK (SEQ ID NO: 7) or a variant thereof. In some aspects, the variant can a sequence 50%, 55%, 65%, 70%, 75%, 80%, 90%, 95%, 96%, 97%, 98%, or 99% identical to the sequence of SEQ ID NO:7. The two cysteine to serine mutations are shown at positions 11 and 14. A lysine to alanine mutation is shown at position 107. The cysteine mutations allow for the Fc fragment to remain monomeric and not dimerize with another Fc fragment. The lysine to alanine mutation ablates C1q binding to the Fc fragment.

In some aspects, the amino acid sequence of a monomeric Fc fragment of a mouse IgG2a can be EPRGPTIKPSPPSKSPAPNLLGGPSVFIFPPKIKDVLMISLSPIVTCVVVDVSEDDPDVQIS WFVNNVEVHTAQTQTHREDYNSTLRVVSALPIQHQDWMSGKAFACAVNNKDLPAPIE RTISKPKGSVRAPQVYVLPPPEEEMTKKQVTLTCMVTDFMPEDIYVEWTNNGKTELNY KNTEPVLDSDGSYFMYSKLRVEKKNWVERNSYSCSVVHEGLHNHHTTKSFSRTPGK (SEQ ID NO:33) or a sequence 50%, 55%, 65%, 70%, 75%, 80%, 90%, 95%, 96%, 97%, 98%, or 99% identical to the sequence of (SEQ ID NO:33). The bold underlined amino acids represent a mutation from cysteine to serine to generate a single chain Fc.

In some aspects, the monomeric Fc fragment of an immunoglobulin recognized by an FcRn comprises a full-length Fc region of an immunoglobulin. In some aspects, the monomeric Fc fragment of an immunoglobulin recognized by an FcRn comprises at least the CH2 and CH3 domains of an Fc region of an immunoglobulin. For example, the monomeric Fc fragment of an immunoglobulin recognized by an FcRn comprises one or more of a full-length CH2 and CH3 domain of IgG. In some aspects, the monomeric Fc fragment of an immunoglobulin recognized by an FcRn comprises at least a portion of the one or more CH2 and CH3 domains so long as the portions of the one or more CH2 and CH3 domains retain the ability to be recognized by FcRn.

In some instances, the monomeric Fc fragment of an immunoglobulin recognized by an FcRn is conjugated to the amino or carboxy-terminal end of the SARS-CoV-2 antigen. For example, the SARS-CoV-2 antigen can be the spike protein or a fragment thereof. In some instances, the monomeric Fc fragment of an immunoglobulin recognized by an FcRn is conjugated to the amino or carboxy terminal end of a trimerization domain. For example, the trimerization domain can be foldon. The conjugation can be direct or indirect. Indirect conjugation can be due to the presence of a linker in between the SARS-CoV-2 antigen or trimerization domain and the monomeric Fc fragment of an immunoglobulin recognized by a FcRn. Indirect conjugation can be due to the presence of another peptide in between the SARS-CoV-2 antigen or trimerization domain and the monomeric Fc fragment of an immunoglobulin recognized by an FcRn.

In some aspects, the monomeric Fc fragment of an immunoglobulin recognized by a FcRn can be derived from IgG. In some aspects, the IgG can be any IgG subtype. For example, the monomeric Fc fragment of an immunoglobulin recognized by a FcRn can be derived from IgG1, IgG2, IgG3, or IgG4.

ii. Trimerization Domain

The disclosed peptides have a trimerization domain.

The SARS-COV-2 S protein naturally exists as a trimer. Thus, disclosed herein are trimerization domains that allow the disclosed peptides, comprising one or more of the SARS-COV-2 S proteins, to trimerize. For example, three of the disclosed peptides can trimerize to form a peptide complex as disclosed herein.

In some instances, the trimerization domain is a T4 bacteriophage fibritin trimerization domain. For example, the T4 bacteriophage fibritin trimerization domain can be foldon which is present at the C-terminus of T4 bacteriophage fibritin. In some instances, the wild-type amino acid sequence of foldon is GYIPEAPRDGQAYVRKDGEWVLLSTFL. In some instances, the amino acid sequence of foldon is 50%, 55%, 65%, 70%, 75%, 80%, 90%, 95%, 96%, 97%, 98%, or 99% identical to the wild type foldon sequence. In some aspects, the nucleic acid sequence of foldon can be represented by the sequence

GGCTACATCCCCGAGGCCCCCAGAGACGGCCAGGCCTACGTGAGAAAGGA
CGGCGAGTGGGTGCTGCTGAGCACCTTCCTG.

In some instances, the trimerization domain can be but is not limited to the transcription factor GCN4pII trimerization motif (MKQIEDKIEEILSKIYHIENEIARIKKLIGEV), or human collagen XV trimerization domain. In some instances, the trimerization domain can be an amino acid sequence that is 50%, 55%, 65%, 70%, 75%, 80%, 90%, 95%, 96%, 97%, 98%, or 99% identical to transcription factor GCN4pII trimerization motif or human collagen XV trimerization domain.

In some aspects, the trimerization domain is between the monomeric Fc fragment recognized by FcRn and the SARS-CoV-2 antigen. In some aspects, the trimerization domain is on the C-terminal end of the SARS-CoV-2 S protein. In some aspects, the trimerization domain is on the N-terminal end of the monomeric Fc fragment recognized by FcRn.

iii. Coronavirus Antigen

In some aspects, the disclosed peptides can comprise a monomeric Fc fragment recognized by FcRn, a trimerization domain, and a coronavirus antigen. In some aspects, a coronavirus antigen can be any region of a coronavirus that can generate an immune response. In some aspects, a coronavirus antigen can be all or a portion of the coronavirus spike (S) protein. In some aspects, the coronavirus S protein is the soluble portion of the coronavirus S protein. For example, the transmembrane domain and cytoplasmic domain are not present in the soluble portion of the coronavirus S protein. In some aspects, the coronavirus is Middle East respiratory syndrome coronavirus (MERS-CoV), Human Coronavirus (HcoV), SARS-CoV, or SARS-CoV-2. Thus, in some aspects, the coronavirus spike protein can be a MERS-CoV, HCoV, SARS-CoV, or SARS-CoV-2 spike protein, or antigenic fragment thereof. In some aspects, the coronavirus spike protein can be a SARS-CoV-2 spike protein from an omicron or delta variant.

In some aspects, the disclosed peptides can comprise a monomeric Fc fragment recognized by FcRn, a trimerization domain, and a SARS-COV-2 antigen. In some aspects, a SARS-COV-2 antigen can be any region of SARS-COV-2 that can generate an immune response. In some aspects, a SARS-COV-2 antigen can be all or a portion of the SARS-COV-2 S protein. In some aspects, the SARS-COV-2 S protein is the soluble portion of the SARS-COV-2 S protein. For example, the transmembrane domain and cytoplasmic domain are not present in the soluble portion of the SARS-COV-2 S protein.

In some aspects, a SARS-CoV-2 S protein can be derived from wild type SARS-CoV-2 (Wuhan strain) or from a variant strain, such as, but not limited to, the variants of D614G (originally found in China/Germany), B.1.1.7 or 20I/501Y.V1 (originally found in the United Kingdom), B.1.351 or 20H/501.V2 (originally found in South Africa), P.1 or 203/501Y.V3 (originally found in Japan/Brazil), 20C/S:452R (originally found in California), Cluster 5 Variant (originally found in Denmark), XBB.1.5 (e.g. 1.5.70, 1.5.68, 1.5.72, 1.5.10, 1.5.59, 1.5.1), XBB.1.16 (e.g. 1.16.6, 1.16.11, 1.16.15, 1.6.1), EG.5, XBB.1.9 (e.g. 1.9.1, 1.9.2), XBB.2.3, BA.1, BA.5, BA.2.86, HV,1, FL,1.5.1, HK.3, JD.1.1, JF.1, GK.1.1., HF.1, BA.2 (e.g. 2.111, 2.12.2), BAA, BA.5, BA.527, BA.529, GE.1, XBB, GK.2, EG.6.1, XBB.1.42.2, CH.1.1, XBB.2.3.8, FD.1.1, FE.1.1, EU.1.1, B.1.1.529, B.1.617.2, XBB.128, BQ.1 (e.g. 1.1), JN.1. In some aspects, a SARS-CoV-2 S protein can be derived from any of the SARS-CoV-2 strains of a, J, 7, 6, or P (original or subvariant strains).

In some aspects, SARS-CoV-2 S protein can have mutations that stabilize the S antigen in the prefusion form. In some aspects, the SARS-CoV-2 S protein can have at least one, two, three, four, five, or six mutations that stabilize the S antigen in the prefusion form. In some aspects, the SARS-CoV-2 S protein can have at least three mutations that stabilize the S antigen in the prefusion form. In some aspects, the at least three mutations in the SARS-CoV-2 S antigen correspond to at least three of positions Phe 817, Ala 892, Ala 899, Ala 942, Lys 986, or Val 987 of the SARS-CoV-2 S antigen sequence (SEQ ID NO:8). In some aspects, the mutations can be any amino acid substituted for a proline. In some aspects, the amino acid substitution can be any conservative amino acid substitution of proline. In some aspects, the chemical properties of proline discourage helix formation and promote local rigidity in a polypeptide chain. In some aspects, the helix structure can be used by the postfusion of a viral membrane protein for initiating fusion with the cell membrane, Thus, in some aspects, any substitution that prevents helix formation can be used. Thus, in some aspects, the SARS-CoV-2 S protein can have at least one, two, three, four, five, or six prolines not present in the wild type SARS-CoV-2 S protein. In some aspects, a SARS-CoV-2 S protein having two proline mutations can comprise proline mutations at Lys 986 or Val 987 of the SARS-CoV-2 S antigen sequence (SEQ ID NO:8). In some aspects, a SARS-CoV-2 S protein having four proline mutations can comprise proline mutations at F817P, A892P, A899P, and A942P of the SARS-CoV-2 S antigen sequence (SEQ ID NO:8). In some aspects, a SARS-CoV-2 S protein having six proline mutations can comprise proline mutations at F817P, A892P, A899P, A942P, K986P, and V987P of the SARS-CoV-2 S antigen sequence (SEQ ID NO:8). In some aspects, a SARS-CoV-2 S protein having at least two, three, four, five, or six mutations can have a combination of any of the mutations at F817P, A892P, A899P, A942P, K986P, and V987P.

In some aspects, the soluble portion of the SARS-COV-2 S protein is amino acids 1-1213 of the full length wild type S protein. Specifically, the soluble portion of the SARS-COV-2 S protein comprises the sequence

(SEQ ID NO: 8)
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLS

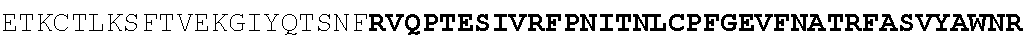

KRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERD
ISTEIYQAGSTPCNGVEGENCYFPLQSYGFQPTNGVGYQPYRVVVLSFELLHAPAT
VCGPKKSTNLVKNKCVNFNFNGLTGTGVLTESNKKFLPFQQFGRDIADTTDAVRDPQ
TLEILDITPCSFGGVSVITPGTNTSNQVAVLYQDVNCTEVPVAIHADQLTPTWRVYSTGS
NVFQTRAGCLIGAEHVNNSYECDIPIGAGICASYQTQTNSPRRAASVASQSIIAYTMSLG
AENSVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKASFIEDLL
FNKVTLADAGFIKQYGDCLGDIAARDLICAQKFNGLTVLPPLLTDEMIAQYTSALLAGTI
TSGWTFGAGAALQIPFAMQMAYRENGIGVTQNVLYENQKLIANQFNSAIGKIQDSLSST
ASALGKLQDVVNQNAQALNTLVKQLSSNFGAISSVLNDILSRLDPPEAEVQIDRLITGRL
QSLQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVN
IQKEIDRLNEVAKNLNESLIDLQELGKYEQYIKWP

or a variant thereof. In aspects, the variant can be a sequence 50%, 55%, 65%, 70%, 75%, 80%, 90%, 95%, 96%, 97%, 98%, or 99% identical to the sequence of SEQ ID NO:8. The underlined sequence represents a native signal peptide of S protein. The bold shaded sequence represents the RBD of S1. The bold underline sequence represents the mutated S1/S2 cleavage site (R685A in italics, no change in S686). The bold letter and bold underline sequence represents a mutation at the S2′ cleavage site (R815A in italics, no change in S816). The bold, italics, and shaded sequence represents K986P and V987P mutations which allow the S protein to keep the Pre-fusion conformation.

In some aspects, the SARS-COV-2 S protein is the soluble portion of the D614G variant S protein. Specifically, the S protein of the D614G variant can comprise the sequence

(SEQ ID NO: 11)
MFVFLVLLPLVSSQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGKLADYNYKLPDDFTGCVIAWNSNNLDSKVGGNYN
YLYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQgVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

(with the mutation of D614G shown in lowercase) or a variant thereof. In some aspects, the variant can be a sequence 50%, 55%, 65%, 75%, 80%, 90%, 95%, 96%, 97%, 98%, or 99% identical to the sequence of SEQ ID NO:11. Amino acids 1-1213 of SEQ ID NO:11 represent the soluble portion of the protein. Thus, amino acids 1214-1273 (shown here in underline) represent the transmembrane and cytoplasmic tail of SEQ ID NO:11.

In some aspects, the SARS-COV-2 S protein is the soluble portion of the B.1.1.7 variant S protein. In some aspects, the B.1.1.7 variant S protein comprises deletions at amino acids 69, 70, and 144 and the following substitutions: N501Y, A570D, D614G, P6811H, T7161, S982A, D1118H (numbers are based on position prior to the deletion of amino acids 69, 70, and 144). Specifically, the S protein of the B.1.1.7 variant can comprise the sequence

(SEQ ID NO: 12)
MFVFLVLLPLVSSQCVNLTTRTQLPPAYTNSFTRGVYYPDKVFRSSVLHS
TQDLFLPFFSNVTWFHAISGTNGTKRFDNPVLPFNDGVYFASTEKSNIIR
GWIFGTTLDSKTQSLLIVNNATNVVIKVCEFQFCNDPFLGVYHKNNKSWM
ESEFRVYSSANNCTFEYVSQPFLMDLEGKQGNFKNLREFVFKNIDGYFKI
YSKHTPINLVRDLPQGFSALEPLVDLPIGINITRFQTLLALHRSYLTPGD
SSSGWTAGAAAYYVGYLQPRTFLLKYNENGTITDAVDCALDPLSETKCTL
KSFTVEKGIYQTSNFRVQPTESIVRFPNITNLCPFGEVENATRFASVYAW
NRKRISNCVADYSVLYNSASFSTFKCYGVSPTKLNDLCFTNVYADSFVIR
GDEVRQIAPGQTGKIADYNYKLPDDFTGCVIAWNSNNLDSKVGGNYNYLY
RLFRKSNLKPFERDISTEIYQAGSTPCNGVEGFNCYFPLQSYGFQPTYGV
GYQPYRVVVLSFELLHAPATVCGPKKSTNLVKNKCVNFNFNGLTGTGVLT
ESNKKFLPFQQFGRDIDDTTDAVRDPQTLEILDITPCSFGGVSVITPGTN
TSNQVAVLYQGVNCTEVPVAIHADQLTPTWRVYSTGSNVFQTRAGCLIGA
EHVNNSYECDIPIGAGICASYQTQTNSHRRARSVASQSIIAYTMSLGAEN
SVAYSNNSIAIPINFTISVTTEILPVSMTKTSVDCTMYICGDSTECSNLL
LQYGSFCTQLNRALTGIAVEQDKNTQEVFAQVKQIYKTPPIKDFGGFNFS
QILPDPSKPSKRSFIEDLLFNKVTLADAGFIKQYGDCLGDIAARDLICAQ
KFNGLTVLPPLLTDEMIAQYTSALLAGTITSGWTFGAGAALQIPFAMQMA
YRFNGIGVTQNVLYENQKLIANQFNSAIGKIQDSLSSTASALGKLQDVVN
QNAQALNTLVKQLSSNFGAISSVLNDILARLDKVEAEVQIDRLITGRLQS
LQTYVTQQLIRAAEIRASANLAATKMSECVLGQSKRVDFCGKGYHLMSFP
QSAPHGVVFLHVTYVPAQEKNFTTAPAICHDGKAHFPREGVFVSNGTHWF
VTQRNFYEPQIITTHNTFVSGNCDVVIGIVNNTVYDPLQPELDSFKEELD
KYFKNHTSPDVDLGDISGINASVVNIQKEIDRLNEVAKNLNESLIDLQEL
GKYEQYIKWPWYIWLGFIAGLIAIVMVTIMLCCMTSCCSCLKGCCSCGSC
CKFDEDDSEPVLKGVKLHYT

(substitutions shown in lowercase) or a variant thereof. In some aspects, the variant can be a sequence 50%, 55%, 65%, 70%, 75%, 80%, 90%, 95%, 96%, 97%, 98%, or 99% identical to the sequence of SEQ ID NO:12. Amino acids 1-1210 of SEQ ID NO:12 represent the soluble portion of the protein. Thus, amino acids 1211-1270 (shown here in underline) represent the transmembrane and cytoplasmic tail of SEQ ID NO:12.

In some aspects, the SARS-COV-2 S protein is the soluble portion of the B.1.351 variant S protein. In some aspects, the B.1.351 variant S protein comprises the following substitutions D80A, D215G, K417N, A701V, N501Y, E484K. Specifically, the S protein of the B.1.351 variant can comprise the sequence

(SEQ ID NO: 13)
MFVFLVLLPLVSSQCVNLTTRTQLPPAYTNSFTRGVYYPDKVFRSSVLHS
TQDLFLPFFSNVTWFHAIHVSGTNGTKRFANPVLPFNDGVYFASTEKSNI
IRGWIFGTTLDSKTQSLLIVNNATNVVIKVCEFQFCNDPFLGVYYHKNNK
SWMESEFRVYSSANNCTFEYVSQPFLMDLEGKQGNFKNLREFVFKNIDGY
FKIYSKHTPINLVRGLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGNIADYNYKLPDDFTGCVIAWNSNNLDSKVGGNYN
YLYRLFRKSNLKPFERDISTEIYQAGSTPCNGVKGFNCYFPLQSYGFQPT
YGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQdVNCTEVPVAIHADQLTPTWRVYSTGSNVFQTRAGCL
IGAEHVNNSYECDIPIGAGICASYQTQTNSPRRARSVASQSIIAYTMSLG
VENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRENGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
AFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

(substitutions shown in lowercase) or a variant thereof. In some aspects, the variant can be a sequence 50%, 55%, 65%, 70%, 75%, 80%, 90%, 95%, 96%, 97%, 98%, or 99% identical to the sequence of SEQ ID NO:13. Amino acids 1-1213 of SEQ ID NO:13 represent the soluble portion of the protein. Thus, amino acids 1214-1273 (shown here in underline) represent the transmembrane and cytoplasmic tail of SEQ ID NO:13.

In some aspects, the SARS-COV-2 S protein is the soluble portion of the P.1 variant S protein. In some aspects, the P.1 variant S protein comprises the following substitutions L18F, T20N, P26S, D138Y, R190S, K417T, E484K, N501Y, H655Y, T1027L Specifically, the S protein of the P.1 variant can comprise the sequence

(SEQ ID NO: 14)
MFVFLVLLPLVSSQCVNFTNRTQLPSAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNYPFLGVYYHKNNK
SWMESEFRVYSSANNCTFEYVSQPFLMDLEGKQGNFKNLSEFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGTIADYNYKLPDDFTGCVIAWNSNNLDSKVGGNYN
YLYRLFRKSNLKPFERDISTEIYQAGSTPCNGVKGFNCYFPLQSYGFQPT
YGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQdVNCTEVPVAIHADQLTPTWRVYSTGSNVFQTRAGCL
IGAEYVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAAiKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

(substitutions shown in lowercase) or a variant thereof. In some aspects, the variant can be a sequence 50%, 55%, 65%, 70%, 75%, 80%, 90%, 95%, 96%, 97%, 98%, or 99% identical to the sequence of SEQ ID NO:14. Amino acids 1-1213 of SEQ ID NO:14 represent the soluble portion of the protein. Thus, amino acids 1214-1273 (shown here in underline) represent the transmembrane and cytoplasmic tail of SEQ ID NO:14.

In some aspects, the SARS-COV-2 S protein is the soluble portion of the 20C/S:452R variant S protein. In some aspects, the 20C/S:452R variant S protein comprises the following substitutions 5131I, W152C, L452R. Specifically, the S protein of the 20C/S:452R variant can comprise the sequence

(SEQ ID NO: 15)
MFVFLVLLPLVSIQCVNLTTRTQLPPAYTNSFTRGVYYPDKVFRSSVLHS
TQDLFLPFFSNVTWFHAIHVSGTNGTKRFDNPVLPFNDGVYFASTEKSNI
IRGWIFGTTLDSKTQSLLIVNNATNVVIKVCEFQFCNDPFLGVYYHKNNK
SCMESEFRVYSSANNCTFEYVSQPFLMDLEGKQGNFKNLREFVFKNIDGY
FKIYSKHTPINLVRDLPQGFSALEPLVDLPIGINITRFQTLLALHRSYLT
PGDSSSGWTAGAAAYYVGYLQPRTFLLKYNENGTITDAVDCALDPLSETK
CTLKSFTVEKGIYQTSNFRVQPTESIVRFPNITNLCPFGEVENATRFASV
YAWNRKRISNCVADYSVLYNSASFSTFKCYGVSPTKLNDLCFTNVYADSF
VIRGDEVRQIAPGQTGKIADYNYKLPDDFTGCVIAWNSNNLDSKVGGNYN
YRYRLFRKSNLKPFERDISTEIYQAGSTPCNGVEGFNCYFPLQSYGFQPT
NGVGYQPYRVVVLSFELLHAPATVCGPKKSTNLVKNKCVNFNFNGLTGTG
VLTESNKKFLPFQQFGRDIADTTDAVRDPQTLEILDITPCSFGGVSVITP
GTNTSNQVAVLYQDVNCTEVPVAIHADQLTPTWRVYSTGSNVFQTRAGCL
IGAEHVNNSYECDIPIGAGICASYQTQTNSPRRARSVASQSIIAYTMSLG
AENSVAYSNNSIAIPTNFTISVTTEILPVSMTKTSVDCTMYICGDSTECS
NLLLQYGSFCTQLNRALTGIAVEQDKNTQEVFAQVKQIYKTPPIKDFGGF
NFSQILPDPSKPSKRSFIEDLLFNKVTLADAGFIKQYGDCLGDIAARDLI
CAQKFNGLTVLPPLLTDEMIAQYTSALLAGTITSGWTFGAGAALQIPFAM
QMAYRFNGIGVTQNVLYENQKLIANQFNSAIGKIQDSLSSTASALGKLQD
VVNQNAQALNTLVKQLSSNFGAISSVLNDILSRLDKVEAEVQIDRLITGR
LQSLQTYVTQQLIRAAEIRASANLAATKMSECVLGQSKRVDFCGKGYHLM
SFPQSAPHGVVFLHVTYVPAQEKNFTTAPAICHDGKAHFPREGVFVSNGT
HWFVTQRNFYEPQIITTDNTFVSGNCDVVIGIVNNTVYDPLQPELDSFKE
ELDKYFKNHTSPDVDLGDISGINASVVNIQKEIDRLNEVAKNLNESLIDL
QELGKYEQYIKWPWYIWLGFIAGLIAIVMVTIMLCCMTSCCSCLKGCCSC
GSCCKFDEDDSEPVLKGVKLHYT

(substitutions shown in lowercase) or a variant thereof. In some aspects, the variant can be a sequence 50%, 55%, 65%, 70%, 75%, 80%, 90%, 95%, 96%, 97%, 98%, or 99% identical to the sequence of SEQ ID NO:15. Amino acids 1-1213 of SEQ ID NO:15 represent the soluble portion of the protein. Thus, amino acids 1214-1273 (shown here in underline) represent the transmembrane and cytoplasmic tail of SEQ ID NO:15.

In some aspects, the SARS-COV-2 S protein is the soluble portion of the cluster 5 variant S protein. In some aspects, the cluster 5 variant S protein comprises deletions at amino acids 69, 70 and the following substitutions U453F, 1692V, and M1229I. Specifically, the S protein of the cluster 5 variant can comprise the sequence

(SEQ ID NO: 16)
MFVFLVLLPLVSSQCVNLTTRTQLPPAYTNSFTRGVYYPDKVFRSSVLHS
TQDLFLPFFSNVTWFHAISGTNGTKRFDNPVLPFNDGVYFASTEKSNIIR
GWIFGTTLDSKTQSLLIVNNATNVVIKVCEFQFCNDPFLGVYYHKNNKSW
MESEFRVYSSANNCTFEYVSQPFLMDLEGKQGNFKNLREFVFKNIDGYFK
IYSKHTPINLVRDLPQGFSALEPLVDLPIGINITRFQTLLALHRSYLTPG
DSSSGWTAGAAAYYVGYLQPRTFLLKYNENGTITDAVDCALDPLSETKCT
LKSFTVEKGIYQTSNFRVQPTESIVRFPNITNLCPFGEVFNATRFASVYA
WNRKRISNCVADYSVLYNSASFSTFKCYGVSPTKLNDLCFTNVYADSFVI
RGDEVRQIAPGQTGKIADYNYKLPDDFTGCVIAWNSNNLDSKVGGNYNYL
FRLFRKSNLKPFERDISTEIYQAGSTPCNGVEGENCYFPLQSYGFQPTNG
VGYQPYRVVVLSFELLHAPATVCGPKKSTNLVKNKCVNFNFNGLTGTGVL
TESNKKFLPFQQFGRDIADTTDAVRDPQTLEILDITPCSFGGVSVITPGT
NTSNQVAVLYQDVNCTEVPVAIHADQLTPTWRVYSTGSNVFQTRAGCLIG
AEHVNNSYECDIPIGAGICASYQTQTNSPRRARSVASQSIVAYTMSLGAE
NSVAYSNNSIAIPTNFTISVTTEILPVSMTKTSVDCTMYICGDSTECSNL
LLQYGSFCTQLNRALTGIAVEQDKNTQEVFAQVKQIYKTPPIKDFGGFNF
SQILPDPSKPSKRSFIEDLLFNKVTLADAGFIKQYGDCLGDIAARDLICA
QKFNGLTVLPPLLTDEMIAQYTSALLAGTITSGWTFGAGAALQIPFAMQM
AYRFNGIGVTQNVLYENQKLIANQFNSAIGKIQDSLSSTASALGKLQDVV
NQNAQALNTLVKQLSSNFGAISSVLNDILSRLDKVEAEVQIDRLITGRLQ
SLQTYVTQQLIRAAEIRASANLAATKMSECVLGQSKRVDFCGKGYHLMSF
PQSAPHGVVFLHVTYVPAQEKNFTTAPAICHDGKAHFPREGVFVSNGTHW
FVTQRNFYEPQIITTDNTFVSGNCDVVIGIVNNTVYDPLQPELDSFKEEL
DKYFKNHTSPDVDLGDISGINASVVNIQKEIDRLNEVAKNLNESLIDLQE
LGKYEQYIKWPWYIWLGFIAGLIAIVMVTIMLCCMTSCCSCLKGCCSCGS
CCKFDEDDSEPVLKGVKLHYT

(substitutions shown in lowercase) or a variant thereof. In some aspects, the variant can be a sequence 50%, 55%, 65%, 70%, 75%, 80%, 90%, 95%, 96%, 97%, 98%, or 99% identical to the sequence of SEQ ID NO:16. Amino acids 1-1210 of SEQ ID NO:16 represent the soluble portion of the protein. Thus, amino acids 1214-1270 (shown here in underline) represent the transmembrane and cytoplasmic tail of SEQ ID NO:16.

In some aspects, the SARS-COV-2 S protein is the soluble portion of the omicron XBB 1.5 S protein. Specifically, the S protein of the omicron strain can comprise the sequence MFVFLVLLPLVSSQCVNLITRTQSYTNSFTRGVYYPDKVFRSSVLHSTQDLFLPFFSNVT WFHAIHVSGTNGTKRFDNPALPFNDGVYFASTEKSNIIRGWIFGTTLDSKTQSLLIVNNA TNVVIKVCEFQFCNDPFLDVYQKNNKSWMESEFRVYSSANNCTFEYVSQPFLMDLEGK EGNFKNLREFVFKNIDGYFKIYSKHTPINLERDLPQGFSALEPLVDLPIGINITRFQTLLAL HRSYLTPVDSSSGWTAGAAAYYVGYLQPRTFLLKYNENGTITDAVDCALDPLSETKCT LKSFTVEKGIYQTSNFRVQPTESIVRFPNITNLCPFHEVFNATTFASVYAWNRKRISNCV ADYSVIYNFAPFFAFKCYGVSPTKLNDLCFTNVYADSFVIRGNEVSQIAPGQTGNIADYN YKLPDDFTGCVIAWNSNKLDSKPSGNYNYLYRLFRKSKLKPFERDISTEIYQAGNKPCN GVAGPNCYSPLQSYGFRPTYGVGHQPYRVVVLSFELLHAPATVCGPKKSTNLVKNKCV NFNFNGLTGTGVLTESNKKFLPFQQFGRDIADTTDAVRDPQTLEILDITPCSFGGVSVITP GTNTSNQVAVLYQGVNCTEVPVAIHADQLTPTWRVYSTGSNVFQTRAGCLIGAEYVNN SYECDIPIGAGICASYQTQTKSHRAAASVASQSIIAYTMSLGAENSVAYSNNSIAIPTNFTI SVTTEILPVSMTKTSVDCTMYICGDSTECSNLLLQYGSFCTQLKRALTGIAVEQDKNTQE VFAQVKQIYKTPPIKYFGGFNFSQILPDPSKPSKASFIEDLLFNKVTLADAGFIKQYGDCL GDIAARDLICAQKFNGLTVLPPLLTDEMIAQYTSALLAGTITSGWTFGAGAALQIPFAM QMAYRFNGIGVTQNVLYENQKLIANQFNSAIGKIQDSLSSTASALGKLQDVVNHNAQA LNTLVKQLSSKFGAISSVLNDILSRLDPPEAEVQIDRLITGRLQSLQTYVTQQLIRAAEIRA SANLAATKMSECVLGQSKRVDFCGKGYHLMSFPQSAPHGVVFLHVTYVPAQEKNFTT APAICHDGKAHFPREGVFVSNGTHWFVTQRNFYEPQIITTDNTFVSGNCDVVIGIVNNT VYDPLQPELDSFKEELDKYFKNHTSPDVDLGDISGINASVVNIQKEIDRLNEVAKNLNES LIDLQELGKYEQYIKWP (SEQ ID NO:30) or variant thereof. In some aspects, the variant can be a sequence 50%, 55%, 65%, 70%, 75%, 80%, 90%, 95%, 96%, 97%, 98%, or 99% identical to the sequence of SEQ ID NO:30.

In some aspects, the SARS-COV-2 S protein can be cleaved into S1 and S2 subunits by proteases. In some aspects, S1 comprises the receptor-binding domain (RBD) which allows viruses to directly bind to the ACE2 receptor. In some aspects, S2 can mediate membrane fusion, with the help of a protease, in cells. In some aspects, the SARS-COV-2 S protein (“S protein”) is the full-length soluble S protein, the S1 subunit, the S2 subunit, or the RBD. In some aspects, the SARS-COV-2 S protein is a portion of full length soluble S protein, the S1 subunit, the S2 subunit, or the RBD. In some aspects, the SARS-COV-2 S protein is a variant of a wild type sequence and thus, is 50, 55, 60, 65, 70, 75, 80, 85, 90, 95, 96, 97, 98, 99, or 100% identical to the wild type full length S protein, the S1 subunit, the S2 subunit, or the RBD. In some aspects, a variant SARS-COV-2 S protein can comprise a modified amino acid or a non-naturally occurring amino acid.

In some aspects, the complete wild type amino acid sequence of SARS-COV-2 can be found in Genbank as accession number MN908947. The S protein is nucleic acids 21563-25384 of accession number MN908947.

In some aspects, the S protein is the full length S protein. Because the S protein can be cleaved by proteases, in some aspects, the disclosed SARS-COV-2 S protein can be altered or mutated to remove the cleavage sites and produce a non-cleavable S protein. In some aspects, the mutations that remove the cleavage site are R685A and R816A of the full length wild type S protein. For example, the cleavage sites of R685A and R816A are at positions 685 and 816, respectively, of SEQ ID NO:8.

In some aspects, the S protein can be further altered or mutated so that the S protein retains its prefusion state. In some aspects, mutations that maintain the S protein in a prefusion state can be K986P and V987P.

iv. Linkers

Disclosed are peptides comprising a monomeric Fc fragment of an immunoglobulin recognized by FcRn; a SARS-CoV-2 antigen; and a trimerization domain, wherein the peptide further comprises one or more linkers.

In some instances, at least one of the one or more linkers is on the N-terminus end of the monomeric Fc fragment of an immunoglobulin recognized by a FcRn. In some instances, at least one of the one or more linkers is on the C-terminus end of the monomeric Fc fragment of an immunoglobulin recognized by a FcRn.

In some instances, at least one of the one or more linkers is located between the SARS-CoV-2 antigen and the monomeric Fc fragment of an immunoglobulin recognized by a FcRn. In some instances, at least one of the one or more linkers is located between the trimerization domain and the monomeric Fc fragment of an immunoglobulin recognized by a FcRn. In some instances, at least one of the one or more linkers is located between the trimerization domain and the SARS-CoV-2 antigen.

In some instances, the one or more linkers are small, nonpolar, amino acid linkers. For example, the linker can be a GS-linker. The number of glycine, serine, and glycine/serine repeats can vary in the one or more linkers. Examples of GS linkers can be GSGSGS and GSGGGGSGGGGSGS.

v. Additional elements

In some aspects, the disclosed peptides comprise a signal peptide. In some aspects, a signal peptide is any short peptide (about 10-30 amino acids) that helps translocate the peptide to the cell membrane. In some aspects, the signal peptide is present on the N-terminal end of the SARS-CoV-2 antigen. In some aspects, the signal peptide is derived from the coronavirus antigen. In some aspects, the signal peptide is derived from the SARS-CoV-2 antigen. For example, the native signal peptide found on SARS-CoV-2 S protein can be present in the disclosed peptides. In some aspects, the native signal peptide can comprise the amino acid sequence of MFVFLVLLPLVSSQC from SARS-CoV-2 S protein. In some aspects, a signal peptide can comprise one or more of the sequences present in Table 1.

TABLE 1
Exemplary signal peptide sequences.

| Signal | | SEQ |
|---|---|---|
| Sequence Name | Sequence | ID NO: |
| Human OSM | MGVLLTQRTLLSLVLALLFPSMASM | 34 |
| VSV-G | MKCLLYLAFLFIGVNC | 35 |
| Mouse Ig Kappa | METDTLLLWVLLLWVPGSTGD | 36 |
| Human IgG2 H | MGWSCIILFLVATATGVHS | 37 |
| BM40 | MRAWIFFLLCLAGRALA | 38 |
| Secrecon | MWWRLWWLLLLLLLLWPMVWA | 39 |
| Human IgKVIII | MDMRVPAQLLGLLLLWLRGARC | 40 |
| CD33 | MPLLLLLPLLWAGALA | 41 |
| tPA | MDAMKRGLCCVLLLCGAVFVSPS | 42 |
| Human | MAFLWLLSCWALLGTTFG | 43 |
| Chymotrypsinogen | | |
| Human | MNLLLILTFVAAAVA | 44 |
| trypsinogen-2 | | |
| Human IL-2 | MYRMQLLSCIALSLALVTNS | 45 |
| Gaussia luc | MGVKVLFALICIAVAEA | 46 |
| Albumin(HSA) | MKWVTFISLLFSSAYS | 47 |
| Influenza | MKTIIALSYIFCLVLG | 48 |
| Haemagglutinin | | |
| Human insulin | MALWMRLLPLLALLALWGPDPAAA | 49 |
| Silkworm Fibroin | MKPIFLVLLVVTSAYA | 50 |
| LC | | |
| Human CD5 | MPMGSLQPLATLYLLGMLVASCLG | 51 |

In some instances, the disclosed peptides can further comprise cleavage sites or tag sequences.

In some instances, a cleavage site can be present in the disclosed peptides. Cleavage sites can allow for cleavage of the monomeric Fc fragment of an immunoglobulin recognized by FcRn away from the SARS-CoV-2 antigen. In some instances, a cleavage site can be recognized by a protease or a chemical compound. In some instances, a cleavage site can be a site recognized by, but not limited to, enterokinase, pepsin, factor Xa, tobacco etch virus protease, or thrombin.

In some instances, a tag sequence can be present in the disclosed peptides. In some instances, a tag sequence can be a detection label/label sequence or a purification tag. As used herein, a detection label or label sequence is any molecule that can be associated with a nucleic acid or peptide, directly or indirectly, and which results in a measurable, detectable signal, either directly or indirectly. Many such labels for incorporation into nucleic acids or coupling to nucleic acids or peptides are known to those of skill in the art. Examples of detection labels can be, but are not limited to, radioactive isotopes, fluorescent molecules, phosphorescent molecules, enzymes, antibodies, and ligands.

Examples of suitable fluorescent labels include fluorescein (FITC), 5,6-carboxymethyl fluorescein, Texas red, nitrobenz-2-oxa-1,3-diazol-4-yl (NBD), coumarin, dansyl chloride, rhodamine, 4′-6-diamidino-2-phenylinodole (DAPI), and the cyanine dyes Cy3, Cy3.5, Cy5, Cy5.5 and Cy7. Preferred fluorescent labels are fluorescein (5-carboxyfluorescein-N-hydroxysuccinimide ester) and rhodamine (5,6-tetramethyl rhodamine). Preferred fluorescent labels for combinatorial multicolor coding are FITC and the cyanine dyes Cy3, Cy3.5, Cy5, Cy5.5 and Cy7. The absorption and emission maxima, respectively, for these fluors are: FITC (490 nm; 520 nm), Cy3 (554 nm; 568 nm), Cy3.5 (581 nm; 588 nm), Cy5 (652 nm: 672 nm), Cy5.5 (682 nm; 703 nm) and Cy7 (755 nm; 778 nm), thus allowing their simultaneous detection. The fluorescent labels can be obtained from a variety of commercial sources, including Molecular Probes, Eugene, OR and Research Organics, Cleveland, Ohio.

In some instances, a label sequence can be, but is not limited to, an isotope marker, colorimetric biosensors, or fluorescent labels. For example, fluorescent markers can be, but are not limited to, green fluorescent protein (GFP) or rhodamine fluorescent protein (RFP). Other label sequences can include biotin, streptavidin, horseradish peroxidase, or luciferase.

In some instances, a tag sequence can be a purification tag. In some instances, a purification tag can be, but is not limited to, histidine, glutathione-S-transferase, albumin-binding protein, FLAG epitope, galactose-binding protein, myc, or hemagglutinin.

In some aspects, the compositions or peptides disclosed herein can further comprise an adjuvant. In some aspects, the adjuvant is immunostimulatory oligonucleotides containing unmethylated CpG dinucleotides (“CpG”). CpGs are known in the art as being adjuvants when administered by both systemic and mucosal routes (WO 96/02555, EP 468520, Davis et al., J. Immunol, 1998, 160(2): 870-876, McCluskie and Davis, J. Immunol., 1998, 161(9): 4463-6). CpG is an abbreviation for cytosineguanosinc dinucicotide motifs present in DNA. Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, 1995, Nature 374, p. 546. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the dinucleotide CG motif is not methylated, but other unmethylated CpG sequences are known to be immunostimulatory and may be used in the present invention.

vi. Example Peptides

Disclosed are peptides comprising a monomeric Fc fragment of an immunoglobulin recognized by an FcRn; a SARS-CoV-2 soluble S protein; and a trimerization domain, wherein the SARS-CoV-2 soluble S protein is from an omicron strain. For example, disclosed are peptides comprising the amino acid sequence of

MFVFLVLLPLVSSQCVNLITRTQSYTNSFTRGVYYPDKVFRSSVLHSTQDLFLPFFSNVT
WFHAIHVSGTNGTKRFDNPALPFNDGVYFASTEKSNIIRGWIFGTTLDSKTQSLLIVNNA
TNVVIKVCEFQFCNDPFLDVYQKNNKSWMESEFRVYSSANNCTFEYVSQPFLMDLEGK
EGNFKNLREFVFKNIDGYFKIYSKHTPINLERDLPQGFSALEPLVDLPIGINITRFQTLLAL
HRSYLTPVDSSSGWTAGAAAYYVGYLQPRTFLLKYNENGTITDAVDCALDPLSETKCTL
KSFTVEKGIYQTSNFRVQPTESIVRFPNITNLCPFHEVFNATTFASVYAWNRKRISNCVAD
YSVIYNFAPFFAFKCYGVSPTKLNDLCFTNVYADSFVIRGNEVSQIAPGQTGNIADYYNYK
LPDDFTGCVIAWNSNKLDSKPSGNYNYLYRLFRKSKLKPFERDISTEIYQAGNKPCNGVA
GPNCYSPLQSYGFRPTYGVGHQPYRVVVLSFELLHAPATVCGPKKSTNLVKNKCVNFNF
NGLTGTGVLTESNKKFLPFQQFGRDIADTTDAVRDPQTLEILDITPCSFGGVSVITPGTNT
SNQVAVLYQGVNCTEVPVAIHADQLTPTWRVYSTGSNVFQTRAGCLIGAEYVNNSYECD

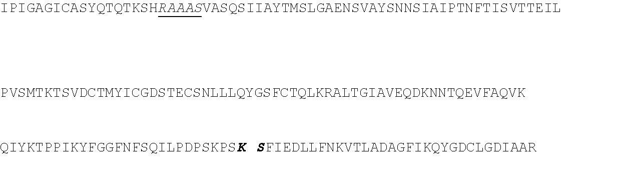

DLICAQKFNGLTVLPPLLTDEMIAQYTSALLAGTITSGWTFGAGAALQIPFAMQMAYRF
NGIGVTQNVLYENQKLIANQFNSAIGKIQDSLSSTASALGKLQDVVNHNAQALNTLVKQ

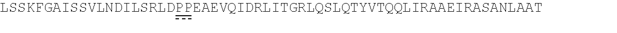

KMSECVLGQSKRVDFCGKGYHLMSFPQSAPHGVVFLHVTYVPAQEKNFTTAPAICHDG
KAHFPREGVFVSNGTHWFVTQRNFYEPQIITTDNTFVSGNCDVVIGIVNNTVYDPLQPEL
DSFKEELDKYFKNHTSPDVDLGDISGINASVVNIQKEIDRLNEVAKNLNESLIDLQELGK

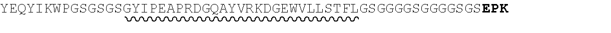

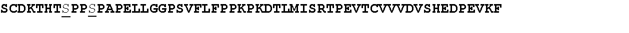

NWYVDGVEVHNAKTKPREEQYNSTYRVVSVLTVLHQDWLNGKEYKCAVSNKALP
APIEKTISKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHYTQKSLS
LSPGK (SEQ ID NO: 31)

or a variant thereof. In some aspects, the variant can be a sequence 50%, 55%, 65%, 70%, 75%, 80%, 90%, 95%, 96%, 97%, 98%, or 99% identical to the sequence of SEQ ID NO:31. The underlined sequence represents a native signal peptide of S protein. The bold sequence is human IgG1 Fc. The underlined bold sequence S represents a cysteine to serine mutation in human IgG1 to produce a monomer human IgG1. The shaded sequence is a 6GS linker. The squiggle underline represents the foldon domain from T4 fibrin. The underlined italicized RAAAS shows the mutated S1/S2 cleavage site (from RRARS to RAAAS). The bold italicized KAS shows the mutated S2′ cleavage site (from KRS to KAS. The bold underlined A in the IgG1 Fc shows a mutation preventing complement binding (K to A). The double underline in the S protein shows mutations keeping Pre-fusion conformation.

Disclosed are peptides comprising a monomeric Fc fragment of an immunoglobulin recognized by an FcRn; a SARS-CoV-2 soluble S protein; and a trimerization domain. For example, disclosed are peptides comprising the amino acid sequence of

(SEQ ID NO: 1)
MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQ
TLLALHRSYLTPGDSSSGWTAGAAAYYVGYLQPRTFLLKYNENGTITDAVDCALDPLS

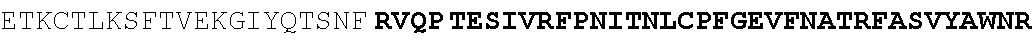

KRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERD
ISTEIYQAGSTPCNGVEGENCYFPLQSYGFQPTNGVGYQPYRVVVLSFELLHAPAT
VCGPKKSTNLVKNKCVNFNFNGLTGTGVLTESNKKFLPFQQFGRDIADTTDAVRDPQ
TLEILDITPCSFGGVSVITPGTNTSNQVAVLYQDVNCTEVPVAIHADQLTPTWRVYSTGS
NVFQTRAGCLIGAEHVNNSYECDIPIGAGICASYQTQTNSPRRAASVASQSIIAYTMSLG
AENSVAYSNNSIAIPTNFTISVTTEILPVSMTKTSVDCTMYICGDSTECSNLLLQYGSFCT
QLNRALTGIAVEQDKNTQEVFAQVKQIYKTPPIKDFGGFNFSQILPDPSKPSKASFIEDLL
FNKVTLADAGFIKQYGDCLGDIAARDLICAQKFNGLTVLPPLLTDEMIAQYTSALLAGTI
TSGWTFGAGAALQIPFAMQMAYRENGIGVTQNVLYENQKLIANQFNSAIGKIQDSLSST
ASALGKLQDVVNQNAQALNTLVKQLSSNFGAISSVLNDILSRLDPPEAEVQIDRLITGRL
QSLQTYVTQQLIRAAEIRASANLAATKMSECVLGQSKRVDFCGKGYHLMSFPQSAPHG
VVFLHVTYVPAQEKNFTTAPAICHDGKAHFPREGVFVSNGTHWFVTQRNFYEPQIITTD
NTFVSGNCDVVIGIVNNTVYDPLQPELDSFKEELDKYFKNHTSPDVDLGDISGINASVVN

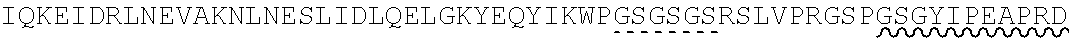

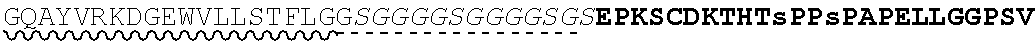

FLFPPKPKDTLMISRTPEVTCVVVDVSHEDPEVKFNWYVDGVEVHNAKTKPREEQ
YNSTYRVVSVLTVLHQDWLNGKEYKCαVSNKALPAPIEKTISKAKGQPREPQVYTL
PPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK

or a variant thereof. In some aspects, the variant can be a sequence 50%, 55%, 65%, 70%, 75%, 80%, 90%, 95%, 96%, 97%, 98%, or 99% identical to the sequence of SEQ ID NO:1. The underlined sequence represents a native signal peptide of S protein. The bold shaded sequence represents the RBD of S1. The bold underline sequence represents the mutated S1/S2 cleavage site (R685A of S protein in italics, no change in S686 of S protein). The bold letter and bold underline sequence represents a mutation at the S2′ cleavage site (R816A of S protein in italics, no change in S817 of S protein). The bold, italics, and shaded sequence represents K986P and V987P mutations (of the S protein) which allow the S protein to keep the Pre-fusion conformation. The dotted underline and shaded sequence represents a 6GS (glycine-serine) linker. The double squiggle underline represents the foldon domain from T4 fibrin. The dotted underline, italicized shaded sequence represents a 14GS (glycine-serine) linker. The bold sequence is human IgG1. The dotted underline lowercase sequence S represents a cysteine to serine mutation (C226S of human IgG1, Ser at position 1283 of SEQ ID NO:1) in human IgG1 to produce a monomer human IgG1. The dotted underline lowercase sequence S represents a cysteine to serine mutation (C229S of human IgG1, Ser at position 1286 of SEQ ID NO:1) in human IgG1 to produce a monomer human IgG1. The italicized, underlined lowercase sequence represents a mutation preventing complement binding (K322A of human IgG1, Ala at position 1379 of SEQ ID NO:1) in human IgG1. Amino acids 16 to 1213 represent the SARS-Cov-2 spike protein. Amino acids 1229 to 1257 represent the foldon domain of T4 fibrin. Amino acids 1273 to 1504 represent a monomeric Fc IgG1 fragment.

Disclosed are peptides comprising a monomeric Fc fragment of an immunoglobulin recognized by an FcRn; a SARS-CoV-2 S1 protein; and a trimerization domain. For example, disclosed are peptides comprising the amino acid sequence of

MFVFLVLLPLVSSQCVNLTTRTQLPPAYTNSFTRGVYYPDKVFRSSVLHSTQDLFLPFFS
NVTWFHAIHVSGTNGTKRFDNPVLPFNDGVYFASTEKSNIIRGWIFGTTLDSKTQSLLIV
NNATNVVIKVCEFQFCNDPFLGVYYHKNNKSWMESEFRVYSSANNCTFEYVSQPFLMD
LEGKQGNFKNLREFVFKNIDGYFKIYSKHTPINLVRDLPQGFSALEPLVDLPIGINITRFQ
TLLALHRSYLTPGDSSSSGWTAGAAAYYVGYLQPRTFLLKYNENGTITDAVDCALDPLS

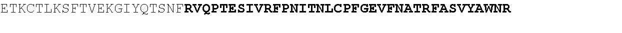

KRISNCVADYSVLYNSASFSTFKCYGVSPTKLNDLCFTNVYADSFVIRGDEVRQIAP
GQTGKIADYNYKLPDDFTGCVIAWNSNNLDSKVGGNYNYLYRLFRKSNLKPFERD
D
ISTEIYQAGSSTPCNGVEGFNCYFPLQSYGFQPTNGVGYQPYRVVVLSFELLHAPAT
VCGPKKSTNLVKNKCVNFNFNGLTGTGVLTESNKKFLPFQQFGRDIADTTDAVRDPQ
TLEILDITPCSFGGVSVITPGTNTSVQVAVLYQDVNCTEVPVAIHADQLTPTWRVYSTGS

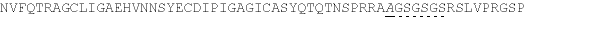

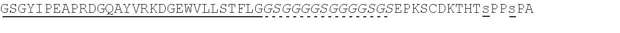

PELLGGPSVFLFPPKPKDTLMISRTPEVTCVVVDVSHEDPEVKFNWYVDGVEVHNAKT
KPREEQYNSSTYRVVSVLTVLHQDWLNGKEYKCaVSNKALPAPIEKTISKAKGQPREPQV
YTLPPSRDELTKNQVSLTCLVKGFYPSDIAVEWESNGQPENNYKTTPPVLDSDGSFFLYS
KLTVDKSRWQQGNVFSCSVMHEALHNHYTQKSLSLSPGK (SEQ ID NO: 3)

or a variant thereof. In some aspects, the variant can a sequence 50%, 55%, 65%, 70%, 75%, 80%, 90%, 95%, 96%, 97%, 98%, or 99% identical to the sequence of SEQ ID NO:3. The underlined sequence represents a native signal peptide of S protein. The bold shaded sequence represents the RBD of S1. The bold underline sequence represents the mutated S1/S2 cleavage site (R685A in italics). The dotted underline and shaded sequence represents a 6GS (glycine-serine) linker. The double squiggle underline represents the foldon domain from T4 fibrin. The dotted underline, italicized shaded sequence represents a 14GS (glycine-serine) linker. The bold sequence is human IgG1. The dotted underline lowercase sequence S represents a cysteine to serine mutation (C226S of human IgG1, Ser at position 755 of SEQ ID NO:3) in human IgG1 to produce a monomer human IgG1. The dotted underline lowercase sequence S represents a cysteine to serine mutation (C229S of human IgG1, Ser at position 758 of SEQ ID NO:3) in human IgG1 to produce a monomer human IgG1. The italicized, underlined lowercase sequence represents a mutation preventing complement binding (K322A of human IgG1, Ala at position 851 of SEQ ID NO:3) in human IgG1. Amino acids 16 to 685 represent the SARS-Cov-2 S1 protein. Amino acids 701 to 729 represent the foldon domain of T4 fibrin. Amino acids 745 to 976 represent a monomeric Fc IgG1 fragment.

Disclosed are peptides comprising a monomeric Fc fragment of an immunoglobulin recognized by a FcRn; a SARS-CoV-2 RBD protein; and a trimerization domain. For example, disclosed are peptides comprising the amino acid sequence of

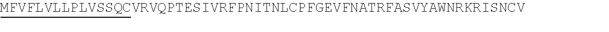

ADYSVLYNSASFSTFKCYGVSPTKLNDLCFTNVYADSFVIRGDEVRQIAPGQTGKIADY
NYKLPDDFTGCVIAWNSNNLDSKVGGNYNYLYRLFRKSNLKPFERDISTEIYQAGSTPC
NGVEGFNCYFPLQSYGFQPTNGVGYQPYRVVVLSFELLHAPATVCGPKKSTNLVKNKC

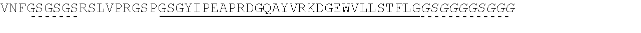

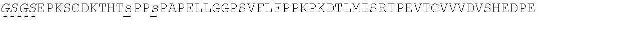

VKFNWYVDGVEVHNAKTKPREEQYNSTYRVVSVLTVLHQDWLNGKEYKCaVSNKAL
PAPIEKTISKAKGQPREPQVYTLPPSRDELTKNQVSLTCLVKGFYPSDIAVEWESNGQPE
NNYKTTPPVLDSDGSFFLYSKLTVDKSRWQQGNVFSCSVMHEALHNHYTQKSLSLSPG
K (SEQ ID NO: 5)

or a variant thereof. In some aspects, the variant can a sequence 50%, 55%, 65%, 70%, 75%, 80%, 90%, 95%, 96%, 97%, 98%, or 99% identical to the sequence of SEQ ID NO:5. The underlined sequence represents a native signal peptide of S protein. The bold shaded sequence represents the RBD of S1. The dotted underline and shaded sequence represents a 6GS (glycine-serine) linker. The double squiggle underline represents the foldon domain from T4 fibrin. The dotted underline, italicized shaded sequence represents a 14GS (glycine-serine) linker. The bold sequence is human IgG1. The dotted underline lowercase sequence S represents a cysteine to serine mutation (C226S of human IgG1, Ser at position 309 of SEQ ID NO:5) in human IgG1 to produce a monomer human IgG1. The dotted underline lowercase sequence S represents a cysteine to serine mutation (C229S of human IgG1, Ser at position 312 of SEQ ID NO:5) in human IgG1 to produce a monomer human IgG1. The italicized, underlined lowercase sequence represents a mutation preventing complement binding (K322A of human IgG1, Ala at position 405 of SEQ ID NO:5) in human IgG1. Amino acids 17 to 239 represent the SARS-Cov-2 RBD protein. Amino acids 255 to 283 represent the foldon domain of T4 fibrin. Amino acids 299 to 530 represent a monomeric Fc IgG1 fragment.

Disclosed are peptides comprising a monomeric Fc fragment of an immunoglobulin recognized by a FcRn; a SARS-CoV-2 S protein comprising 6P mutations; and a trimerization domain. For example, disclosed are peptides comprising the amino acid sequence of

MFVFLVLLPLVSSQCVNLITRTQSYTNSFTRGVYYPDKVFRSSVLHSTQDLFLPFFSNVT
WFHAIHVSGTNGTKRFDNPALPFNDGVYFASTEKSNIIRGWIFGTTLDSKTQSLLIVNNA
TNVVIKVCEFQFCNDPFLDVYQKNNKSWMESEFRVYSSANNCTFEYVSQPFLMDLEGK
EGNFKNLREFVFKNIDGYFKIYSKHTPINLERDLPQGFSALEPLVDLPIGINITRFQTLLAL
HRSYLTPVDSSSGWTAGAAAYYVGYLQPRTFLLKYNENGTITDAVDCALDPLSETKCT
LKSFTVEKGIYQTSNFRVQPTESIVRFPNITNLCPFHEVFNATTFASVYAWNRKRISNCV
ADYSVIYNFAPFFAFKCYGVSPTKLNDLCFTNVYADSFVIRGNEVSQIAPGQTGNIADYN
YKLPDDFTGCVIAWNSNKLDSKPSGNYNYLYRLFRKSKLKPFERDISTEIYQAGNKPCN
GVAGPNCYSPLQSYGFRPTYGVGHQPYRVVVLSFELLHAPATVCGPKKSTNLVKNKCV
NFNFNGLTGTGVLTESNKKFLPFQQFGDIADTTDAVRDPQTLEILDITPCSFGGVSVITP
GTNTSNQVAVLYQGVNCTEVPVAIHADQLTPTWRVYSTGSNVFQTRAGCLIGAEYVNN
SYECDIPIGAGICASYQTQTKSHRAAASVASWSIIAYTMSLGAENSVAYSNNSIAIPTNFTI
SVTTEILPVSMTKTSVDCTMYICGDSTECSNLLLQYGSFCTQLKRALTGIAVEQDKNTQE
VFAQVKQIYKTPPIKYFGGFNFSQILPDPSKPSKASPIEDLLFNKVTLADAGFIKQYGDC
LGDIAARDLICAQKFNGLTVLPPLLTDEMIAQYTSALLAGTITSGWTFGAG[P]ALQIPF[P]
MQMAYRFNGIGVTQNVLYENQKLIANQFNSAIGKIQDSLSST[P]SALGKLQDVVNHNA
QALNTLVKQLSSKFGAISSVLNDILSRLD[PP]EAEVQIDRLITGRLQSLQTYVTQQLIRAA
EIRASANLAATKMSECVLGQSKRVDFCGKGYHLMSFPQSAPHGVVFLHVTYVPAQEKN
FTTAPAICHDGKAHFPREGVFVSNGTHWFVTQRNFYEPQIITTDNTFVSGNCDVVIGIVN
NTVYDPLQPELDSFKEELDKYFKNHTSPDVDLGDISGINASVVNIQKEIDRLNEVAKNLN

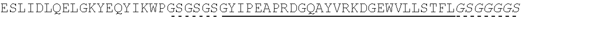

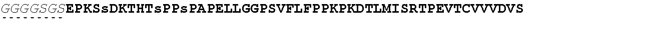

HEDPEVKFNWYVDGVEVHNAKTKPREEQYNSTYRVVSVLTVLHQDWLNGKEYK
CA
AVEWESNGQPENNYKTTPPVLDSDGSFFLYSSKLTVDKSRWQQGNVFSCSVMHEAL
HNHYTQKSLSLSPGK (SEQ ID NO: 52)

or a variant thereof. In some aspects, the variant can be a sequence 50%, 55%, 65%, 70%, 75%, 80%, 90%, 95%, 96%, 97%, 98%, or 99% identical to the sequence of SEQ ID NO:52. The underlined sequence represents a native signal peptide of S protein. The dotted underline sequence represents a 6GS (glycine-serine) linker. The double squiggle underline represents the foldon domain from T4 fibritin. The dotted underline, italicized sequence represents a 14GS (glycine-serine) linker. The bold sequence is human IgG1 fragment. The lowercase sequence S within the IgG1 represents a cysteine to serine mutation (C220S, C226S, C229S of human IgG1, Ser at positions 5, 11 and 14 of the IgG1 fragment herein) in human IgG1 to produce a monomer human IgG1. The [P] sequences represent the six mutations keeping Pre-fusion conformation (F817P, A892P, A899P, A942P, k986P, V987P). The underlined sequence with two bold A's represent mutations in the S1/S2 cleavage site (from RRARS to RAAAS, ie, R683A, R685A). The bold double underlined A represents a mutated S2′ cleavage site (from KRS to KAS, ie, R815A). The double underlined A in the IgG1 fragment represents a mutation preventing complement binding (K322A). Amino acids 1 to 1209 represent the SARS-Cov-2 S protein. Amino acids 1216 to 1243 represent the foldon domain of T4 fibrin. Amino acids 1258 to 1489 represent a monomeric Fc IgG1 fragment.
2. Peptides without a Trimerization Domain

Disclosed are peptides comprising an Fc fragment of an immunoglobulin recognized by an FcRn and a coronavirus antigen. In some aspects, the coronavirus antigen can be any coronavirus spike protein, or antigenic fragment thereof. In some aspects, the coronavirus is Middle East respiratory syndrome coronavirus (MERS-CoV), Human Coronavirus—Erasmus Medical Centre (HCoV-EMC), SARS-CoV, or SARS-CoV-2. Thus, in some aspects, the coronavirus spike protein can be a MERS-CoV, HCoV-EMC, SARS-CoV, or SARS-CoV-2 spike protein, or antigenic fragment thereof. In some aspects, the peptides do not comprise a trimerization domain.

Disclosed are peptides comprising a Fc fragment of an immunoglobulin recognized by a FcRn and a SARS-CoV-2 antigen. In some aspects, the SARS-CoV-2 antigen can be a SARS-CoV-2 spike protein. Thus, disclosed are peptides comprising a Fc fragment of an immunoglobulin recognized by a FcRn and a SARS-CoV-2 spike protein. In some aspects, the peptides do not comprise a trimerization domain.

Disclosed are peptides comprising a Fc fragment of an immunoglobulin recognized by a FcRn and a SARS-CoV-2 RBD protein. Thus, disclosed are peptides comprising a Fc fragment of an immunoglobulin recognized by a FcRn and a SARS-CoV-2 RBD protein.

In some instances, the Fc fragment of an immunoglobulin recognized by a FcRn is conjugated to the amino or carboxy terminal end of a coronavirus antigen. The conjugation can be direct or indirect. Indirect conjugation can be due to the presence of a linker, for example, a linker can be present in between the coronavirus antigen and the Fc fragment of an immunoglobulin recognized by a FcRn. In some aspects, the peptides do not comprise a trimerization domain.

Disclosed are peptides encoded by one or more of the nucleic acid sequences provided herein.

i. Fe Fragment

An Fc fragment of an immunoglobulin recognized by FcRn, as disclosed herein, can be any Fc fragment that can be recognized by an FcRn and is capable of forming a dimeric structure. In some aspects, a Fc fragment of an immunoglobulin recognized by a FcRn can comprise only the Fc portion of an immunoglobulin. In some aspects, a Fc fragment of an immunoglobulin recognized by a FcRn can be a Fc fragment of an immunoglobulin G recognized by a FcRn.

In some aspects, unlike the monomeric Fc fragment of an immunoglobulin recognized by a FcRn, the Fc fragment of an immunoglobulin recognized by a FcRn capable of forming a dimeric structure retains the cysteine residues responsible for dimer formation in native IgG. For example, positions 226 and 229 of the full length sequence of the wild type sequence of human IgG1 are not mutated and thus retain the ability for dimer formation. In some aspects, positions 11 and 14 of a sequence comprising only the hinge region, CH2 and CH3 domains of wild type IgG are not mutated. For example, the cysteine residues at positions 11 and 14 of SEQ ID NO:17 are not mutated. In some aspects, positions 11 and 14 of SEQ ID NO:7 are located in the hinge region of Fc fragments of an immunoglobulin recognized by a FcRn that retains the ability to form dimers. In some aspects, the cysteine residues at positions 5, 11, and 14 of SEQ ID NO:7, which can correspond to positions 220, 226 and 229 of full-length human wild-type IgG1, are not mutated.

In some aspects, the C1q binding site can be ablated in the Fc fragment that retains the ability for dimer formation. This can be effective to help avoid clearance of the Fc fragments via the complement pathway and thus allowing the disclosed peptides comprising a Fc fragment and coronavirus antigen to remain in a subject and provide its therapeutic effect. In some aspects, C1q is known to bind to the CH2 domain of an immunoglobulin, particularly IgG. In some aspects, substituting the lysine at position 322 of wild type human IgG can ablate or eliminate the complement C1q binding site. For example, replacing Lys322 of full-length human IgG with an Ala residue can ablate or eliminate the complement C1q binding site. In some aspects, replacing one or more of Glu318, Lys320, and Lys322 of full-length mouse IgG with an Ala residue can ablate or eliminate the complement C1q binding site. In some aspects, ablating C1q binding to the disclosed monomeric Fc fragments comprises mutation position 107 of a Fc fragment of an immunoglobulin recognized by an FcRn that retains the ability for dimer formation. For example, a mutation of lysine to alanine shown at position 107 of SEQ ID NO:17 can ablate C1q binding to a human Fc fragment of an immunoglobulin recognized by a FcRn.

In some aspects, the FcRn binding sites are known to be His310 and His433 or His310/Gln311 (HQ) and His433/Asn434 (HN) of full-length wild-type IgG. The region of the Fc-fragment of IgG that binds to the FcRn receptor in humans has been described based upon X-ray crystallography (Burmaister, W. P. et al., Nature, 1994; 372:379-378; incorporated by reference in its entirety herein). The major contact area of Fc with the FcRn receptor is near the junction of the CH2 and CH3 domains. Potential contacts are residues 248, 250-257, 272, 285, 288, 290-291, 308-311, and 314 in CH2 and 385-387, 428 and 433-436 in CH3 of wild-type IgG. In some aspects, no mutations would be present in the FcRn binding sites. Given the foregoing information, those of ordinary skill in the art will readily recognize that the monomeric Fc fragment of IgG can be modified according to well-recognized procedures such as site-directed mutagenesis and the like to yield modified monomeric Fc fragments or portions thereof that will be bound by the FcRn receptor. Such modifications include modifications remote from the FcRn contact sites as well as modifications within the contact sites that preserve or even enhance binding.

In some aspects, the Fc fragment of an immunoglobulin recognized by a FcRn that retains the ability for dimer formation can be derived from any isotype that binds FcRn. The Fc-fragment should be chosen from an immunoglobulin known to bind the FcRn in the mucosa of the subject receiving the antigen-Fc vaccine. Immunoglobulin subclasses recognized by FcRn in different epithelial mucosa of animal subjects are known to a person in the art and can be found in Ober, R. J. et al, 2001, Int. Immunol. 13, 1551-9, incorporated by reference in its entirety herein. In some aspects, the Fc fragment of an immunoglobulin recognized by a FcRn is derived from a mammalian immunoglobulin. For example, the Fc fragment of an immunoglobulin recognized by a FcRn can be a human immunoglobulin sequence.

In some aspects, the amino acid sequence of a Fc fragment of a human IgG1 that retains the ability for dimer formation can be EPKSCDKTHTCPPCPAPELLGGPSVFLFPPKPKDTLMISRTPEVTCVVVDVSHEDPEVKF NWYVDGVEVHNAKTKPREEQYNSTYRVVSVLTVLHQDWLNGKEYKCaVSNKALPAPI EKTISKAKGQPREPQVYTLPPSRDELTKNQVSLTCLVKGFYPSDIAVEWESNGQPENNY KTTPPVLDSDGSFFLYSKLTVDKSRWQQGNVFSCSVMHEALHNHYTQKSLSLSPGK (SEQ ID NO:17) or a variant thereof. In some aspects, the variant can a sequence 50%, 55%, 65%, 70%, 75%, 80%, 90%, 95%, 96%, 97%, 98%, or 99% identical to the sequence of SEQ ID NO:17. The two cysteine residues at positions 11 and 14 help retain the ability for dimer formation. A lysine to alanine mutation is shown at position 107. The lysine to alanine mutation ablates C1q binding to the Fc fragment.

In some aspects, the amino acid sequence of a Fc fragment of an immunoglobulin recognized by a FcRn that retains the ability for dimer formation of a mouse IgG2a can be EPRGPTIKPCPPCKSPAPNLLGGPSVFIFPPKIKDVLMISLSPIVTCVVVDVSEDDPDVQIS WFVNNVEVHTAQTQTHREDYNSTLRVVSALPIQHQDWMSGKAFACAVNNKDLPAPIE RTISKPKGSVRAPQVYVLPPPEEEMTKKQVTLTCMVTDFMPEDIYVEWTNNGKTELNY KNTEPVLDSDGSYFMYSKLRVEKKNWVERNSYSCSVVHEGLHNHHTTKSFSRTPGK (SEQ ID NO:18) or a sequence 50%, 55%, 65%, 70%, 75%, 80%, 90%, 95%, 96%, 97%, 98%, or 99% identical to the sequence of (SEQ ID NO:18).

In some aspects, the Fc fragment of an immunoglobulin recognized by an FcRn that retains the ability for dimer formation comprises a full-length Fc region of an immunoglobulin. In some aspects, the monomeric Fc fragment of an immunoglobulin recognized by a FcRn comprises at least the CH2 and CH3 domains of a Fc region of an immunoglobulin. For example, the monomeric Fc fragment of an immunoglobulin recognized by a FcRn comprises one or more of a full length CH2 and CH3 domain of IgG. In some aspects, the monomeric Fc fragment of an immunoglobulin recognized by an FcRn comprises at least a portion of the one or more CH2 and CH3 domains so long as the portions of the one or more CH2 and CH3 domains retains the ability to be recognized by FcRn.

In some aspects, the monomeric Fc fragment of an immunoglobulin recognized by a FcRn is conjugated to the amino or carboxy terminal end of a SARS-CoV-2 antigen. For example, the SARS-CoV-2 antigen can be the spike protein or a fragment thereof, such as RBD. The conjugation can be direct or indirect. Indirect conjugation can be due to the presence of a linker in between the SARS-CoV-2 antigen and the Fc fragment of an immunoglobulin recognized by a FcRn. Indirect conjugation can be due to the presence of another peptide in between the SARS-CoV-2 antigen and the Fc fragment of an immunoglobulin recognized by a FcRn.

In some aspects, the Fc fragment of an immunoglobulin recognized by a FcRn that retains the ability for dimerization can be derived from IgG. In some aspects, the IgG can be any IgG subtype. For example, the monomeric Fc fragment of an immunoglobulin recognized by a FcRn can be derived from IgG1, IgG2, IgG3, or IgG4.

ii. Coronavirus Antigen

In some aspects, the disclosed peptides can comprise a Fc fragment of an immunoglobulin recognized by a FcRn and a coronavirus antigen. In some aspects, a coronavirus antigen can be any region of a coronavirus that can generate an immune response. In some aspects, a coronavirus antigen can be all or a portion of the coronavirus spike (S) protein. In some aspects, the coronavirus S protein is the soluble portion of the coronavirus S protein. For example, the transmembrane domain and cytoplasmic domain are not present in the soluble portion of the coronavirus S protein. In some aspects, the coronavirus is Middle East respiratory syndrome coronavirus (MERS-CoV), Human Coronavirus-Erasmus Medical Centre (HCoV-EMC), SARS-CoV, or SARS-CoV-2. Thus, in some aspects, the coronavirus spike protein can be a MERS-CoV, HCoV-EMC, SARS-CoV, or SARS-CoV-2 spike protein, or antigenic fragment thereof.

In some aspects, the disclosed peptides can comprise a Fc fragment of an immunoglobulin recognized by a FcRn and a SARS-COV-2 antigen. In some aspects, a SARS-COV-2 antigen can be any region of SARS-COV-2 that can generate an immune response. In some aspects, a SARS-COV-2 antigen can be all or a portion of the SARS-COV-2 S protein. In some aspects, the SARS-COV-2 S protein is the soluble portion of the SARS-COV-2 S protein. For example, the transmembrane domain and cytoplasmic domain are not present in the soluble portion of the SARS-COV-2 S protein.

In some aspects, a SARS-CoV-2 S protein can be derived from wild type SARS-CoV-2 or from a variant strain, such as, but not limited to, the variants of D614G (originally found in China/Germany), B.1.1.7 or 20I/501Y.V1 (originally found in the United Kingdom), B.1.351 or 20H/501.V2 (originally found in South Africa), P.1 or 20J/501Y.V3 (originally found in Japan/Brazil), 20C/S:452R (originally found in California), and Cluster 5 Variant (originally found in Denmark) XBB.1.5 (e.g. 1.5.70, 1.5.68, 1.5.72, 1.5.10, 1.5.59, 1.5.1), XBB.1.16 (e.g. 1.16.6, 1.16.11, 1.16.15, 1.6.1), EG.5, XBB.1.9 (e.g. 1.9.1, 1.9.2), XBB.2.3, BA.1, BA.5, BA.2.86, HV.1, FL.1.5.1, HK.3, JD.1.1, JF.1, GK.1.1., HF.1, BA.2 (e.g. 2.12.1, 2.12.2), BA.4, 13A.5, BA.527, BA.529, GE.1, XB3, GK.2, EG.6.1, XBB.1.42.2, CH.1.1, XBB.2.3.8, FD.1.1, FE.1.1, EU.1.1, B.1.1.529, B.1.617.2, XBB.128, BQ.1 (e.g. 1.1). In some aspects, a SARS-CoV-2 S protein can be derived from any of the SARS-CoV-2 strains of α, β, γ, δ, or ε (original or subvariant strains).

In some aspects, SARS-CoV-2 S protein can have mutations that stabilize the S antigen in the prefusion form. In some aspects, the SARS-CoV-2 S protein can have at least one, two, three, four, five, or six mutations that stabilize the S antigen in the prefusion form. In some aspects, the SARS-CoV-2 S protein can at least three mutations that stabilize the S antigen in the prefusion form. In some aspects, the at least three mutations in the SARS-CoV-2 S antigen correspond to at least three of positions Phe 817, Ala 892, Ala 899, Ala 942, Lys 986, or Val 987 of the SARS-CoV-2 S antigen sequence (SEQ ID NO:8). In some aspects, the mutations can be any amino acid substituted for a proline. In some aspects, the chemical properties of proline discourage helix formation and promote local rigidity in a polypeptide chain. In some aspects, the helix structure can be used by the postfusion of a viral membrane protein for initiating fusion with the cell membrane. Thus, in some aspects, any substitution that prevents helix formation can be used. Thus, in some aspects, the SARS-CoV-2 S protein can have at least one, two, three, four, five, or six prolines not present in the wild type SARS-CoV-2 S protein. In some aspects, a SARS-CoV-2 S protein having two proline mutations can comprise proline mutations at Lys 986 or Val 987 of the SARS-CoV-2 S antigen sequence (SEQ ID NO:8). In some aspects, a SARS-CoV-2 S protein having four proline mutations can comprise proline mutations at F817P, A892P, A899P, and A942P of the SARS-CoV-2 S antigen sequence (SEQ ID NO:8). In some aspects, a SARS-CoV-2 S protein having six proline mutations can comprise proline mutations at F817P, A892P, A899P, A942P, K986P, and V987P of the SARS-CoV-2 S antigen sequence (SEQ ID NO:8). In some aspects, a SARS-CoV-2 S protein having at least two, three, four, five, or six mutations can have a combination of any of the mutations at F817P, A892P, A899P, A942P, K986P, and V987P.

In some aspects, the soluble portion of the SARS-COV-2 S protein is amino acids 1-1213 of the full length wild type S protein. Specifically, the soluble portion of the SARS-COV-2 S protein comprises the sequence MFVFLVLLPLVSSQCVNLTTRTQLPPAYTNSFTRGVYYPDKVFRSSVLHSTQDLFLPFFS NVTWFHAIHVSGTNGTKRFDNPVLPFNDGVYFASTEKSNIIRGWIFGTTLDSKTQSLLIV NNATNVVIKVCEFQFCNDPFLGVYYHKNNKSWMESEFRVYSSANNCTFEYVSQPFLMD LEGKQGNFKNLREFVFKNIDGYFKIYSKHTPINLVRDLPQGFSALEPLVDLPIGINITRFQ TLLALHRSYLTPGDSSSGWTAGAAAYYVGYLQPRTFLLKYNENGTITDAVDCALDPLS ETKCTLKSFTVEKGIYQTSNFRVQPTESIVRFPNITNLCPFGEVFNATRFASVYAWNR KRISNCVADYSVLYNSASFSTFKCYGVSPTKLNDLCFTNVYADSFVIRGDEVRQIAP GQTGKIADYNYKLPDDFTGCVIAWNSNNLDSKVGGNYNYLYRLFRKSNLKPFERD ISTEIYQAGSTPCNGVEGFNCYFPLQSYGFQPTNGVGYQPYRVVVLSFELLHAPAT VCGPKKSTNLVKNKCVNFNFNGLTGTGVLTESNKKFLPFQQFGRDIADTTDAVRDPQ TLEILDITPCSFGGVSVITPGTNTSNQVAVLYQDVNCTEVPVAIHADQLTPTWRVYSTGS NVFQTRAGCLIGAEHVNNSYECDIPIGAGICASYQTQTNSPRRAASVASQSIIAYTMSLG AENSVAYSNNSIAIPTNFTISVTTEILPVSMTKTSVDCTMYICGDSTECSNLLLQYGSFCT QLNRALTGIAVEQDKNTQEVFAQVKQIYKTPPIKDFGGFNFSQILPDPSKPSKASFIEDLL FNKVTLADAGFIKQYGDCLGDIAARDLICAQKFNGLTVLPPLLTDEMIAQYTSALLAGTI TSGWTFGAGAALQIPFAMQMAYRFNGIGVTQNVLYENQKLIANQFNSAIGKIQDSLSST ASALGKLQDVVNQNAQALNTLVKQLSSNFGAISSVLNDILSRLDPPEAEVQIDRLITGRL QSLQTYVTQQLIRAAEIRASANLAATKMSECVLGQSKRVDFCGKGYHLMSFPQSAPHG VVFLHVTYVPAQEKNFTTAPAICHDGKAHFPREGVFVSNGTHWFVTQRNFYEPQIITTD NTFVSGNCDVVIGIVNNTVYDPLQPELDSFKEELDKYFKNHTSPDVDLGDISGINASVVN IQKEIDRLNEVAKNLNESLIDLQELGKYEQYIKWP (SEQ ID NO:8) or a variant thereof. In some aspects, the variant can be a sequence 50%, 55%, 65%, 70%, 75%, 80%, 90%, 95%, 96%, 97%, 98%, or 99% identical to the sequence of SEQ ID NO:8. The bold sequence represents the RBD of S1.

In some aspects, the SARS-COV-2 S protein is the soluble portion of the D614G variant S protein. Specifically, the S protein of the D614G variant can comprise the sequence of SEQ ID NO:11 (with the mutation of D614G shown in lowercase) or a variant thereof. In some aspects, the variant can be a sequence 50%, 55%, 65%, 70%, 75%, 80%, 90%, 95%, 96%, 97%, 98%, or 99% identical to the sequence of SEQ ID NO:11. In some aspects, the SARS-COV-2 S protein is the RBD portion of the D614G variant S protein.

In some aspects, the SARS-COV-2 S protein is the soluble portion of the B.1.1.7 variant S protein. Specifically, the S protein of the B.1.1.7 variant can comprise the sequence of SEQ ID NO:12 or a variant thereof. In some aspects, the variant can be a sequence 50%, 55%, 65%, 70%, 75%, 80%, 90%, 95%, 96%, 97%, 98%, or 99% identical to the sequence of SEQ ID NO:12. In some aspects, the SARS-COV-2 S protein is the RBD portion of the B.1.1.7 variant S protein.

In some aspects, the SARS-COV-2 S protein is the soluble portion of the B.1.351 variant S protein. Specifically, the S protein of the B.1.351 variant can comprise the sequence of SEQ ID NO:13 or a variant thereof. In some aspects, the variant can be a sequence 50%, 55%, 65%, 70%, 75%, 80%, 90%, 95%, 96%, 97%, 98%, or 99% identical to the sequence of SEQ ID NO:13. In some aspects, the SARS-COV-2 S protein is the RBD portion of the B.1.351 variant S protein.

In some aspects, the SARS-COV-2 S protein is the soluble portion of the P.1 variant S protein. Specifically, the S protein of the P.1 variant can comprise the sequence of SEQ ID NO:14 or a variant thereof. In some aspects, the variant can be a sequence 50%, 55%, 65%, 70%, 75%, 80%, 90%, 95%, 96%, 97%, 98%, or 99% identical to the sequence of SEQ ID NO:14. In some aspects, the SARS-COV-2 S protein is the RBD portion of the P.1 variant S protein.

In some aspects, the SARS-COV-2 S protein is the soluble portion of the 20C/S:452R variant S protein. Specifically, the S protein of the 20C/S:452R variant can comprise the sequence of SEQ ID NO:15 or a variant thereof. In some aspects, the variant can be a sequence 50%, 55%, 65%, 70%, 75%, 80%, 90%, 95%, 96%, 97%, 98%, or 99% identical to the sequence of SEQ ID NO:15. In some aspects, the SARS-COV-2 S protein is the RBD portion of the 20C/S:452R variant S protein.

In some aspects, the SARS-COV-2 S protein is the soluble portion of the cluster 5 variant S protein. Specifically, the S protein of the cluster 5 variant can comprise the sequence of SEQ ID NO:16 or a variant thereof. In some aspects, the variant can be a sequence 50%, 55%, 65%, 70%, 75%, 80%, 90%, 95%, 96%, 97%, 98%, or 99% identical to the sequence of SEQ ID NO:16. In some aspects, the SARS-COV-2 S protein is the RBD portion of the cluster 5 variant S protein.

In some aspects, the SARS-COV-2 S protein is the soluble portion of the omicron XBB 1.5 S protein. Specifically, the S protein of the omicron strain can comprise the sequence of SEQ ID NO:30 or a variant thereof. In some aspects, the variant can be a sequence 50%, 55%, 65%, 70%, 75%, 80%, 90%, 95%, 96%, 97%, 98%, or 99% identical to the sequence of SEQ ID NO:30. In some aspects, SEQ ID NO:30 is the soluble portion of omicron S protein.

In some aspects, the SARS-COV-2 S protein can be cleaved into S1 and S2 subunits by proteases. In some aspects, S1 comprises the receptor-binding domain (RBD) which allows viruses to directly bind to the ACE2 receptor. In some aspects, S2 can mediate membrane fusion, with the help of a protease, in cells. In some aspects, the SARS-COV-2 S protein (“S protein”) is the full length soluble S protein, the S1 subunit, the S2 subunit, or the RBD. In some aspects, the SARS-COV-2 S protein is a portion of full length soluble S protein, the S1 subunit, the S2 subunit, or the RBD. In some aspects, the SARS-COV-2 S protein is a variant of a wild type sequence and thus, is 50, 55, 60, 65, 70, 75, 80, 85, 90, 95, 96, 97, 98, 99, or 100% identical to the wild type full length S protein, the S1 subunit, the S2 subunit, or the RBD. In some aspects, a variant SARS-COV-2 S protein can comprise a modified amino acid or a non-naturally occurring amino acid.

In some aspects, the complete wild type amino acid sequence of SARS-COV-2 can be found in Genbank as accession number MN908947. The S protein is nucleic acids 21563-25384 of accession number MN908947.

In some aspects, the S protein is the full length S protein. Because the S protein can be cleaved by proteases, in some aspects, the disclosed SARS-COV-2 S protein can be altered or mutated to remove the cleavage sites and produce a non-cleavable S protein. In some aspects, the mutations that remove the cleavage site are R685A and R816A of the full length wild type S protein. For example, the cleavage sites of R685A and R816A are at positions 685 and 816, respectively, of SEQ ID NO:8.

In some aspects, the S protein can be further altered or mutated so that the S protein retains its prefusion state. In some aspects, mutations that maintain the S protein in a prefusion state can be K986P and V987P.

iii. Linkers

Disclosed are peptides comprising a Fc fragment of an immunoglobulin recognized by FcRn that retains the ability for dimer formation and a SARS-CoV-2 antigen, wherein the peptide further comprises one or more linkers.

In some instances, at least one of the one or more linkers is on the N-terminus end of the Fc fragment of an immunoglobulin recognized by a FcRn. In some instances, at least one of the one or more linkers is on the C-terminus end of the Fc fragment of an immunoglobulin recognized by an FcRn.

In some instances, at least one of one or more linkers is located between the SARS-CoV-2 antigen and the Fc fragment of an immunoglobulin recognized by a FcRn.

In some instances, the one or more linkers are small, nonpolar, amino acid linkers. For example, the linker can be a GS-linker. The number of glycine, serine, and glycine/serine repeats can vary in the one or more linkers. Examples of GS linkers can be GSGSGS and GSGGGGSGGGGSGS.

iv. Additional Elements

In some aspects, the disclosed peptides comprise a signal peptide. In some aspects, a signal peptide is any short peptide (about 10-30 amino acids) that helps translocate the peptide to the cell membrane. In some aspects, the signal peptide is present on the N-terminal end of the SARS-CoV-2 antigen (e.g. RBD protein). In some aspects, the signal peptide is derived from the coronavirus antigen. In some aspects, the signal peptide is derived from the SARS-CoV-2 antigen. For example, the native signal peptide found on the SARS-CoV-2 S protein can be present in the disclosed peptides. In some aspects, the native signal peptide can comprise the amino acid sequence of MFVFLVLLPLVSSQC from the SARS-CoV-2 S protein. In some aspects, a signal peptide can comprise one or more of the sequences present in Table 1.

In some aspects, the peptides disclosed herein can further comprise an adjuvant. In some aspects, the adjuvant is immunostimulatory oligonucleotides containing unmethylated CpG dinucleotides (“CpG”). CpGs are known in the art as being adjuvants when administered by both systemic and mucosal routes (WO 96/02555, EP 468520, Davis et al., J. Immunol, 1998, 160(2): 870-876, McCluskie and Davis, J. Immunol., 1998, 161(9): 4463-6). CpG is an abbreviation for cytosineguanosinc dinucicotide motifs present in DNA. Historically, it was observed that the DNA fraction of BCG could exert an anti-tumour effect. In further studies, synthetic oligonucleotides derived from BCG gene sequences were shown to be capable of inducing immunostimulatory effects (both in vitro and in vivo). The authors of these studies concluded that certain palindromic sequences, including a central CG motif, carried this activity. The central role of the CG motif in immunostimulation was later elucidated in a publication by Krieg, 1995, Nature 374, p. 546. Detailed analysis has shown that the CG motif has to be in a certain sequence context, and that such sequences are common in bacterial DNA but are rare in vertebrate DNA. The immunostimulatory sequence is often: Purine, Purine, C, G, pyrimidine, pyrimidine; wherein the dinucleotide CG motif is not methylated, but other unmethylated CpG sequences are known to be immunostimulatory and may be used in the present invention.

In some aspects, disclosed are various mutations in one or more of the components of the disclosed compositions. In some aspects, mutations in one or more of the components can be one or more mutations in the monomeric Fc fragment of an immunoglobulin recognized by an FcRn, SARS-CoV-2 antigen (e.g. S protein), or trimerization domain. For example, certain aspects and mutations are described in US Application Publication Nos. US2019/0060440 (now U.S. Pat. No. 11,376,317), US2019/0359655 (now U.S. Pat. No. 11,591,371), and US2022/0098242, each of which are hereby incorporated by reference for their respective teachings.

### D. Peptide Complexes

Disclosed are peptide complexes comprising three of the disclosed peptides. Thus, disclosed are trimers, comprising three of the disclosed peptides. For example, disclosed are peptide complexes comprising three peptides, wherein each of the three peptides comprises a monomeric Fc fragment of an immunoglobulin recognized by a FcRn; a coronavirus antigen; and a trimerization domain.

Also disclosed are peptide complexes comprising three peptides, wherein each of the three peptides comprises a monomeric Fc fragment of an immunoglobulin recognized by a FcRn; SARS-CoV-2 antigen; and a trimerization domain.

In some aspects, the peptide complexes are formed when the trimerization domain of the disclosed peptides causes trimerization. Thus, the three peptides can oligomerize at the trimerization domain.

In some aspects, disclosed are peptide complexes comprising three monomeric Fc fragments of an immunoglobulin recognized by a FcRn; three SARS-CoV-2 antigens; and three trimerization domains.

In some aspects, three of the disclosed peptides trimerize forming a peptide complex wherein each of the three peptides is oriented in the same direction. For example, the peptides trimerize with all of the monomeric Fc fragments of an immunoglobulin recognized by a FcRn on one end of the peptide complex and all of the SARS-CoV-2 antigens on the other end of the peptide complex.

In some aspects, each peptide of the peptide complex can comprise a different coronavirus antigen. For example, in some aspects, each peptide of the peptide complex can comprise a different SARS-CoV-2 spike protein fragment.

In another aspect, one or more of the peptides comprises an adjuvant instead of a coronavirus antigen. For example, two peptides of the peptide complex can comprise one of the disclosed peptides and the third peptide can be a peptide comprising a monomeric Fc fragment, a trimerization domain, and an adjuvant.

### E. Nucleic Acid Sequences

As this specification discusses various peptide sequences it is understood that the nucleic acids that can encode those peptides are also disclosed. This would include all degenerate sequences related to a specific polypeptide sequence, i.e. all nucleic acids having a sequence that encodes one particular polypeptide sequence as well as all nucleic acids, including degenerate nucleic acids, encoding the disclosed variants and derivatives of the peptides. Thus, while each particular nucleic acid sequence may not be written out herein, it is understood that each and every sequence is in fact disclosed and described herein through the disclosed peptides.

Disclosed are nucleic acid sequences capable of encoding any of the peptides disclosed herein. Further disclosed are nucleic acid constructs comprising the nucleic acid sequences capable of encoding any of the peptides disclosed herein.

Disclosed are vectors comprising a nucleic acid sequence capable of encoding peptides comprising a monomeric Fc fragment of an immunoglobulin recognized by a FcRn; a SARS-CoV-2 antigen; and a trimerization domain. In some instances, the peptide can be any of the peptides disclosed herein.

In some instances, the disclosed vectors can further comprise a nucleic acid sequence capable of encoding a tag (e.g. label or purification tag). In some aspects, the label can be any peptide or protein that is encoded for by a nucleic acid. For example, the labeling moiety can be, but is not limited to, GST, myc, His, or GFP.

In some instances, the labeling moiety can be operably linked to the nucleic acid sequence capable of encoding the peptides comprising a monomeric Fc fragment of an immunoglobulin recognized by a FcRn; a SARS-CoV-2 antigen; and a trimerization domain. Thus, the labeling moiety and the peptide can be transcribed together.

In addition to a nucleic acid sequence capable of encoding the disclosed peptides, the disclosed vectors can carry regulatory sequences that control the expression of the disclosed peptides in a host cell. It will be appreciated by those skilled in the art that the design of the vector, including the selection of regulatory sequences can depend on such factors as the choice of the host cell to be transformed, the level of expression of protein desired, etc. Preferred regulatory sequences for mammalian host cell expression include viral elements that direct high levels of protein expression in mammalian cells, such as promoters and/or enhancers derived from retroviral LTRs, cytomegalovirus (CMV) (such as the CMV promoter/enhancer), Simian Virus 40 (SV40) (such as the SV40 promoter/enhancer), adenovirus, (e.g., the adenovirus major late promoter (AdMLP)), polyoma and strong mammalian promoters such as native immunoglobulin and actin promoters. For further description of viral regulatory elements, and sequences thereof, see e.g., U.S. Pat. Nos. 5,168,062, 4,510,245 and 4,968,615. Methods of expressing polypeptides in bacterial cells or fungal cells, e.g., yeast cells, are also well known in the art.

In some instances, the disclosed vectors further comprise a promoter operably linked to the nucleic acid sequence capable of encoding the disclosed peptides. In some instances, the promoter can be an inducible promoter. In some instances, the promoter can be a cell-specific promoter. The nucleic acid sequence capable of encoding the disclosed peptides can be functionally linked to a promoter. By “functionally linked” it is meant such that the promoter can promote expression of the nucleic acid sequence, thus having appropriate orientation of the promoter relative to the nucleic acid sequence.

In some instances, the nucleic acid sequence of a monomeric Fc fragment of a human IgG1 can be GAGCCTAAGTCCTGCGACAAGACCCACACAAGCCCACCATCTCCAGCTCCTGAGCT GCTGGGAGGACCAAGCGTGTTCCTGTTTCCTCCAAAGCCTAAGGATACACTGATGA TCTCTCGGACCCCAGAGGTGACATGCGTGGTGGTGGACGTGTCCCACGAGGACCCC GAGGTGAAGTTTAACTGGTACGTGGACGGCGTGGAGGTGCATAATGCTAAGACCAA GCCAAGGGAGGAGCAGTATAACAGCACATACCGGGTGGTGTCTGTGCTGACCGTGC TGCATCAGGATTGGCTGAACGGCAAGGAATACAAGTGCGCTGTGAGCAATAAGGCC CTGCCAGCTCCCATCGAGAAGACAATCTCTAAGGCCAAGGGCCAGCCTAGAGAGCC ACAGGTGTATACCCTGCCACCTTCCCGCGACGAGCTGACCAAGAATCAGGTGAGCC TGACATGTCTGGTGAAGGGCTTCTACCCTAGCGATATCGCTGTGGAGTGGGAGTCTA ACGGCCAGCCAGAGAACAATTATAAGACCACACCACCCGTGCTGGACTCCGATGGC AGCTTCTTTCTGTACAGCAAGCTGACAGTGGACAAGTCTCGGTGGCAGCAGGGCAA CGTGTTCTCCTGCTCCGTGATGCATGAGGCCCTGCACAACCATTACACCCAGAAGAG CCTGTCTCTGTCCCCTGGCAAGtga (SEQ ID NO:9) or a variant thereof. In some aspects, the variant can a sequence 50%, 55%, 65%, 70%, 75%, 80%, 90%, 95%, 96%, 97%, 98%, or 99% identical to the sequence of SEQ ID NO:9. The dotted underline sequence AGC represents a cysteine to serine mutation (C226S in full length human IgG1) to produce a monomer human IgG1. The dotted underline sequence TCT represents a cysteine to serine mutation (C229S in full length human IgG1) to produce a monomer human IgG1. The italicized, underlined sequence represents a mutation preventing complement binding (K322A in full length human IgG1). The lowercase sequence represents a stop codon.

In some instances, the nucleic acid sequence of a monomeric Fc fragment of a mouse IgG2a can be GAGCCCAGAGGGCCCACAATCAAGCCCTCTCCTCCATCCAAATCCCCAGCACCTAA CCTCTTGGGTGGACCATCCGTCTTCATCTTCCCTCCAAAGATCAAGGATGTACTCAT GATCTCCCTGAGCCCCATAGTCACATGTGTGGTGGTGGATGTGAGCGAGGATGACC CAGATGTCCAGATCAGCTGGTTTGTGAACAACGTGGAAGTACACACAGCTCAGACA CAAACCCATAGAGAGGATTACAACAGTACTCTCCGGGTGGTCAGTGCCCTCCCCAT CCAGCACCAGGACTGGATGAGTGGCAAGGCGTTCGCATGCGCGGTCAACAACAAA GACCTCCCAGCGCCCATCGAGAGAACCATCTCAAAACCCAAAGGGTCAGTAAGAGC TCCACAGGTATATGTCTTGCCTCCACCAGAAGAAGAGATGACTAAGAAACAGGTCA CTCTGACCTGCATGGTCACAGACTTCATGCCTGAAGACATTTACGTGGAGTGGACCA ACAACGGGAAAACAGAGCTAAACTACAAGAACACTGAACCAGTCCTGGACTCTGAT GGTTCTTACTTCATGTACAGCAAGCTGAGAGTGGAAAAGAAGAACTGGGTGGAAAG AAATAGCTACTCCTGTTCAGTGGTCCACGAGGGTCTGCACAATCACCACACGACTA AGAGCTTCTCCCGGACTCCGGGTAAA (SEQ ID NO:10) or a sequence 50%, 55%, 65%, 70%, 75%, 80%, 90%, 95%, 96%, 97%, 98%, or 99% identical to the sequence of SEQ ID NO:10. The bold underlined nucleic acids represent a mutation that encodes serine instead of cysteine to generate a single chain Fc.

In some aspects, disclosed are nucleic acid sequences comprising a monomeric Fc fragment of an immunoglobulin recognized by a FcRn sequence; a SARS-CoV-2 soluble S protein sequence; and a trimerization domain sequence. For example, disclosed are nucleic acid sequences comprising the sequence of

GGTACCGCCACCATGTTCGTGTTTCTGGTGCTGCTGCCACTGGTGTCCAGCCAGTGC
GTGAACCTGACCACAAGAACCCAGCTGCCCCCTGCCTATACCAATTCTTTCACAAGA
GGCGTGTACTATCCAGACAAGGTGTTTCGCTCTTCCGTGCTGCACAGCACACAGGAT
CTGTTTCTGCCCTTCTTTTCTAACGTGACCTGGTTCCACGCCATCCACGTGTCCGGCA
CCAATGGCACAAAGAGGTTCGACAATCCTGTGCTGCCCTTCAACGATGGCGTGTAC
TTCGCTTCTACCGAGAAGTCCAACATCATCCGGGGCTGGATCTTTGGCACCACACTG
GACAGCAAGACACAGTCTCTGCTGATCGTGAACAATGCCACCAACGTGGTCATCAA
GGTGTGCGAGTTCCAGTTTTGTAATGATCCTTTCCTGGGCGTGTACTATCATAAGAA
CAATAAGTCCTGGATGGAGAGCGAGTTTCGCGTGTATAGCTCTGCTAACAATTGTAC
ATTTGAGTACGTGAGCCAGCCATTCCTGATGGACCTGGAGGGCAAGCAGGGCAATT
TCAAGAACCTGAGAGAGTTCGTGTTTAAGAATATCGATGGCTACTTCAAGATCTAC
AGCAAGCACACCCCTATCAACCTGGTGCGCGACCTGCCACAGGGCTTCTCTGCCCTG
GAGCCTCTGGTGGATCTGCCAATCGGCATCAACATCACCAGGTTTCAGACACTGCTG
GCTCTGCATCGGTCTTACCTGACACCTGGCGACTCCAGCTCTGGATGGACCGCTGGA
GCTGCTGCTTACTATGTGGGCTATCTGCAGCCAAGAACCTTCCTGCTGAAGTACAAC
GAGAATGGCACCATCACAGACGCCGTGGATTGCGCTCTGGATCCACTGTCCGAGAC
CAAGTGTACACTGAAGAGCTTTACCGTGGAGAAGGGCATCTATCAGACATCCAATT

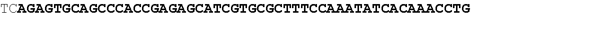

TGCCCCTTTGGCGAGGTGTTCAACGCCACCCGCTTCGCTTCCGTGTACGCCTG
GAATAGAAAGCGCATCTCCAACTGCGTGGCTGACTATAGCGTGCTGTACAACT
CCGCCAGCTTCTCTACCTTTAAGTGCTATGGCGTGTCCCCCACAAAGCTGAAT
GACCTGTGCTTTACCAACGTGTACGCCGATAGCTTCGTGATCAGAGGCGACGA
GGTGCGCCAGATCGCTCCAGGACAGACAGGCAAGATCGCCGACTACAATTATA
AGCTGCCTGACGATTTCACCGGCTGCGTGATCGCTTGGAACTCCAACAATCTG
GATAGCAAAGTGGGCGGCAACTACAATTATCTGTACAGGCTGTTTCGGAAGAG
CAATCTGAAGCCTTTCGAGAGGGACATCTCTACAGAGATCTACCAGGCCGGCT
CCACCCCATGCAATGGCGTGGAGGGCTTTAACTGTTATTTCCCCCTGCAGTCTT
ACGGCTTCCAGCCTACCAACGGCGTGGGCTATCAGCCATACCGGGTGGTGGTG
CTGTCTTTTGAGCTGCTGCACGCTCCAGCTACAGTGTGCGGACCTAAGAAGTC
CACCAATCTGGTGAAGAACAAGTGCGTGAACTTCAACTTCAACGGACTGACCGG
CACAGGCGTGCTGACCGAGAGCAACAAGAAGTTCCTGCCCTTTCAGCAGTTCGGCA
GGGACATCGCTGATACCACAGACGCCGTGCGGGACCCACAGACCCTGGAGATCCTG
GATATCACACCCTGCTCTTTCGGCGGCGTGTCCGTGATCACACCTGGCACCAATACA
TCTAACCAGGTGGCCGTGCTGTATCAGGACGTGAATTGTACCGAGGTGCCTGTGGC
CATCCACGCTGATCAGCTGACCCCAACATGGAGGGTGTACAGCACCGGCTCTAACG
TGTTTCAGACACGGGCTGGATGTCTGATCGGAGCTGAGCATGTGAACAATTCCTATG
AGTGCGACATCCCCATCGGCGCTGGCATCTGTGCCAGCTACCAGACCCAGACAAAC
AGCCCTAGGAGGGCTGCTTCTGTGGCTTCCCAGAGCATCATCGCCTATACCATGTCC
CTGGGCGCTGAGAATAGCGTGGCCTACTCCAACAATAGCATCGCTATCCCAACCAA
CTTCACAATCTCCGTGACCACAGAGATCCTGCCCGTGAGCATGACCAAGACATCTGT
GGACTGCACAATGTATATCTGTGGCGATTCTACCGAGTGCTCCAACCTGCTGCTGCA
GTACGGCAGCTTTTGTACCCAGCTGAATAGGGCTCTGACAGGCATCGCCGTGGAGC
AGGATAAGAACACACAGGAGGTGTTCGCCCAGGTGAAGCAGATCTACAAGACCCC
ACCCATCAAGGACTTTGGCGGCTTCAACTTCTCCCAGATCCTGCCTGATCCATCTAA
GCCCTCCAAGGCTAGCTTTATCGAGGACCTGCTGTTCAACAAGGTGACCCTGGCTG
ATGCCGGCTTCATCAAGCAGTATGGCGATTGCCTGGGCGACATCGCTGCCAGGGAC
CTGATCTGTGCTCAGAAGTTTAATGGCCTGACCGTGCTGCCTCCACTGCTGACAGAT
GAGATGATCGCCCAGTACACATCTGCCCTGCTGGCTGGCACCATCACATCCGGATG
GACCTTCGGCGCTGGAGCTGCCCTGCAGATCCCTTTTGCTATGCAGATGGCCTATCG
GTTCAACGGCATCGGCGTGACCCAGAATGTGCTGTACGAGAACCAGAAGCTGATCG
CTAATCAGTTTAACTCCGCCATCGGCAAGATCCAGGACTCTCTGTCCAGCACAGCTT
CCGCCCTGGGCAAGCTGCAGGATGTGGTGAATCAGAACGCTCAGGCCCTGAATACC
CTGGTGAAGCAGCTGTCTTCCAACTTCGGCGCTATCAGCTCTGTGCTGAATGATATC
CTGAGCAGACTGGACCCACCTGAGGCTGAGGTGCAGATCGACAGGCTGATCACAGG
CCGGCTGCAGAGCCTGCAGACCTACGTGACACAGCAGCTGATCAGAGCTGCCGAGA
TCCGCGCTTCTGCCAACCTGGCTGCCACCAAGATGTCTGAGTGCGTGCTGGGCCAGT
CCAAGCGCGTGGACTTTTGTGGCAAGGGCTATCACCTGATGAGCTTCCCCCAGTCTG
CTCCTCACGGCGTGGTGTTTCTGCATGTGACCTACGTGCCCGCCCAGGAGAAGAACT
TCACCACAGCTCCTGCCATCTGCCACGATGGCAAGGCCCATTTTCCCAGAGAGGGC
GTGTTCGTGTCTAACGGCACCCATTGGTTTGTGACACAGCGCAATTTCTACGAGCCT
CAGATCATCACCACAGACAATACCTTCGTGTCCGGCAACTGTGACGTGGTCATCGG
CATCGTGAACAATACCGTGTATGATCCCCTGCAGCCTGAGCTGGACTCTTTTAAGGA
GGAGCTGGATAAGTACTTCAAGAATCACACCTCCCCAGACGTGGATCTGGGCGACA
TCTCCGGCATCAATGCTAGCGTGGTGAACATCCAGAAGGAGATCGACAGGCTGAAC
GAGGTGGCCAAGAATCTGAACGAGTCTCTGATCGATCTGCAGGAGCTGGGCAAGTA

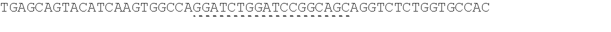

GGGGCTCTCCAGGATCCGGATATATCCCAGAGGCTCCCAGAGACGGACAGGCTTAC

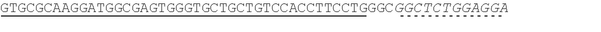

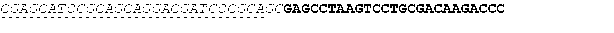

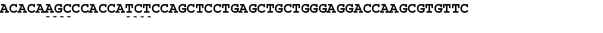

CTGTTTCCTCCAAAGCCTAAGGATACACTGATGATCTCTCGGACCCCAGAGGT
GACATGCGTGGTGGTGGACGTGTCCCACGAGGACCCCGAGGTGAAGTTTAACT
GGTACGTGGACGGCGTGGAGGTGCATAATGCTAAGACCAAGCCAAGGGAGGA
GCAGTATAACAGCACATACCGGGTGGTGTCTGTGCTGACCGTGCTGCATCAGG
ATTGGCTGAACGGCAAGGAATACAAGTGCGCTGTGAGCAATAAGGCCCTGCCA
GCTCCCATCGAGAAGACAATCTCTAAGGCCAAGGGCCAGCCTAGAGAGCCACA
GGTGTATACCCTGCCACCTTCCCGCGACGAGCTGACCAAGAATCAGGTGAGCC
TGACATGTCTGGTGAAGGGCTTCTACCCTAGCGATATCGCTGTGGAGTGGGAG
TCTAACGGCCAGCCAGAGAACAATTATAAGACCACACCACCCGTGCTGGACTC
CGATGGCAGCTTCTTTCTGTACAGCAAGCTGACAGTGGACAAGTCTCGGTGGC
AGCAGGGCAACGTGTTCTCCTGCTCCGTGATGCATGAGGCCCTGCACAACCAT

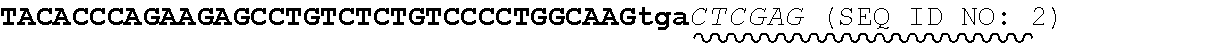

or a variant thereof. In some aspects, the variant can a sequence 50%, 55%, 65%, 70%, 75%, 80%, 90%, 95%, 96%, 97%, 98%, or 99% identical to the sequence of SEQ ID NO:2. The double underlined sequence represents a KpnI cloning site. The shaded sequence represents a Kozak sequence. The underlined sequence represents a native signal peptide of S protein. The bold shaded sequence represents the RBD of S1. The bold underline sequence represents the mutated S1/S2 cleavage site (R685A in italics, no change in S686). The bold letter and bold underline sequence represents a mutation at the S2′ cleavage site (R816A in italics, no change in S817). The bold, italics, and shaded sequence represents K986P and V987P mutations which allow the S protein to keep the Pre-fusion conformation. The dotted underline and shaded sequence represents a 6GS (glycine-serine) linker. The double squiggle underline represents the foldon domain from T4 fibrin. The dotted underline, italicized shaded sequence represents a 14GS (glycine-serine) linker. The bold sequence is human IgG1. The dotted underline sequence AGC represents a cysteine to serine mutation (C226S of full length human IgG1, CIIS of the Fc fragment disclosed herein) in human IgG1 to produce a monomer human IgG1. The dotted underline sequence TCT represents a cysteine to serine mutation (C229S of full length human IgG1, C14S of the Fc fragment disclosed herein) in human IgG1 to produce a monomer human IgG1. The italicized, underlined sequence represents a mutation preventing complement binding (K322A of full length human IgG1, K107A of the Fc fragment disclosed herein) in human IgG1. The bold lowercase sequence represents a stop codon in IgG1. The squiggly underline represents an XhoI cloning site. Nucleic acids 58 to 3594 represent the SARS-Cov-2 spike protein. Nucleic acids 3697-3783 represent the foldon domain of T4 fibrin. Nucleic acids 3829-4527 represent a monomeric Fc IgG1 fragment.

In some aspects, disclosed are nucleic acid sequences comprising a monomeric Fc fragment of an immunoglobulin recognized by a FcRn sequence; a SARS-CoV-2 S1 protein sequence; and a trimerization domain sequence. For example, disclosed are nucleic acid sequences comprising the sequence of

GGTACCGCCACCATGTTCGTGTTTCTGGTGCTGCTGCCACTGGTGTCCAGCCAGTGC
GTGAACCTGACCACAAGAACCCAGCTGCCCCCTGCCTATACCAATTCTTTCACAAGA
GGCGTGTACTATCCAGACAAGGTGTTTCGCTCTTCCGTGCTGCACAGCACACAGGAT
CTGTTTCTGCCCTTCTTTTCTAACGTGACCTGGTTCCACGCCATCCACGGTGGTCCGGCA
CCAATGGCACAAAGAGGTTCGACAATCCTGTGCTGCCCTTCAACGATGGCGTGTAC
TTCGCTTCTACCGAGAAGTCCAACATCATCCGGGGCTGGATCTTTGGCACCACACTG
GACAGCAAGACACAGTCTCTGCTGATCGTGAACAATGCCACCAACGTGGTCATCAA
GGTGTGCGAGTTCCAGTTTTGTAATGATCCTTTCCTGGGCGTGTACTATCATAAGAA
CAATAAGTCCTGGATGGAGAGCGAGTTTCGCGTGTATAGCTCTGCTAACAATTGTAC
ATTTGAGTACGTGAGCCAGCCATTCCTGATGGACCTGGAGGGCAAGCAGGGCAATT
TCAAGAACCTGAGAGAGTTCGTGTTTAAGAATATCGATGGCTACTTCAAGATCTAC
AGCAAGCACACCCCTATCAACCTGGTGCGCGACCTGCCACAGGGCTTCTCTGCCCTG
GAGCCTCTGGTGGATCTGCCAATCGGCATCAACATCACCAGGTTTCAGACACTGCTG
GCTCTGCATCGGTCTTACCTGACACCTGGCGACTCCAGCTCTGGATGGACCGCTGGA
GCTGCTGCTTACTATGTGGGCTATCTGCAGCCAAGAACCTTCCTGCTGAAGTACAAC
GAGAATGGCACCATCACAGACGCCGTGGATTGCGCTCTGGATCCACTGTCCGAGAC
CAAGTGTACACTGAAGAGCTTTACCGTGGAGAAGGGCATCTATCAGACATCCAATT

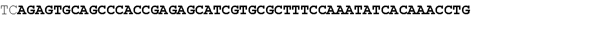

TGCCCCTTTGGCGAGGTGTTCAACGCCACCCGCTTCGCTTCCGTGTACGCCTG
GAATAGAAAGCGCATCTCCAACTGCGTGGCTGACTATAGCGTGCTGTACAACT
CCGCCAGCTTCTCTACCTTTAAGTGCTATGGCGTGTCCCCCACAAAGCTGAAT
GACCTGTGCTTTACCAACGTGTACGCCGATAGCTTCGTGATCAGAGGCGACGA
GGTGCGCCAGATCGCTCCAGGACAGACAGGCAAGATCGCCGACTACAATTATA
AGCTGCCTGACGATTTCACCGGCTGCGTGATCGCTTGGAACTCCAACAATCTG
GATAGCAAAGTGGGCGGCAACTACAATTATCTGTACAGGCTGTTTCGGAAGAG
CAATCTGAAGCCTTTCGAGAGGGACATCTCTACAGAGATCTACCAGGCCGGCT
CCACCCCATGCAATGGCGTGGAGGGCTTTAACTGTTATTTCCCCCTGCAGTCTT
ACGGCTTCCAGCCTACCAACGGCGTGGGCTATCAGCCATACCGGGTGGTGGTG
CTGTCTTTTGAGCTGCTGCACGCTCCAGCTACAGTGTGCGGACCTAAGAAGTC
CACCAATCTGGTGAAGAACAAGTGCGTGAACTTCAACTTCAACGGACTGACCGG
CACAGGCGTGCTGACCGAGAGCAACAAGAAGTTCCTGCCCTTTCAGCAGTTCGGCA
GGGACATCGCTGATACCACAGACGCCGTGCGGGACCCACAGACCCTGGAGATCCTG
GATATCACACCCTGCTCTTTCGGCGGCGTGTCCGTGATCACACCTGGCACCAATACA
TCTAACCAGGTGGCCGTGCTGTATCAGGACGTGAATTGTACCGAGGTGCCTGTGGC
CATCCACGCTGATCAGCTGACCCCAACATGGAGGGTGTACAGCACCGGCTCTAACG
TGTTTCAGACACGGGCTGGATGTCTGATCGGAGCTGAGCATGTGAACAATTCCTATG
AGTGCGACATCCCCATCGGCGCTGGCATCTGTGCCAGCTACCAGACCCAGACAAAC

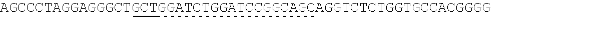

CTCTCCAGGATCCGGATATATCCCAGAGGCTCCCAGAGACGGACAGGCTTACGTGC

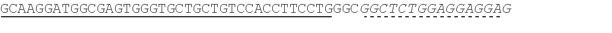

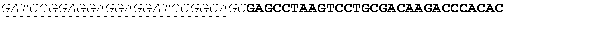

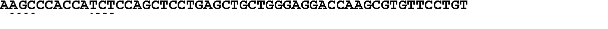

TTCCTCCAAAGCCTAAGGATACACTGATGATCTCTCGGACCCCAGAGGTGACA
TGCGTGGTGGTGGACGTGTCCCACGAGGACCCCGAGGTGAAGTTTAACTGGTA
CGTGGACGGCGTGGAGGTGCATAATGCTAAGACCAAGCCAAGGGAGGAGCAG
TATAACAGCACATACCGGGTGGTGTCTGTGCTGACCGTGCTGCATCAGGATTG
GCTGAACGGCAAGGAATACAAGTGCGCTGTGAGCAATAAGGCCCTGCCAGCTC
CCATCGAGAAGACAATCTCTAAGGCCAAGGGCCAGCCTAGAGAGCCACAGGTG
TATACCCTGCCACCTTCCCGCGACGAGCTGACCAAGAATCAGGTGAGCCTGAC
ATGTCTGGTGAAGGGCTTCTACCCTAGCGATATCGCTGTGGAGTGGGAGTCTA
ACGGCCAGCCAGAGAACAATTATAAGACCACACCACCCGTGCTGGACTCCGAT
GGCAGCTTCTTTCTGTACAGCAAGCTGACAGTGGACAAGTCTCGGTGGCAGCA
GGGCAACGTGTTCTCCTGCTCCGTGATGCATGAGGCCCTGCACAACCATTACA

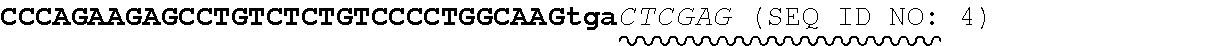

or a variant sequence 50%, 55%, 65%, 70%, 75%, 80%, 90%, 95%, 96%, 97%, 98%, or 99% identical to the sequence of SEQ ID NO:4. The double underlined sequence represents a KpnI cloning site. The shaded sequence represents a Kozak sequence. The underlined sequence represents a native signal peptide of S protein. The bold shaded sequence represents the RBD of S1. The italics and bold underline sequence represents the mutated S1/S2 cleavage site (R685A of S protein in italics). The dotted underline and shaded sequence represents a 6GS (glycine-serine) linker. The double squiggle underline represents the foldon domain from T4 fibrin. The dotted underline, italicized shaded sequence represents a 14GS (glycine-serine) linker. The bold sequence is human IgG1. The dotted underline sequence AGC represents a cysteine to serine mutation (C226S of full length human IgG1, C11S of the Fc fragment disclosed herein) in human IgG1 to produce a monomer human IgG1. The dotted underline sequence TCT represents a cysteine to serine mutation (C229S of full length human IgG1, C14S of the Fc fragment disclosed herein) in human IgG1 to produce a monomer human IgG1. The italicized, underlined sequence represents a mutation preventing complement binding (K322A of full length human IgG1, K107A of the Fc fragment disclosed herein) in human IgG1. The bold lowercase sequence represents a stop codon in IgG1. The squiggly underline represents an XhoI cloning site. Nucleic acids 58 to 2067 represent the SARS-Cov-2 S1 protein sequence. Nucleic acids 2113 to 2199 represent the foldon domain of T4 fibrin. Nucleic acids 2245 to 2943 represent a monomeric Fc IgG1 fragment.

In some aspects, disclosed are nucleic acid sequences comprising a monomeric Fc fragment of an immunoglobulin recognized by a FcRn sequence; a SARS-CoV-2 RBD protein sequence; and a trimerization domain sequence. For example, disclosed are nucleic acid sequences comprising the sequence of

GGTACCGCCACCATGTTCGTGTTTCTGGTGCTGCTGCCACTGGTGTCCAGCCAGTGC

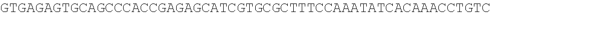

CCCCTTTGGCGAGGTGTTCAACGCCACCCGCTTCGCTTCCGTGTACGCCTGGAATAG
AAAGCGCATCTCCAACTGCGTGGCTGACTATAGCGTGCTGTACAACTCCGCCAGCTT
CTCTACCTTTAAGTGCTATGGCGTGTCCCCCACAAAGCTGAATGACCTGTGCTTTAC
CAACGTGTACGCCGATAGCTTCGTGATCAGAGGCGACGAGGTGCGCCAGATCGCTC
CAGGACAGACAGGCAAGATCGCCGACTACAATTATAAGCTGCCTGACGATTTCACC
GGCTGCGTGATCGCTTGGAACTCCAACAATCTGGATAGCAAAGTGGGCGGCAACTA
CAATTATCTGTACAGGCTGTTTCGGAAGAGCAATCTGAAGCCTTTCGAGAGGGACA
TCTCTACAGAGATCTACCAGGCCGGCTCCACCCCATGCAATGGCGTGGAGGGCTTT
AACTGTTATTTCCCCCTGCAGTCTTACGGCTTCCAGCCTACCAACGGCGTGGGCTAT
CAGCCATACCGGGTGGTGGTGCTGTCTTTTGAGCTGCTGCACGCTCCAGCTACAGTG

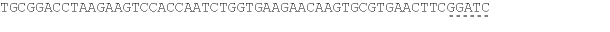

CAGAGGCTCCCAGAGACGGACAGGCTTACGTGCGCAAGGATGGCGAGTGGGTGCT

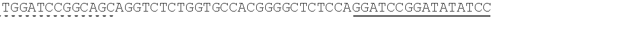

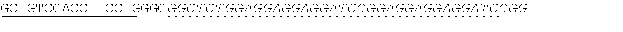

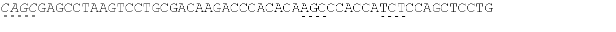

AGCTGCTGGGAGGACCAAGCGTGTTCCTGTTTCCTCCAAAGCCTAAGGATACACTG
ATGATCTCTCGGACCCCAGAGGTGACATGCGTGGTGGTGGACGTGTCCCACGAGGA
CCCCGAGGTGAAGTTTAACTGGTACGTGGACGGCGTGGAGGTGCATAATGCTAAGA
CCAAGCCAAGGGAGGAGCAGTATAACAGCACATACCGGGTGGTGTCTGTGCTGACC
GTGCTGCATCAGGATTGGCTGAACGGCAAGGAATACAAGTGCGCTGTGAGCAATAA
GGCCCTGCCAGCTCCCATCGAGAAGACAATCTCTAAGGCCAAGGGCCAGCCTAGAG
AGCCACAGGTGTATACCCTGCCACCTTCCCGCGACGAGCTGACCAAGAATCAGGTG
AGCCTGACATGTCTGGTGAAGGGCTTCTACCCTAGCGATATCGCTGTGGAGTGGGA
GTCTAACGGCCAGCCAGAGAACAATTATAAGACCACACCACCCGTGCTGGACTCCG
ATGGCAGCTTCTTTCTGTACAGCAAGCTGACAGTGGACAAGTCTCGGTGGCAGCAG
GGCAACGTGTTCTCCTGCTCCGTGATGCATGAGGCCCTGCACAACCATTACACCCAG

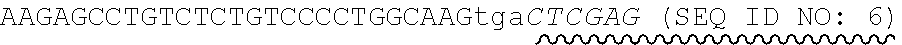

or a variant sequence 50%, 55%, 65%, 70%, 75%, 80%, 90%, 95%, 96%, 97%, 98%, or 99% identical to the sequence of SEQ ID NO:6. The double underlined sequence represents a KpnI cloning site. The shaded sequence represents a Kozak sequence. The underlined sequence represents a native signal peptide of S protein. The bold shaded sequence represents the RBD of S1.

The dotted underline and shaded sequence represents a 6GS (glycine-serine) linker. The double squiggle underline represents the foldon domain from T4 fibrin. The dotted underline, italicized shaded sequence represents a 14GS (glycine-serine) linker. The bold sequence is human IgG1. The dotted underline sequence AGC represents a cysteine to serine mutation (C226S of full length human IgG1, C11S of the Fc fragment disclosed herein) in human IgG1 to produce a monomer human IgG1. The dotted underline sequence TCT represents a cysteine to serine mutation (C229S of full length human IgG1, C14S of the Fc fragment disclosed herein) in human IgG1 to produce a monomer human IgG1. The italicized, underlined sequence represents a mutation preventing complement binding (K322A of full length human IgG1, K107A of the Fc fragment disclosed herein) in human IgG1. The bold lowercase sequence represents a stop codon in IgG1. The squiggly underline represents an XhoI cloning site. Nucleic acids 61 to 729 represent the SARS-Cov-2 RBD protein sequence. Nucleic acids 775 to 861 represent the foldon domain of T4 fibrin. Nucleic acids 907 to 1605 represent a monomeric Fc IgG1 fragment.

In some aspects, disclosed are nucleic acid sequences comprising a monomeric Fc fragment of an immunoglobulin recognized by a FcRn sequence; a SARS-CoV-2 soluble S protein sequence; and a trimerization domain sequence, wherein the SARS-CoV-2 soluble S protein sequence is from an Omicron or delta strain. For example, disclosed are nucleic acid sequences comprising the sequence of

ATGTTCGTGTTTCTGGTGCTGCTGCCCCTGGTGTCCAGCCAGTGCGTGAATCTGATC
ACCAGAACACAGAGCTACACCAACTCTTTCACACGCGGCGTGTACTATCCAGACAA
GGTGTTTAGGTCTTCCGTGCTGCACTCTACACAGGATCTGTTTCTGCCCTTCTTTTCC
AATGTGACCTGGTTCCACGCCATCCATGTGAGCGGCACCAACGGCACAAAGAGGTT
CGACAATCCTGCTCTGCCCTTCAACGATGGCGTGTACTTCGCCTCCACCGAGAAGAG
CAACATCATCAGGGGCTGGATCTTTGGCACCACACTGGACTCTAAGACACAGTCCC
TGCTGATCGTGAACAATGCTACCAACGTGGTCATCAAGGTGTGCGAGTTCCAGTTTT
GTAACGATCCTTTCCTGGACGTGTATCAGAAGAACAATAAGAGCTGGATGGAGTCT
GAGTTTCGCGTGTACAGCTCTGCCAACAATTGTACCTTTGAGTACGTGTCCCAGCCA
TTCCTGATGGACCTGGAGGGCAAGGAGGGCAATTTCAAGAACCTGCGGGAGTTCGT
GTTTAAGAATATCGATGGCTACTTCAAGATCTACAGCAAGCACACCCCAATCAACC
TGGAGAGAGACCTGCCACAGGGCTTCTCTGCCCTGGAGCCTCTGGTGGATCTGCCA
ATCGGCATCAACATCACCAGGTTTCAGACACTGCTGGCTCTGCATCGGAGCTACCTG
ACACCTGTGGACTCCAGCTCTGGATGGACCGCTGGAGCTGCTGCTTACTATGTGGGC
TACCTGCAGCCAAGGACCTTCCTGCTGAAGTATAACGAGAATGGCACCATCACAGA
CGCCGTGGATTGCGCTCTGGATCCACTGAGCGAGACCAAGTGTACACTGAAGTCTTT
TACCGTGGAGAAGGGCATCTACCAGACATCTAATTTCAGAGTGCAGCCCACCGAGT
CCATCGTGCGCTTTCCCAATATCACCAACCTGTGCCCTTTTCATGAGGTGTTCAACG
CCACCACATTCGCTTCCGTGTACGCCTGGAATAGGAAGCGGATCAGCAACTGCGTG
GCTGACTATTCTGTGATCTACAATTTTGCTCCATTCTTTGCCTTCAAGTGCTACGGCG
TGTCCCCCACAAAGCTGAATGACCTGTGCTTTACCAACGTGTATGCCGATAGCTTCG
TGATCCGCGGCAACGAGGTGTCTCAGATCGCTCCAGGCCAGACAGGCAATATCGCC
GACTACAACTATAAGCTGCCCGACGATTTCACCGGCTGCGTGATCGCCTGGAACTC
AATAAGCTGGATAGCAAGCCATCTGGCAACTACAATTATCTGTACAGACTGTTTCGC
AAGAGCAAGCTGAAGCCCTTCGAGAGGGATATCTCTACAGAGATCTACCAGGCTGG
CAATAAGCCTTGCAACGGAGTGGCTGGACCAAACTGTTATTCCCCTCTGCAGAGCT
ACGGCTTCCGCCCTACCTATGGAGTGGGACACCAGCCATATAGGGTGGTGGTGCTG
TCCTTTGAGCTGCTGCATGCCCCTGCTACAGTGTGCGGCCCAAAGAAGAGCACCAA
TCTGGTGAAGAACAAGTGCGTGAACTTCAACTTCAACGGACTGACCGGCACAGGCG
TGCTGACCGAGTCCAACAAGAAGTTCCTGCCCTTTCAGCAGTTCGGCCGGGACATC
GCTGATACCACAGACGCCGTGAGAGACCCACAGACCCTGGAGATCCTGGATATCAC
ACCCTGCTCCTTCGGCGGCGTGAGCGTGATCACACCTGGCACCAATACAAGCAACC
AGGTGGCTGTGCTGTACCAGGGCGTGAATTGTACCGAGGTGCCTGTGGCCATCCAC
GCTGACCAGCTGACCCCAACATGGCGCGTGTATTCCACCGGCAGCAACGTGTTTCA
GACAAGGGCTGGCTGTCTGATCGGCGCCGAGTATGTGAACAATTCCTACGAGTGCG
ATATCCCTATCGGCGCTGGCATCTGTGCCAGCTACCAGACCCAGACAAAGTCTCATA

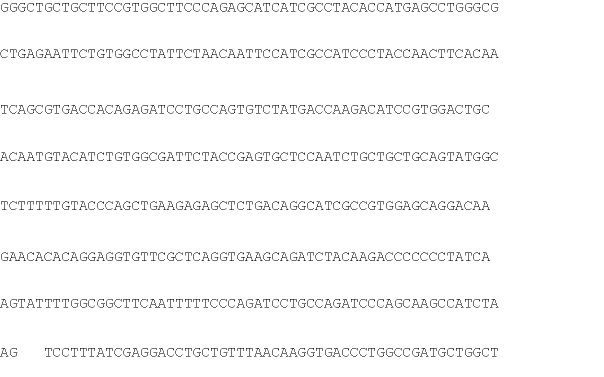

TCATCAAGCAGTACGGCGATTGCCTGGGCGACATCGCTGCTCGGGACCTGATCTGT
GCTCAGAAGTTCAATGGCCTGACCGTGCTGCCACCCCTGCTGACAGATGAGATGAT
CGCCCAGTATACATCCGCCCTGCTGGCTGGCACCATCACAAGCGGATGGACCTTTG
GCGCTGGAGCTGCTCTGCAGATCCCTTTTGCTATGCAGATGGCTTACAGATTCAACG
GCATCGGCGTGACCCAGAATGTGCTGTATGAGAACCAGAAGCTGATCGCTAATCAG
TTCAACTCTGCCATCGGCAAGATCCAGGACTCCCTGTCCAGCACAGCTAGCGCCCTG
GGCAAGCTGCAGGATGTGGTGAATCACAACGCCCAGGCTCTGAATACCCTGGTGAA
GCAGCTGTCTTCCAAGTTTGGCGCTATCAGCTCTGTGCTGAACGATATCCTGTCCCG

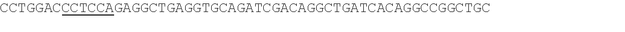

CTGAGAATTCTGTGGCCTATTCTAACAATTCCATCGCCATCCCTACCAACTTCACAA
AGTCTCTGCAGACCTACGTGACACAGCAGCTGATCAGAGCCGCTGAGATCCGCGCC
TCCGCTAACCTGGCCGCTACCAAGATGTCCGAGTGCGTGCTGGGCCAGAGCAAGAG
GGTGGACTTTTGTGGCAAGGGCTACCACCTGATGAGCTTCCCCCAGTCTGCTCCTCA
CGGCGTGGTGTTTCTGCATGTGACCTATGTGCCCGCCCAGGAGAAGAACTTCACCAC
AGCCCCTGCTATCTGCCACGATGGCAAGGCCCATTTTCCCCGGGAGGGCGTGTTCGT
GTCCAATGGCACCCATTGGTTTGTGACACAGAGAAACTTCTACGAGCCTCAGATCAT
CACCACAGACAATACATTTGTGAGCGGCAACTGTGACGTGGTCATCGGTATCGTGA
ACAATACCGTGTACGATCCCCTGCAGCCTGAGCTGGACTCTTTTAAGGAGGAGCTG
GATAAGTATTTCAAGAATCATACCTCCCCTGACGTGGATCTGGGCGACATCAGCGG
CATCAATGCTTCTGTGGTGAACATCCAGAAGGAGATCGACCGGCTGAACGAGGTGG
CCAAGAATCTGAACGAGAGCCTGATCGATCTGCAGGAGCTGGGCAAGTATGAGCAG

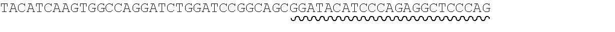

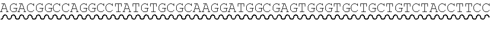

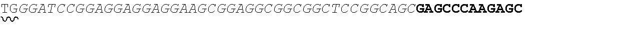

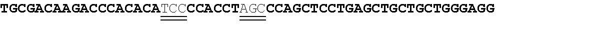

ACCATCCGTGTTCCTGTTTCCACCCAAGCCTAAGGATACACTGATGATCTCTAG
AACCCCTGAGGTGACATGCGTGGTGGTGGACGTGTCCCACGAGGACCCAGAG
GTGAAGTTCAATTGGTACGTGGACGGCGTGGAGGTGCATAACGCCAAGACCAA
GCCTAGGGAGGAGCAGTATAACTCTACATACCGGGTGGTGTCCGTGCTGACCG
TGCTGCATCAGGATTGGCTGAATGGCAAGGAGTATAAGTGCGCTGTGAGCAAC
AAGGCCCTGCCAGCTCCCATCGAGAAGACAATCTCTAAGGCTAAGGGACAGCC
TCGGGAGCCACAGGTGTACACCCTGCCTCCATCCAGAGACGAGCTGACCAAGA
ATCAGGTGAGCCTGACATGTCTGGTGAAGGGCTTCTATCCCTCCGATATCGCT
GTGGAGTGGGAGAGCAACGGCCAGCCTGAGAACAATTACAAGACCACCCCCC
CTGTGCTGGACTCTGATGGCTCCTTCTTTCTGTATTCTAAGCTGACAGTGGACA
AGTCCCGCTGGCAGCAGGGCAACGTGTTCTCCTGCTCCGTGATGCACGAGGCC
CTGCACAACCATTACACCCAGAAGAGCCTGTCTCTGTCCCCTGGCAAGTAA
(SEQ ID NO: 32)

or a variant sequence 50%, 55%, 65%, 70%, 75%, 80%, 90%, 95%, 96%, 97%, 98%, or 99% identical to the sequence of SEQ ID NO:32. The underlined sequence represents a native signal peptide of S protein. The bold sequence is human IgG1 Fc. The double underlined bold sequences represents a cysteine to serine mutation in human IgG1 to produce a monomer human IgG1. The shaded sequence is a 6GS linker and 14GS linker. The squiggle underline represents the foldon domain from T4 fibrin.

In some aspects, disclosed are nucleic acid sequences comprising a monomeric Fc fragment of an immunoglobulin recognized by an FcRn sequence; a SARS-CoV-2 S protein sequence; and a trimerization domain sequence, wherein the SARS-CoV-2 soluble S protein sequence is from XBB.1.5 XXX strain. For example, disclosed are nucleic acid sequences comprising the sequence of the spike gene of XBB.1.5 strain. gccaccATGTTCGTGTTTCTGGTGCTGCTGCCCCTGGTGTCCAGCCAGTGCGTGAATCT GATCACCAGAACACAGAGCTACACCAACTCTTTCACACGCGGCGTGTACTATCCAG ACAAGGTGTTTAGGTCTTCCGTGCTGCACTCTACACAGGATCTGTTTCTGCCCTTCTT TTCCAATGTGACCTGGTTCCACGCCATCCATGTGAGCGGCACCAACGGCACAAAGA GGTTCGACAATCCTGCTCTGCCCTTCAACGATGGCGTGTACTTCGCCTCCACCGAGA AGAGCAACATCATCAGGGGCTGGATCTTTGGCACCACACTGGACTCTAAGACACAG TCCCTGCTGATCGTGAACAATGCTACCAACGTGGTCATCAAGGTGTGCGAGTTCCAG TTTTGTAACGATCCTTTCCTGGACGTGTATCAGAAGAACAATAAGAGCTGGATGGA GTCTGAGTTTCGCGTGTACAGCTCTGCCAACAATTGTACCTTTGAGTACGTGTCCCA GCCATTCCTGATGGACCTGGAGGGCAAGGAGGGCAATTTCAAGAACCTGCGGGAGT TCGTGTTTAAGAATATCGATGGCTACTTCAAGATCTACAGCAAGCACACCCCAATCA ACCTGGAGAGAGACCTGCCACAGGGCTTCTCTGCCCTGGAGCCTCTGGTGGATCTG CCAATCGGCATCAACATCACCAGGTTTCAGACACTGCTGGCTCTGCATCGGAGCTAC CTGACACCTGTGGACTCCAGCTCTGGATGGACCGCTGGAGCTGCTGCTTACTATGTG GGCTACCTGCAGCCAAGGACCTTCCTGCTGAAGTATAACGAGAATGGCACCATCAC AGACGCCGTGGATTGCGCTCTGGATCCACTGAGCGAGACCAAGTGTACACTGAAGT CTTTTACCGTGGAGAAGGGCATCTACCAGACATCTAATTTCAGAGTGCAGCCCACC GAGTCCATCGTGCGCTTTCCCAATATCACCAACCTGTGCCCTTTTCATGAGGTGTTC AACGCCACCACATTCGCTTCCGTGTACGCCTGGAATAGGAAGCGGATCAGCAACTG CGTGGCTGACTATTCTGTGATCTACAATTTTGCTCCATTCTTTGCCTTCAAGTGCTAC GGCGTGTCCCCCACAAAGCTGAATGACCTGTGCTTTACCAACGTGTATGCCGATAGC TTCGTGATCCGCGGCAACGAGGTGTCTCAGATCGCTCCAGGCCAGACAGGCAATAT CGCCGACTACAACTATAAGCTGCCCGACGATTTCACCGGCTGCGTGATCGCCTGGA ACTCTAATAAGCTGGATAGCAAGCCATCTGGCAACTACAATTATCTGTACAGACTGT TTCGCAAGAGCAAGCTGAAGCCCTTCGAGAGGGATATCTCTACAGAGATCTACCAG GCTGGCAATAAGCCTTGCAACGGAGTGGCTGGACCAAACTGTTATTCCCCTCTGCA GAGCTACGGCTTCCGCCCTACCTATGGAGTGGGACACCAGCCATATAGGGTGGTGG TGCTGTCCTTTGAGCTGCTGCATGCCCCTGCTACAGTGTGCGGCCCAAAGAAGAGCA CCAATCTGGTGAAGAACAAGTGCGTGAACTTCAACTTCAACGGACTGACCGGCACA GGCGTGCTGACCGAGTCCAACAAGAAGTTCCTGCCCTTTCAGCAGTTCGGCCGGGA CATCGCTGATACCACAGACGCCGTGAGAGACCCACAGACCCTGGAGATCCTGGATA TCACACCCTGCTCCTTCGGCGGCGTGAGCGTGATCACACCTGGCACCAATACAAGC AACCAGGTGGCTGTGCTGTACCAGGGCGTGAATTGTACCGAGGTGCCTGTGGCCAT CCACGCTGACCAGCTGACCCCAACATGGCGCGTGTATTCCACCGGCAGCAACGTGT TTCAGACAAGGGCTGGCTGTCTGATCGGCGCCGAGTATGTGAACAATTCCTACGAG TGCGATATCCCTATCGGCGCTGGCATCTGTGCCAGCTACCAGACCCAGACAAAGTCT CATAGGGCTGCTGCTTCCGTGGCTTCCCAGAGCATCATCGCCTACACCATGAGCCTG GGCGCTGAGAATTCTGTGGCCTATTCTAACAATTCCATCGCCATCCCTACCAACTTC ACAATCAGCGTGACCACAGAGATCCTGCCAGTGTCTATGACCAAGACATCCGTGGA CTGCACAATGTACATCTGTGGCGATTCTACCGAGTGCTCCAATCTGCTGCTGCAGTA TGGCTCTTTTTGTACCCAGCTGAAGAGAGCTCTGACAGGCATCGCCGTGGAGCAGG ACAAGAACACACAGGAGGTGTTCGCTCAGGTGAAGCAGATCTACAAGACCCCCCCT ATCAAGTATTTTGGCGGCTTCAATTTTTCCCAGATCCTGCCAGATCCCAGCAAGCCA TCTAAGGCCTCCCCCATCGAGGACCTGCTGTTTAACAAGGTGACCCTGGCCGATGCT GGCTTCATCAAGCAGTACGGCGATTGCCTGGGCGACATCGCTGCTCGGGACCTGAT CTGTGCTCAGAAGTTCAATGGCCTGACCGTGCTGCCACCCCTGCTGACAGATGAGAT GATCGCCCAGTATACATCCGCCCTGCTGGCTGGCACCATCACAAGCGGATGGACCT TTGGCGCTGGACCAGCTCTGCAGATCCCTTTTCCAATGCAGATGGCTTACAGATTCA ACGGCATCGGCGTGACCCAGAATGTGCTGTATGAGAACCAGAAGCTGATCGCTAAT CAGTTCAACTCTGCCATCGGCAAGATCCAGGACTCCCTGTCCAGCACACCAAGCGC CCTGGGCAAGCTGCAGGATGTGGTGAATCACAACGCCCAGGCTCTGAATACCCTGG TGAAGCAGCTGTCTTCCAAGTTTGGCGCTATCAGCTCTGTGCTGAACGATATCCTGT CCCGCCTGGACCCTCCAGAGGCTGAGGTGCAGATCGACAGGCTGATCACAGGCCGG CTGCAGTCTCTGCAGACCTACGTGACACAGCAGCTGATCAGAGCCGCTGAGATCCG CGCCTCCGCTAACCTGGCCGCTACCAAGATGTCCGAGTGCGTGCTGGGCCAGAGCA AGAGGGTGGACTTTTGTGGCAAGGGCTACCACCTGATGAGCTTCCCCCAGTCTGCTC CTCACGGCGTGGTGTTTCTGCATGTGACCTATGTGCCCGCCCAGGAGAAGAACTTCA CCACAGCCCCTGCTATCTGCCACGATGGCAAGGCCCATTTTCCCCGGGAGGGCGTGT TCGTGTCCAATGGCACCCATTGGTTTGTGACACAGAGAAACTTCTACGAGCCTCAGA TCATCACCACAGACAATACATTTGTGAGCGGCAACTGTGACGTGGTCATCGGTATC GTGAACAATACCGTGTACGATCCCCTGCAGCCTGAGCTGGACTCTTTTAAGGAGGA GCTGGATAAGTATTTCAAGAATCATACCTCCCCTGACGTGGATCTGGGCGACATCA GCGGCATCAATGCTTCTGTGGTGAACATCCAGAAGGAGATCGACCGGCTGAACGAG GTGGCCAAGAATCTGAACGAGAGCCTGATCGATCTGCAGGAGCTGGGCAAGTATGA GCAGTACATCAAGTGGCCAGGATCTGGATCCGGCAGCGGATACATCCCAGAGGCTC CCAGAGACGGCCAGGCCTATGTGCGCAAGGATGGCGAGTGGGTGCTGCTGTCTACC TTCCT GGGATCCGGAGGAGGAGGAAGCGGAGGCGGCGGCTCCGGCAGCGAGCCCAAG AGCTCCGACAAGACCCACACATCCCCACCTAGCCCAGCTCCTGAGCTGCTGGGAGG ACCATCCGTGTTCCTGTTTCCACCCAAGCCTAAGGATACACTGATGATCTCTAGAAC CCCTGAGGTGACATGCGTGGTGGTGGACGTGTCCCACGAGGACCCAGAGGTGAAGT TCAATTGGTACGTGGACGGCGTGGAGGTGCATAACGCCAAGACCAAGCCTAGGGAG GAGCAGTATAACTCTACATACCGGGTGGTGTCCGTGCTGACCGTGCTGCATCAGGA TTGGCTGAATGGCAAGGAGTATAAGTGCGCTGTGAGCAACAAGGCCCTGCCAGCTC CCATCGAGAAGACAATCTCTAAGGCTAAGGGACAGCCTCGGGAGCCACAGGTGTAC ACCCTGCCTCCATCCAGAGACGAGCTGACCAAGAATCAGGTGAGCCTGACATGTCT GGTGAAGGGCTTCTATCCCTCCGATATCGCTGTGGAGTGGGAGAGCAACGGCCAGC CTGAGAACAATTACAAGACCACCCCCCCTGTGCTGGACTCTGATGGCTCCTTCTTTC TGTATTCTAAGCTGACAGTGGACAAGTCCCGCTGGCAGCAGGGCAACGTGTTCTCCT GCTCCGTGATGCACGAGGCCCTGCACAACCATTACACCCAGAAGAGCCTGTCTCTG TCCCCTGGCAAGTAA (SEQ ID NO:53) or a variant thereof. In some aspects, the variant can a sequence 50%, 55%, 65%, 70%, 75%, 80%, 90%, 95%, 96%, 97%, 98%, or 99% identical to the sequence of SEQ ID NO:53. Nucleic acids 1-3627 represent XBB.1.5 soluble spike. Nucleic acids 3645-3726 represent the foldon domain of T4 fibrin. Nucleic acids 3769-4573 represent a monomeric Fc IgG1 fragment.

In some aspects, disclosed are nucleic acid sequences comprising a monomeric Fc fragment of an immunoglobulin recognized by an FcRn sequence; a SARS-CoV-2 S protein sequence comprising six proline mutations; and a trimerization domain sequence, wherein the SARS-CoV-2 S protein sequence is from XBB1.5 strain. For example, disclosed are nucleic acid sequences comprising the sequence of

GGTACCgccaccATGTTCGTGTTTCTGGTGCTGCTGCCCCTGGTGTCCAGCCAGTGCGT
GAATCTGATCACCAGAACACAGAGCTACACCAACTCTTTCACACGCGGCGTGTACT
ATCCAGACAAGGTGTTTAGGTCTTCCGTGCTGCACTCTACACAGGATCTGTTTCTGC
CCTTCTTTTCCAATGTGACCTGGTTCCACGCCATCCATGTGAGCGGCACCAACGGCA
CAAAGAGGTTCGACAATCCTGCTCTGCCCTTCAACGATGGCGTGTACTTCGCCTCCA
CCGAGAAGAGCAACATCATCAGGGGCTGGATCTTTGGCACCACACTGGACTCTAAG
ACACAGTCCCTGCTGATCGTGAACAATGCTACCAACGTGGTCATCAAGGTGTGCGA
GTTCCAGTTTTGTAACGATCCTTTCCTGGACGTGTATCAGAAGAACAATAAGAGCTG
GATGGAGTCTGAGTTTCGCGTGTACAGCTCTGCCAACAATTGTACCTTTGAGTACGT
GTCCCAGCCATTCCTGATGGACCTGGAGGGCAAGGAGGGCAATTTCAAGAACCTGC
GGGAGTTCGTGTTTAAGAATATCGATGGCTACTTCAAGATCTACAGCAAGCACACC
CCAATCAACCTGGAGAGAGACCTGCCACAGGGCTTCTCTGCCCTGGAGCCTCTGGT
GGATCTGCCAATCGGCATCAACATCACCAGGTTTCAGACACTGCTGGCTCTGCATCG
GAGCTACCTGACACCTGTGGACTCCAGCTCTGGATGGACCGCTGGAGCTGCTGCTTA
CTATGTGGGCTACCTGCAGCCAAGGACCTTCCTGCTGAAGTATAACGAGAATGGCA
CCATCACAGACGCCGTGGATTGCGCTCTGGATCCACTGAGCGAGACCAAGTGTACA
CTGAAGTCTTTTACCGTGGAGAAGGGCATCTACCAGACATCTAATTTCAGAGTGCA
GCCCACCGAGTCCATCGTGCGCTTTCCCAATATCACCAACCTGTGCCCTTTTCATGA
GGTGTTCAACGCCACCACATTCGCTTCCGTGTACGCCTGGAATAGGAAGCGGATCA
GCAACTGCGTGGCTGACTATTCTGTGATCTACAATTTTGCTCCATTCTTTGCCTTCAA
GTGCTACGGCGTGTCCCCCACAAAGCTGAATGACCTGTGCTTTACCAACGTGTATGC
CGATAGCTTCGTGATCCGCGGCAACGAGGTGTCTCAGATCGCTCCAGGCCAGACAG
GCAATATCGCCGACTACAACTATAAGCTGCCCGACGATTTCACCGGCTGCGTGATC
GCCTGGAACTCTAATAAGCTGGATAGCAAGCCATCTGGCAACTACAATTATCTGTA
CAGACTGTTTCGCAAGAGCAAGCTGAAGCCCTTCGAGAGGGATATCTCTACAGAGA
TCTACCAGGCTGGCAATAAGCCTTGCAACGGAGTGGCTGGACCAAACTGTTATTCC
CCTCTGCAGAGCTACGGCTTCCGCCCTACCTATGGAGTGGGACACCAGCCATATAG
GGTGGTGGTGCTGTCCTTTGAGCTGCTGCATGCCCCTGCTACAGTGTGCGGCCCAAA
GAAGAGCACCAATCTGGTGAAGAACAAGTGCGTGAACTTCAACTTCAACGGACTGA
CCGGCACAGGCGTGCTGACCGAGTCCAACAAGAAGTTCCTGCCCTTTCAGCAGTTC
GGCCGGGACATCGCTGATACCACAGACGCCGTGAGAGACCCACAGACCCTGGAGAT
CCTGGATATCACACCCTGCTCCTTCGGCGGCGTGAGCGTGATCACACCTGGCACCAA
TACAAGCAACCAGGTGGCTGTGCTGTACCAGGGCGTGAATTGTACCGAGGTGCCTG
TGGCCATCCACGCTGACCAGCTGACCCCAACATGGCGCGTGTATTCCACCGGCAGC
AACGTGTTTCAGACAAGGGCTGGCTGTCTGATCGGCGCCGAGTATGTGAACAATTC
CTACGAGTGCGATATCCCTATCGGCGCTGGCATCTGTGCCAGCTACCAGACCCAGA
CAAAGTCTCATAGGGCTGCTGCTTCCGTGGCTTCCCAGAGCATCATCGCCTACACC
ATGAGCCTGGGCGCTGAGAATTCTGTGGCCTATTCTAACAATTCCATCGCCATCCCT
ACCAACTTCACAATCAGCGTGACCACAGAGATCCTGCCAGTGTCTATGACCAAGAC
ATCCGTGGACTGCACAATGTACATCTGTGGCGATTCTACCGAGTGCTCCAATCTGCT
GCTGCAGTATGGCTCTTTTTGTACCCAGCTGAAGAGAGCTCTGACAGGCATCGCCGT
GGAGCAGGACAAGAACACACAGGAGGTGTTCGCTCAGGTGAAGCAGATCTACAAG
ACCCCCCCTATCAAGTATTTTGGCGGCTTCAATTTTTCCCAGATCCTGCCAGATCCC
AGCAAGCCATCTAAGGCCTCCcccATCGAGGACCTGCTGTTTAACAAGGTGACCCTG
GCCGATGCTGGCTTCATCAAGCAGTACGGCGATTGCCTGGGCGACATCGCTGCTCG
GGACCTGATCTGTGCTCAGAAGTTCAATGGCCTGACCGTGCTGCCACCCCTGCTGAC
AGATGAGATGATCGCCCAGTATACATCCGCCCTGCTGGCTGGCACCATCACAAGCG
GATGGACCTTTGGCGCTGGAccaGCTCTGCAGATCCCTTTTccaATGCAGATGGCTTAC
AGATTCAACGGCATCGGCGTGACCCAGAATGTGCTGTATGAGAACCAGAAGCTGAT
CGCTAATCAGTTCAACTCTGCCATCGGCAAGATCCAGGACTCCCTGTCCAGCACAcca
AGCGCCCTGGGCAAGCTGCAGGATGTGGTGAATCACAACGCCCAGGCTCTGAATAC
CCTGGTGAAGCAGCTGTCTTCCAAGTTTGGCGCTATCAGCTCTGTGCTGAACGATAT
CCTGTCCCGCCTGGACcctccaGAGGCTGAGGTGCAGATCGACAGGCTGATCACAGGC
CGGCTGCAGTCTCTGCAGACCTACGTGACACAGCAGCTGATCAGAGCCGCTGAGAT
CCGCGCCTCCGCTAACCTGGCCGCTACCAAGATGTCCGAGTGCGTGCTGGGCCAGA
GCAAGAGGGTGGACTTTTGTGGCAAGGGCTACCACCTGATGAGCTTCCCCCAGTCT
GCTCCTCACGGCGTGGTGTTTCTGCATGTGACCTATGTGCCCGCCCAGGAGAAGAAC
TTCACCACAGCCCCTGCTATCTGCCACGATGGCAAGGCCCATTTTCCCCGGGAGGGC
GTGTTCGTGTCCAATGGCACCCATTGGTTTGTGACACAGAGAAACTTCTACGAGCCT
CAGATCATCACCACAGACAATACATTTGTGAGCGGCAACTGTGACGTGGTCATCGG
TATCGTGAACAATACCGTGTACGATCCCCTGCAGCCTGAGCTGGACTCTTTTAAGGA
GGAGCTGGATAAGTATTTCAAGAATCATACCTCCCCTGACGTGGATCTGGGCGACA
TCAGCGGCATCAATGCTTCTGTGGTGAACATCCAGAAGGAGATCGACCGGCTGAAC
GAGGTGGCCAAGAATCTGAACGAGAGCCTGATCGATCTGCAGGAGCTGGGCAAGT

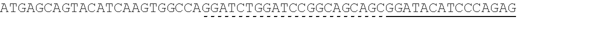

GCTCCCAGAGACGGCCAGGCCTATGTGCGCAAGGATGGCGAGTGGGTGCTGCTGTC

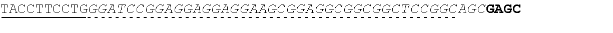

CCAAGAGCtccGACAAGACCCACACAtccCCACCTagcCCAGCTCCTGAGCTGCTG
GGAGGACCATCCGTGTTCCTGTTTCCACCCAAGCCTAAGGATACACTGATGAT
CTCTAGAACCCCTGAGGTGACATGCGTGGTGGTGGACGTGTCCCACGAGGACC
CAGAGGTGAAGTTCAATTGGTACGTGGACGGCGTGGAGGTGCATAACGCCAA
GACCAAGCCTAGGGAGGAGCAGTATAACTCTACATACCGGGTGGTGTCCGTGC

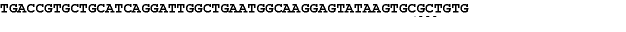

AGCAACAAGGCCCTGCCAGCTCCCATCGAGAAGACAATCTCTAAGGCTAAGGG
ACAGCCTCGGGAGCCACAGGTGTACACCCTGCCTCCATCCAGAGACGAGCTGA
CCAAGAATCAGGTGAGCCTGACATGTCTGGTGAAGGGCTTCTATCCCTCCGAT
ATCGCTGTGGAGTGGGAGAGCAACGGCCAGCCTGAGAACAATTACAAGACCAC
CCCCCCTGTGCTGGACTCTGATGGCTCCTTCTTTCTGTATTCTAAGCTGACAGT
GGACAAGTCCCGCTGGCAGCAGGGCAACGTGTTCTCCTGCTCCGTGATGCACG
AGGCCCTGCACAACCATTACACCCAGAAGAGCCTGTCTCTGTCCCCTGGCAAG
TAA (SEQ ID NO: 54)

or a variant thereof. In some aspects, the variant can a sequence 50%, 65%, 70%, 75%, 80%, 90%, 95%, 96%, 97%, 98%, or 99% identical to the sequence of SEQ ID NO:54. The double underlined sequence represents a KpnI cloning site. The lowercase sequence represents a Kozak sequence. The underlined sequence represents a native signal peptide of S protein. The bold underline sequence represents the mutated S1/S2 cleavage site (from RRARS to RAAAS). The bold letter and double underline sequence represents a mutation at the S2′ cleavage site (from KRS to KAS). The dotted underline sequence represents a 6GS (glycine-serine) linker. The double squiggle underline represents the foldon domain from T4 fibrin. The dotted underline, italicized sequence represents a 14GS (glycine-serine) linker. The bold sequence is human IgG1-Fc. The lowercase bold sequences represent the 6P mutations (F817P, A892P, A899P, A942P, K986P, V987P) the SARS-CoV-2 S protein. The lower case underlined sequence within the IgG1 Fc sequence represents C220S, C226S and C229S (corresponding to amino acid positions 5, 11, and 14 of the Fc fragment herein) mutations for a monomer hIgG1 and avoid cysteine scrambling. The large dashed underlined sequence in the IgG1 Fc fragment represents mutations preventing complement binding (K322A of full length human IgG1, K107A of the Fc fragment disclosed herein). Nucleic acids 1 to 3627 represent the SARS-Cov-2 spike protein. Nucleic acids 3645-3726 represent the foldon domain of T4 fibrin. Nucleic acids 3769-4573 represent a monomeric Fc IgG1 fragment.

### F. Compositions

Disclosed are compositions comprising any of the disclosed peptides, peptide complexes, nucleic acid sequences, or vectors. In some instances, disclosed are compositions comprising a monomeric Fc fragment of an immunoglobulin recognized by a FcRn; a coronavirus antigen; and a trimerization domain. Also disclosed are compositions comprising a monomeric Fc fragment of an immunoglobulin recognized by a FcRn; a SARS-CoV-2 antigen; and a trimerization domain.

In some instances, the composition can be a vaccine.

In some instances, the compositions can further comprise a pharmaceutically acceptable carrier. By “pharmaceutically acceptable” is meant a material or carrier that would be selected to minimize any degradation of the active ingredient and to minimize any adverse side effects in the subject, as would be well known to one of skill in the art. The pharmaceutical carrier employed can be, for example, a solid, liquid, or gas. Examples of solid carriers include lactose, terra alba, sucrose, talc, gelatin, agar, pectin, acacia, magnesium stearate, and stearic acid. Examples of liquid carriers are sugar syrup, peanut oil, olive oil, and water. Examples of gaseous carriers include carbon dioxide and nitrogen. Examples of pharmaceutically acceptable carriers include dimyristoylphosphatidylcholine (DMPC), phosphate buffered saline or a multivesicular liposome. For example, PG:PC:Cholesterol:peptide or PC:peptide can be used as carriers in this invention. Other suitable pharmaceutically acceptable carriers and their formulations are described in Remington: The Science and Practice of Pharmacy (19th ed.) ed. A.R. Gennaro, Mack Publishing Company, Easton, PA 1995. Typically, an appropriate amount of pharmaceutically-acceptable salt is used in the formulation to render the formulation isotonic. Other examples of the pharmaceutically-acceptable carrier include, but are not limited to, saline, Ringer's solution and dextrose solution. The pH of the solution can be from about 5 to about 8, or from about 7 to about 7.5. Further carriers include sustained release preparations such as semi-permeable matrices of solid hydrophobic polymers containing the composition, which matrices are in the form of shaped articles, e.g., films, stents (which are implanted in vessels during an angioplasty procedure), liposomes or microparticles. It will be apparent to those persons skilled in the art that certain carriers may be more preferable depending upon, for instance, the route of administration and concentration of composition being administered. These most typically would be standard carriers for administration of drugs to humans, including solutions such as sterile water, saline, and buffered solutions at physiological pH.

In order to enhance the solubility and/or the stability of the disclosed peptides in pharmaceutical compositions, it can be advantageous to employ α-, β- or γ-cyclodextrins or their derivatives, in particular hydroxyalkyl substituted cyclodextrins, e.g. 2-hydroxypropyl-β-cyclodextrin or sulfobutyl-β-cyclodextrin. Also, co-solvents such as alcohols may improve the solubility and/or the stability of the compounds according to the invention in pharmaceutical compositions.

Pharmaceutical compositions can also include carriers, thickeners, diluents, buffers, preservatives and the like, as long as the intended activity of the polypeptide, peptide, nucleic acid, vector of the invention is not compromised. Pharmaceutical compositions may also include one or more active ingredients (in addition to the composition of the invention) such as antimicrobial agents, anti-inflammatory agents, anesthetics, and the like. The pharmaceutical composition may be administered in a number of ways depending on whether local or systemic treatment is desired, and on the area to be treated.

Preparations of parenteral administration include sterile aqueous or non-aqueous solutions, suspensions, and emulsions. Examples of non-aqueous solvents are propylene glycol, polyethylene glycol, vegetable oils such as olive oil, and injectable organic esters such as ethyl oleate. Aqueous carriers include water, alcoholic/aqueous solutions, emulsions or suspensions, including saline and buffered media. Parenteral vehicles include sodium chloride solution, Ringer's dextrose, dextrose and sodium chloride, lactated Ringer's, or fixed oils. Intravenous vehicles include fluid and nutrient replenishers, electrolyte replenishers (such as those based on Ringer's dextrose), and the like. Preservatives and other additives may also be present such as, for example, antimicrobials, anti-oxidants, chelating agents, and inert gases and the like.

Formulations for optical administration may include ointments, lotions, creams, gels, drops, suppositories, sprays, liquids and powders. Conventional pharmaceutical carriers, aqueous, powder or oily bases, thickeners and the like may be necessary or desirable.

Compositions for oral administration include powders or granules, suspensions or solutions in water or non-aqueous media, capsules, sachets, or tablets. Thickeners, flavorings, diluents, emulsifiers, dispersing aids, or binders may be desirable. Some of the compositions may potentially be administered as a pharmaceutically acceptable acid- or base-addition salt, formed by reaction with inorganic acids such as hydrochloric acid, hydrobromic acid, perchloric acid, nitric acid, thiocyanic acid, sulfuric acid, and phosphoric acid, and organic acids such as formic acid, acetic acid, propionic acid, glycolic acid, lactic acid, pyruvic acid, oxalic acid, malonic acid, succinic acid, maleic acid, and fumaric acid, or by reaction with an inorganic base such as sodium hydroxide, ammonium hydroxide, potassium hydroxide, and organic bases such as mon-, di-, trialkyl and aryl amines and substituted ethanolamines.

Because of the ease in administration, oral administration can be used, and tablets and capsules represent the most advantageous oral dosage unit forms in which case solid pharmaceutical carriers are obviously employed. In preparing the compositions for oral dosage form, any convenient pharmaceutical media can be employed. For example, water, glycols, oils, alcohols, flavoring agents, preservatives, coloring agents and the like can be used to form oral liquid preparations such as suspensions, elixirs and solutions; while carriers such as starches, sugars, microcrystalline cellulose, diluents, granulating agents, lubricants, binders, disintegrating agents, and the like can be used to form oral solid preparations such as powders, capsules and tablets. Because of their ease of administration, tablets and capsules are the preferred oral dosage units whereby solid pharmaceutical carriers are employed. Optionally, tablets can be coated by standard aqueous or nonaqueous techniques.

A tablet containing the compositions of the present invention can be prepared by compression or molding, optionally with one or more accessory ingredients or adjuvants. Compressed tablets can be prepared by compressing, in a suitable machine, the active ingredient in a free-flowing form such as powder or granules, optionally mixed with a binder, lubricant, inert diluent, surface active or dispersing agent. Molded tablets can be made by molding in a suitable machine, a mixture of the powdered compound moistened with an inert liquid diluent.

The disclosed peptides can be formulated and/or administered in or with a pharmaceutically acceptable carrier. As used herein, the term “pharmaceutically acceptable carrier” refers to sterile aqueous or nonaqueous solutions, dispersions, suspensions or emulsions, as well as sterile powders for reconstitution into sterile injectable solutions or dispersions just prior to use. Examples of suitable aqueous and nonaqueous carriers, diluents, solvents or vehicles include water, ethanol, polyols (such as glycerol, propylene glycol, polyethylene glycol and the like), carboxymethylcellulose and suitable mixtures thereof, vegetable oils (such as olive oil) and injectable organic esters such as ethyl oleate. Proper fluidity can be maintained, for example, by the use of coating materials such as lecithin, by the maintenance of the required particle size in the case of dispersions and by the use of surfactants. These compositions can also contain adjuvants such as preservatives, wetting agents, emulsifying agents and dispersing agents. Prevention of the action of microorganisms can be ensured by the inclusion of various antibacterial and antifungal agents such as paraben, chlorobutanol, phenol, sorbic acid and the like. It can also be desirable to include isotonic agents such as sugars, sodium chloride and the like. Prolonged absorption of the injectable pharmaceutical form can be brought about by the inclusion of agents, such as aluminum monostearate and gelatin, which delay absorption. Injectable depot forms are made by forming microencapsule matrices of the drug (e.g. peptide) in biodegradable polymers such as polylactide-polyglycolide, poly(orthoesters) and poly(anhydrides). Depending upon the ratio of drug to polymer and the nature of the particular polymer employed, the rate of drug release can be controlled. Depot injectable formulations are also prepared by entrapping the drug in liposomes or microemulsions that are compatible with body tissues. The injectable formulations can be sterilized, for example, by filtration through a bacterial-retaining filter or by incorporating sterilizing agents in the form of sterile solid compositions which can be dissolved or dispersed in sterile water or other sterile injectable media just prior to use. Suitable inert carriers can include sugars such as lactose. Desirably, at least 95% by weight of the particles of the active ingredient have an effective particle size in the range of 0.01 to 10 micrometers.

Thus, the compositions disclosed herein can comprise lipids such as liposomes, such as cationic liposomes (e.g., DOTMA, DOPE, DC-cholesterol) or anionic liposomes. Liposomes can further comprise proteins to facilitate targeting a particular cell, if desired. Administration of a composition comprising a peptide and a cationic liposome can be administered to the blood, to a target organ, or inhaled into the respiratory tract to target cells of the respiratory tract. For example, a composition comprising a peptide or nucleic acid sequence described herein and a cationic liposome can be administered to a subject's lung cells. Regarding liposomes, see, e.g., Brigham et al. Am. J. Resp. Cell. Mol. Biol. 1:95 100 (1989); Felgner et al. Proc. Natl. Acad. Sci USA 84:7413 7417 (1987); U.S. Pat. No. 4,897,355. Furthermore, the compound can be administered as a component of a microcapsule that can be targeted to specific cell types, such as macrophages, or where the diffusion of the compound or delivery of the compound from the microcapsule is designed for a specific rate or dosage.

In some instances, disclosed are pharmaceutical compositions comprising any of the disclosed peptides, peptide complexes, nucleic acid sequences or vectors described herein, or a pharmaceutically acceptable salt or solvate thereof, and a pharmaceutically acceptable carrier, buffer, or diluent. In various aspects, the peptide of the pharmaceutical composition is encapsulated in a delivery vehicle. In a further aspect, the delivery vehicle is a liposome, a microcapsule, or a nanoparticle. In a still further aspect, the delivery vehicle is PEG-ylated.

In the methods described herein, delivery of the compositions to cells can be via a variety of mechanisms. As defined above, disclosed herein are compositions comprising any one or more of the peptides described herein and can also include a carrier such as a pharmaceutically acceptable carrier. For example, disclosed are pharmaceutical compositions, comprising the peptides disclosed herein, and a pharmaceutically acceptable carrier. In one aspect, disclosed are pharmaceutical compositions comprising the disclosed peptides, peptide complexes, nucleic acid sequences or vectors. That is, a pharmaceutical composition can be provided comprising a therapeutically effective amount of at least one disclosed peptide or at least one product of a disclosed method and a pharmaceutically acceptable carrier.

In certain aspects, the disclosed pharmaceutical compositions comprise the disclosed peptides (including pharmaceutically acceptable salt(s) thereof) as an active ingredient, a pharmaceutically acceptable carrier, and, optionally, other therapeutic ingredients or adjuvants. The instant compositions include those suitable for nasal, oral, rectal, topical, and parenteral (including subcutaneous, intramuscular, and intravenous) administration, although the most suitable route in any given case will depend on the particular host, and nature and severity of the conditions for which the active ingredient is being administered. The pharmaceutical compositions can be conveniently presented in unit dosage form and prepared by any of the methods well known in the art of pharmacy.

In practice, the peptides described herein, or pharmaceutically acceptable salts thereof, of this invention can be combined as the active ingredient in intimate admixture with a pharmaceutical carrier according to conventional pharmaceutical compounding techniques. The carrier can take a wide variety of forms depending on the form of preparation desired for administration, e.g., oral or parenteral (including intravenous). Thus, the pharmaceutical compositions of the present invention can be presented as discrete units suitable for oral administration such as capsules, cachets or tablets each containing a predetermined amount of the active ingredient. Further, the compositions can be presented as a powder, as granules, as a solution, as a suspension in an aqueous liquid, as a non-aqueous liquid, as an oil-in-water emulsion or as a water-in-oil liquid emulsion. In addition to the common dosage forms set out above, the compounds of the invention, and/or pharmaceutically acceptable salt(s) thereof, can also be administered by controlled release means and/or delivery devices. The compositions can be prepared by any of the methods of pharmacy. In general, such methods include a step of bringing into association the active ingredient with the carrier that constitutes one or more necessary ingredients. In general, the compositions are prepared by uniformly and intimately admixing the active ingredient with liquid carriers or finely divided solid carriers or both. The product can then be conveniently shaped into the desired presentation.

The peptides, peptide complexes, nucleic acid sequences, or vectors described herein, or pharmaceutically acceptable salts thereof, can also be included in pharmaceutical compositions in combination with one or more other therapeutically active compounds.

Pharmaceutical compositions of the present invention suitable for parenteral administration can be prepared as solutions or suspensions of the active compounds in water. A suitable surfactant can be included such as, for example, hydroxypropylcellulose. Dispersions can also be prepared in glycerol, liquid polyethylene glycols, and mixtures thereof in oils. Further, a preservative can be included to prevent the detrimental growth of microorganisms.

Pharmaceutical compositions of the present invention suitable for injectable use include sterile aqueous solutions or dispersions. Furthermore, the compositions can be in the form of sterile powders for the extemporaneous preparation of such sterile injectable solutions or dispersions. Typically, the final injectable form should be sterile and should be effectively fluid for easy syringability. The pharmaceutical compositions should be stable under the conditions of manufacture and storage; thus, preferably should be preserved against the contaminating action of microorganisms such as bacteria and fungi. The carrier can be a solvent or dispersion medium containing, for example, water, ethanol, polyol (e.g., glycerol, propylene glycol and liquid polyethylene glycol), vegetable oils, and suitable mixtures thereof.

Injectable solutions, for example, can be prepared in which the carrier comprises saline solution, glucose solution or a mixture of saline and glucose solution. Injectable suspensions may also be prepared in which case appropriate liquid carriers, suspending agents and the like may be employed. Also included are solid form preparations that are intended to be converted, shortly before use, to liquid form preparations.

Pharmaceutical compositions of the present invention can be in a form suitable for topical use such as, for example, an aerosol, cream, ointment, lotion, dusting powder, mouth washes, gargles, and the like. Further, the compositions can be in a form suitable for use in transdermal devices. These formulations can be prepared, utilizing a compound of the invention, or pharmaceutically acceptable salts thereof, via conventional processing methods. As an example, a cream or ointment is prepared by mixing hydrophilic material and water, together with about 5 wt % to about 10 wt % of the compound, to produce a cream or ointment having a desired consistency.

In the compositions suitable for percutaneous administration, the carrier optionally comprises a penetration enhancing agent and/or a suitable wetting agent, optionally combined with suitable additives of any nature in minor proportions, which additives do not introduce a significant deleterious effect on the skin. Said additives may facilitate the administration to the skin and/or may be helpful for preparing the desired compositions. These compositions may be administered in various ways, e.g., as a transdermal patch, as a spot on, as an ointment.

Pharmaceutical compositions of this invention can be in a form suitable for rectal administration wherein the carrier is a solid. It is preferable that the mixture forms unit dose suppositories. Suitable carriers include cocoa butter and other materials commonly used in the art. The suppositories can be conveniently formed by first admixing the composition with the softened or melted carrier(s) followed by chilling and shaping in molds.

In addition to the aforementioned carrier ingredients, the pharmaceutical formulations described above can include, as appropriate, one or more additional carrier ingredients such as diluents, buffers, flavoring agents, binders, surface-active agents, thickeners, lubricants, preservatives (including anti-oxidants) and the like. Furthermore, other adjuvants can be included to render the formulation isotonic with the blood of the intended recipient. Compositions containing a disclosed peptide, and/or pharmaceutically acceptable salts thereof, can also be prepared in powder or liquid concentrate form.

The exact dosage and frequency of administration depends on the particular disclosed peptide, a product of a disclosed method of making, a pharmaceutically acceptable salt, solvate, or polymorph thereof, a hydrate thereof, a solvate thereof, a polymorph thereof, or a stereochemically isomeric form thereof; the particular condition being treated and the severity of the condition being treated; various factors specific to the medical history of the subject to whom the dosage is administered such as the age; weight, sex, extent of disorder and general physical condition of the particular subject, as well as other medication the individual may be taking; as is well known to those skilled in the art. Furthermore, it is evident that said effective daily amount may be lowered or increased depending on the response of the treated subject and/or depending on the evaluation of the physician prescribing the compositions.

Depending on the mode of administration, the pharmaceutical composition will comprise from 0.05 to 99% by weight, preferably from 0.1 to 70% by weight, more preferably from 0.1 to 50% by weight of the active ingredient, and, from 1 to 99.95% by weight, preferably from 30 to 99.9% by weight, more preferably from 50 to 99.9% by weight of a pharmaceutically acceptable carrier, all percentages being based on the total weight of the composition.

### G. Methods

Disclosed are methods for eliciting a protective immune response against coronavirus, methods of treating or preventing coronavirus infection and methods of reducing coronavirus viral titers in a subject infected with coronavirus. Disclosed are methods for eliciting a protective immune response against coronavirus, methods of treating or preventing SARS-COV-2 infection and methods of reducing coronavirus viral titers in a subject infected with an omicron or delta variant of SARS-COV-2. In some aspects, each of these methods comprise administering an effective amount of a composition comprising any of the peptides, peptide complexes, nucleic acids or vectors disclosed herein. As an example, each of these methods is further described below with regards to the coronavirus being SARS-CoV-2 and using the specific coronavirus antigen, a SARS-CoV-2 S antigen. In some aspects, the SARS-CoV-2 spike antigen is from an Omicron or delta strain of SARS-CoV-2.

Disclosed are methods for eliciting a protective immune response against SARS-CoV-2 comprising administering to a subject an effective amount of a composition comprising any of the peptides, peptide complexes, nucleic acids or vectors disclosed herein.

Disclosed are methods for eliciting a protective immune response against SARS-CoV-2 comprising administering to a subject an effective amount of a composition comprising a monomeric Fc fragment of an immunoglobulin recognized by a FcRn; a SARS-CoV-2 antigen; and a trimerization domain, wherein the administering is to a mucosal epithelium. In some aspects, the SARS-CoV-2 is an Omicron or delta strain of SARS-CoV-2.

Disclosed are methods for eliciting a protective immune response against SARS-CoV-2 comprising administering to a subject an effective amount of a composition comprising a peptide complex, wherein the peptide complex comprises three peptides forming a trimer, wherein each of the three peptides comprises a monomeric Fc fragment of an immunoglobulin recognized by a FcRn; a SARS-CoV-2 antigen; and a trimerization domain, wherein the administering is to a mucosal epithelium. In some aspects, the SARS-CoV-2 is an Omicron or delta strain of SARS-CoV-2.

Disclosed are methods for eliciting a protective immune response against SARS-CoV-2 comprising administering to a subject an effective amount of a composition comprising a peptide complex, wherein the peptide complex comprises three peptides forming a trimer, wherein each of the three peptides comprises a monomeric Fc fragment of an immunoglobulin recognized by a neonatal receptor (FcRn); a SARS-CoV-2 S antigen, wherein the SARS-CoV-2 S antigen comprises at least three mutations, wherein the mutations stabilize the S antigen in the prefusion form; and a trimerization domain, wherein the administering is to a mucosal epithelium. In some aspects, the monomeric Fc fragment of an immunoglobulin recognized by an FcRn comprises at least one, two, or three mutations in cysteines responsible for dimer formation. In some aspects, the SARS-CoV-2 S antigen comprises three or more mutations at the following positions: F817P, A892P, A899P, A942P, K986P, and V987P.

Disclosed are methods for eliciting a protective immune response against SARS-CoV-2 comprising administering to a subject an effective amount of a composition comprising a peptide complex, wherein the peptide complex comprises three peptides forming a trimer, wherein each of the three peptides comprises a monomeric Fc fragment of an immunoglobulin recognized by an FcRn, wherein the monomeric Fc fragment of an immunoglobulin recognized by an FcRn comprises at least three mutations in cysteine residues responsible for dimer formation; a SARS-CoV-2 S antigen, wherein the SARS-CoV-2 S antigen comprises at least two mutations, wherein the mutations stabilize the S antigen in the prefusion form; and a trimerization domain. In some aspects, the SARS-CoV-2 S antigen can have at least one, two, three, four, five, or six proline mutations corresponding to any of positions F817P, A892P, A899P, A942P, K986P, and V987P.

Disclosed are methods of treating or preventing SARS-CoV-2 infection in a subject. In some aspects, the SARS-CoV-2 is an Omicron or delta strain of SARS-CoV-2. Disclosed are methods of treating a subject exposed to SARS-CoV-2 or at risk of being exposed to SARS-CoV-2 comprising administering to a subject an effective amount of a composition comprising any of the peptides, peptide complexes, nucleic acids or vectors disclosed herein. In some aspects, the SARS-CoV-2 is an Omicron or delta strain of SARS-CoV-2.

Disclosed are methods of treating a subject exposed to SARS-CoV-2 or at risk of being exposed to SARS-CoV-2 comprising administering to the subject an effective amount of a composition comprising a peptide complex, wherein the peptide complex comprises three peptides forming a trimer, wherein each of the three peptides comprises a monomeric Fc fragment of an immunoglobulin recognized by a FcRn; a SARS-CoV-2 antigen; and a trimerization domain, wherein the administering is to a mucosal epithelium. In some aspects, the SARS-CoV-2 is an Omicron or delta strain of SARS-CoV-2.

Disclosed are methods of treating a subject exposed to SARS-CoV-2 or at risk of being exposed to SARS-CoV-2 comprising administering to a subject an effective amount of a composition comprising a monomeric Fc fragment of an immunoglobulin recognized by a FcRn; a SARS-CoV-2 antigen; and a trimerization domain, wherein the administering is to a mucosal epithelium. In some aspects, the SARS-CoV-2 spike antigen is from an Omicron or delta strain and/or the SARS-CoV-2 virus infection is a SARS-CoV-2 omicron or delta strain infection. A subject at risk of being exposed to SARS-CoV-2 can be a first responder, a healthcare worker, a teacher, or anyone knowingly or unknowingly coming in contact with a person infected with SARS-CoV-2. In some aspects, treating a subject at risk of being exposed to SARS-CoV-2 can result in preventing SARS-CoV-2 infection. In some aspects, treating a subject at risk of being exposed to SARS-CoV-2 can result in preventing serious symptoms or side-effects of a SARS-CoV-2 infection, such as but not limited to, pneumonia, organ failure, cytokine storm, or death.

Disclosed are methods of treating a subject exposed to SARS-CoV-2 or at risk of being exposed to SARS-CoV-2 comprising administering to a subject an effective amount of a composition comprising a peptide complex, wherein the peptide complex comprises three peptides forming a trimer, wherein each of the three peptides comprises a monomeric Fc fragment of an immunoglobulin recognized by a neonatal receptor (FcRn); a SARS-CoV-2 S antigen, wherein the SARS-CoV-2 S antigen comprises at least three mutations, wherein the mutations stabilize the S antigen in the prefusion form; and a trimerization domain, wherein the administering is to a mucosal epithelium. In some aspects, the monomeric Fc fragment of an immunoglobulin recognized by an FcRn comprises at least one, two, or three mutations in cysteines responsible for dimer formation. In some aspects, the SARS-CoV-2 S antigen comprises three or more mutations at the following positions: F817P, A892P, A899P, A942P, K986P, and V987P.

Disclosed are methods of treating a subject exposed to SARS-CoV-2 or at risk of being exposed to SARS-CoV-2 comprising administering to a subject an effective amount of a composition comprising a peptide complex, wherein the peptide complex comprises three peptides forming a trimer, wherein each of the three peptides comprises a monomeric Fc fragment of an immunoglobulin recognized by an FcRn, wherein the monomeric Fc fragment of an immunoglobulin recognized by an FcRn comprises at least three mutations in cysteine residues responsible for dimer formation; a SARS-CoV-2 S antigen, wherein the SARS-CoV-2 S antigen comprises at least two mutations, wherein the mutations stabilize the S antigen in the prefusion form; and a trimerization domain. In some aspects, the SARS-CoV-2 S antigen can have at least one, two, three, four, five, or six proline mutations corresponding to any of positions F817P, A892P, A899P, A942P, K986P, and V987P.

Disclosed are methods of reducing SARS-CoV-2 viral titers in a subject infected with SARS-CoV-2 comprising administering to a subject an effective amount of a composition comprising any of the peptides, peptide complexes, nucleic acids or vectors disclosed herein. In some aspects, the SARS-CoV-2 spike antigen is from an Omicron or delta strain and/or the SARS-CoV-2 virus infection is a SARS-CoV-2 omicron or delta strain infection. Disclosed are methods of reducing SARS-CoV-2 viral titers in a subject infected with SARS-CoV-2 comprising administering to a subject an effective amount of a composition comprising a monomeric Fc fragment of an immunoglobulin recognized by a FcRn; a SARS-CoV-2 antigen; and a trimerization domain, wherein the administering is to a mucosal epithelium. In some aspects, the SARS-CoV-2 spike antigen is from an Omicron or delta strain and/or the SARS-CoV-2 virus infection is a SARS-CoV-2 omicron or delta strain infection.

Disclosed are methods of reducing SARS-CoV-2 viral titers in a subject infected with SARS-CoV-2 comprising administering to a subject an effective amount of a composition comprising a peptide complex, wherein the peptide complex comprises three peptides forming a trimer, wherein each of the three peptides comprises a monomeric Fc fragment of an immunoglobulin recognized by a neonatal receptor (FcRn); a SARS-CoV-2 S antigen, wherein the SARS-CoV-2 S antigen comprises at least three mutations, wherein the mutations stabilize the S antigen in the prefusion form; and a trimerization domain, wherein the administering is to a mucosal epithelium. In some aspects, the monomeric Fc fragment of an immunoglobulin recognized by an FcRn comprises at least one, two, or three mutations in cysteines responsible for dimer formation. In some aspects, the SARS-CoV-2 S antigen comprises three or more mutations at the following positions: F817P, A892P, A899P, A942P, K986P, and V987P.

Disclosed are methods of reducing SARS-CoV-2 viral titers in a subject infected with SARS-CoV-2 comprising administering to a subject an effective amount of a composition comprising a peptide complex, wherein the peptide complex comprises three peptides forming a trimer, wherein each of the three peptides comprises a monomeric Fc fragment of an immunoglobulin recognized by an FcRn, wherein the monomeric Fc fragment of an immunoglobulin recognized by an FcRn comprises at least three mutations in cysteine residues responsible for dimer formation; a SARS-CoV-2 S antigen, wherein the SARS-CoV-2 S antigen comprises at least two mutations, wherein the mutations stabilize the S antigen in the prefusion form; and a trimerization domain. In some aspects, the SARS-CoV-2 S antigen can have at least one, two, three, four, five, or six proline mutations corresponding to any of positions F817P, A892P, A899P, A942P, K986P, and V987P.

Disclosed are methods of treating a subject at risk for infection with coronavirus comprising administering an effective amount of a composition comprising any of the peptides, peptide complexes, nucleic acids or vectors disclosed herein.

Disclosed are methods of preventing the spread of coronavirus from a subject infected with a coronavirus to a non-infected subject comprising administering an effective amount of a composition comprising any of the peptides, peptide complexes, nucleic acids or vectors disclosed herein.

Disclosed are methods of preventing coronavirus infection in a subject comprising: administering an effective amount of a composition comprising any of the peptides, peptide complexes, nucleic acids or vectors disclosed herein.

Disclosed herein are methods of reducing coronavirus copy number per cell comprising administering an effective amount of a composition comprising any of the peptides, peptide complexes, nucleic acids or vectors disclosed herein.

In some aspects, the disclosed methods are directed to treating a subject having a coronavirus, reducing coronavirus titer or copy number, preventing infection with a coronavirus, eliciting an immune response against coronavirus, wherein the coronavirus can be any SARS-CoV-2 strains of α, β, γ, δ, or ε (original or subvariant strains). In some aspects, the coronavirus can be SARS-CoV-2 or from a variant strain, such as, but not limited to, the variants of D614G, B.1.1.7 or 20I/501Y.V1 (B.1.351 or 20H/501.V2, P.1 or 20J/501Y.V3, 20C/S:452R, Cluster 5 Variant, XBB.1.5 (e.g. 1.5.70, 1.5.68, 1.5.72, 1.5.10, 1.5.59, 1.5.1), XBB.1.16 (e.g. 1.16.6, 1.16.11, 1.16.15, 1.6.1), EG.5, XBB.1.9 (e.g. 1.9.1, 1.9.2), XBB.2.3, BA.1, BA.5, BA.2.86, HV.1, FL.1.5.1, HK.3, JD.1.1, JF.1, GK.1,1., HF.1, BA.2 (e.g. 2.12.1, 2.12.2), BAA, BA.5, BA.527, BA.529, GE.1, XBB, GK.2, EG.6.1, XBB.1.42.2, CH.1.1, XBB.2.3.8, FD.1.1, FE.1.1, EU.1.1, B.1.1.529, B.1.617.2, XBB.128, or BQ.1 (e.g. 1.1).

Disclosed are methods for eliciting a protective immune response against SARS-CoV-2 comprising administering to a subject an effective amount of a composition comprising any of the peptides, peptide complexes, nucleic acids or vectors disclosed herein, wherein the SARS-CoV-2 is an Omicron or delta strain.

Disclosed are methods for eliciting a protective immune response against SARS-CoV-2 comprising administering to a subject an effective amount of a composition comprising a monomeric Fc fragment of an immunoglobulin recognized by a FcRn; a SARS-CoV-2 antigen; and a trimerization domain, wherein the administering is to a mucosal epithelium, wherein the SARS-CoV-2 is an Omicron or delta strain.

Disclosed are methods for eliciting a protective immune response against SARS-CoV-2 comprising administering to a subject an effective amount of a composition comprising a peptide complex, wherein the peptide complex comprises three peptides forming a trimer, wherein each of the three peptides comprises a monomeric Fc fragment of an immunoglobulin recognized by a FcRn; a SARS-CoV-2 antigen; and a trimerization domain, wherein the administering is to a mucosal epithelium, wherein the SARS-CoV-2 is an Omicron or delta strain.

Disclosed are methods of treating or preventing SARS-CoV-2 infection in a subject, wherein the SARS-CoV-2 is an Omicron or delta strain. Disclosed are methods of treating a subject exposed to SARS-CoV-2 or at risk of being exposed to SARS-CoV-2 comprising administering to a subject an effective amount of a composition comprising any of the peptides, peptide complexes, nucleic acids or vectors disclosed herein, wherein the SARS-CoV-2 is an Omicron or delta strain.

Disclosed are methods of treating a subject exposed to SARS-CoV-2 or at risk of being exposed to SARS-CoV-2 comprising administering to the subject an effective amount of a composition comprising a peptide complex, wherein the peptide complex comprises three peptides forming a trimer, wherein each of the three peptides comprises a monomeric Fc fragment of an immunoglobulin recognized by a FcRn; a SARS-CoV-2 antigen; and a trimerization domain, wherein the administering is to a mucosal epithelium, wherein the SARS-CoV-2 is an Omicron or delta strain.

Disclosed are methods of treating a subject exposed to SARS-CoV-2 or at risk of being exposed to SARS-CoV-2 comprising administering to a subject an effective amount of a composition comprising a monomeric Fc fragment of an immunoglobulin recognized by a FcRn; a SARS-CoV-2 antigen; and a trimerization domain, wherein the administering is to a mucosal epithelium, wherein the SARS-CoV-2 is an Omicron or delta strain. A subject at risk of being exposed to SARS-CoV-2 can be a first responder, a healthcare worker, a teacher, or anyone knowingly or unknowingly coming in contact with a person infected with SARS-CoV-2. In some aspects, treating a subject at risk of being exposed to SARS-CoV-2 can result in preventing SARS-CoV-2 infection. In some aspects, treating a subject at risk of being exposed to SARS-CoV-2 can result in preventing serious symptoms or side-effects of a SARS-CoV-2 infection, such as but not limited to, pneumonia, organ failure, cytokine storm, or death.

Disclosed are methods of reducing SARS-CoV-2 viral titers in a subject infected with SARS-CoV-2 comprising administering to a subject an effective amount of a composition comprising any of the peptides, peptide complexes, nucleic acids or vectors disclosed herein, wherein the SARS-CoV-2 is an Omicron or delta strain. Disclosed are methods of reducing SARS-CoV-2 viral titers in a subject infected with SARS-CoV-2 comprising administering to a subject an effective amount of a composition comprising a monomeric Fc fragment of an immunoglobulin recognized by a FcRn; a SARS-CoV-2 antigen; and a trimerization domain, wherein the administering is to a mucosal epithelium, wherein the SARS-CoV-2 is an Omicron or delta strain.

Disclosed are methods of treating a subject at risk for infection with coronavirus comprising administering an effective amount of a composition comprising any of the peptides, peptide complexes, nucleic acids or vectors disclosed herein, wherein the SARS-CoV-2 is an Omicron or delta strain.

Disclosed are methods of preventing the spread of coronavirus from a subject infected with a coronavirus to a non-infected subject comprising administering an effective amount of a composition comprising any of the peptides, peptide complexes, nucleic acids or vectors disclosed herein, wherein the SARS-CoV-2 is an Omicron or delta strain.

Disclosed are methods of preventing coronavirus infection in a subject comprising: administering an effective amount of a composition comprising any of the peptides, peptide complexes, nucleic acids or vectors disclosed herein, wherein the SARS-CoV-2 is an Omicron or delta strain.

Disclosed herein are methods of reducing coronavirus copy number per cell comprising administering an effective amount of a composition comprising any of the peptides, peptide complexes, nucleic acids or vectors disclosed herein, wherein the SARS-CoV-2 is an Omicron or delta strain.

In some instances, the mucosal epithelium is selected from the group consisting of: lungs, intestines, trachea, colon, nasal tissue, and vaginal tissue. In some aspects, administering is administering to a mucosal epithelium and can be a direct or indirect administration of the disclosed peptides, peptide complexes, nucleic acid sequences or vectors to one or more of the mucosal epithelium described herein.

In some instances, administering is intranasal administering. In some instances, any form of administering that allows for delivery to a mucosal epithelium can be used.

In some instances, an adjuvant is further administered with the composition. In some instances, an adjuvant can be formulated with the peptide into the disclosed compositions. In some instances, the disclosed compositions or peptides can further comprise an adjuvant. Thus, the adjuvant can be administered simultaneously with the peptide. In some instances, the adjuvant is separate from the disclosed compositions and therefore can be administered simultaneously with the composition or separate from the composition. The adjuvant can be, for example, but is not limited to, CpG, MPL, poly[di(sodium carboxylatoethylphenoxy)phosphazene](PCEP), poly[di(sodium carboxylatophenoxy)phosphazene](PCPP), the Cholera Toxin-Derived CTA1-DD, Flagellin, IDR1002, α-Galactosylceramide, or saponins. The term “adjuvant” is intended to include any substance which is incorporated into or administered simultaneously with the peptides of the invention and which nonspecifically potentiates the immune response in the subject. Adjuvants include aluminum compounds, e.g., gels, aluminum hydroxide and aluminum phosphate, and Freund's complete or incomplete adjuvant (in which the fusion protein is incorporated in the aqueous phase of a stabilized water in paraffin oil emulsion). The paraffin oil may be replaced with different types of oils, e.g., squalene or peanut oil. Other materials with adjuvant properties include, flagellin, BCG (attenuated Mycobacterium tuberculosis), calcium phosphate, levamisole, isoprinosine, polyanions (e.g., poly A:U) leutinan, pertussis toxin, cholera toxin, lipid A, saponins and peptides, e.g. muramyl dipeptide. dimethyl dioctadecyl-ammonium bromide (DDA); monophosphoryl lipid A (MPL); LTK63, lipophilic quaternary ammonium salt-DDA, Trehalose dimycolate and synthetic derivatives, DDA-MPL, DDA-TDM, DDA-TDB, IC-31, aluminum salts, aluminum hydroxyide, aluminum phosphate, potassium aluminum phosphate, Montanide ISA-51, ISA-720, microparticles, immunostimulatory complexes, liposomes, virosomes, virus-like particles, CpG oligonucleotides, cholera toxin, heat-labile toxin from E. coli, lipoproteins, dendritic cells, IL-12, GM-CSF, nanoparticles including calcium phosphate nanoparticles, combination of soybean oil, emulsifying agents, and ethanol to form a nanoemulsion; AS04, ZADAXIN, or combinations thereof. Rare earth salts, e.g., lanthanum and cerium, may also be used as adjuvants. The amount of adjuvants depends on the subject and the particular peptide used and can be readily determined by one skilled in the art without undue experimentation.

In some aspects, eliciting a protective immune response comprises eliciting neutralizing antibodies. In some aspects, eliciting a protective immune response comprises activating T cells and B cells. In some aspects, the activated T cells and B cells provide a cellular and humoral response, respectively.

In some aspects, an effective amount is that amount of the disclosed peptides, peptide complexes or compositions that will alone, or together with further doses, stimulate an immune response as desired. This may involve the stimulation of a humoral antibody response resulting in an increase in antibody titer in serum, improved mucosal immunity, a clonal expansion of cytotoxic T lymphocytes or tolerance to an antigen, including a self-antigen. It is believed that doses ranging from 1 nanogram/kilogram to 100 milligrams/kilogram, depending upon the mode of administration, will be effective. In some aspects, the preferred range is believed to be between about 500 nanograms and 500 micrograms/kilogram, and most preferably between 1 microgram and 100 micrograms/kilogram. The absolute amount will depend upon a variety of factors, including the peptide, peptide complex, or composition selected, the immune modulation desired, whether the administration is in a single or multiple doses, and individual patient parameters including age, physical condition, size and weight. These factors are well known to those of ordinary skill in the art and can be addressed with no more than routine experimentation.

### H. Combination Therapy

Any of the disclosed methods described herein can be performed in combination with one or more of the known standards of care for coronavirus infection. Thus, in some aspects, the methods comprising administering one or more of the disclosed peptide complexes, peptides, compositions or nucleic acids can be combined with an antibody, or antibody cocktail, nanobody, antiviral small molecules, macromolecules of sulfated polysaccharides, and polypeptides. Frequent targets are the viral spike protein, the host angiotensin converting enzyme 2, the host transmembrane protease serine 2, and clathrin-mediated endocytosis. For example, disclosed methods of using TTFields can be performed in combination with one or more of remdesivir (Veklury), Nafamostat, Avigan (favilavir), bamlanivimab, Olumiant and Baricinix (baricitinib), hydroxychloroquine/chloroquine, Casirivimab and imdevimab (formerly REGN-COV2), PTC299, Leronlimab (PRO 140), Bamlanivimab (LY-CoV555), Lenzilumab, Ivermectin, RLF-100 (aviptadil), Metformin (Glucophage, Glumetza, Riomet), AT-527, Actemra (tocilizumab), Niclocide (niclosamide), Convalescent plasma, Pepcid (famotidine), Kaletra (lopinavir-ritonavir), Remicade (infliximab), AZD7442, AZD7442, CT-P59, Heparin (UF and LMW), VIR-7831 (GSK4182136), JS016, Kevzara (sarilumab), SACCOVID (CD24Fc), Humira (adalimumab), COVI-GUARD (STI-1499), Dexamethasone (Dextenza, Ozurdex, others), PB1046, Galidesivir, Bucillamine, PF-00835321 (PF-07304814), Eliquis (Apixaban), Takhzyro (lanadelumab), Hydrocortisone, Ilaris (canakinumab), Colchicine (Mitigare, Colcrys), BLD-2660, Avigan (favilavir/avifavir), Rhu-pGSN (gelsolin), MK-4482, TXA127, LAM-002A (apilimod dimesylate), DNL758 (SAR443122), INOpulse, ABX464, AdMSCs, Losmapimod, Mavrilimumab, or Calquence (acalabrutinib), quinoline-based antimalarials ((hydroxy)-chloroquine and others), RAAS modifiers (captopril, losartan, and others), statins (atorvastatin and simvastatin), guanidino-based serine protease inhibitors (camostat and nafamostat), antibacterials (macrolides, clindamycin, and doxycycline), antiparasitics (ivermectin and niclosamide), cardiovascular drugs (amiodarone, verapamil, and tranexamic acid), antipsychotics (chlorpromazine), antivirals (umifenovir and oseltamivir), DPP-4 inhibitors (linagliptin), JAK inhibitors (baricitinib and others), sulfated glycosaminoglycans (UFH and LMWHs) and polypeptides such as the enzymes DAS181 and rhACE2. They also include the viral spike protein-targeting monoclonal antibodies REGN10933 and REGN10987.

In some aspects, the additional therapeutic agents are selected based on the disease or symptom to be treated. A description of the various classes of suitable pharmacological agents and drugs may be found in Goodman and Gilman, The Pharmacological Basis of Therapeutics, (11th Ed., McGraw-Hill Publishing Co.) (2005). In some aspects, an additional therapeutic agent can be CpG which helps overcome any possible immune tolerance. In some aspects, an additional therapeutic agent can be an anti-viral or any known SARS-CoV-2 therapeutic.

In some aspects, an additional therapeutic agent can be MPL (Monophosphoryl Lipid A) or C-di-GMP (Cyclic diguanylate monophosphate, CpG). In some aspects, an additional therapeutic agent can be a toll-like receptor (TLR) agonist, which represent different adjuvants, CpG and MPL are examples.

In some aspects, supplementary immune potentiating agents, such as cytokines, can be delivered in conjunction with the disclosed peptide complexes, peptides and nucleic acids of the invention. The cytokines contemplated are those that will enhance the beneficial effects that result from administering the peptide complexes, peptides and nucleic acids according to the invention. Cytokines are factors that support the growth and maturation of cells, including lymphocytes. It is believed that the addition of cytokines will augment cytokine activity stimulated in vivo by carrying out the methods of the invention. The preferred cytokines are interleukin (IL)-1, IL-2, gamma-interferon and tumor necrosis factor α. Other useful cytokines are believed to be IL-3, IL-4, IL-5, IL-6, IL-7, IL-8, IL-9, IL-10, IL-11, IL-12, IL-13, erythropoietin, leukemia inhibitory factor, oncostatin-M, ciliary neurotrophic factor, growth hormone, prolactin, CD40-ligand, CD27-ligand, CD30-ligand, alpha-interferon, beta-interferon, and tumor necrosis factor 3. Other cytokines known to modulate T-cell activity in a manner likely to be useful according to the invention are colony stimulating factors and growth factors including granulocyte and/or macrophage stimulating factors (GM-CSF, G-CSF and CSF-1) and platelet derived, epidermal, insulin-like, transforming and fibroblast growth factors. The selection of the particular cytokines will depend upon the particular modulation of the immune system that is desired. The activity of cytokines on particular cell types is known to those of ordinary skill in the art.

### I. Kits

The compositions and materials described above as well as other materials can be packaged together in any suitable combination as a kit useful for performing, or aiding in the performance of, the disclosed method. It is useful if the kit components in a given kit are designed and adapted for use together in the disclosed method. For example, disclosed are kits for producing the disclosed peptides, the kit comprising monomeric Fc fragments of an immunoglobulin recognized by a FcRn and a SARS-CoV-2 antigen. The kits also can contain vectors.

### EXAMPLES

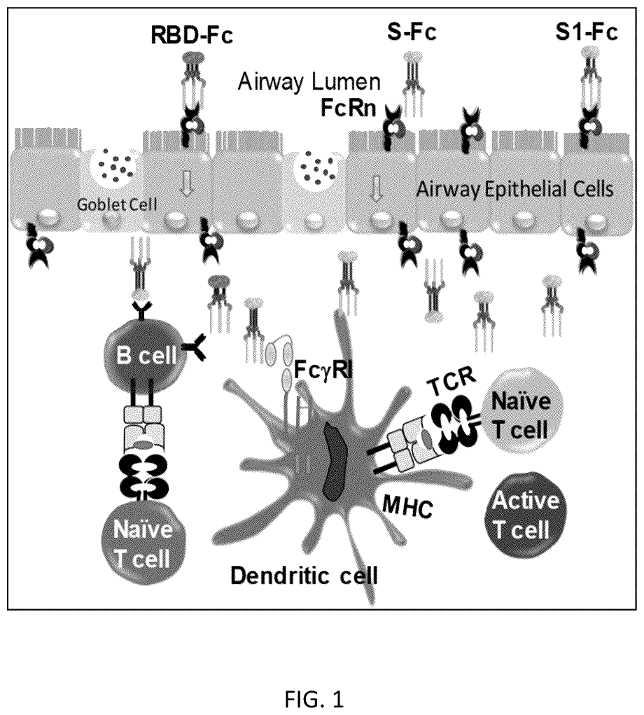

FcRn mediates the transfer of IgG across polarized respiratory epithelial cells and prolongs IgG half-life. Described herein is the use of the FcRn to deliver SARS-CoV-2 spike antigens to induce protective immunity against SARS-CoV-2 virus infection. Intranasal immunization (i.n.) with the trimeric spike proteins that target to FcRn plus a mucosal adjuvant conferred significant protection against lethal virus challenge in human ACE2 transgenic mice. The results demonstrate that FcRn can effectively deliver trimeric spike antigens in the respiratory tract and elicit potent protection against lethal SARS-CoV-2 infection. Therefore, FcRn-mediated respiratory immunization can efficiently induce protective respiratory immunity to SARS-COV-2 infection and COVID-19 disease (FIG. 1).

### 1. Importance of Developing a Nasal Spray Vaccine Against SARS-CoV-2 Infection and Transmission

Currently, nucleic acid-, viral vector-, and subunit-based vaccines, are in progress or on the market. However, there is still a need to develop a COVID-19 vaccine inducing a high degree of mucosal immunity to block viral spread. The strategy disclosed herein is based on the following: 1) By exploiting a natural IgG transfer pathway, we proved the concept that FcRn-targeted intranasal immunization of mice with trimeric influenza HA-Fc protein induced both local and systemic immune responses and protected mice from infection. We reason that FcRn mucosal delivery could also enhance mucosal uptake of Fc-fused SARS-CoV-2 S antigens through intranasal delivery. After epithelial transport, S antigens efficiently bind to Fcγ receptors on dendritic cells. 2). The property of FcRn in protecting IgG from degradation could similarly extend the half-life of S-Fc antigens. This would allow professional antigen presenting cells (APCs), dendritic cell, macrophages, and B cells to sample and present S antigens for a long time in APCs that enhance T cell activation. 3). The full-length proteins S, S1, or RBD in SARS-CoV-2 have been proposed as major vaccine antigens because they induce neutralizing antibodies that prevent host cell attachment and infection by virus. 4). We have produced a trimeric form of SARS-CoV-2 S-Fc, S1-Fc, RBD-Fc antigens, the mice intranasally immunized with trimeric S-Fc, S1-Fc or RBD-Fc antigens developed specific neutralizing antibodies. 5). FcRn-mediated IgG transport is well-conserved across species, human FcRn is expected to transport SARS-CoV-2 antigens in humans.

### 2. Developing an Effective Mucosal Vaccine Against SARS-CoV-2

SARS-CoV-2 seems more contagious for quickly and easily spreading among people. The virus can spread via droplets or aerosol from the infected individuals with or without symptoms. Given the main cause of patient death is pneumonia, therefore achieving an effective and long-lasting immunity in the respiratory tract would better prevent or control the SARS-CoV-2 spread and infection in the community. However, to elicit resident memory T and B cells in the lung, vaccine antigens must be delivered into the lungs. It has been shown that FcRn mucosal delivery can induce potent protection from influenza infection. FcRn can similarly deliver SARS-CoV-2 S antigens across the respiratory barrier, thus inducing protective respiratory immunity to SARS-CoV-2 viruses. It is expected that mucosal immunity can prevent nasal infection or shedding of the virus. FcRn-targeted delivery represents an important path for developing a mucosal vaccine against SARS-CoV-2.

### 3. Developing a Safe SARS-CoV-2 Mucosal Vaccine in the Young or Elderly Population

Elderly people are most likely to develop severe forms of COVID-19, however, achieving immune protection by a vaccine may be challenging in the elderly. Also, although infected children have less symptoms, the immunization of the young population would reduce viral transmission. Since vaccine preparation mainly contains Spike proteins, FcRn mucosal delivery would mitigate the risk and develop an effective and safe immunity in both young and elderly. Overall, FcRn-targeted mucosal vaccination can help control the COVID-19 pandemic but not only preventing the disease severity in individuals, but also stopping viral infection and spread among people.

### 4. Expression of SARS-COV-2 S, S1, or RBD Antigen that is Fused to Human IgG 1 Fc

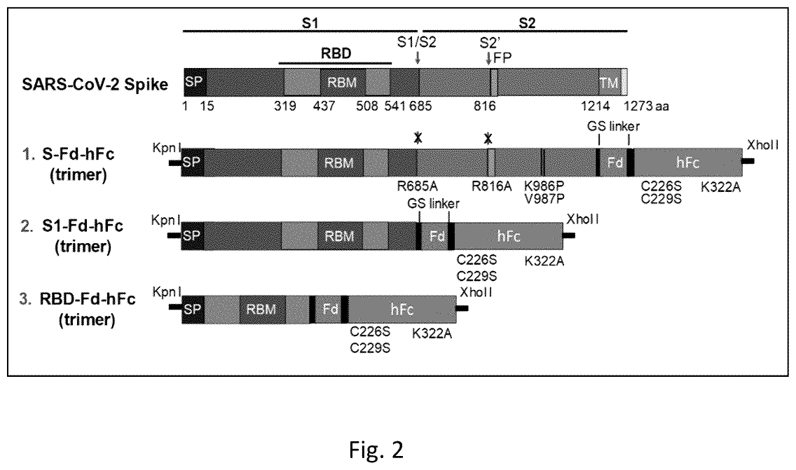

The rationale for using human IgG1 is consistent with the fact that it has the highest affinity for activating FcγRI, but the lowest affinity for inhibitory FcγRIIB. Because IgG Fc normally forms a disulfide-bonded dimer, a monomeric Fc was created by substituting cysteines 226 and 229 of human IgG1 with serine to eliminate the disulfide bonds. In IgG Fc, the complement C1q-binding motif was eliminated (K322A) (FIG. 2), allowing production of a non-lytic vaccine antigen.

The entire amino acid (aa) sequence of the SARS-COV-2 was retrieved from Genbank (MN908947). the S gene of SARS-COV-2. The S gene was cloned into eukaryotic expression plasmid pcDNA3 to generate the envelope recombinant plasmids pcDNA3-S (FIG. 2). During SARS-COV-2 infection, the S precursor is cleaved into S1 and S2. To produce a non-cleavable S protein, mutagenesis was performed at the cleavage site (R685A/R816A) of the S gene to keep the S protein in pre-cleavage conformation. The maintenance of a native conformational structure of SARS-COV-2 Spike antigen in a prefusion state would be critical for maximizing the immunogenicity induced by intranasal vaccination. To maintain the S protein in a prefusion state, two mutations (K986P and V987P) were introduced.

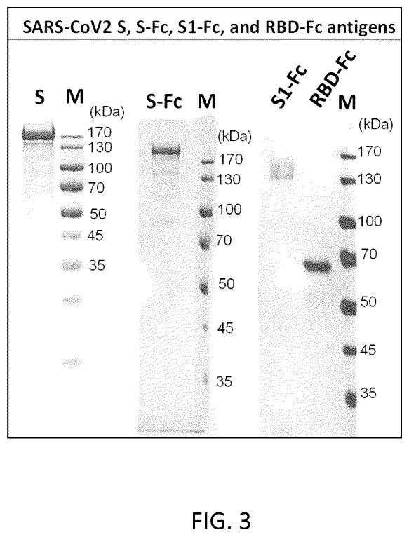

The SARS-CoV-2 S protein naturally exists as a trimer. To facilitate the trimerization of S protein, a foldon domain from T4 bacteriophage fibritin protein was engineered to the C-terminus of S (residues 15-1214), S1 (residues 15-672), and RBD (residues 319-540) genes. As described above, the monomeric human IgG1 Fc/wt was fused in frame with the S-foldon, S1-foldon, and RBD-foldon, generating S-Fc ((FIG. 2, construct #1), S1-Fc (construct #2) and RBD-Fc (construct #3), respectively. In a Coomassie blue staining, the S, S-Fc, S1-Fc, and RBD-Fc proteins were secreted from 293T or CHO cells (FIG. 3). In a Western blot, the secreted S-Fc/wt, S1-Fc/wt, RBD-Fc/wt proteins were monomers under non-reducing conditions. This confirmed that removal of the disulfide bonds eliminated Fc dimerization. To determine whether S-Fc protein binds to FcRn, it was tested whether S-Fc interacts with Protein A because of the IgG Fc binding sites for both FcRn and Protein A overlap. The S-Fc interacted with Protein A strongly indicating that S-Fc proteins maintain the structure required to interact with FcRn.

### 5. Intranasal Immunization of Mouse with S-Fc, S1-Fc or RBD-Fc Induced S-Specific Antibody Immune Responses

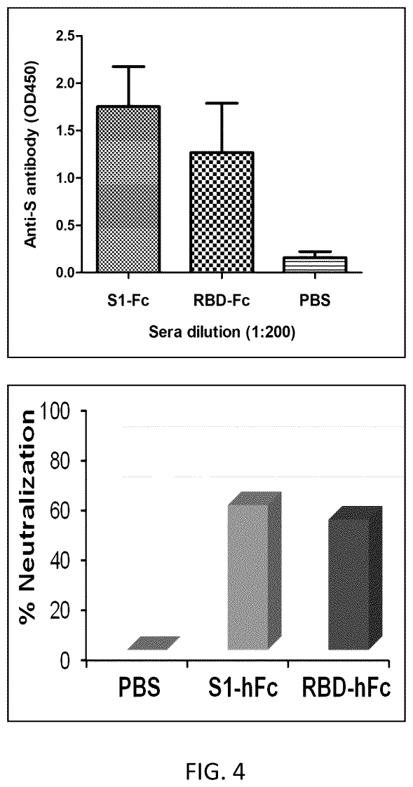

Whether mice intranasally (i.n.) immunized with IgG Fc-fused S1 and RBD proteins can develop antibody immune responses was tested. CpG1826 was co-administrated to overcome possible mucosal tolerance. Briefly, mice were i.n. immunized with 10 μg of affinity-purified S1-Fc, RBD-Fc protein, or PBS in combination with 10 μg CpG, and boosted 2 weeks later with the same dose. Significantly higher titers of total IgG in sera, measured by ELISA, were detected in the S1-Fc or RBD-Fc immunized mice when compared with PBS-immunized mice (FIG. 4, left panel).

SARS-CoV-2 neutralization was measured using SARS-CoV-2-FBLuc in a single-cycle pseudovirus neutralization assay in ACE2/293T cells. Pseudovirions were produced by cotransfection Lenti-X 293T cells with pMLV-gag-pol, pFBluc, and pcDNA 3.1 SARS-CoV-2 S (BEI Resources) using Lipofectamine 3000. The supernatant was harvested at 72 hr after transfection. For the neutralization assay, 50 μl of SARS-CoV-2 S pseudovirions was preincubated with an equal volume of medium, containing serum at varying dilutions at room temperature for 1 hour; then, virus-antibody mixtures were added to ACE2/293T cells. Cells were lysed 72 hour later, and luciferase activity was measured using luciferin-containing substrate. The average percent inhibitions by mouse intranasal vaccination are shown in FIG. 4 (right panel). Control sera (control) did not neutralize SARS-CoV-2 in this assay. Sera generated by S1 and RBD showed 50 to 60% virus neutralization after vaccination.

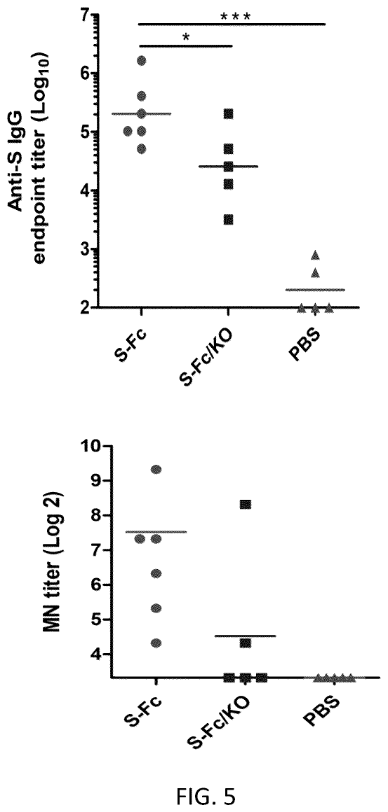

Whether FcRn-dependent respiratory transport augments the immune responses of S antigen was also tested. Wild-type mice (N=6) or FcRn knockout mice (KO) (N=5) were intranasally (i.n.) immunized with 10 μg of S-Fc, or PBS in combination with 10 μg CpG, and boosted 2 weeks later with the same dose. Significantly higher titers of S-specific IgG in sera were seen in the S-Fc immunized mice when compared with that of S-Fc-immunized FcRn KO mice or PBS-treated groups of mice 2 weeks after the boost (FIG. 5, Left). Moreover, sera from the S-Fc/wt immunized mice exhibited strong neutralizing activity relative to FcRn KO or PBS control groups (FIG. 5, Right). Overall, the data indicate that Fc-fused S, S1 or RBD antigens administered via the intranasal route can induce the S-specific neutralizing antibody, this immune response should depend on FcRn transport.

### 6. FcRn-Targeted Nasal Vaccination Leads to Increased Protection Against Lethal SARS-Cov-2 Infection

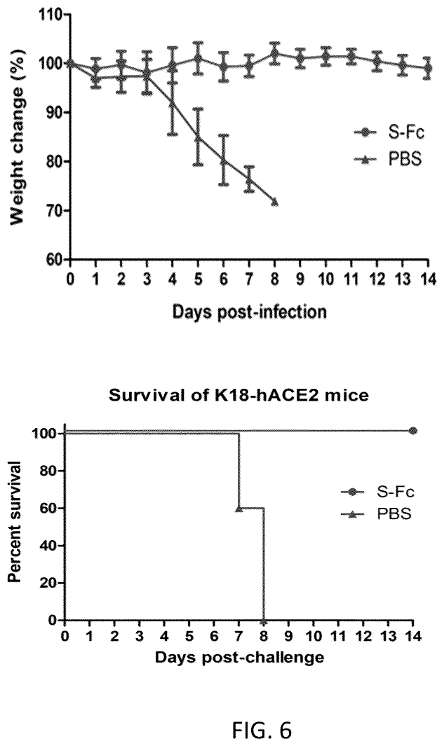

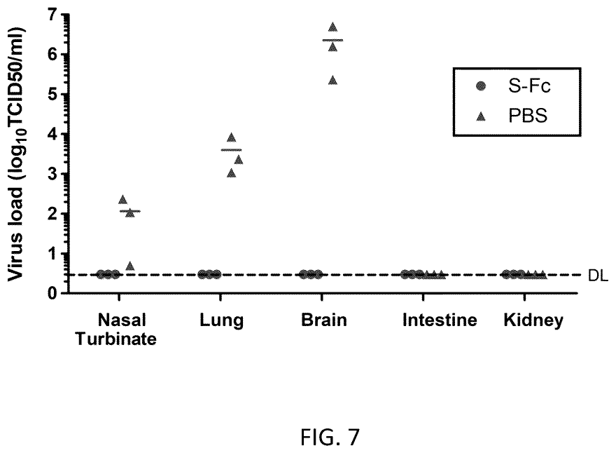

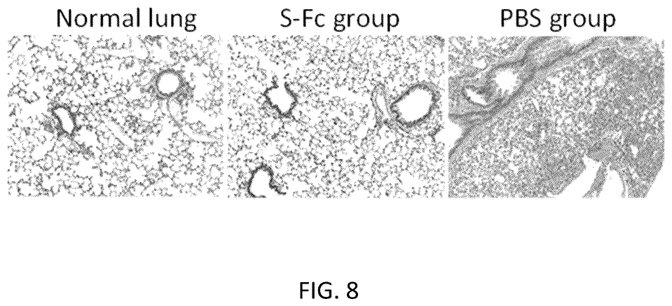

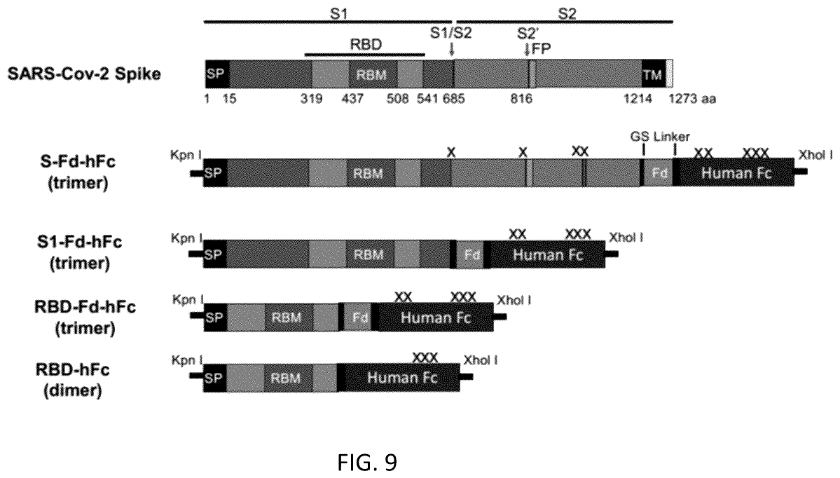

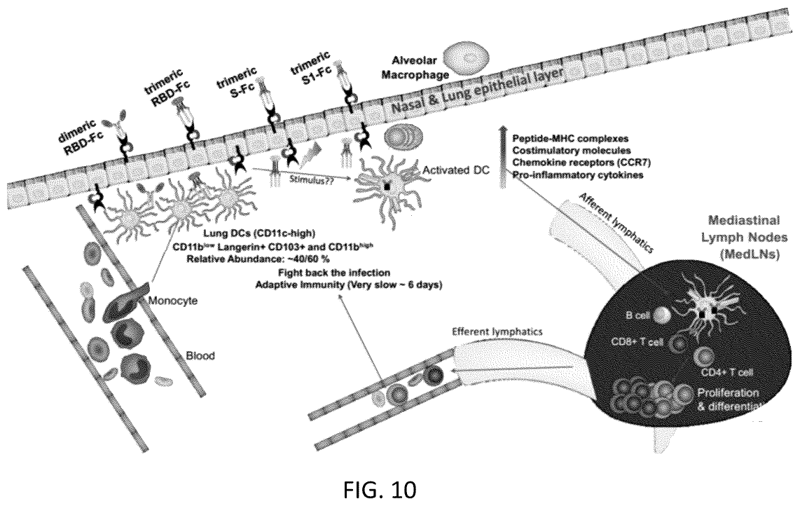

SARS-CoV-2 virus infects human ACE2 transgenic mice. To test whether the immune responses elicited by FcRn-targeted intranasal vaccination provide protection, 8-10-week-old human ACE2 transgenic mice were i.n. immunized intranasally (i.n.) with 10 μg of S-Fc, or PBS in combination with 10 μg CpG, and boosted 2 weeks later with the same dose. The mice were challenged i.n. with a lethal dose (2.5×10⁴ TCID50) of SARS-CoV-2 virus two weeks after the boost in BSL-3 facility. Mice were monitored and weighed daily for a 14-day period and were euthanized after 25% body weight loss as endpoint. All mice in the PBS groups had weight loss (up to 25%) within 8 days after the challenge and either succumbed to infection or euthanized. In contrast, all the S-Fc-immunized mice had no body-weight loss (FIG. 6, Left). Hence, the trimeric S-Fc protein-immunized mice led to a full protection (FIG. 6, right). Also, virus replicating was assessed in different tissues by 5 days after challenge (FIG. 7). Virus was not detected in tissues, including lung, of trimeric S-Fc-immunized mice. However, different titers of virus were detected in the nasal turbinate, lung, and brain of the PBS group (FIG. 7), indicating these control mice failed to contain viral replication. To further confirm the protection, histopathology was performed and the extent of lung inflammation was determined. The mouse lungs in PBS control mice showed remarkable infiltration of monocytes and lymphocytes after challenge, resulting in high levels of inflammation (FIG. 8, right). In contrast, mice immunized with the trimeric S-Fc/wt protein had significantly lower lung inflammation scores (FIG. 8, middle), which was comparable to the lung structure of uninfected mouse (FIG. 8, left). Overall, the findings show that FcRn-mediated intranasal delivery of the trimeric S-Fc/wt conferred significant protection against lethal SARS-CoV-2 virus challenge, resulting in decreased mortality, viral replication, and pulmonary inflammation.

### 7. An FcRn-Targeted Mucosal Vaccine Against SARS-CoV-2 Infection and Transmission

### i. Introduction

SARS-CoV-2, a virus causing the COVID-19 pandemic, is highly infectious, circulates rapidly worldwide, and mutates constantly. The SARS-CoV-2 variants include Alpha (B.1.1.7), Beta (B.1.351), Gamma (P.1), Delta (B.1.617.2), and Omicron (B.1.1.529, BA.4/BA.5, XBB.1.5). These variants evade the immunity induced by current vaccines and prior infections. Through the receptor-binding domain (RBD) of its spike (S) protein, SARS-CoV-2 binds to the angiotensin-converting enzyme 2 (ACE2). The S protein undergoes a structural change upon binding and is cleaved by host proteases, such as the transmembrane serine protease 2 (TMPRSS2), allowing it to fuse with the cellular membrane for host cell entry. Because of its vital role in mediating receptor binding and infection initiation, the S protein is the primary target for developing vaccines.

SARS-CoV-2 can be shed from individuals with asymptomatic infections and spread predominantly through droplets and airborne aerosols. The virus first enters the nose or mouth and replicates within epithelial cells of the nasopharynx, causing an upper respiratory infection. Hence, the nasal mucosa and nasopharynx are the primary sites of exposure to SARS-CoV-2 before dissemination to the lungs and other tissues/organs. The currently authorized intramuscular vaccines can effectively prevent severe diseases and deaths caused by COVID-19. However, they cannot effectively elicit protective mucosal immunity in the upper respiratory tract. This shortcoming allows opportunistic breakthrough infections in those who received vaccinations. Hence, the SARS-CoV-2 can linger in the nasal mucosa even after clearing infection in the lungs in vaccinated individuals. The emergence of the SARS-CoV-2 variants, especially the Omicron, exacerbates the situation. This evolution of SARS-CoV-2 necessitates a safe and protective mucosal vaccine to block the viral entry and reduce or eliminate the viral spread, thus preventing lung and systemic infection and breakthrough infection. Nasal-spray vaccines can elicit local secretory IgA antibodies and resident T and B cell responses in the upper respiratory tract and the lungs.

Epithelial cells lining the respiratory tract form a mucosal barrier. The neonatal Fc receptor (FcRn) binds to the Fc portion of IgG and mediates the transfer of IgG across the epithelial cells, a function essential to IgG distribution and homeostasis. Typically, FcRn shows a pH-dependent IgG binding, with a preference to bind IgG at acidic pH (6.0-6.5). Generally, the FcRn-IgG on the cell surface or in the endosome under acidic conditions goes through a non-degradative vesicular transport pathway within epithelial cells. Consequently, FcRn transports its bound IgG across the mucosal barrier and then releases it into the lumen or submucosa upon exposure to physiological pH. Through binding, FcRn also extends the half-life of IgG by reducing lysosomal degradation in cells, such as endothelial cells.

The vaccine must be administered locally in the respiratory tract to establish respiratory immunity with resident memory of T and B cells in the lungs. In this study, the ability of FcRn to deliver an intranasally-administered SARS-CoV-2 S antigen and induce protective mucosal and systemic immunity to SARS-CoV-2 infection is determined. Protective immune responses and mechanisms relevant to this nasal vaccination in the mouse and hamster models are defined. The results show that FcRn-mediated nasal delivery of a prefusion-stabilized SARS-CoV-2 S antigen induces secretory IgA Abs in the nasal lavage and high levels of long-lasting Ab and T-cell responses. The nasal vaccine confers durable protection against SARS-CoV-2 infection and airborne transmission.

### ii. Results

a. Expression and Characterization of SARS-CoV-2 S-Fc Proteins

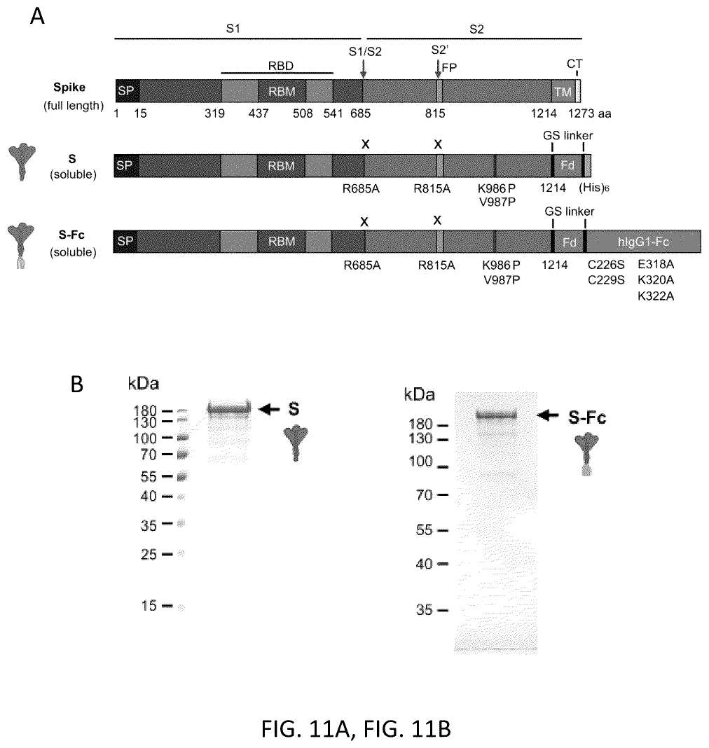

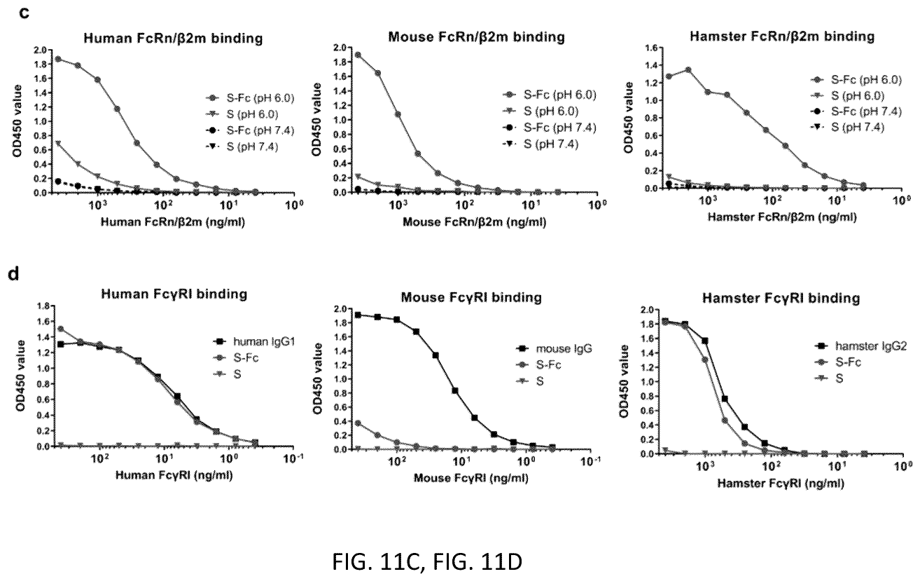

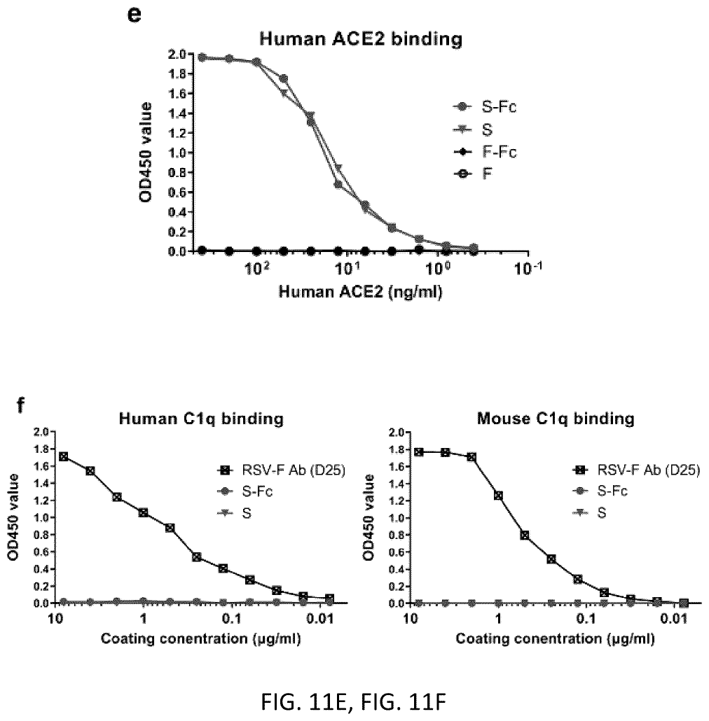

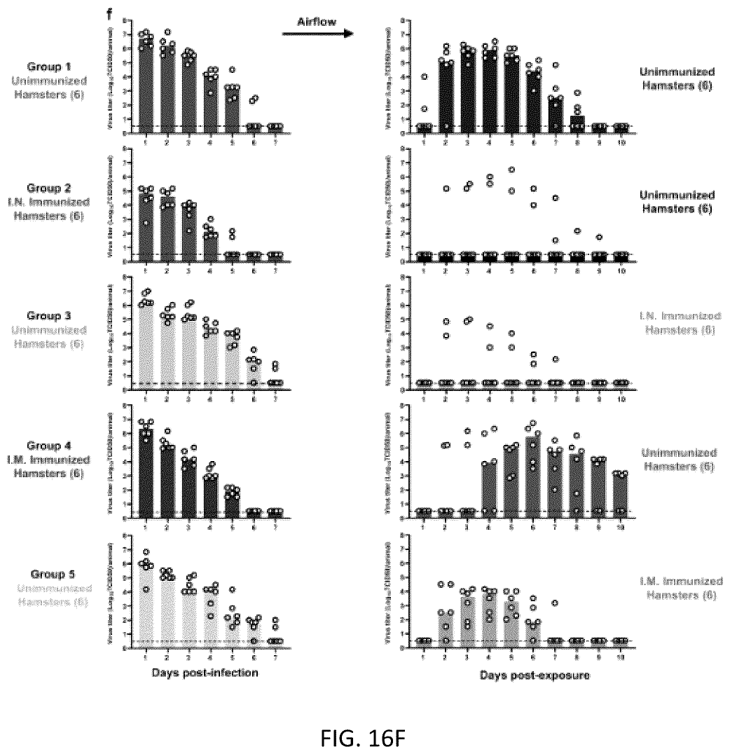

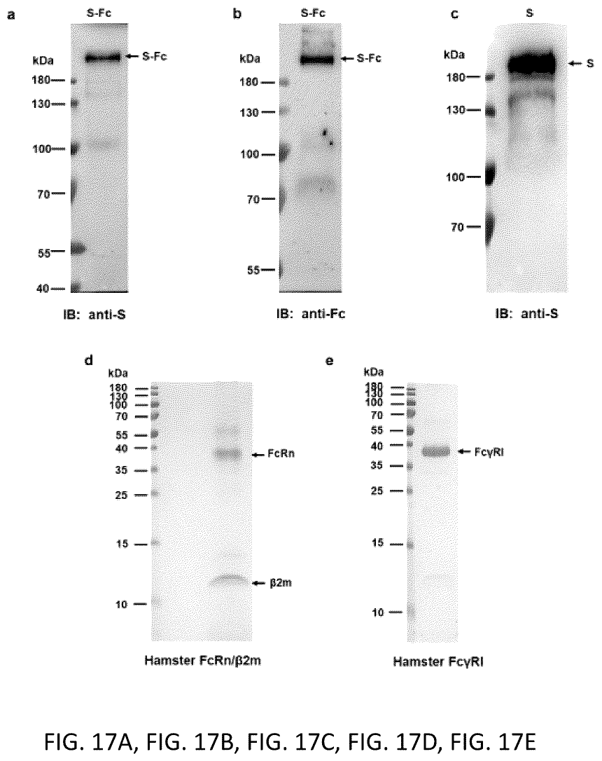

To target antigen to FcRn, a monomeric human IgG1 Fc fused to a prefusion-stabilized, soluble form of S, which contained an R685A mutation at the furin cleavage site, an R815A mutation at S2′ cleavage site, and 2P conversions (FIG. 11A) was expressed along with the T4 fibritin trimerization domain. The C1q binding site in IgG1 Fc was removed. The soluble S or S-Fc protein was secreted from the stable CHO cells (FIG. 11B). Since IgG only binds to FcRn at acidic pH14, it was determined if the S-Fc portion binds to FcRn at either pH 6.0 or 7.4. As shown in FIG. 11C, S-Fc interaction with both human (FIG. 11C) and mouse (FIG. 11C) FcRn/β2m was detected at pH 6.0 condition. Since hamster FcRn/β2m is not available, biotinylated hamster FcRn/β2m was generated (FIG. 17D). The S-Fc bound to hamster FcRn/β2m like human or mouse FcRn/β2m did (FIG. 11C). However, the binding of the S to human, mouse, and hamster FcRn/β2m protein was barely detectable (FIG. 11C). To further show whether the S-Fc binds to FcγRI (CD64), biotinylated hamster FcγRI was also produced (FIG. 17E). As expected, the S protein did not bind to mouse, human, and hamster FcγRI (FIG. 11D). As shown in FIG. 11D, the S-Fc, human IgG1, or hamster IgG2 bound to human and hamster FcγRI similarly in an ELISA assay. However, the S-Fc did not bind mouse FcγRI with high affinity (FIG. 11D), although mouse IgG was shown to bind mouse FcγRI strongly. This result could be explained by the fact that human IgG1 does not interact with mouse FcγRI with high affinity.

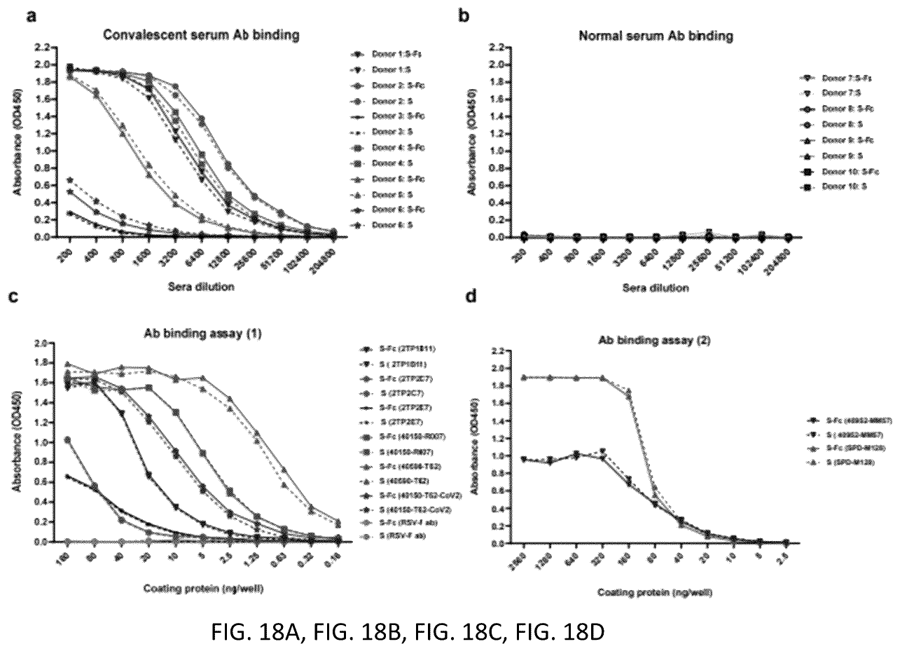

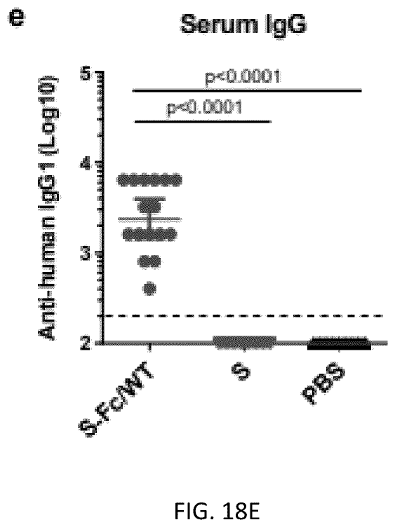

It was next determined if the S portion of the S-Fc binds to human ACE-2. In an ELISA assay, the S-Fc and S protein bound to human ACE-2 similarly (FIG. 11E), indicating the Fc fusion doesn't affect the conformation of S in the S-Fc protein. As a negative control, RSV F protein with or without Fc-fusion did not bind to human ACE-2 (FIG. 11E). In contrast to an RSV-F specific mAb (D25), the S-Fc could not bind to human or mouse C1q protein (FIG. 11F) in vitro. It was further determined if the S portion of the S-Fc interacts with convalescent serum Abs compared to the S alone. Normal human sera from different healthy donors were used as a negative control. Indeed, the sera from convalescent COVID-19 patients (FIG. 18A) were able to recognize both purified S-Fc (solid lines) and S (dashed lines) proteins with varying binding efficiencies. Most importantly, S-Fc and S showed the same binding levels for each tested serum sample, indicating that Fc-fusion with S didn't alter its conformation. Normal sera (FIG. 18B) from healthy individuals showed no binding activity. The results were further confirmed by the interactions of both S-Fc and S with various spike-specific mAbs (FIG. 18C, D). Together, the S portion of the S-Fc protein maintains the correct conformational structure, allowing for binding to the ACE-2 and S-specific Abs, and possesses the function to engage with FcRn and human or hamster FcγR1.

b. FcRn-Mediated Respiratory Delivery Enhances S-Specific Immune Responses

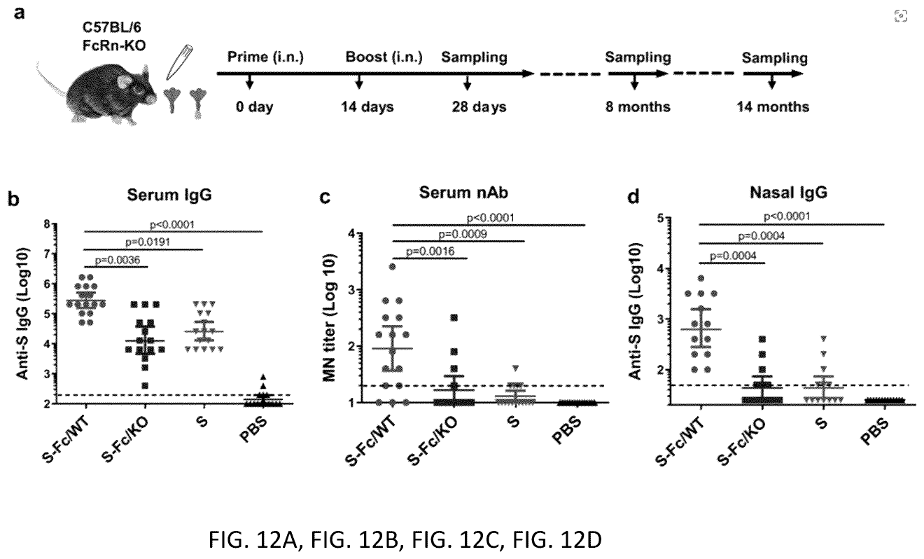

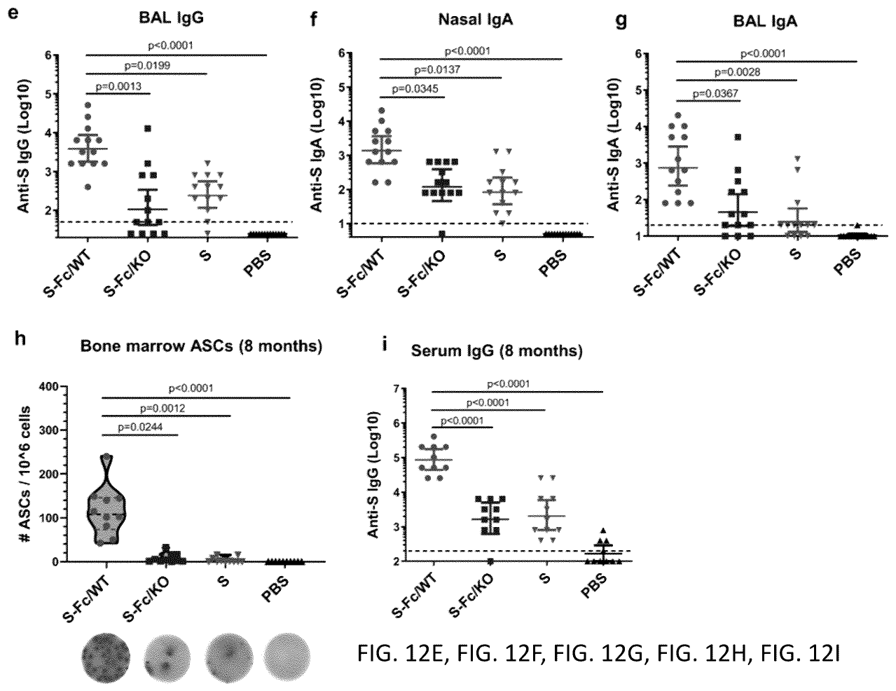

Whether FcRn-dependent transport augments the immune responses to the S protein was tested. C57BL/6 mice were i.n. immunized with 10 μg of S-Fc, S protein (equal molar amount), or PBS in 10 μg CpG adjuvant, the mice were boosted after 2 weeks (FIG. 12A). FcRn-knockout (KO) mice were used as a control to test the FcRn-mediated immunity enhancement. Using S protein alone allowed the evaluation of FcRn-independent effects in vivo and determine the magnitude of enhanced immune responses conferred by targeting the S-Fc to FcRn. As shown in FIG. 12, significantly higher titers of total serum IgG (FIG. 12B) were detected in the S-Fc immunized WT mice compared with the S-Fc-immunized FcRn KO, the S-immunized or PBS-treated WT mice. The Ab titers were measured by coating ELISA plates with S protein. The IgG titers were measured in mouse sera (n=16 for S-Fc/WT and S groups, n=15 for S-Fc/KO and PBS groups). The data represent a geometric mean with 95% CI. Moreover, sera from the S-Fc immunized mice exhibited much stronger neutralizing activity than the control groups (FIG. 12C). Sera from mice (n=16 for S-Fc/WT and S groups, n=15 for S-Fc/KO and PBS groups) were heat-inactivated and serially diluted two-fold. The ancestral SARS-CoV-2 strain (100 TCID50) was added and incubated at 37° C. for 1 h. The mixture was added to Vero-E6 cells and incubated at 37° C. for 96 hrs. The neutralization Ab titers were determined and are expressed as the reciprocal of the highest dilution preventing the cytopathic effect (CPE). Likewise, the S-Fc induced higher levels of IgG (FIG. 12D, E) and IgA (FIG. 12F, G) Abs in nasal washes (FIG. 12D, F) and bronchoalveolar lavage fluids (BAL) (FIG. 12E, G) than those of the WT mice immunized by the S alone, PBS or the FcRn KO mice immunized by the S-Fc. The data represent a geometric mean with 95% CI. h S-specific Ab-secreting cells in the bone marrow. Bone marrow cells isolated 8 months after the boost were placed on S-coated plates and quantified by ELISpot analysis of IgG-secreting plasma cells. Data were pooled from two experiments (n=10 for S-Fc/WT, S-Fc/KO, and PBS groups and n=11 for S group). The graphs were plotted based on each experiment's average spot number from the duplicated wells. A violin plot with 25th, 50th, and 75th percentiles was shown. i Anti-S-specific IgG Ab titers in sera 8 months after the booster (n=10 for S-Fc/WT, S-Fc/KO, and PBS groups and n=11 for S group). Since human IgG1 Fc was utilized in producing S-Fc, as expected, low levels of anti-Fc IgG were detected in the mouse sera after the i.n. boost (FIG. 18E).

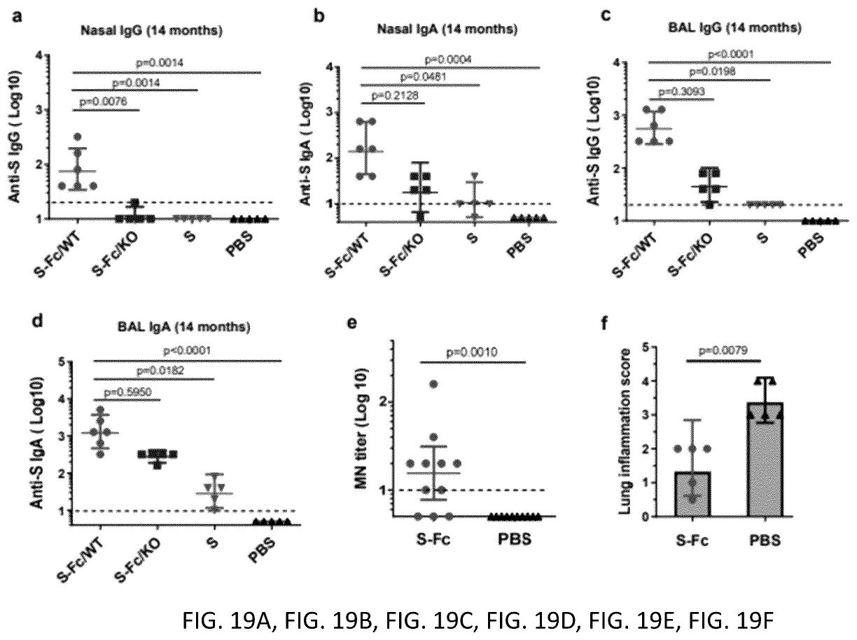

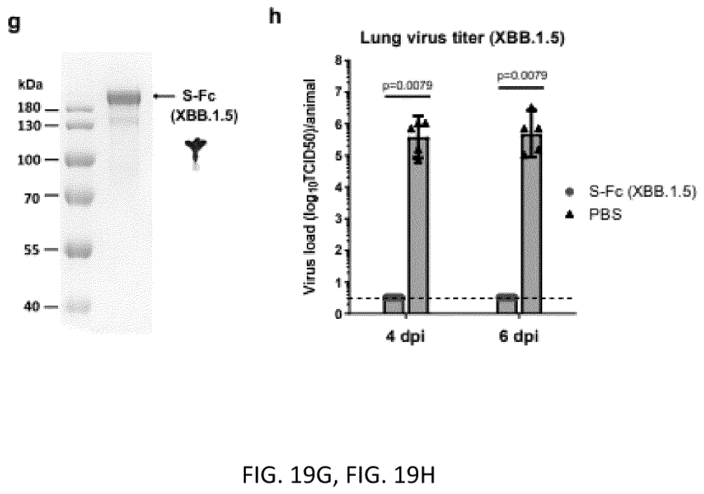

Activated B cells can differentiate into plasma cells that secrete antibodies at a high rate and reside in niches within the bone marrow. To determine whether antigen targeting to FcRn also elicits plasma cells that secret S-specific Abs, the number of IgG-secreting plasma cells in the bone marrow was measured 8 months after the boost by ELISpot. High numbers of S-specific IgG-secreting cells were present in the bone marrow of mice immunized with S-Fc compared with other groups (FIG. 12H). To show whether the increased IgG-secreting plasma cells correspond to a rise in Ab production and maintenance, IgG Abs in the sera were measured 8 months after the boost. High titers of S-specific IgG Abs were maintained in mice immunized with the S-Fc, but not the S alone (FIG. 12I). Also, a significant level of S-specific IgA or IgG was present in the nasal washes or BAL of mice immunized with S-Fc compared with other groups 14 months later (FIG. 19A-D). Immunization with the S-Fc was more effective than immunization with S alone, indicating that S-specific Abs persisted much longer after FcRn-targeted mucosal immunization. The data demonstrate that an Fc-fused, soluble, prefusion-stabilized S protein delivered through FcRn is much more potent in triggering S-specific Ab responses.

c. Intranasal Immunization by S-Fc Protects Mice from SARS-CoV-2 Infection

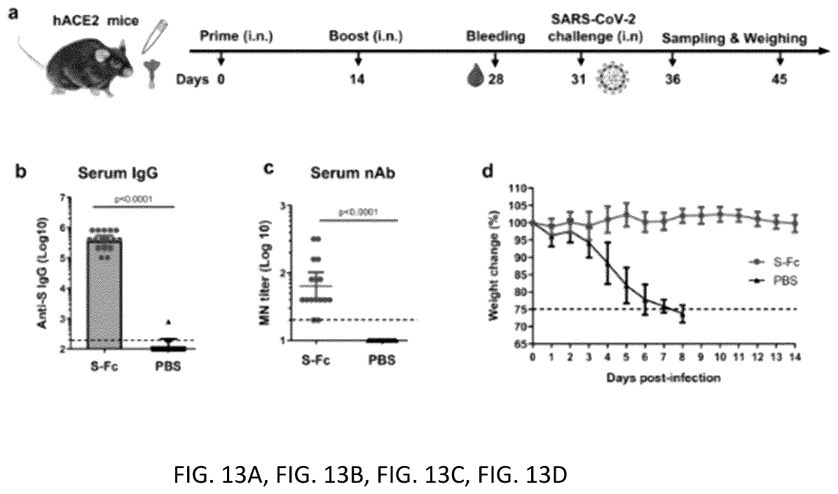

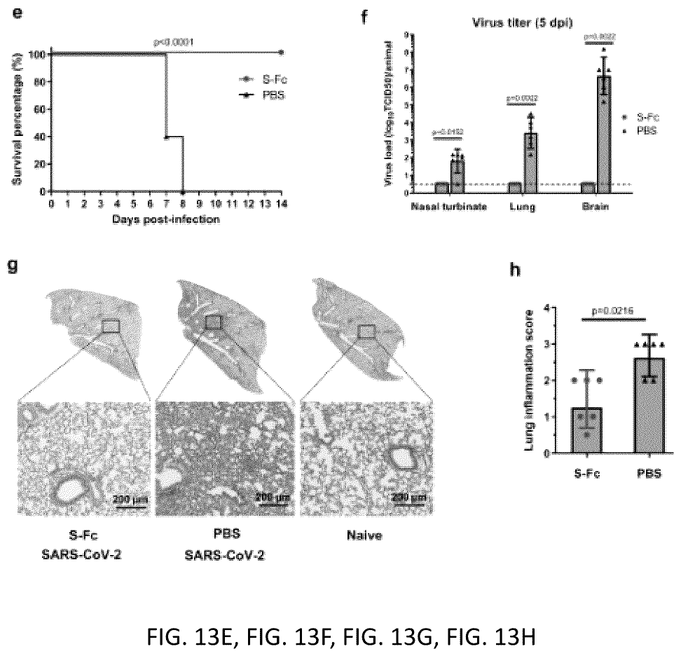

Human ACE2 transgenic mice are highly susceptible to SARS-CoV-2 intranasal challenges when high virus doses are used. In the study, hACE2 mice were i.n. immunized with g of S-Fc or PBS in 10 μg CpG and boosted in a 2-week interval (FIG. 13A). Mice were boosted 14 days after primary immunization. Six mice in each group were euthanized at 5 days post-infection (dpi) for sampling and virus titration. The remaining 10 mice in each group are subjected to the body weight loss and survival analysis. Significantly higher titers of serum IgG (FIG. 13B) and neutralizing antibodies (nAbs) (FIG. 13C) were detected in the hACE2 mice i.n. immunized with the S-Fc when compared with PBS-treated mice. To test whether the immune responses elicited by the intranasal (i.n.) vaccination with the S-Fc provide protection, all immunized mice were i.n. challenged with a lethal dose (2.5×10⁴ TCID50) of ancestral SARS-CoV-2 virus 2-3 weeks after the boost (FIG. 13A). Mice were monitored and weighed daily for 14 days. All mice in the control groups exhibited rapid weight loss following the challenge, either succumbing to infection within 8 days post-infection (dpi) or being subjected to euthanasia. In contrast, the S-Fc-immunized mice did not experience significant body-weight loss (FIG. 13D) and had complete protection with significantly higher survival rates (100%) than those of the PBS control group (FIG. 13E). Seventeen days after the boost, groups of mice (S-Fc group, n=10; PBS group, n=10) were i.n. challenged with the ancestral SARS-CoV-2 (2.5×10⁴ TCID50) and weighed daily for 14 days. Mice were humanely euthanized at the end of the experiment or when a humane endpoint was reached. As expected, all the PBS-treated mice had high virus loads in the lung at 5 dpi. In contrast, a significant reduction of virus load in the nasal turbinate, lungs, and brain tissue was seen in the S-Fc immunized mice when compared to the control animals (FIG. 13F). The virus titers in the samples of the immunized and control mice (n=6) were determined by CPE in Vero E6 cells cultured for 4 days. The viral titers were shown as TCID50 from each animal sample. The hACE2 mice that were i.n. immunized with the S-Fc protein (FIG. 19G) derived from the Omicron XBB.1.5 variant were also challenged. Similarly, a significant reduction of XBB.1.5 virus load in the lungs was seen in the XBB.1.5 S-Fc immunized mice compared to the PBS control animals (FIG. 19H). Brain tissues exhibited the highest levels of virus load in the PBS control mice at 5 dpi. Therefore, the S-Fc-immunized hACE2 mice essentially contained viral replication in tissues/organs of the viral entry and prevented the viral spreading to other tissues/organs.

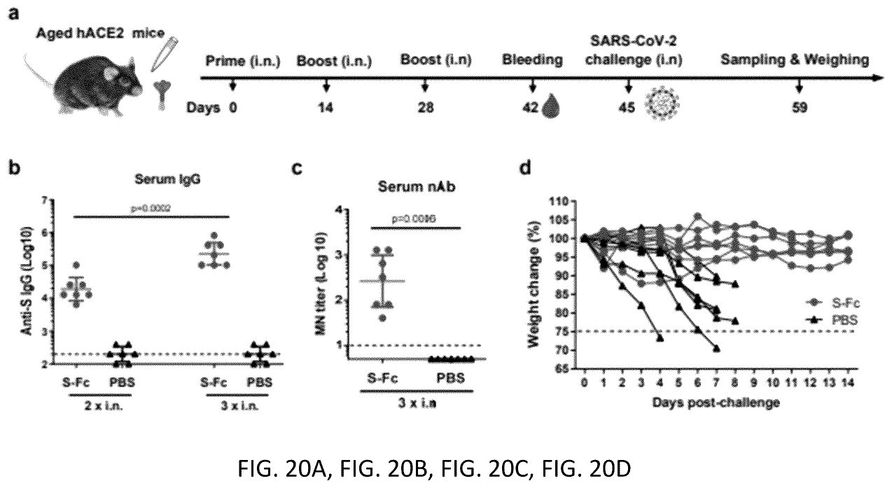

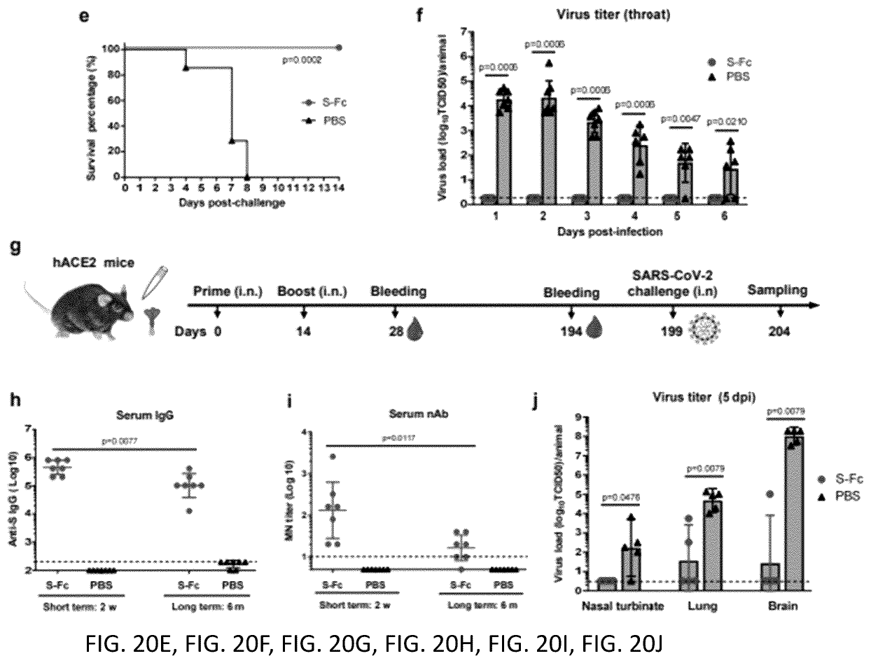

Lungs were collected at 5 dpi to show protection for further histopathological analysis. The lungs of uninfected mice were used as normal control (FIG. 13G). Before the virus challenge, no apparent alterations were observed in the lung structure and histology of the S-Fc immunized mice and normal mice, indicating that the S-Fc did not induce inflammation. In contrast, focal inflammatory cell infiltration, pneumonia, peribronchiolitis, and perivasculitis were found in the lungs of PBS control mice after virus infection (FIG. 13G). The alveolitis was not observed. Hence, the mice immunized with S-Fc had a significantly lower lung inflammation score than those in the PBS group (FIG. 13H, p<0.05). It was also found that i.n. immunization with the S-Fc protected the aged hACE2 mice from lethal SARS-CoV-2 infection (FIG. 20A-F) and elicited durable protection in hACE2 mice (FIG. 20G-J). These findings demonstrate that FcRn-mediated delivery of the S-Fc confers significant protection against lethal SARS-CoV-2 challenge, resulting in decreased mortality, viral replication, and pulmonary inflammation in a hACE2 mouse model.

The mortality and fatality of COVID-19 are highly skewed toward older adults, and age is negatively correlated with immune responses after vaccination. The aged mice develop more severe lung damage than the young adult mice upon SARS-CoV-2 infection or re-infection. To assess the immune response and protective efficacy of our vaccine in aged mice, we performed i.n. immunization with two doses of S-Fc (10 μg/mouse) at a 2-week interval (FIG. 20A) and used PBS-immunized mice as controls. As shown in FIG. 20B, although high SARS-CoV-2-specific IgG was elicited in all S-Fc immunized mice two weeks after the boost, the levels of serum IgG antibodies produced in the aged mice were generally lower than those of the young adult mice. After the immunized mice received a second boost (FIG. 20A), the serum IgG and neutralizing Ab levels elicited in the aged mice were comparable to those of the young adult mice receiving twice i.n. immunizations (FIG. 20B, C). The immunized aged mice were i.n. challenged with ancestral SARS-CoV-2 virus (5×10³ TCID50). All aged mice in the PBS control group displayed high susceptibility to virus infection and suffered fast weight loss, resulting in 100% death. In contrast, the S-Fc-immunized aged mice did not exhibit obvious body-weight loss and clinical signs (FIG. 20D). All mice were fully protected, leading to 100% survival after the challenge (FIG. 20E). Strikingly, no live virus can be measured in the throat swabs of the S-Fc-immunized mice throughout the infection period of 1-6 dpi in comparison to the active virus replication in the throat samples from the PBS control mice. (FIG. 20F). These results indicated the complete blockage of virus amplification and shedding from the airways of the infected animal by S-Fc immunization after three times immunizations.

Although the authorized booster vaccinations in adults elicit high levels of neutralizing Abs against the SARS-CoV-2, antibody levels can wane substantially 3-4 months after vaccination. To address whether the i.n. immunization with the S-Fc can sustain long-term protection (FIG. 20G), the serum IgG and neutralizing Abs were measured in the hACE2 mouse sera six months after the boost. Most of the immunized mice maintained a significant level of S-specific IgG Ab (FIG. 20H) and the neutralizing Ab activity (FIG. 20I) in their sera compared to PBS control animals, although the levels were lower than those in the immunized sera collected two weeks following the boost. To investigate long-term protection, mice were further challenged with ancestral SARS-CoV-2 virus (2.5×10⁴ TCID50) six months after the boost (FIG. 20J). Following the challenge, mice immunized with the S-Fc exhibited significantly reduced viral replications in nasal turbinate, lung, and brain tissues (FIG. 20J), while the PBS control mice displayed a considerably higher level of viral replications. Overall, intranasal delivery of the SARS-CoV-2 vaccine engendered an effective and long-term memory immune response and provided sustained protection against challenges.

d. Intranasal Immunization with S-Fc Protects Infections of SARS-CoV-2 Variants

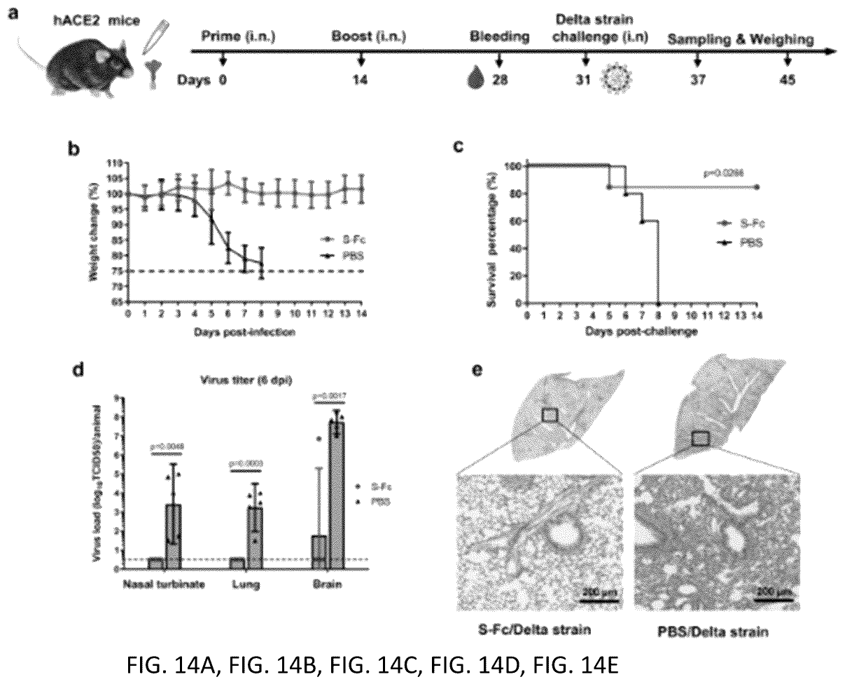

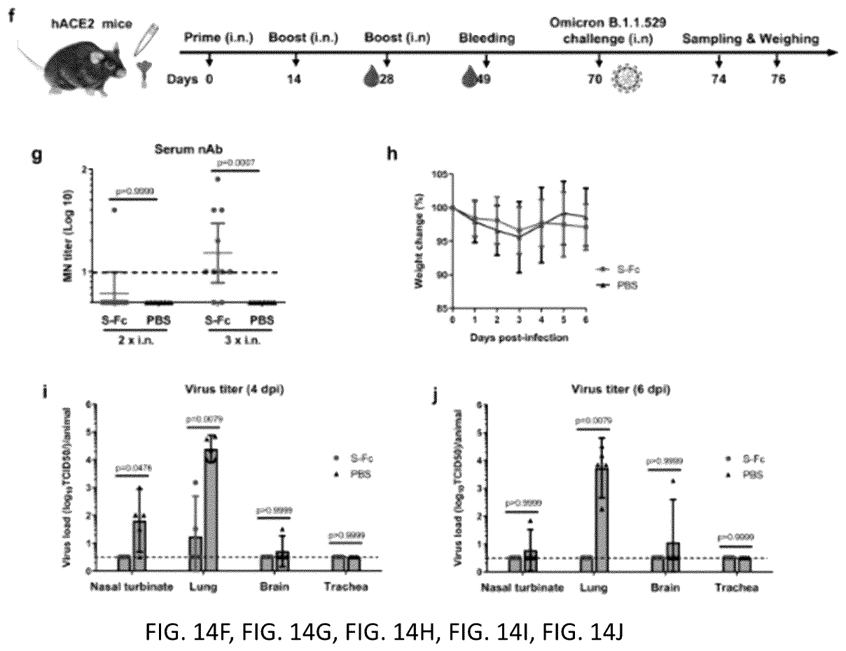

The SARS-CoV-2 is rapidly evolving via mutagenesis, significantly impacting transmissibility, morbidity, reinfection, and mortality while lengthening the pandemic. Since the S portion of the S-Fc is derived from ancestral SARS-CoV-2, the effectiveness and neutralizing activity elicited by the S-Fc vaccine was determined against Delta and Omicron variants. First, hACE2 mice were i.n. immunized twice with 10 μg of S-Fc adjuvanted in 10 μg CpG (FIG. 14A) and tested the protection against the SARS-CoV-2 Delta strain. Most immunized mice developed nAbs against the Delta strain after the boost (FIG. 19E, p<0.01). To show protection, all immunized mice were challenged with a lethal dose (2.5×10⁴ TCID50) of the Delta strain 17 days after the boost. All mice in the control group experienced rapid weight loss (FIG. 14B), labor breathing, and ataxia before finally dying from the viral infection or were humanely euthanized. In contrast, the S-Fc-immunized hACE2 mice did not show significant body-weight loss (FIG. 14B) or clinical signs. The majority (83.3%) of the S-Fc-immunized mice survived, which was significantly higher than the survival rates of the PBS control group, where all mice died from Delta virus infection (FIG. 14C). Furthermore, viral replication was measured in the nasal turbinate, lung, and brain tissues 6 dpi (FIG. 14D). Live Delta virus was detected in the nasal turbinates, lungs, and brain tissues of the PBS control mice. Still, no live virus was found in the S-Fc-immunized animals' nasal turbinates and lung tissues (FIG. 14D). Only one mouse in the S-Fc-immunized group showed reduced virus load for brain samples.

In contrast, the others had no virus detected. Additionally, no prominent inflammation was observed in the lungs of the S-Fc immunized mice. In contrast, focal perivascular, peribronchial inflammation and thickening of the alveolar septa were found in the lung of PBS control mice (FIG. 14E). The mice immunized with S-Fc had a significantly lower inflammatory score compared to the challenged mice in PBS control group (FIG. 19F, p<0.01).

Three i.n. immunizations were necessary to produce the Omicron-specific nAbs in the sera of most mice (FIG. 14F, G). Mice were i.n. challenged with the Omicron B.1.1.529 (1×10⁶ TCID50) 42 days after the boost. Two weeks after the first or 3 weeks after the second boost, sera from 10 mice per group were measured for the neutralizing Ab titers (FIG. 14G). Since the Omicron strain possesses abundant mutations in its S protein, this implies that Omicron can escape nAbs elicited by the ancestral S protein. Noticeably, when the S-Fc immunized mice were challenged with a dose (1×10⁶ TCID50) of the Omicron B.1.1.529 strain after the 2nd boost, there was no difference in visual clinical symptoms and body-weight loss between the S-Fc-immunized and the PBS control mice throughout the experiments (FIG. 14H), which can be attributed to the low virulence of the Omicron strain in mice. No live Omicron virus was found in nasal turbinate and brain tissues (FIG. 14I, J) in all S-Fc-immunized mice at 4 and 6 dpi. For lung tissues, live Omicron virus was only identified in 2 mice immunized with S-Fc at 4 dpi at low levels (around 1030 and 22 folds of virus reduction compared to the average virus titer in PBS groups) but not 6 dpi, indicating limited viral replication in these mice. In contrast to the ancestral and Delta strains, the Omicron B.1.1.529 infection in mice did not cause body-weight loss and mortality. However, the S-Fc nasal immunization remarkably attenuated replication of the Omicron variant in the respiratory tract and the brain, substantially reducing lung inflammation in the hACE2 mice.

e. Intranasal Immunization with S-Fc Induces Local Immunity to Viral Infections

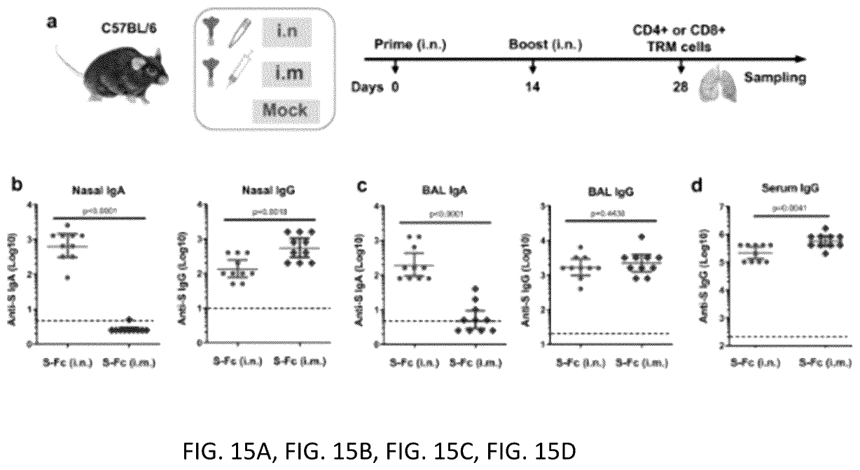

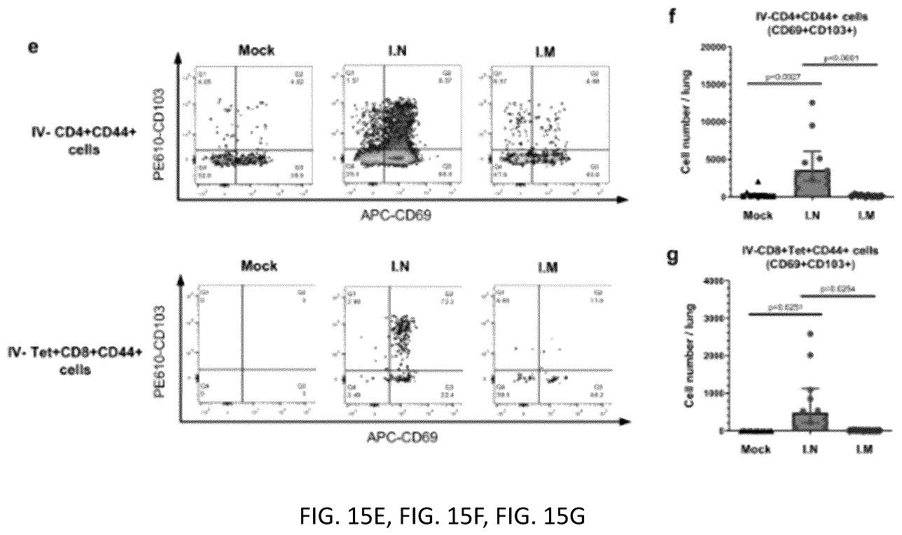

Because SARS-CoV-2 initiates its infection in the upper respiratory tract, a nasal vaccine must elicit anti-viral IgA Abs in the nasal secretions and BAL of the lungs. First, to determine the ability of the respiratory immunization by the S-Fc to induce local humoral immune responses, S-specific Abs were examined in mucosal secretions, which were compared to mice that received intramuscular (i.m.) immunization with the same amount of the S-Fc and CpG (FIG. 15A). Mice were boosted 14 days after primary immunization. An additional group of mice (n=10) that were intramuscularly (i.m.) immunized in 10 μg S-Fc with 10 μg of CpG was included as an i.m. route control. Tissues and blood were collected at the indicated time points. The nasal washes and BAL were collected 14 days following the boost and analyzed for S-specific IgA and IgG by ELISA. As shown in FIG. 15, the levels of S-specific IgA Abs significantly increased in the nasal washes and BAL (FIG. 15B, C) in the i.n. immunized mice. In contrast, the mice immunized with the S-Fc via the i.m. route showed much lower levels of S-specific IgA in both the nasal washes and BAL (FIG. 15B, C, p<0.0001). The mice that were i.m. immunized with the S-Fc had higher levels of S-specific IgG in the nasal washes than that of the i.n. immunized mice (FIG. 15D, p<0.01). However, both i.n. and i.m. S-Fc-immunized mice exhibited similar levels of IgG Ab in the BAL (FIG. 15C). The mice that were i.m. immunized with the S-Fc also developed higher levels of IgG Ab in the sera than those of the i.n. immunized mice (FIG. 15D, p<0.01); this may reflect the full deposit of the S-Fc protein in the tissue by the needle injection, compared to i.n. immunization, which usually results in a lower-than-desired dose.

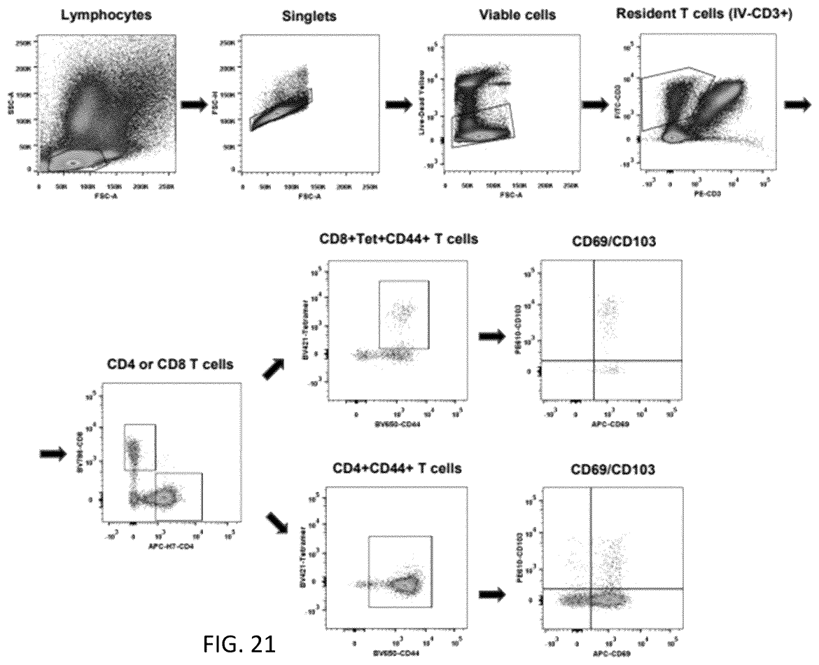

Tissue-resident memory (TRM) T cells are found in the nasal cavity and lungs during SARS-CoV-2 infection, essential to limit disease severity and viral replication. Thus, it was next determined whether intranasal delivery of the S-Fc protein can induce TRM T cells in the lungs. The TRM T cells in the lungs were assessed 14 days after the boost by FACS (FIG. 15E and FIG. 21). Compared to the PBS control, a notably higher percentage of IV-CD4+CD44+CD69+CD103+ TRM cells (FIG. 15f) and IV-CD8+Tet+CD44+CD69+CD103+ TRM cells (FIG. 15G) were detected in the lungs of the i.n. immunized, but not in the i.m. immunized mice with the S-Fc. Together, these data indicate that the intranasal, but not intramuscular, immunization with the S-Fc protein induces CD4+ and CD8+ TRM cells in the lung. The IV-CD4+CD44+CD69+CD103+ or IV-CD8+Tet+CD44+CD69+CD103+ TRM T cells in the lungs were assessed 14 days after the boost by FACS. Flow cytometry plots represent results from two independent experiments with 5 individual samples per group. Numbers in the top right quadrants (FIG. 15E) or the graph (FIG. 15F, G) indicate the percentage or number of TRM CD4+ or CD8+T lymphocytes following i.n. or i.m. immunizations. The data in (FIG. 15F) and (FIG. 15G) represent a geometric mean with 95% CI.

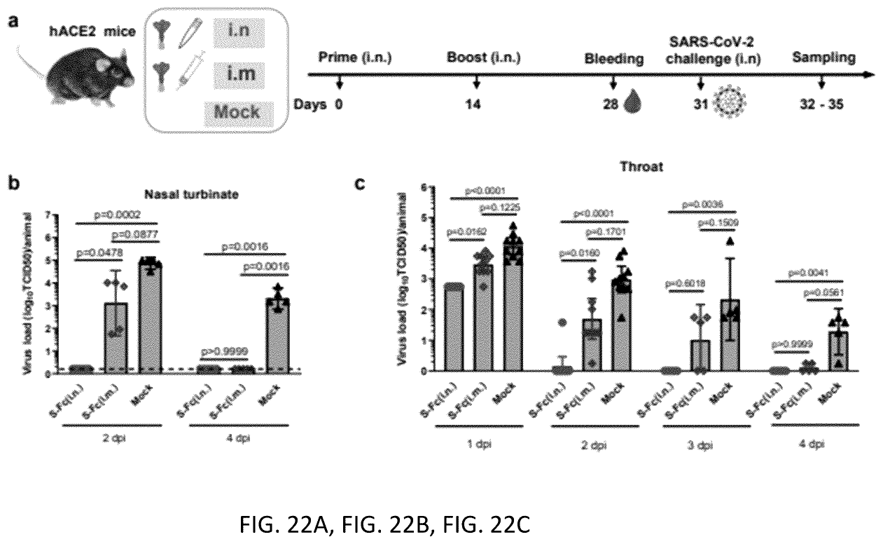

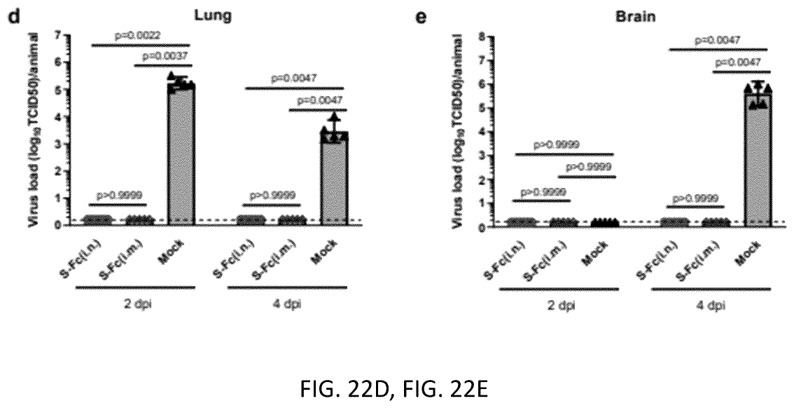

Finally, to demonstrate the protective efficacy of the nasal vaccination, we challenged the i.n. or i.m. immunized hACE2 mice with the SARS-CoV-2 (2.5×10⁴ TCID50) (FIG. 22A). Then, we measured viral titers in nasal turbinates, throat, lungs, and brain tissues from the early to the middle phase of infection (1-4 dpi). Virus titers on 1-2 dpi in the nasal turbinates and throat of the animals who received i.n. immunizations were significantly lower than the virus titers from the animals that received i.m. immunizations (FIG. 22B, C, p<0.05), respectively. Contrary to the PBS group, hACE2 mice immunized with S-Fc via either i.n. or i.m. routes showed complete inhibition of virus growth and dissemination in the lung (FIG. 22D) and brain (FIG. 22E) at 2 and 4 dpi. Overall, these data indicate that the i.n. delivery of the S-Fc vaccine induces local humoral and cellular immune responses, providing more efficacious protection in the upper respiratory tract against SARS-CoV-2 infection than the i.m. route.

f. Intranasal Immunization with S-Fc Reduces Viral Transmission in Hamsters

SARS-CoV-2 is highly contagious; airborne transmission is a major concern in public health, as inhalation represents a primary transmission route for COVID-19. Hence, a vaccine can prevent or reduce SARS-CoV-2 airborne transmission from vaccinated individuals to unvaccinated and vice versa is highly desirable. Hamster-to-hamster or hamster-to-human transmissions of SARS-CoV-2 occur.

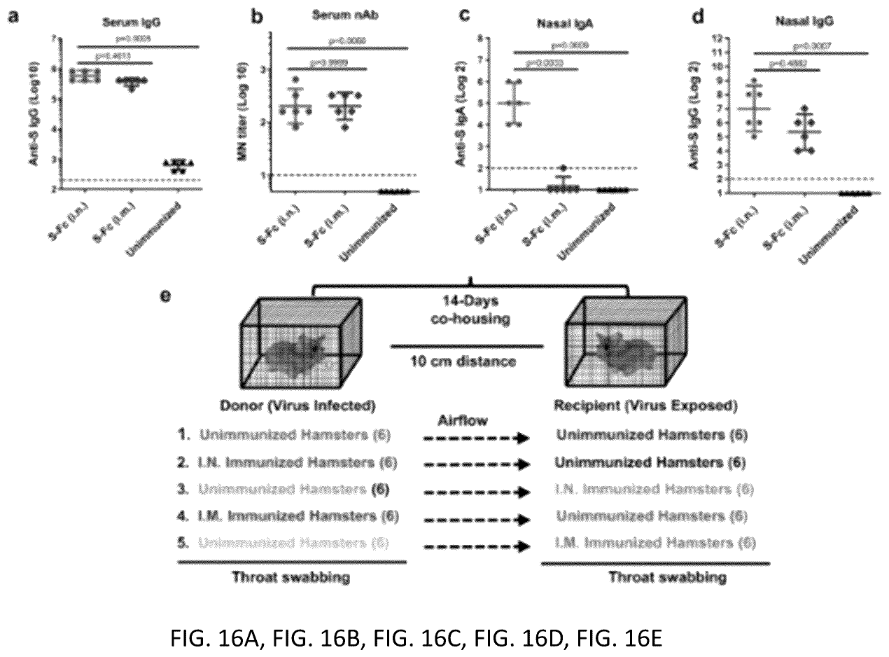

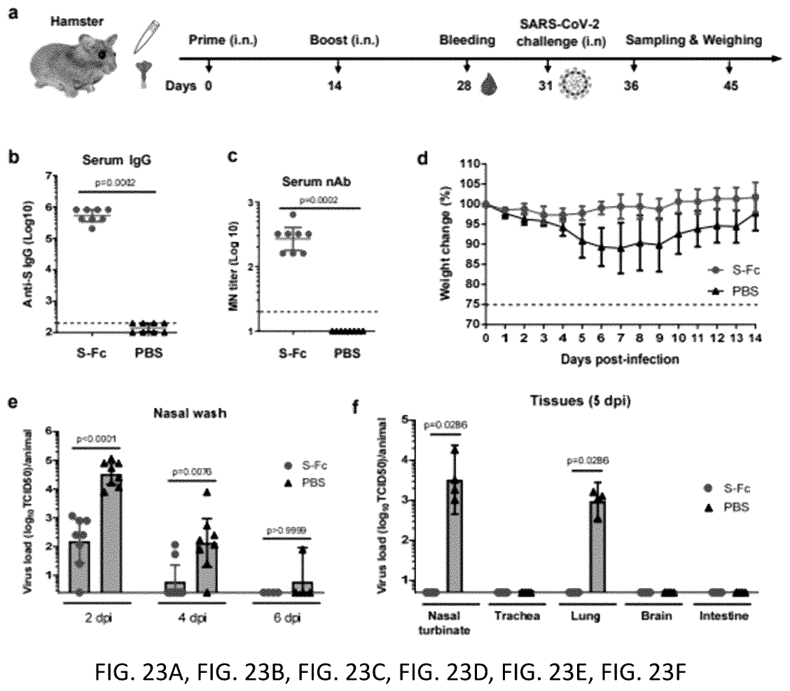

Hamsters were first immunized and hamsters showed that were i.n. immunized with S-Fc were protected from ancestral SARS-CoV-2 infection (FIG. 23). Hamsters that were either i.n. or i.m. immunized by the S-Fc (FIG. 23A) developed significantly higher levels of IgG (FIG. 16A) and nAb (FIG. 16B) in the sera in comparison to the PBS control hamsters. However, the hamsters that were immunized with the S-Fc via the i.n. route, compared to the i.m. route, had much higher levels of S-specific IgA Ab (FIG. 16C, p<0.01). The IgG Ab (FIG. 16D) in the nasal washes didn't show big difference between i.n. and i.m. groups. To test the transmissibility, a unidirectional airflow chamber was used. Naive hamsters were exposed to the i.n. immunized hamsters that were infected with SARS-CoV-2, and vice versa (FIG. 16E). Briefly, 5 μgroups of hamsters are included, and each group has 6 hamsters for virus donors and 6 hamsters for virus recipients. The donor hamsters were unimmunized or immunized with S-Fc plus CpG via i.n or i.m. routes. Then the donor hamsters were infected with 1×10⁵ TCID50 ancestral SARS-CoV-2 strain. Fourteen hours later, donor hamsters in the wire cages were separately cohoused with 6 unimmunized or immunized hamsters within the same isolator. Throat swabbing was performed for 7 days for donor hamsters (1-7 dpi) and 10 days for recipient hamsters (1-10 days post-exposure). As a control, the naive hamsters were also exposed to infected naive hamsters. The i.m. immunized hamsters by the S-Fc were used as a control. Subsequently, hamsters were i.n. infected with a high titer of SARS-CoV-2 (1×10⁵ TCID50/hamster) to replicate a breakthrough infection (FIG. 16F). Both naive and immunized hamsters were exposed to infected immunized or naive hamsters in the unidirectional airflow chamber for 10-14 days.

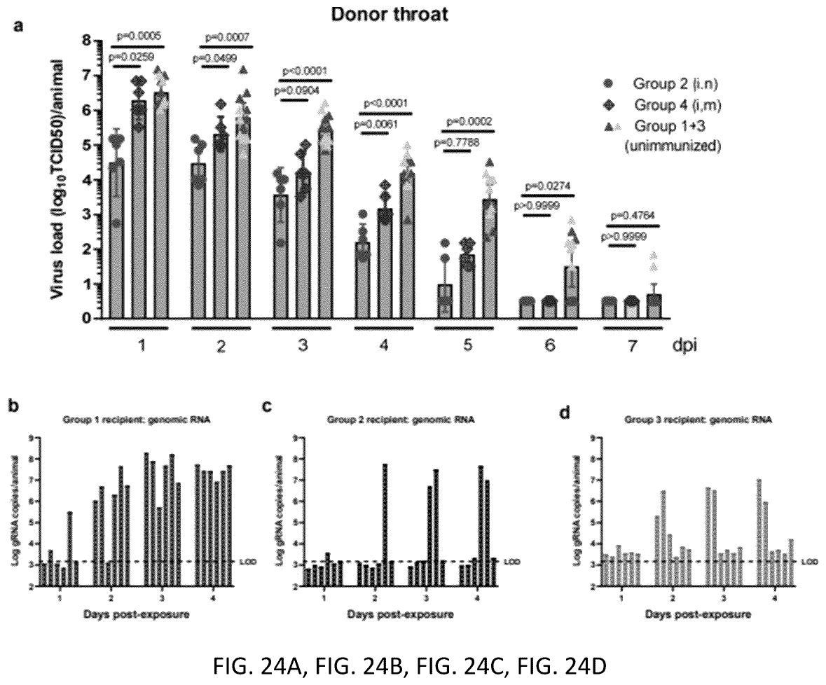

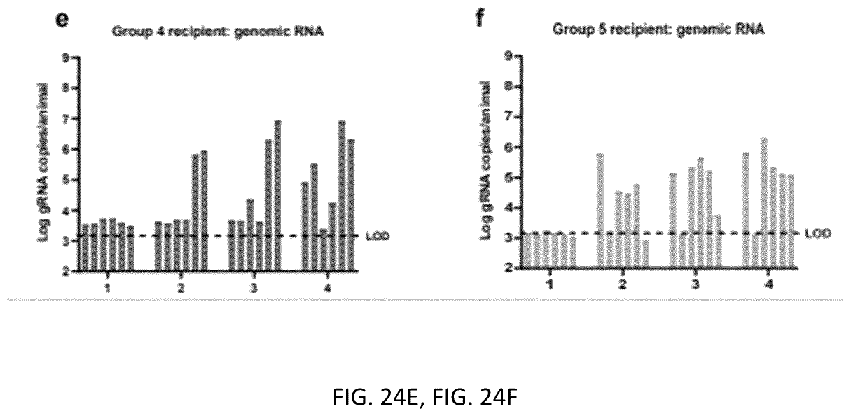

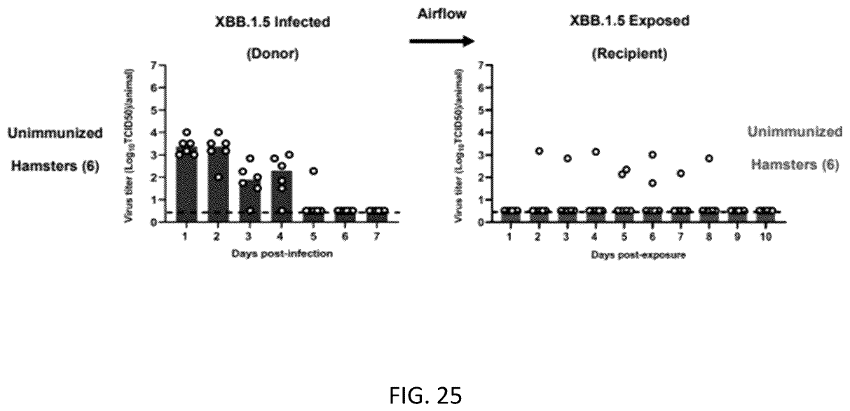

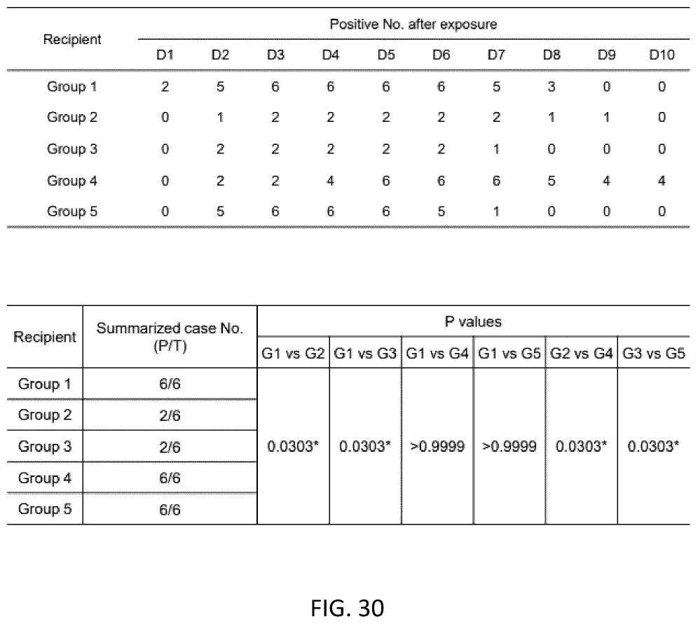

All six unimmunized hamsters exposed to the SARS-CoV-2-infected unimmunized hamsters had a high titer of live virus detected in their throats, which peaked at 3-6 dpi (FIG. 16F, group 1), indicating that SARS-CoV-2 infection and airborne transmission was successful. All six i.n. immunized hamsters infected with the virus exhibited much less body-weight loss. They had a lower level of live virus detection in their throat swabs (FIG. 16F, group 2) than the infected naive hamsters (FIG. 16F, group 1), verifying again that the i.n. immunizations with the S-Fc induced protective immunity at viral entry sites. In the exposed animals, four of the six naive unimmunized hamsters exposed to the i.n. immunized, SARS-CoV-2-infected hamsters had no detectable live virus during the 10 days (FIG. 16F, group 2). Similarly, four of the six i.n. immunized hamsters exposed to SARS-CoV-2-infected naive unimmunized hamsters had no live virus detectable during the 10 days (FIG. 16F, group 3). However, the live virus was detected in two of the i.n. immunized hamsters, the virus titers were significantly lower (FIG. 16F, group 3). In contrast, all six naive unimmunized hamsters exposed to the virally infected hamsters that were i.m. immunized with the S-Fc protein displayed a high titer of live SARS-CoV-2 virus in their throats (FIG. 16F, group 4). However, the virus detection was delayed 2-3 days, indicating that i.m. immunized animals can spread the virus. In addition, all six i.m. immunized hamsters exposed to SARS-CoV-2-infected naive unimmunized hamsters had live viruses detected in their throats during the 10 days (FIG. 16F, group 5), indicating they all failed to prevent viral transmission. Viral load was also quantified as SARS-CoV-2 N gene RNA in throat swab fluid on 1-4 dpe (days post-exposure). The results from the N RNA detection were consistent with the results of the live-virus detection (FIG. 24B-F). Together, the study unambiguously demonstrates that the i.n. immunization by the S-Fc protects hamsters by decreasing viral shedding and preventing airborne SARS-CoV-2 transmission (FIG. 30).

Golden Syrian hamsters are highly susceptible to the SARS-CoV-2 virus and exhibit disease symptoms like humans, including severe lung inflammation. It was shown that hamster FcRn bound human IgG1 and the S-Fc (FIG. 1C). Hence, hamsters were i.n. immunized with 30 g of the S-Fc protein or PBS together with 30 μg CpG and boosted 2 weeks later FIG. 23A). As shown in FIG. 23, the S-Fc immunized hamsters induced significantly higher levels of IgG (FIG. 23b, p<0.001) and neutralizing Ab (FIG. 23C, p<0.001) titers in comparison with the PBS-immunized hamsters. Next, it was investigated whether hamsters i.n. immunized by the S-Fc resist to SARS-CoV-2 infection. Groups of hamsters (8 hamsters/group) administered intranasally with S-Fc or PBS were challenged with 1×10⁶ TCID50 of ancestral SARS-CoV-2 virus 17 days after the boost. Hamsters within the PBS group underwent mild to moderate body weight loss (FIG. 23D), but none succumbed to viral infection. In contrast, the S-Fc-immunized hamsters did not show weight loss (FIG. 23D). Meanwhile, the S-Fc immunized hamsters had significantly reduced the number of viruses in nasal washes compared to that of PBS-immunized mice at 2 and 4 dpi (FIG. 23E, p<0.01-0.0001), indicating the decline of virus shedding from these animals. Third, viral replications were measured in the nasal turbinate, trachea, lung, brain, and intestine 5 dpi (FIG. 23F). High titers of live SARS-CoV-2 virus were detected in the nasal turbinate and lung tissues of the PBS-immunized control hamsters; on the contrary, no live virus was isolated from the nasal turbinate and lung tissues of animals who received i.n. immunization with the S-Fc. Hence, virus titers in the nasal turbinates and lungs of the animals that received the i.n. immunization on day 5 post-infection were significantly lower than the virus titers in the animals that received PBS at the corresponding time postinfection (FIG. 23F, p<0.05) for the virus titers in the nasal turbinates and lungs, respectively. In addition, no live virus was found in the trachea and brain tissues of either PBS- or the S-Fc-immunized hamsters. These data indicate that the i.n. immunization with S-Fc provides protective immunity against SARS-CoV-2 infection in hamsters.

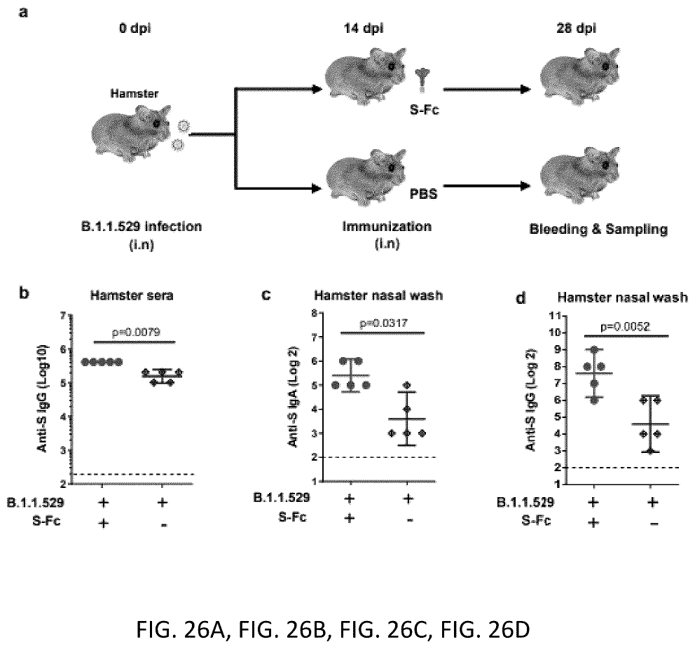

To examine the efficacy of S-Fc for boosting pre-existing immunity conferred by infection with a heterologous SARS-CoV-2 variant, we intranasally infected 10 hamsters with the Omicron B.1.529 strain (1×10³ TCID50). After two weeks, five infected hamsters were i.n. immunized with S-Fc plus CpG, while the other five received only CpG. Two weeks after the boost, their IgG or IgA levels were measured in the blood samples and nasal washes from all the animals. As shown in FIG. 26, the S-Fc intranasal immunization significantly boosted pre-existing serum IgG levels and the levels of nasal IgG and IgA from the hamsters previously infected with the Omicron B.1.529 strain.

FIG. 18 shows the specific binding was detected by the ELISA method. Ten g of S-Fe, S (with the equivalent molar number), or PBS in combination with 10 μg of CpG was i.n. administered into 6-8-week-old wild-type (WT) C57BL/6 mice. Mice were boosted 14 days after the primary immunization. The IgG1 Fc-specific Ab titers were measured by coating the plates with human IgG1 in ELISA. The IgG titers in mouse sera (n=16 for S-Fc/WT and S groups, N=15 for PBS group) were measured (FIG. 18)

FIG. 21 shows gating strategy for identifying TRM T cells in the lungs. The lymphocyte population was first gated from the lung tissue's mixed cells in the plot of FSC vs. SSC. Then an FSC-H vs. FSC-A plot was used to select singlets, followed by a viability dye staining to exclude the dead cells. An intravenous (IV) staining strategy using PE-CD3 was applied to distinguish resident cells from others to rule out the positively stained circulating T cells. The negative population (IV-) showing the positive reaction for the in-vitro FITC-CD3 staining was characterized as a T subset in the lung. This subset was further separated into CD4+ and CD8+T cells and subjected to the CD44-Ab staining and S-specific tetramer staining. Finally, after the staining with antibodies against CD69 or CD103, the representative phenotypes of TRM were defined.

FIG. 22A shows ten g of S-Fc with 10 μg of CpG was i.n. or i.m. administered into 6-8 week-old hACE2 mice (n=11 for i.n group and n=10 for i.m group). Mice were boosted 14 days after primary immunization. A group of mice (n=10) were mock immunized as a negative control. Throat swabbing was performed daily from 1-4 dpi. Half mice in each group were euthanized at 2 and 4 dpi, respectively, for harvesting tissues and titrating the virus.

FIG. 23 shows intranasal immunizations with the S-Fc protein protect Golden Syrian hamsters from SARS-CoV-2 infection. 23a 30 μg of the S-Fc (n=8) or PBS (n=8) in combination with 30 μg of CpG was i.n. administered into female hamsters twice in a 2-week interval. Animals were challenged with ancestral SARS-CoV-2 (1×10⁵ TCID50), and nasal washings were collected at 2, 4, and 6 dpi. Four hamsters from each group were euthanized and sampled at 5 dpi for titrating the virus. The 4 hamsters in each group were used for weight loss and survival assay. FIG. 23B shows anti-SARS-CoV-2 S-specific IgG Ab titers in the hamster sera. The S-specific Ab titers were measured by coating with S protein in ELISA 14 days after the boost. The IgG titers from eight hamster sera per group were calculated. FIG. 23C shows the nAb in the immunized hamster sera. Two weeks after the boost, sera sampled from eight hamsters per group were heat-inactivated and serially diluted two-fold in PBS. The micro-neutralization test determined the neutralizing Ab activity in the sera. FIG. 23D shows changes in body weight after virus challenge. Seventeen days after the boost, hamsters were i.n. challenged with 1×10⁵ TCID50 of ancestral SARS-CoV-2 and weighed daily for 14 days. Hamsters were euthanized at the end of the experiment or when a humane endpoint was met. The data represent mean±SD. FIG. 23E shows shedding of SARS-CoV-2 virus in nasal wash samples of the immunized and controlled hamsters. The virus levels were determined by a TCID50 assay. FIG. 23F shows the viral titers in the nasal turbinate, trachea, lung, brain, and intestine at 5 dpi. Four animals from each group were euthanized for virus titration 5 days after the challenge. Supernatants of the nasal turbinate, trachea, lung, brain, and intestine homogenates were added onto Vero-E6 cells and incubated for four days. The data in B, C, E, F represent a geometric mean with 95% CI. The viral titers were shown as TCID50 from each animal swab. The statistical analyses were determined by Mann-Whitney test (two-tailed) (23B, C, F, E at 6 dpi) and unpaired T-test (two-tailed) (e at 2 dpi and 4 dpi). Dashed lines represent LOD or humane endpoint (D).

FIG. 24 shows a comparison of virus load and RNA levels in the throat swab. FIG. 24A shows a comparison of virus load in the throat swab of the donor hamsters from different groups after the SARS-CoV-2 challenge (n=6 for group 2 and 4, n=12 for group 1+3). The presence of the infectious live virus 1-7 days after infection is determined by TCID50 assay in VAT cells and shown as geometric mean with 95% CI. The ordinary one-way ANOVA followed by Dunnett's multiple comparison test was used for statistical analysis of the data collected at 2, 3, 4 dpi, while Kruskal-Wallis test followed by Dunn's multiple comparisons tests was used for data collected 1, 5, 6, and 7 dpi. FIG. 24B-E show SARS-CoV-2 RNA levels in throat swabs. Total RNAs were isolated and subjected to the one-step qRT-PCR analysis. Viral loads were quantified as SARS-CoV-2 N gene RNA in throat swab fluid on days 1-4 after exposure. Viral RNA was expressed as N gene RNA copy numbers from each swab of animal, based on an RNA standard included in the assay.

FIG. 25 shows transmission of Omicron XBB.1.5 subvariant in hamsters. The donors (n=6) or recipient (n=6) hamsters were unimmunized. The donor hamsters were i.n. infected with 1×10⁵ TCID50 Omicron XBB.1.5 subvariant. Fourteen hours later, donor hamsters in the wire cages were separately cohoused with 6 recipient hamsters within the same isolator. Throat swabbing was performed for 7 days for donor hamsters (1-7 dpi) and 10 days for recipient hamsters (1-10 days after exposure). Virus loads in the throat swab from the donor and recipient hamsters were measured in VAT cells in a 4-day culture. The viral titers were shown as TCID50 from each animal sample. Each open circle represents one animal, and the bar represents each group's median value at the indicated time points.

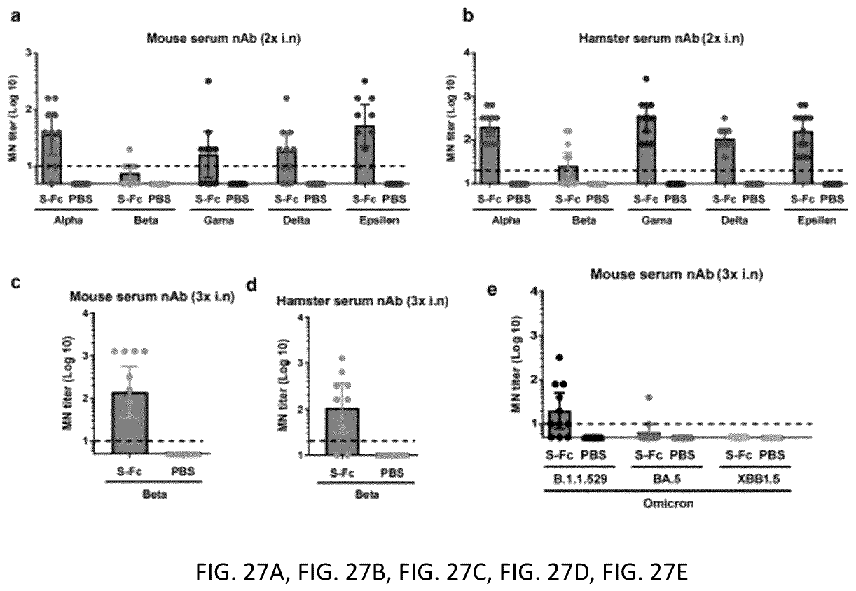

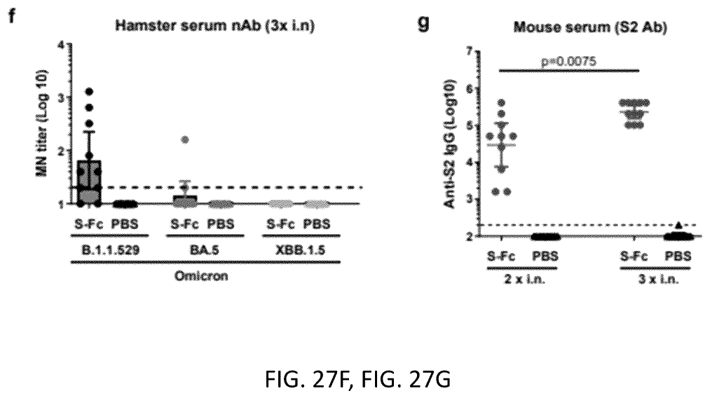

FIG. 27 shows measurement of neutralizing Abs against different SARS-CoV-2 variants or Omicron subvariants. The 6-8-week-old WT C57BL/6 mice (n=10 for 27A, n=11 for 27C and 27E) were i.n. immunized by 10 μg S-Fc, or PBS in combination with 10 μg of CpG); Female hamsters were i.n. immunized with 30 μg of the S-Fc (n=10), or PBS (n=10) in combination with 30 μg of CpG (27B, D, F). Both mice and hamsters were i.n. boosted 14 (2×) and 28 (3×) days, respectively, after the primary immunization. Two weeks after the boosts, sera sampled from the immunized animals were heat-inactivated and serially diluted in PBS. The neutralizing Ab activity in the sera was determined by the micro-neutralization test against different SARS-CoV-2 variants or Omicron subvariants. FIG. 27G shows anti-SARS-CoV-2 S2-specific IgG Ab titers in mouse sera. The S2-specific Ab titers 14 days after the 2nd or 3rd immunization were measured by ELISA using S2 protein to coat the plates. The IgG titers from sera in each group (n=10 for 2× groups, n=11 for 3× groups) were measured and shown as geometric mean with 95% CI.

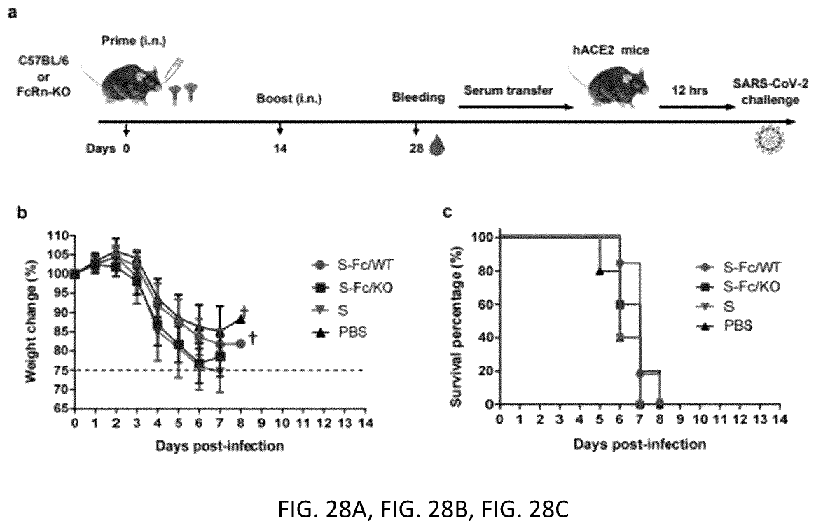

FIG. 28 shows an evaluation of the passive protection by serum transfer from the immunized mice. Ten g of S-Fc, S (with the equivalent molar number), or PBS in combination with 10 μg of CpG was i.n. administered into 6-8-week-old wild-type (WT) or FcRn knockout (KO) mice. Mice were boosted 14 days later after primary immunization. Sera were sampled from all immunized mice, and 200 μl pooled sera from each group were transferred to 8-week-old hACE-2 mice via i.p injection; 12 hours later, all hACE-2 mice (n=5 or 6/group) were i.n. challenged with ancestral SARS-CoV-2 (2.5×10⁴ TCID50) (28A) and weighed daily for 14 days (28B). The survival following the virus challenge is plotted as a Kaplan-Meier curve (28C). Mice were deceased or humanely euthanized if the humane endpoint was reached.

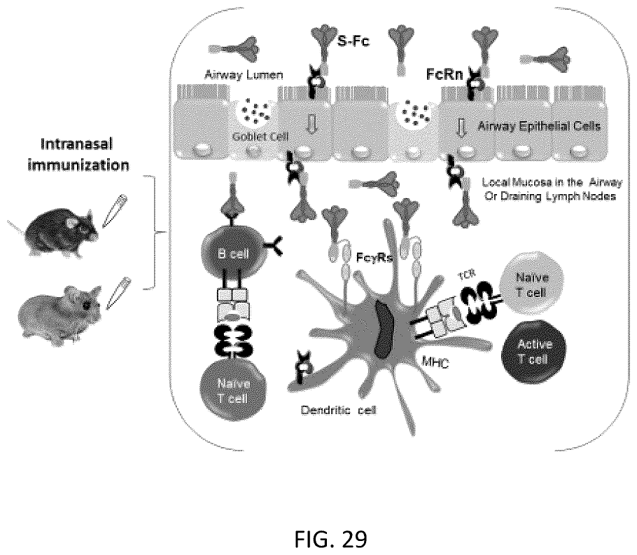

FIG. 29 shows a proposed model of FcRn-targeted mucosal delivery of the S-Fc vaccine antigen. The S-Fc proteins are transported by FcRn across the epithelium and targeted to the mucosal antigen-presenting cells (APC), such as dendritic cells. The S-Fc is taken up by pinocytosis or FcTRs-mediated endocytosis by APCs, then processed and presented to T cells in either lamina propria of the respiratory mucosa or germinal centers of draining lymph nodes.

### iii. Discussion

This study explored a respiratory vaccination strategy by using the mucosa-specific, FcRn-mediated IgG Ab transfer pathway to reduce or eliminate SARS-CoV-2 replication and spread in or through the nose and lungs. The present study produced several lines of evidence. First, the S-Fc-immunized mice had higher IgG and IgA Abs in nasal washes, BAL, and blood compared with those immunized by S alone or FcRn KO mice immunized by S-Fc. Also, the S-Fc-immunized mice exhibited more potent nAb activity than the control animals. Second, as opposed to the PBS control animals, hamsters or hACE2-transgenic mice i.n. immunized by S-Fc developed resistance to the ancestral SARS-CoV-2 infection, exhibiting reduced virus replication in the nasal turbinate, lung, and mouse brain. High levels of live virus were detected in the brains of the infected unimmunized hACE2 mice but not in the infected hamsters. The expression level and localization of human ACE2 in transgenic animals may explain this discrepancy. For example, hamsters expressing human ACE2 also have high virus titers detected in the brain following exposure to SARS-CoV-225. Third, the S-Fc-immunized, hACE2 mice effectively protected against infections by the SARS-CoV-2 Delta or Omicron B.1.1.529 variants. An additional booster was required to protect hACE2 mice from Omicron infection completely. Likewise, to induce broad nAbs against the Omicron strain, it is evident that two boosters are also required for the mRNA vaccinations 26. A test was conducted to assess the efficacy of sera obtained from immunized mice and hamsters in neutralizing different strains of SARS-CoV-2 (FIG. 17a-f). These findings indicate that, while the sera from immunized animals could effectively neutralize SARS-CoV-2 strains a, 3, 7, 6, F, they were found to be ineffective against Omicron strains BA.5 or XBB.1.5. Similarly, studies have shown that the sera from people who received an ancestral SARS-CoV-2 mRNA vaccine booster were unable to effectively neutralize Omicron strains BA.2.12.1, BA.4, BA.527, or an ancestral SARS-CoV-2/BA.5 bivalent mRNA vaccine was unable to induce neutralizing antibodies against Omicron strains BQ.1, BQ.1.1, XBB, and XBB.128. To ensure the efficacy of the S-Fc antigen against the latest Omicron strains, the Spike from XBB.1.5 was used. One objective is to create a nasal vaccine that can actively elicit broad-spectrum protective immune responses against various SARS-CoV-2 variants. Fourth, remarkably, most S-Fc-immunized animals exhibited significantly reduced inflammation in the lungs of hACE2 mice.

In the airborne transmission study, in striking contrast to the unimmunized hamsters or hamsters immunized by the i.m. route, most i.n. S-Fc immunized hamsters infected with the virus did not spread the virus to unimmunized hamsters. Similarly, most i.n. S-Fc immunized hamsters were not infected when placed with the infected unimmunized hamsters. An incomplete block of transmission by intranasal immunization may result from high virus doses during donor hamster infection and extended housing periods for infected and exposed animals. It would be interesting to know if hamsters received i.n. S-Fc immunization could prevent the spread of Omicron subvariants. The low efficiency of airborne transmission of Omicron BA.1, BA.2.12.2, BA.529, and XBB.1.5 (FIG. 25) in hamsters makes it challenging to evaluate the nasal vaccines in blocking Omicron airborne transmission. However, Omicron variants can also be transmitted via fomites and a COVID-19 nasal vaccine should protect against airborne and fomite transmission.

Several mechanisms may account for protection against SARS-CoV-2 infection and transmission. FcRn binds S-Fc in acidic pH conditions. A slightly acidic pH in the respiratory tract is expected to facilitate FcRn to retain antigens in airway mucosal surfaces and transfer them across the airway barrier. The local Ab immune responses can represent a primary barrier of immune defense against viral infections of the respiratory tract, characterized by the presence of sIgA in the nasal mucosa or IgG in the BAL10. The natural SARS-CoV-2 infection induces mucosal sIgA, which dominates the early nAb response to SARS-CoV-2 with potent neutralizing activity. Furthermore, the levels of mucosal Abs determine the viral load and the time required to recover from systemic symptoms. In this study, the mice and hamsters that were i.n. immunized by S-Fc developed high levels of IgA Abs in the nasal washes and BAL. These results are consistent with the findings of previous studies where FcRn-targeted respiratory immunizations against viral mucosal infections and adenovirus-vectored SARS-CoV-2 nasal vaccine elicit high Ab responses. In addition, the mice or hamsters i.m. immunized by S-Fc failed to produce significantly increased levels of IgA Abs in the nasal washes, despite high IgG Abs in the BAL. A robust SARS-CoV-2 infection in nasal turbinates is readily detectable after the adoptive transfer of the nAbs. This observation is further supported by findings that hACE2 mice succumb to nasal infection following an adoptive transfer of serum Abs from i.n. immunized mice by S-Fc (FIG. 28). It is possible that passive Ab transfer may not protect hACE2 mice from SARS-CoV-2 infection because the Abs may not be able to pass through the blood-endothelial barrier and reach the olfactory mucosa. Indeed, i.m. vaccination fails to reduce viral load in nasal swabs or turbinate significantly. Considering SARS-CoV-2 also infects cells via a cell-to-cell fashion, it may be difficult for serum IgG Abs to block viral entry and spread in the upper respiratory tract, or a high level of IgG Abs is required. In addition, high levels of S2-specific IgG Abs were found in the immunized mice (FIG. 27g). It is important to note that S2 is more conserved across different versions of SARS-CoV-2,

FcRn-mediated respiratory delivery of vaccine antigen facilitates the production of memory immune responses. The induction of S-specific memory immune responses is crucial for a vaccine to provide sustained protection. The S- or RBD-specific nasal Abs can last 9 months in COVID-19 patients. FcRn-mediated respiratory vaccination with S-Fc induces and sustains high levels of S-specific IgA and IgG and plasma cells 8 months after the boost, which they resist lethal SARS-CoV-2 infection at least 6 months after the boost. TRM T cells in the lung can promote rapid viral clearance at the site of infection and mediate survival against lethal viral infections, especially in severe COVID-19 patients. This study demonstrated that a higher percentage of CD4+ or CD8+ TRM T cells were present in the lungs of mice that received i.n. S-Fc immunization than in those of PBS control animals. Furthermore, the i.n immunization induced significantly more lung-resident TRM cells than the i.m. route. This is consistent with previous findings by directly delivering viral antigens into the airway. Additionally, FcRn prolongs IgG half-life. Thus, FcRn binding to S-Fc is expected to stabilize S-Fc in vivo, enhancing FcγRs-mediated uptake of S-Fc by antigen-presenting cells.

First, i.m. vaccination couldn't effectively induce local immunity in the respiratory mucosa. Indeed, the i.n., but not i.m., immunizations with S-Fc induce nasal IgA and TRM T cells in the lung. Second, individuals infected by SARS-CoV-2 through the nasal epithelial cells can asymptomatically shed the infectious virus for an unknown time. Hence, the nasal vaccination can be an essential complementary strategy to the i.m. vaccines that are authorized or undergoing clinical trials. Because FcRn can transfer the immune complexes across the mucosal epithelial barrier with or without pre-existing Abs, the efficacy of the FcRn-targeted mucosal vaccination is not expected to be influenced by pre-existing SARS-CoV-2 immunity. An instance of the S-Fc i.n. immunization of hamsters previously infected by Omicron B.1.1.529 resulted in a significant enhancement of pre-existing serum IgG levels, along with increased levels of both nasal IgA and IgG (FIG. 26). The FcRn-mediated nasal vaccine can be used as the first dose for an unimmunized person or booster for those who have been immunized, even previously infected by SARS-CoV-2. The combination of mucosal and parenteral vaccines has been proven effective at mucosal entry against SARS-CoV-2 infections.

In addition to pneumonia and acute respiratory distress, some symptomatic or asymptomatic COVID-19 patients, including those with breakthrough infections, are reported to experience a long COVID. The exact causes of the long COVID-19 are elusive; they may involve direct effects of viral infection or indirect influences on the brain. In humans, the nasal olfactory epithelium expresses a high level of ACE2 receptor. Although debatable, SARS-CoV-2 may enter the brain by crossing the neural-mucosal interface in olfactory mucosa or infecting the olfactory epithelium or bulb, or both. Indeed, SARS-CoV-2 is identified to replicate in brain astrocytes. Alternatively, inflammatory cytokines derived from inflamed lungs or other organs may cross the blood-brain barrier. Consequently, a virus or cytokine may cause encephalitis or necrosis, leading to a long COVID. The hACE2 mice are highly susceptible to SARS-CoV-2 infection with the virus detected in the brain. This study confirms this and shows that FcRn-mediated respiratory vaccination can prevent brain viral infection in hACE2 mice. Likely, the sIgA or TRM T cells induced by S-Fc can efficiently block the viral replication in nasal turbinates, reducing viral infection in the olfactory epithelium or access to the olfactory bulb.

A respiratory vaccine was developed using pre-stabilized SARS-CoV-2 S antigen that stimulates local and systemic protective immunity. According to this model (FIG. S13), mucosal antigen-presenting cells that express FcγR, like dendritic cells, absorb the S-Fc protein that is transported across respiratory epithelial cell monolayer by FcRn and then migrate to mediastinal draining lymph nodes where they activate CD4+ T cells and trigger the B cell response in the germinal centers. By increasing the persistence of the S-Fc in tissue and circulation, the vaccination may further enhance the development of long-term humoral and cellular immunity. The substantially longer half-life of IgG in humans compared to mice or hamsters (21 vs. 6 days) would ensure high and persisting levels of S-Fc in human immunization. Also, the S-Fc and human IgG1 had a lower affinity to the mouse FcγRI, but it exhibits a high affinity with human FcγRI (FIG. 1d), suggesting our S-Fc vaccine may work more efficiently in humans. Although the i.n. administered viral vaccines induce protection in the respiratory tracts, a protein-based nasal vaccine may be preferred, especially in young, elderly, and immunocompromised populations. It is also interesting to compare the protective immunity in the upper respiratory tract and protection against infection and transmission induced by the protein-based nasal vaccine and the licensed mRNA vaccine. Together, these results indicate that FcRn-mediated respiratory immunization can be an effective and safe strategy for maximizing the efficacy of vaccinations against infection and transmission of SARS-CoV-2 and its emerging variants.

SARS-CoV-2 constantly evolves, resulting in variants of concern (VOCs). Omicron subvariants are not only highly contagious but also resistant to nAbs. To show whether the sera from the mice or hamsters that were i.n. immunized by S-Fc neutralize SARS-CoV-2 variants, a neutralization test was also conducted. The results showed that mice or hamsters that received two doses of intranasal immunizations with S-Fc in a two-week interval were able to neutralize SARS-CoV-2 strains, α, β, γ, δ, and ε, as compared to the control group (FIG. 27a, b). Interestingly, the level of nAbs against strain 3 was lower in the immunized animals. However, after receiving three doses of i.n. immunizations, most mice and hamsters developed higher levels of nAbs against strain R (FIG. 27c, d).

Whether animal sera of 3-dose intranasal immunizations could neutralize Omicron variants was further examined. Although most immunized mice or hamsters developed nAbs against B.1.1.529, they did not produce strong Abs against BA.5 or XBB.1.5 (FIG. 27e, f). These results indicate that Abs induced by ancestral SARS-CoV-2 spike antigens are ineffective at neutralizing Omicron BA.5 or XBB.1.5.

### iv. Methods

a. Mice and Golden Syrian hamsters

All research experiments in this study comply with all relevant ethical regulations. The animals were acclimatized at the animal facility for 4-6 days before initiating experiments. Animals from different litters were randomly assigned to experimental groups, and investigators were not blinded to allocation during experiments and outcome assessment. Transgenic mice expressing human ACE2 by the human cytokeratin 18 promoter (K18-hACE2) represent a susceptible rodent model. Specific-pathogen-free, 6-8-week-old, female and male B6.Cg-Tg(K18-ACE2)2Prlmn/J (Stock No: 034860, K18-hACE2) hemizygous C57BL/6 mice and control C57BL6 mice (non-carriers) were purchased and used for breeding pairs to generate pups for research. All the offspring were subjected to genotyping, and only the hemizygous K18-hACE2 mice were chosen for future use. Seven-week-old male/female Golden Syrian hamsters were obtained. Animals have been maintained in individually ventilated cages at ABSL-2 for noninfectious studies or in isolators within the ABSL-3 facility for studies involving SARS-CoV-2 viruses. Immunization and virus inoculation were performed under anesthesia. Volatile chemical anesthetics known to increase the permeability of the respiratory epithelial barrier in nasal immunizations were avoided. All mice were anesthetized with an intraperitoneal (i.p.) injection of fresh Avertin at 10-12.5 μl of working solution (40 mg/ml) per gram of body weight- and laid down in a dorsal recumbent position to allow for recovery. Hamsters were sedated with Dexmedetomidine (50-250 μg/kg) via subcutaneous injection for immunization and virus infection, or they were anesthetized with isoflurane for collecting blood, nasal washes, and throat swabs.

b. Cells, Antibodies, and Viruses

Vero E6 (with high expression of endogenous ACE2, Cat No. NR-53726) and VAT (Vero E6-TMPRSS2-T2A-ACE2, Cat No.NR-54970) were from Biodefense and Emerging Infections Research Resources Repository (BEI Resources, Manassas, VA). Chinese hamster ovary (CHO) cells were purchased from the American Tissue Culture Collection (ATCC, Manassas, VA). Vero E6, VAT, and CHO cells were maintained in complete Dulbecco's Minimal Essential Medium (DMEM) (Invitrogen Life Technologies), supplemented with 10% fetal bovine serum (FBS), 2 mM L-glutamine, nonessential amino acids, and antibiotic and antifungal (100 units/ml of penicillin, 100 μg/ml of streptomycin, and 250 ng/ml of amphotericin B). Vero E6, VAT, and CHO cells routinely tested negative for Mycoplasma sp. by real-time PCR. Recombinant CHO cells were grown in a complete medium with G418 (Invitrogen, 1 mg/ml). All cells were grown at 37° C. in 5% CO2.

The insect cell-expressed SARS-CoV-2 Spike S2 (Cat #NR-53799), S-specific mAbs 2TP1B11, 2TP2C7, and 2TP22E7 were procured from the BEI Resources. S-specific mAbs 40150-R007, 40952-MM57, and SPD-M128, as well as anti-S polyclonal antibodies 40590-T62 and 40150-T62-Cov2, were acquired from Sino Biologicals. Hamster IgG2 was purchased from BD (Cat #553294). Motavizumab, an antibody against the respiratory syncytial virus (RSV) F protein, was acquired from Cambridge Biologics (Brookline, MA). The sera from the convalescent COVID-19 patients or health persons were a gift from the Biotech Laboratories (Rockville, MD). The horseradish peroxidase (HRP)-conjugated anti-human IgG (Cat #2081-05), anti-goat IgG (Cat #6160-05), streptavidin (Cat #7100-05), and biotin-labeled goat anti-mouse IgA (Cat #1040-08) were obtained from Southern Biotech (Birmingham, AL). HRP-conjugated anti-mouse IgG Fab (Cat #A9917) and anti-human IgG Fab (Cat #SAB4200791) were from Sigma. HRP-conjugated anti-mouse IgG (Cat #PA1-28568) and anti-hamster IgA (sab3003a) were obtained from Invitrogen (Waltham, MA) and Brookwood Biomedical (Jemison, AL), respectively. Goat anti-hamster IgG (Cat #NB1207141) was acquired from Novus (Centennial, CO). Recombinant biotinylated human and biotinylated mouse FcRn/β2m (Cat #FCM-H82W4 and FCM-M82W6) and biotinylated human and mouse FcγRI proteins (Cat #FCA-H82E8 and CD4-M82E7), and biotinylated human ACE2 protein (Cat #AC2-H82E6) were purchased from AcroBiosystems (Newark, DE). Human C1q protein was a gift (Complement Technology, Cat #A099). Mouse C1q protein (Cat #M099) was procured from Complement Technology (Tyler, TX).

The following reagents were obtained through BEI Resources, NIAID, NIH: SARS-CoV-2 ancestral isolate hCoV-19/USA/NY-PV08410/2020 (abbreviated as NY strain, Cat #NR-53514), Alpha isolate (B.1.1.7) hCoV-19/USA/OR-OHSU-PHL00037/2021 (Cat #NR55461), Beta isolate (B.1.351) hCoV-19/USA/MD-HP01542/2021 (Cat #55282), Gamma isolate (P.1) hCoV-19/Japan/TY7-503/2021 (Cat #NR-54982), Delta isolate (B.1.167.2) hCoV-19/USA/PHC658/2021 (Cat #NR-55611), Epsilon isolate hCoV-19/USA/CA/VRLC014/2021 (Cat #NR-55309), Omicron B.1.1.529 strain hCoV-19/USA/MD-HP20874/2021 (Cat #: NR-56461), BA.5 subvariant hCoV-19/USA/COR-22-063113/2022 (Cat #NR-58616), XBB.1.5 subvariant hCoV-19/USA/MD-HP40900/2022 (Cat #NR-59104). Viruses from the BEI were passed in either Vero E6 (for ancestral and Delta strains) or VAT cells (for Omicron strain). At 72 hr post-infection, tissue culture supernatants were collected and clarified before being aliquoted and stored at −80° C. The virus stock was titrated using the Median Tissue Culture Infectious Dose (TCID50) assay.

c. Construction of a Plasmid Expressing SARS-CoV-2 Spike (S) or S-Fc

The entire amino acid (aa) sequence corresponding to the S protein of the ancestral SARS-CoV-2 strain Wuhan-Hu-1 or Omicron XBB.1.5 was retrieved from Genbank. An S gene of SARS-CoV-2 was designed with some modifications described below and synthesized it from GenScript (Piscataway, NJ). The S protein precursor has two well-defined cleavage sites: S1/S2 and S2′. To produce a non-cleavable S protein, we performed mutagenesis at the cleavage site S1/S2 (R685A) and S2′(R815A) of the S gene to keep the S protein in a pre-cleavage conformation. On the surface of coronaviruses, S glycoprotein exists predominantly in the prefusion form. A prefusion structure of SARS-CoV-2 S is critical for maximizing immunogenicity. To produce a prefusion form of S antigen, we made a pair of point mutations to proline (K986P and V987P), which prevents the formation of a helix associated with the post-fusion conformation (FIG. 1a). The S protein also naturally exists as a trimer on the virions or virally infected cells. To facilitate the trimerization of soluble S protein, we engineered a foldon domain from T4 bacteriophage fibritin protein18 to the C-terminus of the extracellular domain of the S (residues 1-1213) gene to facilitate the trimerization. To target FcRn for delivery, human IgG1 Fc was selected. The rationale for using human IgG1 is consistent with the fact that it has the highest affinity for activating FcγRI but the lowest affinity for inhibiting FcγRIIB. In IgG1 Fc, the complement C1q-binding motif was eliminated (E318A/K320A/K322A) (FIG. 1a) to prevent the binding of human and mouse C1q. Because human IgG1 Fc typically forms a disulfide-bonded dimer, a monomeric Fc was created by substituting cysteines 226 and 229 with serine (C226S and C229S) to eliminate the disulfide bonds. The modified S gene (named by S-Fc) encodes the extracellular domain of the S protein, followed by a trimeric motif (foldon) and a monomeric human IgG1 Fc segment. The S-Fc gene was further codon-optimized for optimal expression in CHO cells and synthesized and cloned into the eukaryotic expression plasmid pcDNA3.1 via Kpn I and Xho I sites to generate the recombinant plasmids pcDNA3.1-S-Fc (FIG. 1a).

A control plasmid, pcDNA3.1-S, was produced by replacing the human IgG1 Fc portion with the 6× His tag sequence. For this purpose, an inverse PCR was performed using pcDNA3.1-S-Fc as a template, and the primer pairs: 5′-CACCTTCCTGGGCCATCATCACCATCACCATTGACTCGAGTCTAGAGGGCCCG-3′ (SEQ ID NO:19) and 5′-ATGGTGATGGTGATGATGGCCCAGGAAGGTGGACAGCAGCACCCACTCGCCAT-3′ (SEQ ID NO:20). After transforming the inverse PCR segment into the competent E. coli strain, homologous recombination allows for the generation of the circular plasmid pcDNA3.1-S, which encodes a trimeric soluble S protein with the foldon domain and 6× His tag.

d. Generation of a Plasmid Expressing Hamster FcRn/β2m or FcγRI

To construct plasmid for the expression of hamster FcRn and 02m, the DNA sequences encoding the extracellular domain of hamster FcRn (1-300 aa) or full-length hamster 02m were firstly amplified from hamster lung tissue by RT-PCR. The primer pairs for hamster FcRn were 5′-GCGGGTACCGCCACCATGGGGATGCCCCAGCCC-3′ (SEQ ID NO:21), 5′-TATCTCGAGTTACTCGTGCCACTCGATCTTCTGGGCCTCGAAGATGTCGTTCAGGCC GTGGTGATGGTGGTGATGGTGATGCGAAGATCTGGCTGGAGCA-3′ (SEQ ID NO:22). The primer pairs for hamster 02m amplification were 5′-TATGTCGACGCCACCATGGCTCGCTCCGTGGCCG-3′ (SEQ ID NO:23), 5′-GCGTCTAGACTATTTTTCGAACTGCGGGTGGCTCCACATGTCTCGTTCCCAGGTGAC-3′ (SEQ ID NO:24). The corresponding FcRn cDNA sequence comprises a coding sequence for 8× His tag and Avi tag at the 3′ end for protein purification and site-specific biotinylation, respectively, while the 02m cDNA included a Strep II tag at its 3′ end. Then, the FcRn and 02m cDNAs were subsequently cloned into a dual-expression vector pBud-CE4.1 (Invitrogen) via the Kpn I/Xho I sites (for FcRn) and Sal I/Xba I sites (β2m) to generate a plasmid pBud-haFcRn/β2m.

An RT-PCR was performed to amplify the gene sequence encoding the ectodomain of FcγRI from hamster lung tissue to create a plasmid expressing hamster FcγRI. The primer pairs were 5′-GCGGGTACCGCCACCATGTGGCTCCTAACAACCCTG-3′ (SEQ ID NO:25) and 5′-TATCTCGAGTTACTCGTGCCACTCGATCTTCTGGGCCTCGAAGATGTCGTTCAGGCC GTGGTGATGGTGGTGATGGTGATGAGGGCCTGATGACTGAGGAC-3′ (SEQ ID NO:26). As described above, the tandem 8× His tag- and Avi tag-encoding sequence at the 3′ end of the FcγRI segment was designed for affinity purification and site-specific biotin labeling. Next, the hamster FcγRI segment was digested with Kpn I and Xho I and inserted into the pBud-CE4.1 vector to generate a plasmid pBud-haFcγRI.

e. Characterization of the Prefusion S and S-Fc Proteins

The pcDNA3.1 plasmids encoding S and S-Fc were transfected into CHO cells using PEI MAX 40000 (Fisher Scientific, Cat #NC1038561). Stable cell lines were selected and maintained under G418 (1 mg/ml). Immunofluorescence assay, SDS-PAGE, and Western blotting analysis determined the expression and secretion of the S or S-Fc fusion proteins. We produced the soluble S or S-Fc proteins by culturing CHO cells in a complete medium containing 5% FBS with ultra-low IgG. The proteins were captured by Protein A column (ThermoFisher Scientific, Cat #20356) for the S-Fc protein or Histidine-tagged Protein Purification Resin (R&D Systems, Cat #IP999) for the S protein, eluted with 0.1 M Glycine (pH 2.5), and neutralized with 1 M Tris-HCl (pH 8.0). Glycine and Tris-HCl in the protein solution were replaced with PBS three times using centrifugation with Amicon Ultra-15 Centrifugal Filter Unit (50 K) (Millipore, Cat #UFC905024). Protein concentrations were determined using a NanoDrop spectrophotometer (Thermo Scientific).

f. Purification of Hamster FcRn/β2m or FcγRI Proteins

To express hamster FcRn/β2m or FcγRI proteins, 293T cells were transfected with recombinant plasmid pBud-haFcRn/β2m or pBud-haFcγRI. The supernatants from the cell culture were harvested after 48 hrs, loaded onto an anti-His resin column, and eluted with 0.1 M glycines (pH 2.5). The eluted proteins were neutralized with 1 M Tris-HCl (pH 8.0), and the protein buffer was replaced with PBS using Amicon Ultra-15 Centrifugal Filter Unit (10 K). The purified proteins were visualized by SDS-PAGE and Coomassie staining. The site-specific biotinylation was then conducted by using a BirA biotin-protein ligase standard reaction kit (Cat #BirA500) from Avidity Biosciences (Aurora, CO).

g. SDS-PAGE Gel and Western Blotting

Protein quality was assessed by 8-15% SDS-PAGE gel under reducing conditions. Proteins in gels were either stained with Coomassie blue dye in gel or used for transfer onto nitrocellulose membranes (GE Healthcare). The membranes were blocked with 5% milk in PBST (PBS and 0.05% Tween-20) and incubated with appropriate primary and HRP-conjugated secondary antibodies, as indicated in the Figures. The immobilon Western chemiluminescent HRP substrate (Millipore, Cat #WBKLS0100) was used to visualize protein bands in membranes and images captured by the Chemi-Doc™ XRS system (BioRad).

h. Samplings of Nasal Washes, Bronchoalveolar Lavage, and Throat Swabs

The nasal washes and BAL fluids were collected from the mouse under euthanasia with over-dosage of Avertin. For sampling nasal washes, the lower jaw was removed. A small incision was made over the ventral aspect of the trachea; then, a syringe with a blunt plastic tip was inserted into the trachea toward the nasal cavity. 1 ml PBS was gently injected into the nasopharynx and collected when it flowed from the external nares. For BAL collection, the syringe was inserted into the trachea but toward the lungs, and 1 ml of PBS was carefully injected into the lungs by keeping the syringe in position. The PBS was retrieved back to obtain BAL fluids. The nasal washes and BAL fluids were centrifuged to remove cellular debris, concentrated to 350 μl with Amicon 0.5 ml centrifugal filter unit (10 K) (Millipore, Cat #UFC501096), and the supernatants were stored at −20° C.

To collect nasal washes in hamsters, animals were anesthetized with isoflurane, and small-sized feeding needles (20 G) were used to inject 500 μl sterile PBS into the nostrils (250 μl each side). The outflows were collected in a petri dish as expelled by the hamster. The volume was increased to 0.5 ml with the addition of cold PBS. To collect throat swabs from the anesthetized hamsters, the swab was first moistened in 650 μl DMEM media with 1% inactivated FBS and then placed into the throat, where it was gently rubbed around ten times. These swabs were then soaked for 5 minutes in the vials containing the remainder of the media. Subsequently, the throat swabs were removed, and the samples were vortexed and stored at −80° C. for further virological analysis.

i. Enzyme-Linked Immunosorbent Assay (ELISA) or Spot (ELIspot)

IgA and IgG antibodies in nasal washes, BAL fluid, and sera reactive to SARS-CoV-2 spike antigens were quantified by ELISA. In brief, 96-well plates (Maxisorp, Nunc) were coated with 1 μg/ml of the S protein described above in 100 μl coating buffer (PBS, pH 7.4) per well and incubated overnight at 4° C. Plates were washed four times with 0.05% Tween 20 in PBS (PBST) and blocked with blocking buffer (2% bovine serum albumin in PBST) for 2 h at room temperature. Sera were heat-inactivated at 56° C. for 30 min before serial dilutions starting at 1:200 in dilution buffer. The serially diluted specimens (nasal wash, BAL, or sera) from animals were added to each well and incubated for 2 hrs. After washing six times with PBST, the detection antibodies were added and incubated for 1.5 hr at room temperature. HRP-conjugated rabbit anti-mouse IgG (1:20,000, Invitrogen, Cat #PA1-28568) was used for measuring mouse IgG, while biotin-labeled goat anti-mouse IgA Ab (1:5000, Southern Biotech, Cat #1040-08) plus HRP-conjugated streptavidin (1:7000, Southern Biotech, Cat #7100-05) were used for measuring mouse IgA antibody. To detect S-specific hamster IgG, goat anti-hamster IgG (1:4000, Novus, Cat #NB1207141) plus HRP-conjugated rabbit anti-goat IgG (1:5000, Southern Biotech, Cat #61-6065) were used. To determine S-specific hamster IgA, HRP-conjugated anti-hamster IgA (1:250, Brookwood Biomedical, Cat #sab3003a) was used. One hundred microliter TMB (tetramethylbenzidine) (BD, Cat #555214) was used as a substrate to visualize the signals. The reactions were stopped after 5 min by adding 100 μl of 1 M sulfuric acid before reading on a Victor III microplate reader (Perkin Elmer) at OD450 nm. Antibody endpoint titers were determined by non-linear regression as the reciprocal of the highest sample dilution with an absorbance above two times the absorbance of blank wells.

For measuring S-specific antibody-secreting cells (ASCs) in bone marrow, an ELISpot kit (MabTech, Cat #3825-2H) was used. The 96-well ELISpot plates (MabTech, Cat #3654-TP-10) were pre-wetted with 35% ethanol and washed five times with sterile water plus 1 with PBS. The plates were then coated with S protein at 20 μg/ml overnight at 4° C. (100 μl/well) and blocked with RPMI 1640 complete medium with 10% FBS for 2 h at room temperature. Bone marrow cells from femurs and tibias were collected in RPMI 1640, filtered through a 70 m strainer, and subjected to ACK lysis. Serial dilutions of single-cell suspensions were prepared in RPMI 1640 and added to the coated wells for 18-24 h at 37° C. in 5% CO2. After incubation, the plates were emptied and washed five times with PBS, then incubated with biotinylated anti-mouse IgG (0.5 μg/ml) for 2 h at room temperature. After washing with PBS, HRP-streptavidin (1:700) was added and incubated for 1 h. The samples were developed with TMB substrate until distinct spots emerged. After washing, the plates were stored upside down in the dark to dry overnight at room temperature. Spots were counted with an ELISpot reader (AID, Germany).

j. Characterization of the S-Fc or S Proteins

The ELISA assays were also used to measure interactions of the S-Fc or S with (1) human, mouse, or hamster FcRn/β2m heterodimer (ACROBiosystems, Cat #FCM-H82W4 for human FcRn/β2m; Cat #FCM-M82W6 for mouse FcRn/β2m; at the acidic pH (6.0) and neutral pH (7.4) conditions; (2) human, mouse, or hamster FcγRI (ACROBiosystems, Cat #FCA-H82E8 for human FcγRI; Cat #CD4-M82E7 for mouse FcγRI); (3) human ACE2 (ACROBiosystems, Cat #AC2-H82E6); and (4) human and mouse C1q protein (Complement Technology, Cat #A099, and #M099). All FcRn/β2M heterodimer, FcγRI, ACE2, and C1q proteins were conjugated with biotin to facilitate detection. In brief, ELISA plates were coated with S-Fc or S protein in PBS (1 μg/well for FcRn/β2M binding or 200 ng/well for FcγRI and hACE2 binding) overnight at 4° C. After blocking for 2 h, the 2-fold serial diluted target proteins (4-4000 ng/ml of FcRn/β2m, 0.4-400 ng/ml of FcγRI and hACE2) were added and incubated for 2 h at room temperature. For the C1q binding assay, S-Fc or S proteins were used to coat plates at a serial dilution (800-7.8 ng/well), and a biotin-conjugated human or mouse C1q (2 μg/ml) was used for detection. For all assays, the streptavidin-HRP was from Southern Biotech (1:5000), and TMB was used to visualize the colorimetric signals. The Victor III microplate reader read the plates at 450 nm to assess optical density (OD).

k. Quantification of SARS-CoV-2 Virus and RNA

The number of infectious virus particles in the specimen of ancestral SARS-CoV-2 or Delta strain infected animals was determined in Vero E6 cells by 50% tissue culture infectious dose (TCID50) endpoint dilution assay. The quantification of the Omicron strain was performed in VAT cells. To increase the sensitivity, VAT cells were also used in detecting ancestral viruses and Omicron variants in the throat swab samples, as the overexpression of the hACE2 and TMPRSS2 in VAT cells enhances the replication efficiency of the SARS-CoV-263. Briefly, cells were plated at 15,000 cells/well in DMEM with 10% FBS and incubated overnight at 37° C. with 5.0% CO2. Media was aspirated and replaced with DMEM with 1% inactivated FBS for virus infection. Animal tissues, including nasal turbinate, lung, brain, intestine, and kidney, were homogenized in the TissueLyser LT (Qiagen). After centrifuging at high speed (18407× g, 10 min), the 10-fold serial dilutions of supernatants were used to infect the cell monolayers in 96-well plates, and the CPE was checked after 4 days. Positive (virus stock of known infectious titer) and negative (medium only) controls were included in each assay. The virus titer was expressed as TCID50/ml (50% infectious dose (ID50) per milliliter) by using the Reed-Muench method62.

To monitor viral RNA levels in virus-infected animal samples (throat swabs), total RNAs were isolated by using the PureLink RNA mini kit (Invitrogen, Cat #12183018A) and subjected to the one-step quantitative real-time reverse transcription-PCR assay (qRT-PCR) using TaqMan Fast Virus 1-Step Master Mix (ThermoFisher, Cat #4444432) as described previously 64,65. A SARS-CoV-2 nucleocapsid (N) specific primers and probe sets were used: Forward primer: 5′-GACCCCAAAATCAGCGAAAT-3′ (SEQ ID NO:27); Reverse primer: 5′-TCTGGTTACTGCCAGTTGAATCTG-3′ (SEQ ID NO:28); and probe: 5′-FAM-ACCCCGCATTACGTTTGGTGGACC-BHQ1-3′ (SEQ ID NO:29)). Briefly, viral RNA was expressed as N gene RNA copy numbers from each swab or animal, based on an RNA standard included in the assay, which was created via the in vitro T7-DNA-dependent RNA transcription of a linearized DNA molecule containing the target region of the N gene full-length by using MEGAscript T7 Transcription Kit (ThermoFisher, Cat #AM1334), and purified with MEGAclear Transcription Clean-Up Kit (ThermoFisher, Cat #AM1908). The amplifications of qRT-PCR were performed with a CFX96 Touch Real-Time PCR System (Bio-Rad) using the following conditions: reverse transcription at 50° C. for 5 min, initial denaturation at 95° C. for 20 s, then 40 cycles of denaturing and annealing/extending at 95° C. for 3 s and 60° C. for 30 s. The lower limit of detection (LOD) was 101.5 copies per reaction.

l. Microneutralization (MN) Assay

Neutralizing antibodies were measured by a standard microneutralization (MN) assay on Vero-E6 (for ancestral and Delta strains) or VTA cells (for Omicron strain). The sera were heat-inactivated at 56° C. for 30 min and followed by 2-fold serial dilution, after which the diluted sera were incubated with 100 TCID50 of SARS-CoV-2 virus (ancestral, Delta, and Omicron strains) for 1 h at 37° C., respectively. The virus-serum mixtures were added to Vero-E6 or VAT cell monolayers in 96-well plates and incubated for 1 h at 37° C. After removing the mixture, DMEM with 1% inactivated FBS was added to each well and incubated for 4 days at 37° C. for daily CPE observation. Neutralizing Ab titers are expressed as the reciprocal of the highest serum dilution preventing the appearance of CPE.

m. Immunizations of Mice and Golden Syrian Hamsters and SARS-CoV-2 Challenge

Six- to eight-week-old female/male C57BL/6 mice, FcRn KO mice, and K18-hACE2 transgenic mice were intranasally (i.n.) immunized with 10 μg S-Fc, equal molar of recombinant S, or PBS in 10 μg CpG adjuvant (ODN1826, Invivogen, Cat #vac-1826-1) in a total volume of 20 μl. CpG does not increase the permeability of the airway respiratory barrier66, and it is recognized by TLR-9 of plasmacytoid dendritic cells (pDCs) and B cells, leading to the activation of a Th1 immune response and priming the induction of long-lasting memory immune responses. For intramuscular (i.m.) immunizations, mice were injected bilaterally in the quadriceps femoris with a 50 μl volume containing 10 μg S-Fc antigen in 10 μg CpG. Six- to eight-week-old female/male hamsters (n=6-8 per group) were vaccinated via i.n. or i.m. route with an 80 μl volume containing 30 μg S-Fc and 30 μg CpG. The mice or hamsters were boosted with the same vaccine formulations 2 or 3 weeks later.

Blood was collected from each animal 2 or 3 weeks after the boost; 3 days later, the animals were transferred to the ABSL-3+ facility for virus challenge. The K18-hACE2 mice were i.n. infected with lethal doses of SARS-CoV-2 virus in a total volume of 50 μl (2.5×10⁴ TCID50 ancestral SARS-CoV-2 and Delta strains, or 1×10⁶ TCID50 for Omicron strain). Due to their high susceptibility, the aged K18-hACE2 mice were challenged with 5×10³ TCID50 ancestral strain. The hamsters were i.n. infected with 1×10⁵ TCID50 of ancestral SARS-CoV-2 strain with a final volume of 100 μl. After infection, animals were monitored daily for morbidity (weight loss), mortality (survival), and other clinical signs of illness for 14 days. Animals losing above 25% of their body weight following infection or reaching the humane endpoint were humanely euthanized.

At the indicated time points after the virus infection, nasal washes or throat swabs were sampled to monitor the virus shedding from the upper respiratory tract. To further measure the virus replication and tissue lesion in vivo, 50% of the animals in each group were euthanized at 4 or 5 dpi, and different organs and tissues, including nasal turbinate, trachea, lung, brain, heart, and intestine, were harvested. The left lung lobe was fixed in a 10% neutral buffered formalin solution for histopathology analyses, while the right lung lobes and other tissues were homogenized in DMEM by Tissue Lyser (Qiagen). The homogenates were cleaned by centrifugation (16,100×g for 10 minutes), and supernatants were collected to measure viral load.

n. Lung Pathology

To examine the lung pathology, lungs were removed from mice in each group and fixed in 10% neutral buffered formalin solution 3 days before transferring the tissues from the ABSL-3 facility. The lungs were then paraffin-embedded, sectioned in five-micron thickness, and stained with Hematoxylin and Eosin (H & E) by Histoserv Inc (Germantown, MD). Stained lung sections were scanned using a high-definition whole-slide imaging system (Histoserv, Germantown, MD).

To determine the level of pulmonary inflammation, the lung inflammation was evaluated and scored by a board-certified veterinary pathologist blinded to the experimental design. A semi-quantitative scoring system, ranging from 0 to 5, was used to assess the following parameters: alveolitis, parenchymal pneumonia, inflammatory cell infiltration, peribronchiolitis, perivasculitis, and lung edema68. The inflammatory scores are as follows: 0, normal; 1, very mild; 2, mild; 3, moderate; 4, marked; and 5, severe. An increment of 0.5 was assigned if the inflammatory score fell between two.

o. Intravascular Labeling and Flow Cytometry

To discriminate the tissue-resident memory T cells (TRM) in the lung from the circulating T cells in the blood, the S-Fc immunized, or PBS control mice were anesthetized and intravenously injected with 3 μg of PE-CD3 Ab in 100 μl PBS through the retro-orbital route. After 5 min labeling, the treated mice were euthanized and bled. Single cells were isolated from the lung. The lungs were perfused with 10 ml PBS, minced with scissors, and incubated in a digestion solution (RPMI with 1 mg/ml of collagenase IV, 5 mM of CaCl₂), 10 μg/ml DNase I) for 45-60 min at 37° C. on a rotating rocker. The digestion was stopped with 5 mM EDTA, and then the cells were filtered through a 70 m cell strainer and treated with ACK buffer to lyse the red blood cells. Next, cells were purified via the 37% Percoll centrifugation to remove most epithelial cells and cell debris. After washing and resuspension with PBS, cells were stained with a Fixable Live/Dead Yellow staining dye (Invitrogen, Cat #501121527) to differentiate between live and dead cells. Single-cell suspensions were incubated with Fc block (anti-mouse CD16/CD32, 1 μg for 1×10⁶ cells, BD Biosciences, Cat #553142) at 4° C. for 30 min. After washing with FACS buffer (2% FBS and 2 mM EDTA in PBS), cells were stained with BV421-Spike tetramer (NIH tetramer center, Cat #63745) 40 min at room temperature, then stained with FITC-CD3 Ab (BD Bioscience, Cat #BDB555275), APC-H7-CD4 Ab (BD Bioscience, Cat #590181), BV786-CD8a Ab (Biolegend, Cat #100749), BV650-CD44 Ab (Biolegend, Cat #103049), BV421-Spike tetramer (NIH tetramer center, Cat #63745), APC-CD69 Ab (BD Bioscience, Cat #BDB560689), and PE-eFluor610-CD103 Ab (eBioscience, Cat #61103182) for 1 h at 4° C. After washing, cells were fixed with 2% paraformaldehyde for 20 min and resuspended in 350 μl 1% BSA/PBS for phenotyping by FACSCelesta cytometer (BD Biosciences). Abc total antibody compensation beads (Invitrogen, Cat #A10497) and Arc amine reactive compensation beads (Invitrogen, Cat #A10346) were used to set up the compensation control. The acquired data were analyzed using the FlowJo software (Tree Star).

p. Airborne Transmission Experiments in Hamsters

Airborne transmission of the SAS-CoV-2 is more efficient than fomite transmission in hamsters. The capacity of the i.n. immunization to reduce airborne transmission of the ancestral SARS-CoV-2 between animals was examined. The i.m. immunized hamsters were used as controls. To this end, the hamsters were either i.n. or i.m. immunized with S-Fc or left unimmunized, as described above. All hamsters were single-housed in stainless steel wire cages in an isolator, where they were grouped into donors (infected) and recipients (exposed). The isolators provided a unidirectional airflow from the donors to the recipients at an air speed of 78 L/min. The airborne transmission study was conducted following the booster. All donor hamsters at anesthesia were i.n. inoculated with 1×10⁵ TCID50 of SARS-CoV-2 (100 μl) and placed upstream of the airflow location in an isolator. Fourteen hours later, the recipient hamsters were placed downstream of the airflow in the same isolator. The donor and recipient cages were seated at 10 cm to avoid direct contact with animals and the effect of dust particles generated from the bedding material. After infection, the hamsters' body weight and clinical signs were monitored daily for 14 consecutive days. Throat swabs were sampled daily for 7 days in donor hamsters, while the recipient hamsters were sampled for 10 days as the onset of infection in the recipients was a few days later than that in the donors.

q. Statistics Analysis

All data were analyzed with the Prism 9.0 software (GraphPad). The unpaired T-test (two-tailed) was used to compare the means between two groups, while one-way ANOVA was used to compare the difference if three or more groups were involved. Meanwhile, a Post Hoc test was applied after one-way ANOVA. Dunnett's multiple comparisons test was used to compare means from different treatment groups against a single control group. If the data distribution is not normal, the Mann-Whitney test (two-tailed) (for two groups) or Kruskal-Wallis test (for more than 2 μgroups) will be used. To compare the Kaplan-Meier survival curves, the Log-rank (Mantel-Cox) test was used. Fisher's exact test (one-tailed) was conducted to compare transmission capacities among various groups. All statistical methods used in each experiment are indicated in the Figure legends. The level of statistical significance was assigned when p values were <0.05. The statistical significance was further classified into four levels: p<0.05, p<0.01, p<0.001, and p<0.0001.

### 8. Expression of SARS-CoV-2 Omicron XBB.1.5 S-Fc Protein Having Six Proline Mutations in the S Protein and Three Cysteine Mutations in the Fc

The S-Fc was designed by the fusion of SARS-CoV-2 Omicron XBB.1.5 Spike with the T4 fibritin foldon domain (Fd) and human IgG 1 Fc to create a soluble, prefusion-stabilized, and trimeric S-Fc fusion protein. Mutations were made in the SARS-CoV-2 (strain XBB.1.5) spike by replacing Arg 683, Arg 685 and Arg 815, respectively, with an Ala residue to remove the cleavage site and replacing Phe 817, Ala 892, Ala 899, Ala 942, Lys 986, and Val 987, respectively, with a Pro residue to create a prefusion-stabilized form. Mutations were also made in the IgG1 Fc fragment by replacing Cys 220, Cys 226, and Cys 229, respectively, with a Ser residue to abolish Fc dimerization, and replacing Lys 322 with Ala residues to remove the complement C1q binding site.

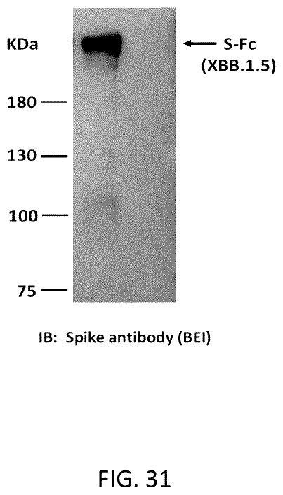

The plasmids were transfected into 293T cells. The S-Fc fusion protein in the supernatant was identified by SDS-PAGE and Western blot analyses (FIG. 31). The visualization of the S-Fc proteins was detected by anti-SARS-CoV-2 Spike, HRP-conjugated secondary antibodies, and the ECL method.

### 9. Expression and Characterization of SARS-CoV-2 Omicron XBB.1.5 S-Fc Proteins

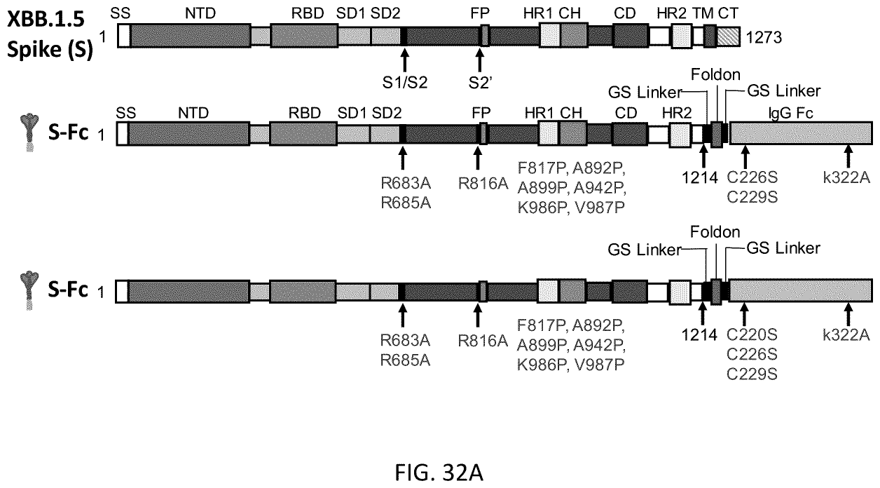

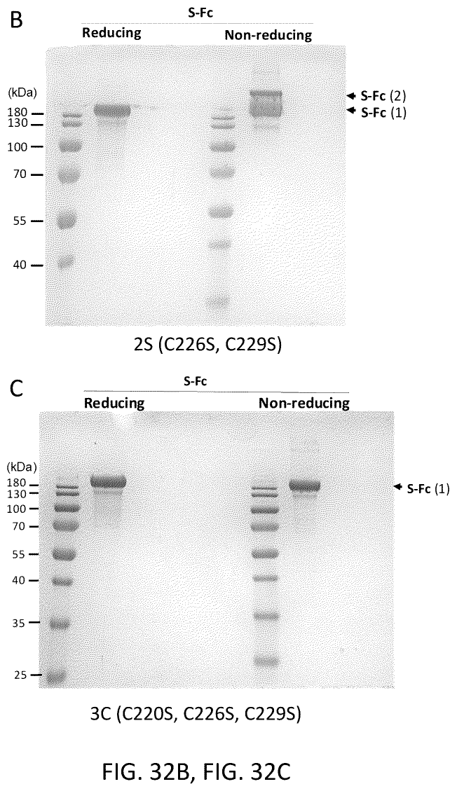

To target antigen to FcRn, we expressed a monomeric human IgG1 Fc fused to a prefusion-stabilized, soluble form of Spike (S) from Omicron XBB.1.5, which contained R683A, R685A mutations at the furin cleavage site, an R815A mutation at S2′ cleavage site, and 6P conversions (FIG. 32A, top) along with the T4 fibritin trimerization domain. These 6P mutations (F817P, A892P, A899P, A942P, K986P, and V987P) were intended to make the SARS-CoV-2 Omicron spike proteins more stable in their prefusion state.

The human IgG1 consists of two heavy chains that are connected by two disulfide bonds through two cysteine pairs located in the hinge region. To alter the structure, two cysteine residues (C226 and C229) were substituted with two serine residues as shown in FIG. 32A (middle). Additionally, we eliminated the C1q binding site in IgG1 Fc.

We demonstrated that the soluble S-Fc protein was secreted from 293F cells after transfection with protein expression plasmids pCDNA3 S-Fc (as shown in FIG. 32B). The proteins were purified and underwent 15% SDS-AGE gel electrophoresis, followed by visualization through Coomassie blue staining. However, the S-Fc protein containing 2S mutations (C226S and C229S) showed an additional minor band (band 2 in FIG. 32B) under non-reducing conditions, in contrast to the major band that appeared under reducing conditions.

We have discovered another cysteine residue (C220) in the hinge region of human IgG 1 Fe, and we believe that it is responsible for the additional band appearance (band 2) shown in FIG. 32B. To confirm this hypothesis, we replaced the additional cysteine residue C220 with a serine residue, creating a pCDNA3 expression plasmid that encodes the S-Fc protein containing 3S mutations (C220S, C226S, and C229S) (as shown in FIG. 32A, bottom). The S-Fc protein containing 3S mutations showed a single major band under both reducing and non-reducing conditions (as seen in FIG. 32C), indicating that the cysteine residue C220 is responsible for the appearance of band 2 under non-reducing conditions (FIG. 32B). The presence of the S-Fc protein was verified in a Western blot assay using a Spike-specific antibody (FIG. 31).

### 10. Intranasal Immunization by S-Fc Protects Mice from SARS-CoV-2 Infection

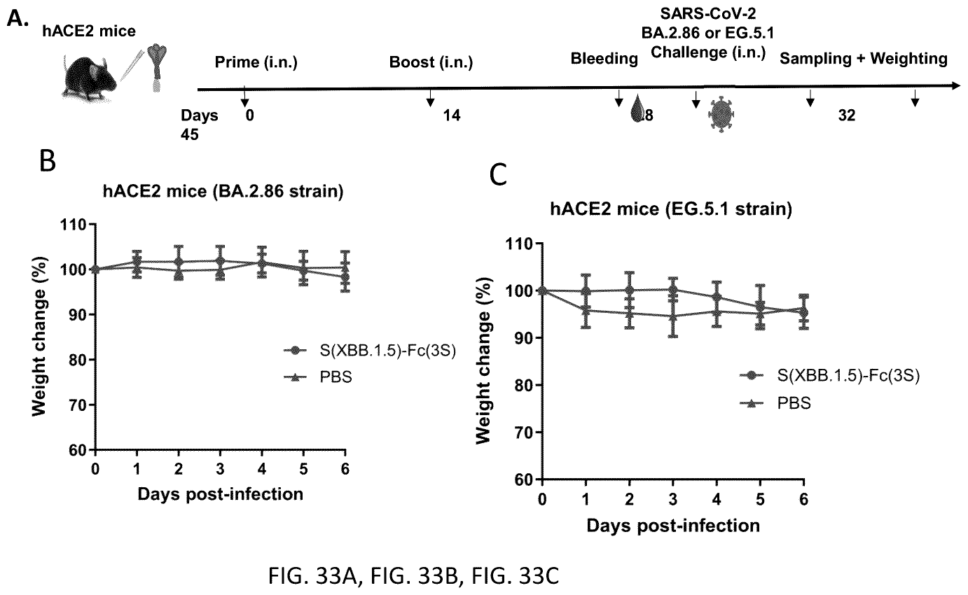

In our previous study, we found that human ACE2 transgenic mice are highly vulnerable to SARS-CoV-2 intranasal challenges. The study involved intranasal (i.n.) immunization of hACE2 transgenic mice with 10 μg of S-Fc (as seen in FIG. 32C) or PBS in g CpG, followed by a boost after a 2-week interval (as shown in FIG. 33A). To determine whether the intranasal vaccination with the S-Fc induces protective immune responses, all immunized mice (n=5-8) were intranasally challenged with a dose (2.5×10⁴ TCID50) of SARS-CoV-2 Omicron virus EG.5.1 or BA.2.86, two weeks after the boost (as illustrated in FIG. 33A). The mice were monitored daily for 14 days and weighed every day. The study revealed that none of the mice in either the S-Fc-immunized or PBS control groups experienced significant body-weight loss (as depicted in FIGS. 33B and 33C) after the virus challenge.

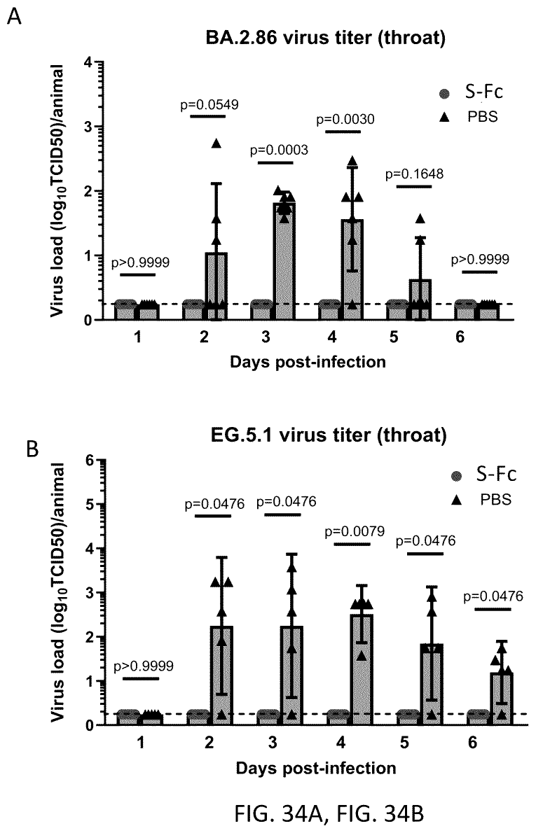

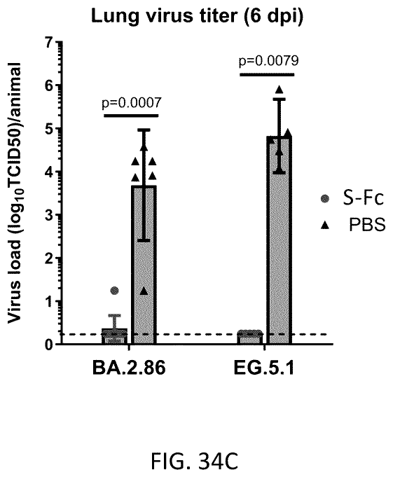

To further show whether the i.n. immunization of the S-Fc protein blocks virus infection, we collected throat swab samples in the challenged mice and measured viral replication in the throat (FIG. 34). As expected, all the PBS-treated mice had high BA.2.86 at 3-4 days post infections (dpi) (FIG. 3A) or EG.5.1 μloads in the throat at 2-6 dpi (FIG. 34B). In contrast, a significant reduction of EG.5.1 or BA.2.86 virus load in the throat was seen in the S-Fc-immunized mice when compared to the control animals (FIGS. 34A+34B).

In addition, we conducted a study to measure the viral replication in lung tissues of mice infected with BA.2.86 or EG.5.1 virus at 6 days post-infection (dpi). The lung tissues of the control mice treated with PBS showed detectable live virus. However, we were unable to find any live virus in most of the lung tissues of the S-Fc-immunized mice (FIG. 34C). Only one mouse in the S-Fc-immunized group showed a reduced BA.2.86 virus load in the lung sample. Therefore, the S-Fc-immunized hACE2 transgenic mice effectively contained viral replication in tissues and organs of viral entry and prevented the virus from spreading to other tissues and organs.

Those skilled in the art will recognize, or be able to ascertain using no more than routine experimentation, many equivalents to the specific embodiments of the method and compositions described herein. Such equivalents are intended to be encompassed by the following claims.

1.-61. (canceled)

62. A peptide comprising
a monomeric Fc fragment of an immunoglobulin recognized by a neonatal receptor (FcRn);
a SARS-CoV-2 spike (S) antigen, wherein the SARS-CoV-2 S antigen comprises at least four mutations, wherein the mutations stabilize the S antigen in the prefusion form; and
a trimerization domain.

63. The peptide of claim 62, wherein the at least four mutations in the SARS-CoV-2 S antigen correspond to at least four of positions Phe 817, Ala 892, Ala 899, Ala 942, Lys 986, or Val 987 of the SARS-CoV-2 S antigen sequence.

64. The peptide of claim 62, wherein the at least four mutations in the SARS-CoV-2 S antigen are proline substitutions.

65. The peptide of claim 62, wherein the at least four mutations in the SARS-CoV-2 S antigen comprise F817P, A892P, A899P, and A942P;

66. The peptide of claim 62, wherein the SARS-CoV-2 S antigen comprises six mutations.

67. The peptide of claim 66, wherein the six mutations in the SARS-CoV-2 S antigen are F817P, A892P, A899P, A942P, K986P, and V987P.

68. The peptide of claim 62, wherein the monomeric Fc fragment of an immunoglobulin comprises at least one mutation, at least two mutations, or at least three mutations in a cysteine residue responsible for dimer formation.

69. The peptide of claim 68, wherein the cysteine residues responsible for dimer formation are at positions 11 and 14 of SEQ ID NO:7 or at position 5, 11 and 14 of SEQ ID NO:7.

70. The peptide of any one of claims 68-69, wherein the at least one mutation is a cysteine to serine substitution.

71. The peptide of claim 62, wherein the SARS-CoV-2 antigen is from a Wuhan, Alpha, Beta, Gamma, Delta, or Omicron strain

72. The peptide of claim 62, wherein the SARS-CoV-2 S antigen is full length soluble SARS-CoV-2 S protein, a S1 subunit of the SARS-CoV-2 S protein, a S2 subunit of the SARS-CoV-2 S protein or a receptor binding domain (RBD) of the S1 subunit of the SARS-CoV-2 S protein.

73. The peptide of claim 62, wherein C1q motif of the Fc fragment of FcRn has been mutated such that it renders the fragment non-lytic.

74. The peptide of claim 62, wherein the monomeric Fc fragment of an immunoglobulin recognized by a FcRn comprises a CH2 domain and a CH3 domain.

75. The peptide of claim 74, wherein the monomeric Fc fragment of an immunoglobulin comprises one or more mutations in the CH2 domain, wherein the one or more mutations in the CH2 domain ablate C1q binding to the monomeric Fc fragment.

76. A peptide complex comprising three peptides, wherein each of the peptides is the peptide of claim 62.

77. A composition comprising one or more of the peptide complexes of claim 76.

78. A method for eliciting a protective immune response against SARS-CoV-2 comprising administering to a subject an effective amount of one or more of the compositions of claim 77.

79. A method for eliciting a protective immune response against SARS-CoV-2 comprising administering to a subject an effective amount of a composition comprising a peptide complex, wherein the peptide complex comprises three peptides forming a trimer, wherein each of the three peptides comprises a monomeric Fc fragment of an immunoglobulin recognized by a neonatal receptor (FcRn); a SARS-CoV-2 spike (S) antigen, wherein the SARS-CoV-2 S antigen comprises at least three mutations, wherein the mutations stabilize the S antigen in the prefusion form; and a trimerization domain, wherein the administering is to a mucosal epithelium.

80. A method of treating a subject exposed to SARS-CoV-2 or at risk of being exposed to SARS-CoV-2 comprising administering to the subject an effective amount of one or more of the compositions of claim 77.

81. A method of treating a subject exposed to SARS-CoV-2 or at risk of being exposed to SARS-CoV-2 comprising administering to the subject an effective amount of a composition comprising a peptide complex, wherein the peptide complex comprises three peptides forming a trimer, wherein each of the three peptides comprises a monomeric Fc fragment of an immunoglobulin recognized by a neonatal receptor (FcRn); a SARS-CoV-2 spike (S) antigen, wherein the SARS-CoV-2 S antigen comprises at least three mutations, wherein the mutations stabilize the S antigen in the prefusion form; and a trimerization domain, wherein the administering is to a mucosal epithelium.


SEQUENCE LISTING (54 sequences)

SEQ ID NO: 1 - protein, 1504 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKASFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGSGSGSRSLVPRGSPGSGYIPEAPRDGQAYVRKDGEWVLLSTFLGGSGGGGSGGGGSGSEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 2 - DNA, 4533 bp, synthetic construct
  source 1..4533: mol_type = other DNA
ggtaccgccaccatgttcgtgtttctggtgctgctgccactggtgtccagccagtgcgtgaacctgaccacaagaacccagctgccccctgcctataccaattctttcacaagaggcgtgtactatccagacaaggtgtttcgctcttccgtgctgcacagcacacaggatctgtttctgcccttcttttctaacgtgacctggttccacgccatccacgtgtccggcaccaatggcacaaagaggttcgacaatcctgtgctgcccttcaacgatggcgtgtacttcgcttctaccgagaagtccaacatcatccggggctggatctttggcaccacactggacagcaagacacagtctctgctgatcgtgaacaatgccaccaacgtggtcatcaaggtgtgcgagttccagttttgtaatgatcctttcctgggcgtgtactatcataagaacaataagtcctggatggagagcgagtttcgcgtgtatagctctgctaacaattgtacatttgagtacgtgagccagccattcctgatggacctggagggcaagcagggcaatttcaagaacctgagagagttcgtgtttaagaatatcgatggctacttcaagatctacagcaagcacacccctatcaacctggtgcgcgacctgccacagggcttctctgccctggagcctctggtggatctgccaatcggcatcaacatcaccaggtttcagacactgctggctctgcatcggtcttacctgacacctggcgactccagctctggatggaccgctggagctgctgcttactatgtgggctatctgcagccaagaaccttcctgctgaagtacaacgagaatggcaccatcacagacgccgtggattgcgctctggatccactgtccgagaccaagtgtacactgaagagctttaccgtggagaagggcatctatcagacatccaatttcagagtgcagcccaccgagagcatcgtgcgctttccaaatatcacaaacctgtgcccctttggcgaggtgttcaacgccacccgcttcgcttccgtgtacgcctggaatagaaagcgcatctccaactgcgtggctgactatagcgtgctgtacaactccgccagcttctctacctttaagtgctatggcgtgtcccccacaaagctgaatgacctgtgctttaccaacgtgtacgccgatagcttcgtgatcagaggcgacgaggtgcgccagatcgctccaggacagacaggcaagatcgccgactacaattataagctgcctgacgatttcaccggctgcgtgatcgcttggaactccaacaatctggatagcaaagtgggcggcaactacaattatctgtacaggctgtttcggaagagcaatctgaagcctttcgagagggacatctctacagagatctaccaggccggctccaccccatgcaatggcgtggagggctttaactgttatttccccctgcagtcttacggcttccagcctaccaacggcgtgggctatcagccataccgggtggtggtgctgtcttttgagctgctgcacgctccagctacagtgtgcggacctaagaagtccaccaatctggtgaagaacaagtgcgtgaacttcaacttcaacggactgaccggcacaggcgtgctgaccgagagcaacaagaagttcctgccctttcagcagttcggcagggacatcgctgataccacagacgccgtgcgggacccacagaccctggagatcctggatatcacaccctgctctttcggcggcgtgtccgtgatcacacctggcaccaatacatctaaccaggtggccgtgctgtatcaggacgtgaattgtaccgaggtgcctgtggccatccacgctgatcagctgaccccaacatggagggtgtacagcaccggctctaacgtgtttcagacacgggctggatgtctgatcggagctgagcatgtgaacaattcctatgagtgcgacatccccatcggcgctggcatctgtgccagctaccagacccagacaaacagccctaggagggctgcttctgtggcttcccagagcatcatcgcctataccatgtccctgggcgctgagaatagcgtggcctactccaacaatagcatcgctatcccaaccaacttcacaatctccgtgaccacagagatcctgcccgtgagcatgaccaagacatctgtggactgcacaatgtatatctgtggcgattctaccgagtgctccaacctgctgctgcagtacggcagcttttgtacccagctgaatagggctctgacaggcatcgccgtggagcaggataagaacacacaggaggtgttcgcccaggtgaagcagatctacaagaccccacccatcaaggactttggcggcttcaacttctcccagatcctgcctgatccatctaagccctccaaggctagctttatcgaggacctgctgttcaacaaggtgaccctggctgatgccggcttcatcaagcagtatggcgattgcctgggcgacatcgctgccagggacctgatctgtgctcagaagtttaatggcctgaccgtgctgcctccactgctgacagatgagatgatcgcccagtacacatctgccctgctggctggcaccatcacatccggatggaccttcggcgctggagctgccctgcagatcccttttgctatgcagatggcctatcggttcaacggcatcggcgtgacccagaatgtgctgtacgagaaccagaagctgatcgctaatcagtttaactccgccatcggcaagatccaggactctctgtccagcacagcttccgccctgggcaagctgcaggatgtggtgaatcagaacgctcaggccctgaataccctggtgaagcagctgtcttccaacttcggcgctatcagctctgtgctgaatgatatcctgagcagactggacccacctgaggctgaggtgcagatcgacaggctgatcacaggccggctgcagagcctgcagacctacgtgacacagcagctgatcagagctgccgagatccgcgcttctgccaacctggctgccaccaagatgtctgagtgcgtgctgggccagtccaagcgcgtggacttttgtggcaagggctatcacctgatgagcttcccccagtctgctcctcacggcgtggtgtttctgcatgtgacctacgtgcccgcccaggagaagaacttcaccacagctcctgccatctgccacgatggcaaggcccattttcccagagagggcgtgttcgtgtctaacggcacccattggtttgtgacacagcgcaatttctacgagcctcagatcatcaccacagacaataccttcgtgtccggcaactgtgacgtggtcatcggcatcgtgaacaataccgtgtatgatcccctgcagcctgagctggactcttttaaggaggagctggataagtacttcaagaatcacacctccccagacgtggatctgggcgacatctccggcatcaatgctagcgtggtgaacatccagaaggagatcgacaggctgaacgaggtggccaagaatctgaacgagtctctgatcgatctgcaggagctgggcaagtatgagcagtacatcaagtggccaggatctggatccggcagcaggtctctggtgccacggggctctccaggatccggatatatcccagaggctcccagagacggacaggcttacgtgcgcaaggatggcgagtgggtgctgctgtccaccttcctgggcggctctggaggaggaggatccggaggaggaggatccggcagcgagcctaagtcctgcgacaagacccacacaagcccaccatctccagctcctgagctgctgggaggaccaagcgtgttcctgtttcctccaaagcctaaggatacactgatgatctctcggaccccagaggtgacatgcgtggtggtggacgtgtcccacgaggaccccgaggtgaagtttaactggtacgtggacggcgtggaggtgcataatgctaagaccaagccaagggaggagcagtataacagcacataccgggtggtgtctgtgctgaccgtgctgcatcaggattggctgaacggcaaggaatacaagtgcgctgtgagcaataaggccctgccagctcccatcgagaagacaatctctaaggccaagggccagcctagagagccacaggtgtataccctgccaccttcccgcgacgagctgaccaagaatcaggtgagcctgacatgtctggtgaagggcttctaccctagcgatatcgctgtggagtgggagtctaacggccagccagagaacaattataagaccacaccacccgtgctggactccgatggcagcttctttctgtacagcaagctgacagtggacaagtctcggtggcagcagggcaacgtgttctcctgctccgtgatgcatgaggccctgcacaaccattacacccagaagagcctgtctctgtcccctggcaagtgactcgag

SEQ ID NO: 3 - protein, 976 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAAGSGSGSRSLVPRGSPGSGYIPEAPRDGQAYVRKDGEWVLLSTFLGGSGGGGSGGGGSGSEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 4 - DNA, 2949 bp, synthetic construct
  source 1..2949: mol_type = other DNA
ggtaccgccaccatgttcgtgtttctggtgctgctgccactggtgtccagccagtgcgtgaacctgaccacaagaacccagctgccccctgcctataccaattctttcacaagaggcgtgtactatccagacaaggtgtttcgctcttccgtgctgcacagcacacaggatctgtttctgcccttcttttctaacgtgacctggttccacgccatccacgtgtccggcaccaatggcacaaagaggttcgacaatcctgtgctgcccttcaacgatggcgtgtacttcgcttctaccgagaagtccaacatcatccggggctggatctttggcaccacactggacagcaagacacagtctctgctgatcgtgaacaatgccaccaacgtggtcatcaaggtgtgcgagttccagttttgtaatgatcctttcctgggcgtgtactatcataagaacaataagtcctggatggagagcgagtttcgcgtgtatagctctgctaacaattgtacatttgagtacgtgagccagccattcctgatggacctggagggcaagcagggcaatttcaagaacctgagagagttcgtgtttaagaatatcgatggctacttcaagatctacagcaagcacacccctatcaacctggtgcgcgacctgccacagggcttctctgccctggagcctctggtggatctgccaatcggcatcaacatcaccaggtttcagacactgctggctctgcatcggtcttacctgacacctggcgactccagctctggatggaccgctggagctgctgcttactatgtgggctatctgcagccaagaaccttcctgctgaagtacaacgagaatggcaccatcacagacgccgtggattgcgctctggatccactgtccgagaccaagtgtacactgaagagctttaccgtggagaagggcatctatcagacatccaatttcagagtgcagcccaccgagagcatcgtgcgctttccaaatatcacaaacctgtgcccctttggcgaggtgttcaacgccacccgcttcgcttccgtgtacgcctggaatagaaagcgcatctccaactgcgtggctgactatagcgtgctgtacaactccgccagcttctctacctttaagtgctatggcgtgtcccccacaaagctgaatgacctgtgctttaccaacgtgtacgccgatagcttcgtgatcagaggcgacgaggtgcgccagatcgctccaggacagacaggcaagatcgccgactacaattataagctgcctgacgatttcaccggctgcgtgatcgcttggaactccaacaatctggatagcaaagtgggcggcaactacaattatctgtacaggctgtttcggaagagcaatctgaagcctttcgagagggacatctctacagagatctaccaggccggctccaccccatgcaatggcgtggagggctttaactgttatttccccctgcagtcttacggcttccagcctaccaacggcgtgggctatcagccataccgggtggtggtgctgtcttttgagctgctgcacgctccagctacagtgtgcggacctaagaagtccaccaatctggtgaagaacaagtgcgtgaacttcaacttcaacggactgaccggcacaggcgtgctgaccgagagcaacaagaagttcctgccctttcagcagttcggcagggacatcgctgataccacagacgccgtgcgggacccacagaccctggagatcctggatatcacaccctgctctttcggcggcgtgtccgtgatcacacctggcaccaatacatctaaccaggtggccgtgctgtatcaggacgtgaattgtaccgaggtgcctgtggccatccacgctgatcagctgaccccaacatggagggtgtacagcaccggctctaacgtgtttcagacacgggctggatgtctgatcggagctgagcatgtgaacaattcctatgagtgcgacatccccatcggcgctggcatctgtgccagctaccagacccagacaaacagccctaggagggctgctggatctggatccggcagcaggtctctggtgccacggggctctccaggatccggatatatcccagaggctcccagagacggacaggcttacgtgcgcaaggatggcgagtgggtgctgctgtccaccttcctgggcggctctggaggaggaggatccggaggaggaggatccggcagcgagcctaagtcctgcgacaagacccacacaagcccaccatctccagctcctgagctgctgggaggaccaagcgtgttcctgtttcctccaaagcctaaggatacactgatgatctctcggaccccagaggtgacatgcgtggtggtggacgtgtcccacgaggaccccgaggtgaagtttaactggtacgtggacggcgtggaggtgcataatgctaagaccaagccaagggaggagcagtataacagcacataccgggtggtgtctgtgctgaccgtgctgcatcaggattggctgaacggcaaggaatacaagtgcgctgtgagcaataaggccctgccagctcccatcgagaagacaatctctaaggccaagggccagcctagagagccacaggtgtataccctgccaccttcccgcgacgagctgaccaagaatcaggtgagcctgacatgtctggtgaagggcttctaccctagcgatatcgctgtggagtgggagtctaacggccagccagagaacaattataagaccacaccacccgtgctggactccgatggcagcttctttctgtacagcaagctgacagtggacaagtctcggtggcagcagggcaacgtgttctcctgctccgtgatgcatgaggccctgcacaaccattacacccagaagagcctgtctctgtcccctggcaagtgactcgag

SEQ ID NO: 5 - protein, 530 aa, synthetic construct
MFVFLVLLPLVSSQCVRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFGSGSGSRSLVPRGSPGSGYIPEAPRDGQAYVRKDGEWVLLSTFLGGSGGGGSGGGGSGSEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 6 - DNA, 1611 bp, synthetic construct
  source 1..1611: mol_type = other DNA
ggtaccgccaccatgttcgtgtttctggtgctgctgccactggtgtccagccagtgcgtgagagtgcagcccaccgagagcatcgtgcgctttccaaatatcacaaacctgtgcccctttggcgaggtgttcaacgccacccgcttcgcttccgtgtacgcctggaatagaaagcgcatctccaactgcgtggctgactatagcgtgctgtacaactccgccagcttctctacctttaagtgctatggcgtgtcccccacaaagctgaatgacctgtgctttaccaacgtgtacgccgatagcttcgtgatcagaggcgacgaggtgcgccagatcgctccaggacagacaggcaagatcgccgactacaattataagctgcctgacgatttcaccggctgcgtgatcgcttggaactccaacaatctggatagcaaagtgggcggcaactacaattatctgtacaggctgtttcggaagagcaatctgaagcctttcgagagggacatctctacagagatctaccaggccggctccaccccatgcaatggcgtggagggctttaactgttatttccccctgcagtcttacggcttccagcctaccaacggcgtgggctatcagccataccgggtggtggtgctgtcttttgagctgctgcacgctccagctacagtgtgcggacctaagaagtccaccaatctggtgaagaacaagtgcgtgaacttcggatctggatccggcagcaggtctctggtgccacggggctctccaggatccggatatatcccagaggctcccagagacggacaggcttacgtgcgcaaggatggcgagtgggtgctgctgtccaccttcctgggcggctctggaggaggaggatccggaggaggaggatccggcagcgagcctaagtcctgcgacaagacccacacaagcccaccatctccagctcctgagctgctgggaggaccaagcgtgttcctgtttcctccaaagcctaaggatacactgatgatctctcggaccccagaggtgacatgcgtggtggtggacgtgtcccacgaggaccccgaggtgaagtttaactggtacgtggacggcgtggaggtgcataatgctaagaccaagccaagggaggagcagtataacagcacataccgggtggtgtctgtgctgaccgtgctgcatcaggattggctgaacggcaaggaatacaagtgcgctgtgagcaataaggccctgccagctcccatcgagaagacaatctctaaggccaagggccagcctagagagccacaggtgtataccctgccaccttcccgcgacgagctgaccaagaatcaggtgagcctgacatgtctggtgaagggcttctaccctagcgatatcgctgtggagtgggagtctaacggccagccagagaacaattataagaccacaccacccgtgctggactccgatggcagcttctttctgtacagcaagctgacagtggacaagtctcggtggcagcagggcaacgtgttctcctgctccgtgatgcatgaggccctgcacaaccattacacccagaagagcctgtctctgtcccctggcaagtgactcgag

SEQ ID NO: 7 - protein, 232 aa, synthetic construct
EPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 8 - protein, 1213 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRAASVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKASFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 9 - DNA, 699 bp, synthetic construct
  source 1..699: mol_type = other DNA
gagcctaagtcctgcgacaagacccacacaagcccaccatctccagctcctgagctgctgggaggaccaagcgtgttcctgtttcctccaaagcctaaggatacactgatgatctctcggaccccagaggtgacatgcgtggtggtggacgtgtcccacgaggaccccgaggtgaagtttaactggtacgtggacggcgtggaggtgcataatgctaagaccaagccaagggaggagcagtataacagcacataccgggtggtgtctgtgctgaccgtgctgcatcaggattggctgaacggcaaggaatacaagtgcgctgtgagcaataaggccctgccagctcccatcgagaagacaatctctaaggccaagggccagcctagagagccacaggtgtataccctgccaccttcccgcgacgagctgaccaagaatcaggtgagcctgacatgtctggtgaagggcttctaccctagcgatatcgctgtggagtgggagtctaacggccagccagagaacaattataagaccacaccacccgtgctggactccgatggcagcttctttctgtacagcaagctgacagtggacaagtctcggtggcagcagggcaacgtgttctcctgctccgtgatgcatgaggccctgcacaaccattacacccagaagagcctgtctctgtcccctggcaagtga

SEQ ID NO: 10 - DNA, 699 bp, synthetic construct
  source 1..699: mol_type = other DNA
gagcccagagggcccacaatcaagccctctcctccatccaaatccccagcacctaacctcttgggtggaccatccgtcttcatcttccctccaaagatcaaggatgtactcatgatctccctgagccccatagtcacatgtgtggtggtggatgtgagcgaggatgacccagatgtccagatcagctggtttgtgaacaacgtggaagtacacacagctcagacacaaacccatagagaggattacaacagtactctccgggtggtcagtgccctccccatccagcaccaggactggatgagtggcaaggcgttcgcatgcgcggtcaacaacaaagacctcccagcgcccatcgagagaaccatctcaaaacccaaagggtcagtaagagctccacaggtatatgtcttgcctccaccagaagaagagatgactaagaaacaggtcactctgacctgcatggtcacagacttcatgcctgaagacatttacgtggagtggaccaacaacgggaaaacagagctaaactacaagaacactgaaccagtcctggactctgatggttcttacttcatgtacagcaagctgagagtggaaaagaagaactgggtggaaagaaatagctactcctgttcagtggtccacgagggtctgcacaatcaccacacgactaagagcttctcccggactccgggtaaa

SEQ ID NO: 11 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 12 - protein, 1270 aa, synthetic construct
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLYRLFRKSNLKPFERDISTEIYQAGSTPCNGVEGFNCYFPLQSYGFQPTYGVGYQPYRVVVLSFELLHAPATVCGPKKSTNLVKNKCVNFNFNGLTGTGVLTESNKKFLPFQQFGRDIDDTTDAVRDPQTLEILDITPCSFGGVSVITPGTNTSNQVAVLYQGVNCTEVPVAIHADQLTPTWRVYSTGSNVFQTRAGCLIGAEHVNNSYECDIPIGAGICASYQTQTNSHRRARSVASQSIIAYTMSLGAENSVAYSNNSIAIPINFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILARLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTHNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 13 - protein, 1273 aa, synthetic construct
MFVFLVLLPLVSSQCVNLTTRTQLPPAYTNSFTRGVYYPDKVFRSSVLHSTQDLFLPFFSNVTWFHAIHVSGTNGTKRFANPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRGLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGNIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGVENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 14 - protein, 1273 aa, synthetic construct
MFVFLVLLPLVSSQCVNFTNRTQLPSAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNYPFLGVYYHKNNKSWMESEFRVYSSANNCTFEYVSQPFLMDLEGKQGNFKNLSEFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGTIADYNYKLPDDFTGCVIAWNSNNLDSKVGGNYNYLYRLFRKSNLKPFERDISTEIYQAGSTPCNGVKGFNCYFPLQSYGFQPTYGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEYVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAAIKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 15 - protein, 1273 aa, synthetic construct
MFVFLVLLPLVSIQCVNLTTRTQLPPAYTNSFTRGVYYPDKVFRSSVLHSTQDLFLPFFSNVTWFHAIHVSGTNGTKRFDNPVLPFNDGVYFASTEKSNIIRGWIFGTTLDSKTQSLLIVNNATNVVIKVCEFQFCNDPFLGVYYHKNNKSCMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYRYRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIIAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 16 - protein, 1271 aa, synthetic construct
MFVFLVLLPLVSSQCVNLTTRTQLPPAYTNSFTRGVYYPDKVFRSSVLHSTQDLFLPFFSNVTWFHAISGTNGTKRFDNPVLPFNDGVYFASTEKSNIIRGWIFGTTLDSKTQSLLIVNNATNVVIKVCEFQFCNDPFLGVYYHKNNKSWMESEFRVYSSANNCTFEYVSQPFLMDLEGKQGNFKNLREFVFKNIDGYFKIYSKHTPINLVRDLPQGFSALEPLVDLPIGINITRFQTLLALHRSYLTPGDSSSGWTAGAAAYYVGYLQPRTFLLKYNENGTITDAVDCALDPLSETKCTLKSFTVEKGIYQTSNFRVQPTESIVRFPNITNLCPFGEVFNATRFASVYAWNRKRISNCVADYSVLYNSASFSTFKCYGVSPTKLNDLCFTNVYADSFVIRGDEVRQIAPGQTGKIADYNYKLPDDFTGCVIAWNSNNLDSKVGGNYNYLFRLFRKSNLKPFERDISTEIYQAGSTPCNGVEGFNCYFPLQSYGFQPTNGVGYQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQDVNCTEVPVAIHADQLTPTWRVYSTGSNVFQTRAGCLIGAEHVNNSYECDIPIGAGICASYQTQTNSPRRARSVASQSIVAYTMSLGAENSVAYSNNSIAIPTNFTISVTTEILPVSMTKTSVDCTMYICGDSTECSNLLLQYGSFCTQLNRALTGIAVEQDKNTQEVFAQVKQIYKTPPIKDFGGFNFSQILPDPSKPSKRSFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNQNAQALNTLVKQLSSNFGAISSVLNDILSRLDKVEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPWYIWLGFIAGLIAIVMVTIMLCCMTSCCSCLKGCCSCGSCCKFDEDDSEPVLKGVKLHYT

SEQ ID NO: 17 - protein, 232 aa, synthetic construct
EPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 18 - protein, 233 aa, synthetic construct
EPRGPTIKPCPPCKSPAPNLLGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 19 - DNA, 53 bp, synthetic construct
  source 1..53: mol_type = other DNA
caccttcctgggccatcatcaccatcaccattgactcgagtctagagggcccg

SEQ ID NO: 20 - DNA, 53 bp, synthetic construct
  source 1..53: mol_type = other DNA
atggtgatggtgatgatggcccaggaaggtggacagcagcacccactcgccat

SEQ ID NO: 21 - DNA, 33 bp, synthetic construct
  source 1..33: mol_type = other DNA
gcgggtaccgccaccatggggatgccccagccc

SEQ ID NO: 22 - DNA, 100 bp, synthetic construct
  source 1..100: mol_type = other DNA
tatctcgagttactcgtgccactcgatcttctgggcctcgaagatgtcgttcaggccgtggtgatggtggtgatggtgatgcgaagatctggctggagca

SEQ ID NO: 23 - DNA, 34 bp, synthetic construct
  source 1..34: mol_type = other DNA
tatgtcgacgccaccatggctcgctccgtggccg

SEQ ID NO: 24 - DNA, 57 bp, synthetic construct
  source 1..57: mol_type = other DNA
gcgtctagactatttttcgaactgcgggtggctccacatgtctcgttcccaggtgac

SEQ ID NO: 25 - DNA, 36 bp, synthetic construct
  source 1..36: mol_type = other DNA
gcgggtaccgccaccatgtggctcctaacaaccctg

SEQ ID NO: 26 - DNA, 101 bp, synthetic construct
  source 1..101: mol_type = other DNA
tatctcgagttactcgtgccactcgatcttctgggcctcgaagatgtcgttcaggccgtggtgatggtggtgatggtgatgagggcctgatgactgaggac

SEQ ID NO: 27 - DNA, 20 bp, synthetic construct
  source 1..20: mol_type = other DNA
gaccccaaaatcagcgaaat

SEQ ID NO: 28 - DNA, 24 bp, synthetic construct
  source 1..24: mol_type = other DNA
tctggttactgccagttgaatctg

SEQ ID NO: 29 - DNA, 24 bp, synthetic construct
  source 1..24: mol_type = other DNA
accccgcattacgtttggtggacc

SEQ ID NO: 30 - protein, 1209 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRAAASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKASFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWP

SEQ ID NO: 31 - protein, 1488 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRAAASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKASFIEDLLFNKVTLADAGFIKQYGDCLGDIAARDLICAQKFNGLTVLPPLLTDEMIAQYTSALLAGTITSGWTFGAGAALQIPFAMQMAYRFNGIGVTQNVLYENQKLIANQFNSAIGKIQDSLSSTASALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGSGSGSGYIPEAPRDGQAYVRKDGEWVLLSTFLGSGGGGSGGGGSGSEPKSCDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - DNA, 4467 bp, synthetic construct
  source 1..4467: mol_type = other DNA
atgttcgtgtttctggtgctgctgcccctggtgtccagccagtgcgtgaatctgatcaccagaacacagagctacaccaactctttcacacgcggcgtgtactatccagacaaggtgtttaggtcttccgtgctgcactctacacaggatctgtttctgcccttcttttccaatgtgacctggttccacgccatccatgtgagcggcaccaacggcacaaagaggttcgacaatcctgctctgcccttcaacgatggcgtgtacttcgcctccaccgagaagagcaacatcatcaggggctggatctttggcaccacactggactctaagacacagtccctgctgatcgtgaacaatgctaccaacgtggtcatcaaggtgtgcgagttccagttttgtaacgatcctttcctggacgtgtatcagaagaacaataagagctggatggagtctgagtttcgcgtgtacagctctgccaacaattgtacctttgagtacgtgtcccagccattcctgatggacctggagggcaaggagggcaatttcaagaacctgcgggagttcgtgtttaagaatatcgatggctacttcaagatctacagcaagcacaccccaatcaacctggagagagacctgccacagggcttctctgccctggagcctctggtggatctgccaatcggcatcaacatcaccaggtttcagacactgctggctctgcatcggagctacctgacacctgtggactccagctctggatggaccgctggagctgctgcttactatgtgggctacctgcagccaaggaccttcctgctgaagtataacgagaatggcaccatcacagacgccgtggattgcgctctggatccactgagcgagaccaagtgtacactgaagtcttttaccgtggagaagggcatctaccagacatctaatttcagagtgcagcccaccgagtccatcgtgcgctttcccaatatcaccaacctgtgcccttttcatgaggtgttcaacgccaccacattcgcttccgtgtacgcctggaataggaagcggatcagcaactgcgtggctgactattctgtgatctacaattttgctccattctttgccttcaagtgctacggcgtgtcccccacaaagctgaatgacctgtgctttaccaacgtgtatgccgatagcttcgtgatccgcggcaacgaggtgtctcagatcgctccaggccagacaggcaatatcgccgactacaactataagctgcccgacgatttcaccggctgcgtgatcgcctggaactctaataagctggatagcaagccatctggcaactacaattatctgtacagactgtttcgcaagagcaagctgaagcccttcgagagggatatctctacagagatctaccaggctggcaataagccttgcaacggagtggctggaccaaactgttattcccctctgcagagctacggcttccgccctacctatggagtgggacaccagccatatagggtggtggtgctgtcctttgagctgctgcatgcccctgctacagtgtgcggcccaaagaagagcaccaatctggtgaagaacaagtgcgtgaacttcaacttcaacggactgaccggcacaggcgtgctgaccgagtccaacaagaagttcctgccctttcagcagttcggccgggacatcgctgataccacagacgccgtgagagacccacagaccctggagatcctggatatcacaccctgctccttcggcggcgtgagcgtgatcacacctggcaccaatacaagcaaccaggtggctgtgctgtaccagggcgtgaattgtaccgaggtgcctgtggccatccacgctgaccagctgaccccaacatggcgcgtgtattccaccggcagcaacgtgtttcagacaagggctggctgtctgatcggcgccgagtatgtgaacaattcctacgagtgcgatatccctatcggcgctggcatctgtgccagctaccagacccagacaaagtctcatagggctgctgcttccgtggcttcccagagcatcatcgcctacaccatgagcctgggcgctgagaattctgtggcctattctaacaattccatcgccatccctaccaacttcacaatcagcgtgaccacagagatcctgccagtgtctatgaccaagacatccgtggactgcacaatgtacatctgtggcgattctaccgagtgctccaatctgctgctgcagtatggctctttttgtacccagctgaagagagctctgacaggcatcgccgtggagcaggacaagaacacacaggaggtgttcgctcaggtgaagcagatctacaagaccccccctatcaagtattttggcggcttcaatttttcccagatcctgccagatcccagcaagccatctaaggcctcctttatcgaggacctgctgtttaacaaggtgaccctggccgatgctggcttcatcaagcagtacggcgattgcctgggcgacatcgctgctcgggacctgatctgtgctcagaagttcaatggcctgaccgtgctgccacccctgctgacagatgagatgatcgcccagtatacatccgccctgctggctggcaccatcacaagcggatggacctttggcgctggagctgctctgcagatcccttttgctatgcagatggcttacagattcaacggcatcggcgtgacccagaatgtgctgtatgagaaccagaagctgatcgctaatcagttcaactctgccatcggcaagatccaggactccctgtccagcacagctagcgccctgggcaagctgcaggatgtggtgaatcacaacgcccaggctctgaataccctggtgaagcagctgtcttccaagtttggcgctatcagctctgtgctgaacgatatcctgtcccgcctggaccctccagaggctgaggtgcagatcgacaggctgatcacaggccggctgcagtctctgcagacctacgtgacacagcagctgatcagagccgctgagatccgcgcctccgctaacctggccgctaccaagatgtccgagtgcgtgctgggccagagcaagagggtggacttttgtggcaagggctaccacctgatgagcttcccccagtctgctcctcacggcgtggtgtttctgcatgtgacctatgtgcccgcccaggagaagaacttcaccacagcccctgctatctgccacgatggcaaggcccattttccccgggagggcgtgttcgtgtccaatggcacccattggtttgtgacacagagaaacttctacgagcctcagatcatcaccacagacaatacatttgtgagcggcaactgtgacgtggtcatcggtatcgtgaacaataccgtgtacgatcccctgcagcctgagctggactcttttaaggaggagctggataagtatttcaagaatcatacctcccctgacgtggatctgggcgacatcagcggcatcaatgcttctgtggtgaacatccagaaggagatcgaccggctgaacgaggtggccaagaatctgaacgagagcctgatcgatctgcaggagctgggcaagtatgagcagtacatcaagtggccaggatctggatccggcagcggatacatcccagaggctcccagagacggccaggcctatgtgcgcaaggatggcgagtgggtgctgctgtctaccttcctgggatccggaggaggaggaagcggaggcggcggctccggcagcgagcccaagagctgcgacaagacccacacatccccacctagcccagctcctgagctgctgggaggaccatccgtgttcctgtttccacccaagcctaaggatacactgatgatctctagaacccctgaggtgacatgcgtggtggtggacgtgtcccacgaggacccagaggtgaagttcaattggtacgtggacggcgtggaggtgcataacgccaagaccaagcctagggaggagcagtataactctacataccgggtggtgtccgtgctgaccgtgctgcatcaggattggctgaatggcaaggagtataagtgcgctgtgagcaacaaggccctgccagctcccatcgagaagacaatctctaaggctaagggacagcctcgggagccacaggtgtacaccctgcctccatccagagacgagctgaccaagaatcaggtgagcctgacatgtctggtgaagggcttctatccctccgatatcgctgtggagtgggagagcaacggccagcctgagaacaattacaagaccaccccccctgtgctggactctgatggctccttctttctgtattctaagctgacagtggacaagtcccgctggcagcagggcaacgtgttctcctgctccgtgatgcacgaggccctgcacaaccattacacccagaagagcctgtctctgtcccctggcaagtaa

SEQ ID NO: 33 - protein, 233 aa, synthetic construct
EPRGPTIKPSPPSKSPAPNLLGGPSVFIFPPKIKDVLMISLSPIVTCVVVDVSEDDPDVQISWFVNNVEVHTAQTQTHREDYNSTLRVVSALPIQHQDWMSGKAFACAVNNKDLPAPIERTISKPKGSVRAPQVYVLPPPEEEMTKKQVTLTCMVTDFMPEDIYVEWTNNGKTELNYKNTEPVLDSDGSYFMYSKLRVEKKNWVERNSYSCSVVHEGLHNHHTTKSFSRTPGK

SEQ ID NO: 34 - protein, 25 aa, synthetic construct
MGVLLTQRTLLSLVLALLFPSMASM

SEQ ID NO: 35 - protein, 16 aa, synthetic construct
MKCLLYLAFLFIGVNC

SEQ ID NO: 36 - protein, 21 aa, synthetic construct
METDTLLLWVLLLWVPGSTGD

SEQ ID NO: 37 - protein, 19 aa, synthetic construct
MGWSCIILFLVATATGVHS

SEQ ID NO: 38 - protein, 17 aa, synthetic construct
MRAWIFFLLCLAGRALA

SEQ ID NO: 39 - protein, 21 aa, synthetic construct
MWWRLWWLLLLLLLLWPMVWA

SEQ ID NO: 40 - protein, 22 aa, synthetic construct
MDMRVPAQLLGLLLLWLRGARC

SEQ ID NO: 41 - protein, 16 aa, synthetic construct
MPLLLLLPLLWAGALA

SEQ ID NO: 42 - protein, 23 aa, synthetic construct
MDAMKRGLCCVLLLCGAVFVSPS

SEQ ID NO: 43 - protein, 18 aa, synthetic construct
MAFLWLLSCWALLGTTFG

SEQ ID NO: 44 - protein, 15 aa, synthetic construct
MNLLLILTFVAAAVA

SEQ ID NO: 45 - protein, 20 aa, synthetic construct
MYRMQLLSCIALSLALVTNS

SEQ ID NO: 46 - protein, 17 aa, synthetic construct
MGVKVLFALICIAVAEA

SEQ ID NO: 47 - protein, 16 aa, synthetic construct
MKWVTFISLLFSSAYS

SEQ ID NO: 48 - protein, 16 aa, synthetic construct
MKTIIALSYIFCLVLG

SEQ ID NO: 49 - protein, 24 aa, synthetic construct
MALWMRLLPLLALLALWGPDPAAA

SEQ ID NO: 50 - protein, 16 aa, synthetic construct
MKPIFLVLLVVTSAYA

SEQ ID NO: 51 - protein, 24 aa, synthetic construct
MPMGSLQPLATLYLLGMLVASCLG

SEQ ID NO: 52 - protein, 1488 aa, synthetic construct
MFVFLVLLPLVSSQCVNLITRTQSYTNSFTRGVYYPDKVFRSSVLHSTQDLFLPFFSNVTWFHAIHVSGTNGTKRFDNPALPFNDGVYFASTEKSNIIRGWIFGTTLDSKTQSLLIVNNATNVVIKVCEFQFCNDPFLDVYQKNNKSWMESEFRVYSSANNCTFEYVSQPFLMDLEGKEGNFKNLREFVFKNIDGYFKIYSKHTPINLERDLPQGFSALEPLVDLPIGINITRFQTLLALHRSYLTPVDSSSGWTAGAAAYYVGYLQPRTFLLKYNENGTITDAVDCALDPLSETKCTLKSFTVEKGIYQTSNFRVQPTESIVRFPNITNLCPFHEVFNATTFASVYAWNRKRISNCVADYSVIYNFAPFFAFKCYGVSPTKLNDLCFTNVYADSFVIRGNEVSQIAPGQTGNIADYNYKLPDDFTGCVIAWNSNKLDSKPSGNYNYLYRLFRKSKLKPFERDISTEIYQAGNKPCNGVAGPNCYSPLQSYGFRPTYGVGHQPYRVVVLSFELLHAPATVCGPKKSTNLVKNKCVNFNFNGLTGTGVLTESNKKFLPFQQFGRDIADTTDAVRDPQTLEILDITPCSFGGVSVITPGTNTSNQVAVLYQGVNCTEVPVAIHADQLTPTWRVYSTGSNVFQTRAGCLIGAEYVNNSYECDIPIGAGICASYQTQTKSHRAAASVASQSIIAYTMSLGAENSVAYSNNSIAIPTNFTISVTTEILPVSMTKTSVDCTMYICGDSTECSNLLLQYGSFCTQLKRALTGIAVEQDKNTQEVFAQVKQIYKTPPIKYFGGFNFSQILPDPSKPSKASPIEDLLFNKVTLADAGFIKQYGDCLGDIAARDLICAQKFNGLTVLPPLLTDEMIAQYTSALLAGTITSGWTFGAGPALQIPFPMQMAYRFNGIGVTQNVLYENQKLIANQFNSAIGKIQDSLSSTPSALGKLQDVVNHNAQALNTLVKQLSSKFGAISSVLNDILSRLDPPEAEVQIDRLITGRLQSLQTYVTQQLIRAAEIRASANLAATKMSECVLGQSKRVDFCGKGYHLMSFPQSAPHGVVFLHVTYVPAQEKNFTTAPAICHDGKAHFPREGVFVSNGTHWFVTQRNFYEPQIITTDNTFVSGNCDVVIGIVNNTVYDPLQPELDSFKEELDKYFKNHTSPDVDLGDISGINASVVNIQKEIDRLNEVAKNLNESLIDLQELGKYEQYIKWPGSGSGSGYIPEAPRDGQAYVRKDGEWVLLSTFLGSGGGGSGGGGSGSEPKSSDKTHTSPPSPAPELLGGPSVFLFPPKPKDTLMISRTPEVTCVVVDVSHEDPEVKFNWYVDGVEVHNAKTKPREEQYNSTYRVVSVLTVLHQDWLNGKEYKCAVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 53 - DNA, 4473 bp, synthetic construct
  source 1..4473: mol_type = other DNA
gccaccatgttcgtgtttctggtgctgctgcccctggtgtccagccagtgcgtgaatctgatcaccagaacacagagctacaccaactctttcacacgcggcgtgtactatccagacaaggtgtttaggtcttccgtgctgcactctacacaggatctgtttctgcccttcttttccaatgtgacctggttccacgccatccatgtgagcggcaccaacggcacaaagaggttcgacaatcctgctctgcccttcaacgatggcgtgtacttcgcctccaccgagaagagcaacatcatcaggggctggatctttggcaccacactggactctaagacacagtccctgctgatcgtgaacaatgctaccaacgtggtcatcaaggtgtgcgagttccagttttgtaacgatcctttcctggacgtgtatcagaagaacaataagagctggatggagtctgagtttcgcgtgtacagctctgccaacaattgtacctttgagtacgtgtcccagccattcctgatggacctggagggcaaggagggcaatttcaagaacctgcgggagttcgtgtttaagaatatcgatggctacttcaagatctacagcaagcacaccccaatcaacctggagagagacctgccacagggcttctctgccctggagcctctggtggatctgccaatcggcatcaacatcaccaggtttcagacactgctggctctgcatcggagctacctgacacctgtggactccagctctggatggaccgctggagctgctgcttactatgtgggctacctgcagccaaggaccttcctgctgaagtataacgagaatggcaccatcacagacgccgtggattgcgctctggatccactgagcgagaccaagtgtacactgaagtcttttaccgtggagaagggcatctaccagacatctaatttcagagtgcagcccaccgagtccatcgtgcgctttcccaatatcaccaacctgtgcccttttcatgaggtgttcaacgccaccacattcgcttccgtgtacgcctggaataggaagcggatcagcaactgcgtggctgactattctgtgatctacaattttgctccattctttgccttcaagtgctacggcgtgtcccccacaaagctgaatgacctgtgctttaccaacgtgtatgccgatagcttcgtgatccgcggcaacgaggtgtctcagatcgctccaggccagacaggcaatatcgccgactacaactataagctgcccgacgatttcaccggctgcgtgatcgcctggaactctaataagctggatagcaagccatctggcaactacaattatctgtacagactgtttcgcaagagcaagctgaagcccttcgagagggatatctctacagagatctaccaggctggcaataagccttgcaacggagtggctggaccaaactgttattcccctctgcagagctacggcttccgccctacctatggagtgggacaccagccatatagggtggtggtgctgtcctttgagctgctgcatgcccctgctacagtgtgcggcccaaagaagagcaccaatctggtgaagaacaagtgcgtgaacttcaacttcaacggactgaccggcacaggcgtgctgaccgagtccaacaagaagttcctgccctttcagcagttcggccgggacatcgctgataccacagacgccgtgagagacccacagaccctggagatcctggatatcacaccctgctccttcggcggcgtgagcgtgatcacacctggcaccaatacaagcaaccaggtggctgtgctgtaccagggcgtgaattgtaccgaggtgcctgtggccatccacgctgaccagctgaccccaacatggcgcgtgtattccaccggcagcaacgtgtttcagacaagggctggctgtctgatcggcgccgagtatgtgaacaattcctacgagtgcgatatccctatcggcgctggcatctgtgccagctaccagacccagacaaagtctcatagggctgctgcttccgtggcttcccagagcatcatcgcctacaccatgagcctgggcgctgagaattctgtggcctattctaacaattccatcgccatccctaccaacttcacaatcagcgtgaccacagagatcctgccagtgtctatgaccaagacatccgtggactgcacaatgtacatctgtggcgattctaccgagtgctccaatctgctgctgcagtatggctctttttgtacccagctgaagagagctctgacaggcatcgccgtggagcaggacaagaacacacaggaggtgttcgctcaggtgaagcagatctacaagaccccccctatcaagtattttggcggcttcaatttttcccagatcctgccagatcccagcaagccatctaaggcctcccccatcgaggacctgctgtttaacaaggtgaccctggccgatgctggcttcatcaagcagtacggcgattgcctgggcgacatcgctgctcgggacctgatctgtgctcagaagttcaatggcctgaccgtgctgccacccctgctgacagatgagatgatcgcccagtatacatccgccctgctggctggcaccatcacaagcggatggacctttggcgctggaccagctctgcagatcccttttccaatgcagatggcttacagattcaacggcatcggcgtgacccagaatgtgctgtatgagaaccagaagctgatcgctaatcagttcaactctgccatcggcaagatccaggactccctgtccagcacaccaagcgccctgggcaagctgcaggatgtggtgaatcacaacgcccaggctctgaataccctggtgaagcagctgtcttccaagtttggcgctatcagctctgtgctgaacgatatcctgtcccgcctggaccctccagaggctgaggtgcagatcgacaggctgatcacaggccggctgcagtctctgcagacctacgtgacacagcagctgatcagagccgctgagatccgcgcctccgctaacctggccgctaccaagatgtccgagtgcgtgctgggccagagcaagagggtggacttttgtggcaagggctaccacctgatgagcttcccccagtctgctcctcacggcgtggtgtttctgcatgtgacctatgtgcccgcccaggagaagaacttcaccacagcccctgctatctgccacgatggcaaggcccattttccccgggagggcgtgttcgtgtccaatggcacccattggtttgtgacacagagaaacttctacgagcctcagatcatcaccacagacaatacatttgtgagcggcaactgtgacgtggtcatcggtatcgtgaacaataccgtgtacgatcccctgcagcctgagctggactcttttaaggaggagctggataagtatttcaagaatcatacctcccctgacgtggatctgggcgacatcagcggcatcaatgcttctgtggtgaacatccagaaggagatcgaccggctgaacgaggtggccaagaatctgaacgagagcctgatcgatctgcaggagctgggcaagtatgagcagtacatcaagtggccaggatctggatccggcagcggatacatcccagaggctcccagagacggccaggcctatgtgcgcaaggatggcgagtgggtgctgctgtctaccttcctgggatccggaggaggaggaagcggaggcggcggctccggcagcgagcccaagagctccgacaagacccacacatccccacctagcccagctcctgagctgctgggaggaccatccgtgttcctgtttccacccaagcctaaggatacactgatgatctctagaacccctgaggtgacatgcgtggtggtggacgtgtcccacgaggacccagaggtgaagttcaattggtacgtggacggcgtggaggtgcataacgccaagaccaagcctagggaggagcagtataactctacataccgggtggtgtccgtgctgaccgtgctgcatcaggattggctgaatggcaaggagtataagtgcgctgtgagcaacaaggccctgccagctcccatcgagaagacaatctctaaggctaagggacagcctcgggagccacaggtgtacaccctgcctccatccagagacgagctgaccaagaatcaggtgagcctgacatgtctggtgaagggcttctatccctccgatatcgctgtggagtgggagagcaacggccagcctgagaacaattacaagaccaccccccctgtgctggactctgatggctccttctttctgtattctaagctgacagtggacaagtcccgctggcagcagggcaacgtgttctcctgctccgtgatgcacgaggccctgcacaaccattacacccagaagagcctgtctctgtcccctggcaagtaa

SEQ ID NO: 54 - DNA, 4479 bp, synthetic construct
  source 1..4479: mol_type = other DNA
ggtaccgccaccatgttcgtgtttctggtgctgctgcccctggtgtccagccagtgcgtgaatctgatcaccagaacacagagctacaccaactctttcacacgcggcgtgtactatccagacaaggtgtttaggtcttccgtgctgcactctacacaggatctgtttctgcccttcttttccaatgtgacctggttccacgccatccatgtgagcggcaccaacggcacaaagaggttcgacaatcctgctctgcccttcaacgatggcgtgtacttcgcctccaccgagaagagcaacatcatcaggggctggatctttggcaccacactggactctaagacacagtccctgctgatcgtgaacaatgctaccaacgtggtcatcaaggtgtgcgagttccagttttgtaacgatcctttcctggacgtgtatcagaagaacaataagagctggatggagtctgagtttcgcgtgtacagctctgccaacaattgtacctttgagtacgtgtcccagccattcctgatggacctggagggcaaggagggcaatttcaagaacctgcgggagttcgtgtttaagaatatcgatggctacttcaagatctacagcaagcacaccccaatcaacctggagagagacctgccacagggcttctctgccctggagcctctggtggatctgccaatcggcatcaacatcaccaggtttcagacactgctggctctgcatcggagctacctgacacctgtggactccagctctggatggaccgctggagctgctgcttactatgtgggctacctgcagccaaggaccttcctgctgaagtataacgagaatggcaccatcacagacgccgtggattgcgctctggatccactgagcgagaccaagtgtacactgaagtcttttaccgtggagaagggcatctaccagacatctaatttcagagtgcagcccaccgagtccatcgtgcgctttcccaatatcaccaacctgtgcccttttcatgaggtgttcaacgccaccacattcgcttccgtgtacgcctggaataggaagcggatcagcaactgcgtggctgactattctgtgatctacaattttgctccattctttgccttcaagtgctacggcgtgtcccccacaaagctgaatgacctgtgctttaccaacgtgtatgccgatagcttcgtgatccgcggcaacgaggtgtctcagatcgctccaggccagacaggcaatatcgccgactacaactataagctgcccgacgatttcaccggctgcgtgatcgcctggaactctaataagctggatagcaagccatctggcaactacaattatctgtacagactgtttcgcaagagcaagctgaagcccttcgagagggatatctctacagagatctaccaggctggcaataagccttgcaacggagtggctggaccaaactgttattcccctctgcagagctacggcttccgccctacctatggagtgggacaccagccatatagggtggtggtgctgtcctttgagctgctgcatgcccctgctacagtgtgcggcccaaagaagagcaccaatctggtgaagaacaagtgcgtgaacttcaacttcaacggactgaccggcacaggcgtgctgaccgagtccaacaagaagttcctgccctttcagcagttcggccgggacatcgctgataccacagacgccgtgagagacccacagaccctggagatcctggatatcacaccctgctccttcggcggcgtgagcgtgatcacacctggcaccaatacaagcaaccaggtggctgtgctgtaccagggcgtgaattgtaccgaggtgcctgtggccatccacgctgaccagctgaccccaacatggcgcgtgtattccaccggcagcaacgtgtttcagacaagggctggctgtctgatcggcgccgagtatgtgaacaattcctacgagtgcgatatccctatcggcgctggcatctgtgccagctaccagacccagacaaagtctcatagggctgctgcttccgtggcttcccagagcatcatcgcctacaccatgagcctgggcgctgagaattctgtggcctattctaacaattccatcgccatccctaccaacttcacaatcagcgtgaccacagagatcctgccagtgtctatgaccaagacatccgtggactgcacaatgtacatctgtggcgattctaccgagtgctccaatctgctgctgcagtatggctctttttgtacccagctgaagagagctctgacaggcatcgccgtggagcaggacaagaacacacaggaggtgttcgctcaggtgaagcagatctacaagaccccccctatcaagtattttggcggcttcaatttttcccagatcctgccagatcccagcaagccatctaaggcctcccccatcgaggacctgctgtttaacaaggtgaccctggccgatgctggcttcatcaagcagtacggcgattgcctgggcgacatcgctgctcgggacctgatctgtgctcagaagttcaatggcctgaccgtgctgccacccctgctgacagatgagatgatcgcccagtatacatccgccctgctggctggcaccatcacaagcggatggacctttggcgctggaccagctctgcagatcccttttccaatgcagatggcttacagattcaacggcatcggcgtgacccagaatgtgctgtatgagaaccagaagctgatcgctaatcagttcaactctgccatcggcaagatccaggactccctgtccagcacaccaagcgccctgggcaagctgcaggatgtggtgaatcacaacgcccaggctctgaataccctggtgaagcagctgtcttccaagtttggcgctatcagctctgtgctgaacgatatcctgtcccgcctggaccctccagaggctgaggtgcagatcgacaggctgatcacaggccggctgcagtctctgcagacctacgtgacacagcagctgatcagagccgctgagatccgcgcctccgctaacctggccgctaccaagatgtccgagtgcgtgctgggccagagcaagagggtggacttttgtggcaagggctaccacctgatgagcttcccccagtctgctcctcacggcgtggtgtttctgcatgtgacctatgtgcccgcccaggagaagaacttcaccacagcccctgctatctgccacgatggcaaggcccattttccccgggagggcgtgttcgtgtccaatggcacccattggtttgtgacacagagaaacttctacgagcctcagatcatcaccacagacaatacatttgtgagcggcaactgtgacgtggtcatcggtatcgtgaacaataccgtgtacgatcccctgcagcctgagctggactcttttaaggaggagctggataagtatttcaagaatcatacctcccctgacgtggatctgggcgacatcagcggcatcaatgcttctgtggtgaacatccagaaggagatcgaccggctgaacgaggtggccaagaatctgaacgagagcctgatcgatctgcaggagctgggcaagtatgagcagtacatcaagtggccaggatctggatccggcagcggatacatcccagaggctcccagagacggccaggcctatgtgcgcaaggatggcgagtgggtgctgctgtctaccttcctgggatccggaggaggaggaagcggaggcggcggctccggcagcgagcccaagagctccgacaagacccacacatccccacctagcccagctcctgagctgctgggaggaccatccgtgttcctgtttccacccaagcctaaggatacactgatgatctctagaacccctgaggtgacatgcgtggtggtggacgtgtcccacgaggacccagaggtgaagttcaattggtacgtggacggcgtggaggtgcataacgccaagaccaagcctagggaggagcagtataactctacataccgggtggtgtccgtgctgaccgtgctgcatcaggattggctgaatggcaaggagtataagtgcgctgtgagcaacaaggccctgccagctcccatcgagaagacaatctctaaggctaagggacagcctcgggagccacaggtgtacaccctgcctccatccagagacgagctgaccaagaatcaggtgagcctgacatgtctggtgaagggcttctatccctccgatatcgctgtggagtgggagagcaacggccagcctgagaacaattacaagaccaccccccctgtgctggactctgatggctccttctttctgtattctaagctgacagtggacaagtcccgctggcagcagggcaacgtgttctcctgctccgtgatgcacgaggccctgcacaaccattacacccagaagagcctgtctctgtcccctggcaagtaa